(19) United States
(12) Patent Application Publication
(10) Pub. No.: US 2022/0071971 A1
(43) Pub. Date: Mar. 10, 2022

(54) MACROCYCLIC COMPOUNDS AND THEIR USE IN THE TREATMENT OF DISEASE

(71) Applicant: NOVARTIS AG, Basel (CH)

(72) Inventors: Mihai Azimioara, San Diego, CA (US); Badry Bursulaya, San Diego, CA (US); Songchun Jiang, San Diego, CA (US); Casey Jacob Nelson MATHISON, San Diego, CA (US); Victor Ivanovich NIKULIN, Carlsbad, CA (US); Truc Ngoc NGUYEN, San Diego, CA (US); Barun OKRAM, San Diego, CA (US); Sejal PATEL, Lexington, MA (US); Dean Paul PHILLIPS, San Marcos, CA (US); Lewis WHITEHEAD, Swampscott, MA (US); Baogen WU, San Diego, CA (US); Shanshan YAN, San Diego, CA (US); Xuefeng ZHU, San Diego, CA (US)

(21) Appl. No.: 17/415,335

(22) PCT Filed: Dec. 18, 2019
(86) PCT No.: PCT/IB2019/061054

§ 371 Date: Jun. 17, 2021

Related U.S. Application Data
(60) Provisional application No. 62/783,270, filed on Dec. 21, 2018.

(51) Int. Cl. A61K 31/439; A61K 45/06; C07D 515/18; C07D 513/22; C07D 513/18; A61K 31/5377

(52) U.S. Cl. CPC A61K 31/439; A61K 45/06; A61K 31/5377; C07D 513/22; C07D 513/18; C07D 515/18

(57) ABSTRACT

The invention relates to heterocyclic compounds of the formula (I), in which all of the variables are as defined in the specification; capable of modulating the activity of CFTR. The invention further provides a method for manufacturing compounds of the invention, and its therapeutic uses. The invention further provides methods to their preparation, to their medical use, in particular to their use in the treatment and management of diseases or disorders including Cystic fibrosis and related disorders.

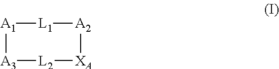

### FIELD OF THE INVENTION

The present invention relates to macrocyclic compounds, and pharmaceutically acceptable salts thereof, which comprise an optionally substituted divalent N-(pyridin-2-yl)pyridinyl-sulfonamide moiety. The present invention further relates to the use of such macrocyclic compounds in the treatment of respiratory diseases. The present invention further relates to the use of such macrocyclic compounds in the treatment of pancreatitis. The present invention further relates to pharmaceutical compositions comprising such macrocyclic compounds, a pharmaceutically acceptable carrier and optionally at least one additional therapeutic agent. The present invention further relates to combinations comprising such macrocyclic compounds and at least one additional therapeutic agent. The present invention further relates to the use of such pharmaceutical compositions and combinations in the treatment of respiratory diseases. The present invention further relates to the use of such pharmaceutical compositions and combinations in the treatment of pancreatitis.

### BACKGROUND OF THE INVENTION

Cystic fibrosis (CF) is an autosomal genetic disease that affects approximately 30,000 people in the United States and approximately 70,000 people worldwide. Approximately 1,000 new cases of CF are diagnosed each year. Most patients are diagnosed with CF by the age of two, and more than half of the CF population is 18 years in age or older. Despite progress in the treatment of CF, there is no cure.

Cystic fibrosis (CF) is caused by loss-of-function mutations in the CF transmembrane conductance regulator (CFTR) protein, a cAMP-regulated chloride channel expressed primarily at the apical plasma membrane of secretory epithelia in the airways, pancreas, intestine, and other tissues. CFTR is a large, multidomain glycoprotein consisting of two membrane-spanning domains, two nucleotide-binding domains (NBD1 and NBD2) that bind and hydrolyze ATP, and a regulatory (R) domain that gates the channel by phosphorylation. Nearly 2000 mutations in the CFTR gene have been identified that produce the loss-of-function phenotype by impairing its translation, cellular processing, and/or chloride channel gating. The F508del mutation, which is present in at least one allele in ˜90% of CF patients, impairs CFTR folding, stability at the endoplasmic reticulum and plasma membrane, and chloride channel gating (Dalemans et al. 1991; Denning et al. 1992; Lukacs et al. 1993; Du et al. 2005). Other mutations primarily alter channel gating (e.g., G551 D), conductance (e.g., R117H), or translation (e.g., G542X) (Welsh and Smith 1993). The fundamental premise of CFTR corrector and potentiator therapy for CF is that correction of the underlying defects in the cellular processing and chloride channel function of CF-causing mutant CFTR alleles will be of clinical benefit. Correctors are principally targeted at F508del cellular misprocessing, whereas potentiators are intended to restore cAMP-dependent chloride channel activity to mutant CFTRs at the cell surface. In contrast to current therapies, such as antibiotics, anti-inflammatory agents, mucolytics, nebulized hypertonic saline, and pancreatic enzyme replacement, which treat CF disease manifestations, correctors and potentiators correct the underlying CFTR anion channel defect.

### SUMMARY OF THE INVENTION

There remains a need for new treatments and therapies for cyctic fibrosis and related disorders, including asthma, COPD, chronic bronchitis and emphysema. In addition, there remains a need for new treatments and therapies for pancreatitis. The invention provides compounds of formula (I), and sub-formulae thereof, pharmaceutically acceptable salts thereof, pharmaceutical compositions thereof and combinations thereof, wherein the compounds formula (I), and sub-formulae thereof, are CFTR correctors. The invention further provides methods of treating, preventing, or ameliorating cyctic fibrosis and related disorders, where the method comprises administering to a subject in need thereof an effective amount of a CFTR corrector of the present invention, either in combination with a CFTR potentiator (dual combination) or in combination with a CFTR potentiator and a different CFTR corrector (triple combination). Various embodiments of the present invention are described herein.

In one aspect of the present invention are compounds having the structure of formula (I), or a pharmaceutically acceptable salt thereof:

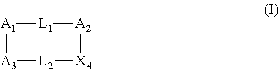

wherein A₁, A₂ A₃, L₁, L₂ and X_{A} are as defined herein.

Another aspect of the present invention are compounds having the structure of formula (I-a), or a pharmaceutically acceptable salt thereof:

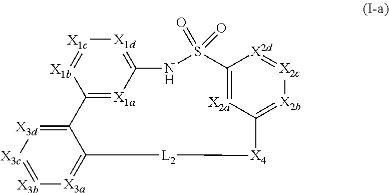

wherein: X₁ₐ, X_{1b}, X_{1c}, X_{1d}, X₂ₐ, X_{2b}, X_{2c}, X_{2d}, X₃ₐ, X_{3b}, X_{3c}, X_{3d}, X₄ and L₂ are as defined herein.

Another aspect of the present invention are compounds having the structure of formula of Formula (I-b), or a pharmaceutically acceptable salt thereof,

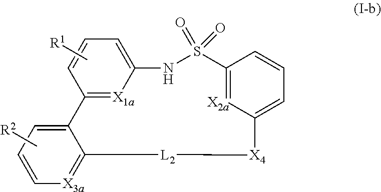

wherein X₁ₐ, X₂ₐ, X₃ₐ, X₄, L₂, R¹ and R² are as defined herein.

In another aspect, the invention provides a pharmaceutical compositions comprising a therapeutically effective amount of a compound of the present invention, or a pharmaceutically acceptable salt thereof, and a pharmaceutically acceptable carrier.

In another aspect, the invention provides a pharmaceutical compositions comprising a compound of the present invention, or a pharmaceutically acceptable salt thereof, and a pharmaceutically acceptable carrier.

In another aspect, the invention provides a method for treating a Cystic Fibrosis Transmembrane Conductance Regulator (CFTR) mediated disease in a subject comprising administering to the subject therapeutically effective amount of a compound of the present invention, or a pharmaceutically acceptable salt thereof.

In another aspect, the invention provides a method for treating a Cystic Fibrosis Transmembrane Conductance Regulator (CFTR) mediated disease in a subject comprising administering to the subject a compound of the present invention, or a pharmaceutically acceptable salt thereof.

In another aspect, the invention provides the use of a compound of the present invention, or a pharmaceutically acceptable salt thereof, in the manufacture of a medicament for the treatment of a Cystic Fibrosis Transmembrane Conductance Regulator (CFTR) mediated disease.

In another aspect, the invention provides the use of a compound of the present invention, or a pharmaceutically acceptable salt thereof, for the treatment of a Cystic Fibrosis Transmembrane Conductance Regulator (CFTR) mediated disease.

In another aspect, the invention provides a compound of the present invention, or a pharmaceutically acceptable salt thereof, for use in the treatment of a Cystic Fibrosis Transmembrane Conductance Regulator (CFTR) mediated disease.

In another aspect, the invention provides a method for treating a Cystic Fibrosis Transmembrane Conductance Regulator (CFTR) mediated disease in a subject comprising administering to the subject a pharmaceutical composition comprising a therapeutically effective amount of a compound of the present invention, or a pharmaceutically acceptable salt thereof.

In another aspect, the invention provides a method for treating a Cystic Fibrosis Transmembrane Conductance Regulator (CFTR) mediated disease in a subject comprising administering to the subject a pharmaceutical composition comprising a compound of the present invention, or a pharmaceutically acceptable salt thereof.

In another aspect, the invention provides the use of a pharmaceutical composition comprising a compound of the present invention, or a pharmaceutically acceptable salt thereof, for the treatment of a Cystic Fibrosis Transmembrane Conductance Regulator (CFTR) mediated disease.

In another aspect, the invention provides a pharmaceutical composition comprising a compound of the present invention, or a pharmaceutically acceptable salt thereof, for use in the treatment of a Cystic Fibrosis Transmembrane Conductance Regulator (CFTR) mediated disease.

In another aspect, the invention provides a pharmaceutical combination comprising a therapeutically effective amount of a compound of the present invention, or a pharmaceutically acceptable salt thereof, and one or more additional therapeutic agents and optionally further comprising a pharmaceutically acceptable carrier.

In another aspect, the invention provides a pharmaceutical combination comprising a compound of the present invention, or a pharmaceutically acceptable salt thereof, and one or more additional therapeutic agents and optionally further comprising a pharmaceutically acceptable carrier.

In another aspect, the invention provides the use of a pharmaceutical combination of the present invention in the treatment of a Cystic Fibrosis Transmembrane Conductance Regulator (CFTR) mediated disease.

### DETAILED DESCRIPTION OF THE INVENTION

Various enumerated embodiments of the present invention are described herein. It will be recognized that features specified in each embodiment may be combined with other specified features to provide further embodiments of the present invention.

### Definitions

The term “alkyl,” as used herein, refers to a saturated branched or straight chain hydrocarbon. In certain embodiments an alkyl group is a “C₁-C₃alkyl”, “C₁-C₄alkyl”, “C₁-C₅alkyl”, “C₁-C₆alkyl”, “C₁-C₇alkyl”, “C₁-C₈alkyl”, “C₁-C₉alkyl” or “C₁-C₁₀alkyl”, wherein the terms “C₁-C₃alkyl”, “C₁-C₄alkyl”, “C₁-C₅alkyl”, “C₁-C₆alkyl”, “C₁-C₇alkyl”, “C₁-C₈alkyl”, “C₁-C₉alkyl” and “C₁-C₁₀alkyl”, as used herein, refer to an alkyl group containing at least 1, and at most 3, 4, 5, 6, 7, 8, 9 or 10 carbon atoms, respectively. Non-limiting examples of alkyl groups include methyl, ethyl, n-propyl, isopropyl, n-butyl, isobutyl, sec-butyl, tert-butyl, n-pentyl, isopentyl, hexyl, heptyl, octyl, nonyl, decyl and the like. In certain embodiments such alkyl groups are optionally substituted.

The term “alkylene,” as used herein, refers to a saturated branched or straight chain divalent hydrocarbon radical derived from an alkyl group. In certain embodiments an alkylene group is a “C₁-C₃alkylene”, “C₁-C₄alkylene”, “C₁-C₅alkylene”, “C₁-C₆alkylene”, “C₁-C₇alkylene”, “C₁-C₈alkylene”, “C₁-C₉alkylene” or “C₁-C₁₀alkylene”, wherein the terms “C₁-C₃alkylene”, “C₁-C₄alkylene”, “C₁-C₅alkylene”, “C₁-C₆alkylene”, “C₁-C₇alkylene” and “C₁-C₈alkylene”, as used herein, refer to an alkylene group containing at least 1, and at most 3, 4, 5, 6, 7, 8, 9 or 10 carbon atoms respectively. Non-limiting examples of alkylene groups as used herein include, methylene, ethylene, n-propylene, isopropylene, n-butylene, isobutylene, sec-butylene, t-butylene, n-pentylene, isopentylene, hexylene, heptylene, octylene, nonylene, decylene and the like. In certain embodiments such alkylene groups are optionally substituted.

The term “alkoxy”, as used herein, refers to —O-alkyl or -alkyl-O—, wherein the “alkyl” group is as as defined herein. In certain embodiments an alkoxy group is a “C₁-C₃alkoxy”, “C₁-C₄alkoxy”, “C₁-C₅alkoxy”, “C₁-C₆alkoxy”, “C₁-C₇alkoxy”, “C₁-C₈alkoxy”, “C₁-C₉alkoxy” or “C₁-C₁₀alkoxy”, wherein the terms “C₁-C₃alkoxy”, “C₁-C₄alkoxy”, “C₁-C₅alkoxy”, “C₁-C₆alkoxy”, “C₁-C₇alkoxy”, “C₁-C₈alkoxy”, “C₁-C₉alkoxy” and “C₁-C₁₀alkoxy”, as used herein refer to —O—C₁-C₃alkyl, —O—C₁-C₄alkyl, —O—C₁-C₅alkyl, —O—C₁-C₆alkyl, —O—C₁-C₇alkyl, —O—C₁-C₈alkyl, —O—C₁-C₉alkyl or —O—C₁-C₁₀alkyl, respectively. Non-limiting examples of “alkoxy” groups include methoxy, ethoxy, n-propoxy, isopropoxy, n-butoxy, isobutoxy, sec-butoxy, tert-butoxy, n-pentoxy, isopentoxy, hexoxy, heptoxy, octoxy, nonoxy, decoxy and the like. In certain embodiments such alkoxy groups are optionally substituted.

The term “alkoxylene”, as used herein, refers —O-alkylene- or -alkylene-O—, which is a divalent radical derived from an alkoxy group, wherein the “alkylene” group is as as defined herein. In certain embodiments an alkoxylene group is a “C₁-C₃alkoxylene”, “C₁-C₄alkoxylene”, “C₁-C₅alkoxylene”, “C₁-C₆alkoxylene”, “C₁-C₇alkoxylene”, “C₁-C₈alkoxylene”, “C₁-C₉alkoxylene” or “C₁-C₁₀alkoxylene”, wherein the terms “C₁-C₃alkoxylene”, “C₁-C₄alkoxylene”, “C₁-C₅alkoxylene”, “C₁-C₆alkoxylene”, “C₁-C₇alkoxylene”, “C₁-C₈alkoxylene”, “C₁-C₉alkoxylene” and “C₁-C₁₀alkoxylene”, as used herein refer to —O—C₁-C₃alkylene, —O—C₁-C₄alkylene, —O—C₁-C₅alkylene, —O—C₁-C₆alkylene, —O—C₁-C₇alkylene, —O—C₁-C₈alkylene, —O—C₁-C₉alkylene or —O—C₁-C₁₀alkylene, respectively. In certain embodiments an alkoxylene group is a “C₁-C₃alkoxylene”, “C₁-C₄alkoxylene”, “C₁-C₅alkoxylene”, “C₁-C₆alkoxylene”, “C₁-C₇alkoxylene”, “C₁-C₈alkoxylene”, “C₁-C₉alkoxylene” or “C₁-C₁₀alkoxylene”, wherein the terms “C₁-C₃alkoxylene”, “C₁-C₄alkoxylene”, “C₁-C₅alkoxylene”, “C₁-C₆alkoxylene”, “C₁-C₇alkoxylene”, “C₁-C₈alkoxylene”, “C₁-C₉alkoxylene” and “C₁-C₁₀alkoxylene”, as used herein refer to —C₁-C₃alkylene-O, —C₁-C₄alkylene-O, —C₁-C₅alkylene-O, —C₁-C₆alkylene-O, —C₁-C₇alkylene-O, —C₁-C₈alkylene-O, —C₁-C₉alkylene-O or —C₁-C₁₀alkylene-O, respectively. Non-limiting examples of “alkoxylene” groups include methoxylene, ethoxylene, n-propoxylene, isopropoxylene, n-butoxylene, isobutoxylene, sec-butoxylene, tert-butoxylene, n-pentyloxylene, isopentyloxylene, hexyloxylene, heptyloxylene, octyloxylene, nonyloxylene, decyloxylene and the like. In certain embodiments such alkoxylene groups are optionally substituted.

The term “alkylene oxide”, as used herein, refers to the following divalent group -alkylene-O-alkylene-, wherein the “alkylene” group is as as defined herein.

The term “aminoalkylene”, as used herein, refers to —NH-alkylene- or -alkylene-NH—, which is a divalent group, wherein the “alkylene” group is as as defined herein. In certain embodiments such aminoalkylene groups are optionally substituted.

The term “aryl,” as used herein, refers to an aromatic monocyclic ring system having 6 carbon atoms as ring members, an aromatic fused bicyclic ring system having 9-10 carbon atoms as ring members, or an aromatic fused tricyclic ring systems having 14 carbon atoms as ring members. Non-limiting examples of an aryl group, as used herein, include phenyl, naphthalenyl, fluorenyl, indenyl, azulenyl, anthracenyl, phenanthrenyl and the like. In certain embodiments such aryl groups are optionally substituted. In preferred embodiments an aryl group is a phenyl.

The term “arylene,” as used herein efers to a divalent group derived from an aryl group as defined herein. Non-limiting examples of an arylene group, as used herein, include phenylene, naphthalenylene, indenylene, azulenylene, anthracenylene, phenanthrenylen and the like. In certain embodiments such arylene groups are optionally substituted. In preferred embodiments an arylene group is a phenylene.

The term “C₃-C₈cycloalkyl” as used herein, refers to a saturated, monocyclic hydrocarbon ring system having 3 to 8 carbon atoms as ring members. Non-limiting examples of such “C₃-C₈cycloalkyl” groups include cyclopropyl, cyclobutyl, cyclopentyl, cyclohexyl, cyclohepyl and cyclooctyl groups. In certain embodiments such cycloalkyl groups are optionally substituted.

The term “C₃-C₈cycloalkylene” as used herein, refers to a divalent saturated, monocyclic hydrocarbon ring system derived from a “C₃-C₈cycloalkyl” as defined herein. Non-limiting examples of such “C₃-C₈cycloalkylene” groups include cyclopropylene, cyclobutylene, cyclopentylene, cyclohexylene, cyclohepylene and cyclooctylene groups. In certain embodiments such cycloalkylene groups are optionally substituted.

The term “deuterium-substituted C₁-C₆alkyl”, as used herein, refers to the respective “C₁-C₆alkyl”, as defined herein, wherein at least one of the hydrogen atoms of the “C₁-C₆alkyl” is replaced by a deuterium atom. The deuterium-substituted C₁-C₆alkyl group can be monodeuterated, wherein one hydrogen atom of the “C₁-C₆alkyl” is replaced by one deuterium atom. The deuterium-substituted C₁-C₆alkyl group can be dideuterated, wherein two hydrogen atoms of the “C₁-C₆alkyl” are each replaced by a deuterium atom. The deuterium-substituted C₁-C₆alkyl groups can be trideuterated, wherein three hydrogen atoms of the “C₁-C₆alkyl” are each replaced by a deuterium atom. Furthermore, the deuterium-substituted C₁-C₆alkyl group can be polydeuterated, wherein four or more hydrogen atoms of the “C₁-C₆alkyl” are each replaced by a deuterium atom. Non-limiting examples of a “deuterium-substituted C₁-C₆alkyl” groups include —CH₂D, —CHD₂, —CD₃, —CH₂CH₂D, —CH₂CHD₂, —CH₂CD₃ and -CD₂CD₃.

The terms “halo-substituted C₁-C₆alkyl” and “C₁-C₆haloalkyl” are used interchangeably herein and as used herein, refer to the respective “C₁-C₆alkyl”, as defined herein, wherein at least one of the hydrogen atoms of the “C₁-C₆alkyl” is replaced by a halo atom. The halo-substituted C₁-C₆alkyl or C₁-C₆haloalkyl groups can be monoC₁-C₆haloalkyl, wherein such C₁-C₆haloalkyl groups have one iodo, one bromo, one chloro or one fluoro. Additionally, the C₁-C₆haloalkyl groups can be diC₁-C₆haloalkyl wherein such C₁-C₆haloalkyl groups can have two halo atoms independently selected from iodo, bromo, chloro or fluoro. Furthermore, the C₁-C₆haloalkyl groups can be polyC₁-C₆haloalkyl wherein such C₁-C₆haloalkyl groups can have two or more of the same halo atoms or a combination of two or more different halo atoms. Such polyC₁-C₆haloalkyl can be perhaloC₁-C₆haloalkyl where all the hydrogen atoms of the respective C₁-C₆alkyl have been replaced with halo atoms and the halo atoms can be the same or a combination of different halo atoms. Non-limiting examples of “halo-substituted C₁-C₆alkyl” and “C₁-C₆haloalkyl” groups include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, trifluoroethyl, difluoropropyl, dichloroethyl and dichloropropyl.

The terms “halo-substituted C₁-C₆alkoxy” and “C₁-C₆haloalkoxy” are used interchangeably herein and as used herein, refer to the respective “C₁-C₆alkoxy”, as defined herein, wherein at least one of the hydrogen atoms of the “C₁-C₆alkyl” of the “C₁-C₆haloalkoxy” is replaced by a halo atom. The halo-substituted C₁-C₆alkoxy or C₁-C₆haloalkoxy groups can be monoC₁-C₆haloalkoxy, wherein such C₁-C₆haloalkoxy groups have one iodo, one bromo, one chloro or one fluoro. Additionally, the C₁-C₆haloalkoxy groups can be diC₁-C₆haloalkoxy wherein such C₁-C₆haloalkoxy groups can have two halo atoms independently selected from iodo, bromo, chloro or fluoro. Furthermore, the C₁-C₆haloalkoxy groups can be polyC₁-C₆haloalkoxy wherein such C₁-C₆haloalkoxy groups can have two or more of the same halo atoms or a combination of two or more different halo atoms. Such polyC₁-C₆haloalkoxy can be perhaloC₁-C₆haloalkoxy where all the hydrogen atoms of the respective C₁-C₆alkoxy have been replaced with halo atoms and the halo atoms can be the same or a combination of different halo atoms. Non-limiting examples of “halo-substituted C₁-C₆alkoxy” and “C₁-C₆haloalkoxy” groups include fluoromethoxy, difluoromethoxy, trifluoromethoxy, chloromethoxy, dichloromethoxy, trichloromethoxy, pentafluoroethoxy, heptafluoropropoxy, difluorochloromethoxy, dichlorofluoromethoxy, difluoroethoxy, trifluoroethoxy, difluoropropoxy, dichloroethoxy and dichloropropoxy.

The terms “halo” or “halogen” as used herein, refer to fluoro, chloro, bromo and iodo.

The term “heteroaryl,” as used herein, refers to i) an aromatic, 5-6 membered monocyclic ring system wherein 1 to 4 ring members are independently selected from the heteroatoms N, O and S, ii) an aromatic, 9-10 membered fused bicyclic ring system wherein 1 to 4 ring members are independently selected from the heteroatoms N, O and S and, iii) an aromatic, 14 membered fused tricyclic ring system wherein 1 to 4 ring members are independently selected from the heteroatoms N, O and S. Non-limiting examples of heteroaryl groups, as used herein, include benzofuranyl, benzofurazanyl, benzoxazolyl, benzopyranyl, benzthiazolyl, benzothienyl, benzazepinyl, benzimidazolyl, benzothiopyranyl, benzo[b]furyl, benzo[b]thienyl, cinnolinyl, furazanyl, furyl, furopyridinyl, imidazolyl, indolyl, indolizinyl, indolin-2-one, indazolyl, isoindolyl, isoquinolinyl, isoxazolyl, isothiazolyl, 1,8-naphthyridinyl, oxazolyl, oxaindolyl, oxadiazolyl, pyrazolyl, pyrrolyl, phthalazinyl, pteridinyl, purinyl, pyridyl, pyridazinyl, pyrazinyl, pyrimidinyl, quinoxalinyl, quinolinyl, quinazolinyl, thiazolyl, thiadiazolyl, thienyl, triazinyl, triazolyl and tetrazolyl. In certain embodiments such heteroaryl groups are optionally substituted. In preferred embodiments a heteroaryl group is a pyridyl.

The term “heteroarylene,” as used herein refers to a divalent group derived from a heteroaryl group as defined herein. Non-limiting examples of an arylene group, as used herein, include phenylene, naphthalenylene, benzofuranylene, benzofurazanylene, benzoxazolylene, benzopyranylene, benzthiazolylene, benzothienylene, benzazepinylene, benzimidazolylene, benzothiopyranylene, benzo[b]furylene, benzo[b]thienylene, cinnolinylene, furazanylene, furylene, furopyridinylene, imidazolylene, indolylene, indolizinylene, indolin-2-one, indazolylene, isoindolylene, isoquinolinylene, isoxazolylene, isothiazolylene, 1,8-naphthyridinylene, oxazolylene, oxaindolylene, oxadiazolylene, pyrazolylene, pyrrolylene, phthalazinylene, pteridinylene, purinylene, pyridylene, pyridazinylene, pyrazinylene, pyrimidinylene, quinoxalinylene, quinolinylene, quinazolinylene, thiazolylene, thiadiazolylene, thienylene, triazinylene, triazolylene and tetrazolylene. In certain embodiments such heteroarylene groups are optionally substituted. In preferred embodiments a heteroarylene group is a pyridylene.

The term “heteroatoms” as used herein, refers to nitrogen (N), oxygen (O) or sulfur (S) atoms.

The term “heterocycloalkyl,” as used herein refers to i) a monocyclic ring structure having 4 to 6 ring members, wherein one to two of the ring members are independently selected from N, NH, NR¹⁶, O or —S—, wherein R¹⁶ is C₁-C₆alkyl and ii) a fused bicyclic ring structure having 8 to 10 ring members, wherein one to two of the ring members are independently selected from N, NH, NR¹⁶, O or —S—, wherein R¹⁶ is C₁-C₆alkyl. Non-limiting examples of 4-6 membered heterocycloalkyl groups, as used herein, include azetadinyl, azetadin-1-yl, azetadin-2-yl, azetadin-3-yl, oxetanyl, oxetan-2-yl, oxetan-3-yl, oxetan-4-yl, thietanyl, thietan-2-yl, thietan-3-yl, thietan-4-yl, pyrrolidinyl, pyrrolidin-1-yl, pyrrolidin-2-yl, pyrrolidin-3-yl, pyrrolidin-4-yl, pyrrolidin-5-yl, tetrahydrofuranyl, tetrahydrofuran-2-yl, tetrahydrofuran-3-yl, tetrahydrofuran-4-yl, tetrahydrofuran-5-yl, tetrahydrothienyl, tetrahydrothien-2-yl, tetrahydrothien-3-yl, tetrahydrothien-4-yl, tetrahydrothien-5-yl, piperidinyl, piperidin-1-yl, piperidin-2-yl, piperidin-3-yl, piperidin-4-yl, piperidin-5-yl, piperidin-6-yl, tetrahydropyranyl, tetrahydropyran-2-yl, tetrahydropyran-3-yl, tetrahydropyran-4-yl, tetrahydropyran-5-yl, tetrahydropyran-6-yl, tetrahydrothiopyranyl, tetrahydrothiopyran-2-yl, tetrahydrothiopyran-3-yl, tetrahydrothiopyran-4-yl, tetrahydrothiopyran-5-yl, tetrahydrothiopyran-6-yl, piperazinyl, piperazin-1-yl, piperazin-2-yl, piperazin-3-yl, piperazin-4-yl, piperazin-5-yl, piperazin-6-yl, morpholinyl, morpholin-2-yl, morpholin-3-yl, morpholin-4-yl, morpholin-5-yl, morpholin-6-yl, thiomorpholinyl, thiomorpholin-2-yl, thiomorpholin-3-yl, thiomorpholin-4-yl, thiomorpholin-5-yl, thiomorpholin-6-yl, oxathianyl, oxathian-2-yl, oxathian-3-yl, oxathian-5-yl, oxathian-6-yl, dithianyl, dithian-2-yl, dithian-3-yl, dithian-5-yl, dithian-6-yl, dioxolanyl, dioxolan-2-yl, dioxolan-4-yl, dioxolan-5-yl, thioxanyl, thioxan-2-yl, thioxan-3-yl, thioxan-4-yl, thioxan-5-yl, dithiolanyl, dithiolan-2-yl, dithiolan-4-yl, dithiolan-5-yl, pyrazolidinyl, pyrazolidin-1-yl, pyrazolidin-2-yl, pyrazolidin-3-yl, pyrazolidin-4-yl, pyrazolidin-5-yl, 2-azabicyclo[4.2.0]octanyl, octahydro-1H-cyclopenta[b]pyridine and decahydroquinoline. In certain embodiments such heterocycloalkyl groups are optionally substituted

The term “heterocycloalkylene” as used herein refers to a divalent group derived from a heterocycloalkyl group as defined herein.

The term “hydroxyl” as used herein, refers to a —OH group.

The term “optionally substituted,” as used herein, means that the referenced group may or may not be substituted with one or more additional group(s) individually and independently selected from alkyl, alkenyl, alkynyl, cycloalkyl, aryl, heteroaryl, heterocycloalkyl, hydroxyl, alkoxy, mercaptyl, cyano, halo, carbonyl, thiocarbonyl, isocyanato, thiocyanato, isothiocyanato, nitro, perhaloalkyl, perfluoroalkyl, and amino, including mono- and disubstituted amino groups, and the protected derivatives thereof. Non-limiting examples of optional substituents include, halo, —CN, ═O, ═N—OH, ═N—OR, ═N—R, —OR, —C(O)R, —C(O)OR, —OC(O)R, —OC(O)OR, —C(O)NHR, —C(O)NR₂, —OC(O)NHR, —OC(O)NR₂, —SR—, —S(O)R, —S(O)₂R, —NHR, —N(R)₂, —NHC(O)R, —NRC(O)R, —NHC(O)OR, —NRC(O)OR, S(O)₂NHR, —S(O)₂N(R)₂, —NHS(O)₂NR₂, —NRS(O)₂NR₂, —NHS(O)₂R, —NRS(O)₂R, C₁-C₈alkyl, C₁-C₈alkoxy, aryl, heteroaryl, cycloalkyl, heterocycloalkyl, halo-substituted C₁-C₈alkyl, and halo-substituted C₁-C₈alkoxy, where each R is independently selected from H, halo, C₁-C₈alkyl, C₁-C₈alkoxy, aryl, heteroaryl, cycloalkyl, heterocycloalkyl, halo-substituted C₁-C₈alkyl, and halo-substituted C₁-C₈alkoxy. The placement and number of such substituent groups is done in accordance with the well-understood valence limitations of each group, for example ═O is a suitable substituent for an alkyl group but not for an aryl group.

The term “polyalkylene oxide”, as used herein, refers to the divalent group -(alkylene-O-alkylene)ₙ—, wherein the “alkylene” group is as as defined herein and n is an integer from 1 to 10.

As used herein, “CFTR” stands for cystic fibrosis transmembrane conductance regulator.

As used herein, “mutations” can refer to mutations in the CFTR gene or the CFTR protein. A “CFTR mutation” refers to a mutation in the CFTR gene, and a “CFTR mutation” refers to a mutation in the CFTR protein. A genetic defect or mutation, or a change in the nucleotides in a gene in general results in a mutation in the CFTR protein translated from that gene.

As used herein, a “F508del mutation” or “F508del” is a specific mutation within the CFTR protein. The mutation is a deletion of the three nucleotides that comprise the codon for amino acid phenylalanine at position 508, resulting in CFTR protein that lacks this phenylalanine residue.

The term “CFTR gating mutation” as used herein means a CFTR mutation that results in the production of a CFTR protein for which the predominant defect is a low channel open probability compared to normal CFTR (Van Goor, F., Hadida S. and Grootenhuis P., “Pharmacological Rescue of Mutant CFTR function for the Treatment of Cystic Fibrosis”, Top. Med. Chem. 3: 91-120 (2008)). Gating mutations include, but are not limited to, G551D, G178R, S549N, S549R, G551S, G970R, G1244E, S1251N, S1255P, and G1349D.

As used herein, a patient who is “homozygous” for a particular mutation, e.g. F508del, has the same mutation on each allele.

As used herein, a patient who is “heterozygous” for a particular mutation, e.g. F508del, has this mutation on one allele, and a different mutation on the other allele.

As used herein, the term “modulator” refers to a compound that increases the activity of a biological compound such as a protein. For example, a CFTR modulator is a compound that increases the activity of CFTR. The increase in activity resulting from a CFTR modulator may be through a corrector mechanism or a potentiator mechanism as described below.

As used herein, the term “CFTR corrector” refers to a compound that increases the amount of functional CFTR protein at the cell surface, resulting in enhanced ion transport.

As used herein, the term “CFTR potentiator” refers to a compound that increases the channel activity of CFTR protein located at the cell surface, resulting in enhanced ion transport.

As used herein, the term “modulating” as used herein means increasing or decreasing by a measurable amount.

As used herein, the term “inducing,” as in inducing CFTR activity, refers to increasing CFTR activity, whether by the corrector, potentiator, or other mechanism.

As used herein “asthma” includes both intrinsic (non-allergic) asthma and extrinsic (allergic) asthma, mild asthma, moderate asthma, severe asthma, bronchitic asthma, exercise-induced asthma, occupational asthma and asthma induced following bacterial infection. Treatment of asthma is also to be understood as embracing treatment of subjects, e.g., of less than 4 or 5 years of age, exhibiting wheezing symptoms and diagnosed or diagnosable as “wheezy infants”, an established patient category of major medical concern and now often identified as incipient or early-phase asthmatics. (For convenience this particular asthmatic condition is referred to as “wheezy-infant syndrome”.) Prophylactic efficacy in the treatment of asthma will be evidenced by reduced frequency or severity of symptomatic attack, e.g., of acute asthmatic or bronchoconstrictor attack, improvement in lung function or improved airways hyperreactivity. It may further be evidenced by reduced requirement for other, symptomatic therapy, i.e., therapy for or intended to restrict or abort symptomatic attack when it occurs, e.g., anti-inflammatory (e.g., cortico-steroid) or bronchodilatory. Prophylactic benefit in asthma may, in particular, be apparent in subjects prone to “morning dipping”. “Morning dipping” is a recognized asthmatic syndrome, common to a substantial percentage of asthmatics and characterized by asthma attack, e.g., between the hours of about 4-6 am, i.e., at a time normally substantially distant from any previously administered symptomatic asthma therapy.

The terms “combination” or “pharmaceutical combination,” as used herein, refers to either a fixed combination in one dosage unit form, or a combined administration where a compound of the present invention and a combination partner (e.g. another drug as explained below, also referred to as “therapeutic agent” or “co-agent”) may be administered independently at the same time or separately within time intervals, especially where these time intervals allow that the combination partners show a cooperative, e.g. synergistic effect. The single components may be packaged in a kit or separately. One or both of the components (e.g., powders or liquids) may be reconstituted or diluted to a desired dose prior to administration. The terms “co-administration” or “combined administration” or the like as utilized herein are meant to encompass administration of the selected combination partner to a single subject in need thereof (e.g. a patient), and are intended to include treatment regimens in which the agents are not necessarily administered by the same route of administration or at the same time. The term “pharmaceutical combination” as used herein means a product that results from the mixing or combining of more than one therapeutic agent and includes both fixed and non-fixed combinations of the therapeutic agents. The term “fixed combination” means that the therapeutic agents, e.g. a compound of the present invention and a combination partner, are both administered to a patient simultaneously in the form of a single entity or dosage. The term “non-fixed combination” means that the therapeutic agents, e.g. a compound of the present invention and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g. the administration of three or more therapeutic agent.

The term “combination therapy” or “in combination with” or “pharmaceutical combination” refers to the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients. Alternatively, such administration encompasses co-administration in multiple, or in separate containers (e.g., capsules, powders, and liquids) for each active ingredient. Powders and/or liquids may be reconstituted or diluted to a desired dose prior to administration. In addition, such administration also encompasses use of each type of therapeutic agent being administered prior to, concurrent with, or sequentially to each other with no specific time limits. In each case, the treatment regimen will provide beneficial effects of the drug combination in treating the conditions or disorders described herein.

As used herein the term “co-administer” refers to the presence of two active agents in the blood of an individual. Active agents that are co-administered can be concurrently or sequentially delivered.

The terms “composition” or “pharmaceutical composition,” as used herein, refers to a compound of the present invention, or a pharmaceutically acceptable salt thereof, together with at least one pharmaceutically acceptable carrier, in a form suitable for oral or parenteral administration.

A “patient,” “subject” or “individual” are used interchangeably and refer to either a human or non-human animal. The term includes mammals such as humans. Typically the animal is a mammal. A subject also refers to for example, primates (e.g., humans, male or female), cows, sheep, goats, horses, dogs, cats, rabbits, rats, mice, fish, birds and the like. In certain embodiments, the subject is a primate. Preferably, the subject is a human.

As used herein, the term “inhibit”, “inhibition” or “inhibiting” refers to the reduction or suppression of a given condition, symptom, or disorder, or disease, or a significant decrease in the baseline activity of a biological activity or process.

As used herein, the term “pharmaceutically acceptable carrier” refers to a substance useful in the preparation or use of a pharmaceutical composition and includes, for example, suitable diluents, solvents, dispersion media, surfactants, antioxidants, preservatives, isotonic agents, buffering agents, emulsifiers, absorption delaying agents, salts, drug stabilizers, binders, excipients, disintegration agents, lubricants, wetting agents, sweetening agents, flavoring agents, dyes, and combinations thereof, as would be known to those skilled in the art (see, for example, Remington The Science and Practice of Pharmacy, 22^{nd} Ed. Pharmaceutical Press, 2013, pp. 1049-1070).

The phrase “pharmaceutically acceptable” indicates that the substance, composition or dosage form must be compatible chemically and/or toxicologically, with the other ingredients comprising a formulation, and/or the mammal being treated therewith.

The term “a subject in need of such treatment”, refers to a subject which would benefit biologically, medically or in quality of life from such treatment.

The term “therapeutically effective amount,” as used herein, refers to an amount of a compound of the present invention that will ameliorate symptoms, alleviate conditions, slow or delay disease progression, prevent a disease, or elicit the biological or medical response of a subject, for example, increasing the amount of functional CFTR protein at the cell surface, resulting in enhanced ion transport or increasing the channel activity of CFTR protein located at the cell surface, resulting in enhanced ion transport.

As used herein, the term “treat”, “treating” or “treatment” of any disease or disorder, refers to the management and care of a patient for the purpose of combating the disease, condition, or disorder and includes the administration of a compound of the present invention to prevent the onset of the symptoms or complications, alleviating the symptoms or complications, or eliminating the disease, condition or disorder.

In addition, the terms “treatment,” “treating,” as used herein, generally mean the improvement of CF or its symptoms or lessening the severity of CF or its symptoms in a subject. “Treatment,” as used herein, includes, but is not limited to, the following: (i) to ameliorating the disease or disorder (i.e., slowing or arresting or reducing the development of the disease or at least one of the clinical symptoms thereof); (ii) to alleviating or ameliorating at least one physical parameter including those which may not be discernible by the patient; or (iii) to preventing or delaying the onset or development or progression of the disease or disorder. (iiii) increased growth of the subject, increased weight gain, reduction of mucus in the lungs, improved pancreatic and/or liver function, reduced cases of chest infections, and/or reduced instances of coughing or shortness of breath. Improvements in or lessening the severity of any of these conditions can be readily assessed according to standard methods and techniques known in the art.

As used herein, the term “prevent”, “preventing” or “prevention” of any disease or disorder refers to the prophylactic treatment of the disease or disorder; or delaying the onset or progression of the disease or disorder.

As used herein, a subject is “in need of” a treatment if such subject would benefit biologically, medically or in quality of life from such treatment.

The compound names provided herein were obtained using ChemDraw Ultra version 14.0 (CambridgeSoft®) or JChem version 17.2.1300.1489 (ChemAxon).

As used herein, the term “a,” “an,” “the” and similar terms used in the context of the present invention (especially in the context of the claims) are to be construed to cover both the singular and plural unless otherwise indicated herein or clearly contradicted by the context.

### Compounds of the Invention

The invention provides a compound having the structure of formula (I):

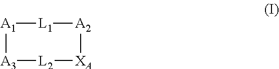

or a pharmaceutically acceptable salt thereof, wherein:
A₁, A₂ and A₃ are each independently selected from an optionally substituted arylene and an optionally substituted heteroarylene;
L₁ is a sulfonamide, an amide, a carbonyl or a urea;
L₂ is an optionally substituted alkylene, an optionally substituted alkoxylene, an optionally substituted polyalkylene oxide, an optionally substituted alkylene oxide, an optionally substituted aminoalkylene or an optionally substituted C₃₋₈ cycloalkylene;
and
X_{A} is an optionally substituted divalent amino, an optionally substituted divalent amide, an optionally substituted heterocycloalkylene or —O—.

Unless specified otherwise, the term “compound of the invention”, “compounds of the invention”, “compound of the present invention” or “compounds of the present invention” refers to a compound or compounds of formula (I), subformulae thereof (such as formula (I-a), formula (I-b), formula (I-c), formula (I-d), formula (I-e), formula (I-f, formula (I-g), formula (I-h), formula (I-i), formula (I-j), formula (I-k), formula (I-l), formula (I-m), formula (I-n), formula (I-o), formula (I-p) and formula (I-q)) and exemplified compounds, and salts thereof, as well as all stereoisomers (including diastereoisomers and enantiomers), rotamers, tautomers and isotopically labeled compounds (including deuterium substitutions), as well as inherently formed moieties (e.g., polymorphs, solvates and/or hydrates).

Certain aspects and examples of the compounds of the present invention are provided in the following listing of additional, enumerated embodiments. It will be recognized that features specified in each embodiment may be combined with other specified features to provide further embodiments of the present invention.

Embodiment 1. The compound of formula (I), or a pharmaceutically acceptable salt thereof,
wherein:
A₁ is an arylene or a heteroarylene, wherein the arylene and heteroarylene of A₁ is unsubstituted or is substituted with 1 to 2 groups independently selected from H, halo, halo-substituted C₁-C₆alkyl, C₁-C₆alkyl, deuterium-substituted C₁-C₆alkyl, nitrile, hydroxyl, C₁-C₆alkoxy and halo-substituted C₁-C₆alkoxy;
A₂ is an arylene or a heteroarylene, wherein the arylene and heteroarylene of A₂ is unsubstituted or is substituted with 1 to 2 groups independently selected from H, halo, halo-substituted C₁-C₆alkyl, C₁-C₆alkyl, deuterium-substituted C₁-C₆alkyl, nitrile, hydroxyl, C₁-C₆alkoxy and halo-substituted C₁-C₆alkoxy;
A₃ is an arylene or a heteroarylene, wherein the arylene and heteroarylene of A₃ is unsubstituted or is substituted with 1 to 2 groups independently selected from H, halo, halo-substituted C₁-C₆alkyl, C₁-C₆alkyl, deuterium-substituted C₁-C₆alkyl, nitrile, hydroxyl, C₁-C₆alkoxy and halo-substituted C₁-C₆alkoxy;
L₁ is —NR^{A}S(O)₁₋₂—*, —S(O)₁₋₂NR^{A}—*, —NR^{A}C(═O)—*, —C(═O)NR^{A}—*, —C(═O)—, or —NR^{A}C(═O)NR^{A}—, where the * indicates the point of attachment to A₂;
L₂ is an alkylene, an alkoxylene, an alkylene oxide, a polyalkylene oxide, an aminoalkylene or a C₃-C₈cycloalkylene, wherein the alkylene, alkoxylene, alkylene oxide, polyalkylene oxide, aminoalkylene and C₃-C₈cycloalkylene of L₂ are unsubstituted or substituted with 1 to 3 groups independently selected from C₁-C₆alkyl, —OH, —(CH₂)ₘC(═O)OR^{A}, C₁-C₆alkoxy, C₁-C₆alkyl substituted with 1 to 6 hydroxyl groups, deuterium, deuterium-substituted C₁-C₆alkyl, and a spiro attached C₃-C₈cycloalkyl;
X_{A} is an optionally substituted divalent amino, an optionally substituted divalent amide, an optionally substituted heterocycloalkylene or —O—;
R^{A} is H or C₁-C₆alkyl,
and
m is 1, 2, 3, 4, 5, or 6.
   Embodiment 2. The compound of formula (I), or a pharmaceutically acceptable salt thereof,
wherein:
A₁ is an arylene or a heteroarylene, wherein the arylene and heteroarylene of A₁ is unsubstituted or is substituted with 1 to 2 groups independently selected from H, halo, halo-substituted C₁-C₆alkyl, C₁-C₆alkyl, deuterium-substituted C₁-C₆alkyl, nitrile, hydroxyl, C₁-C₆alkoxy and halo-substituted C₁-C₆alkoxy;
A₂ is an arylene or a heteroarylene, wherein the arylene and heteroarylene of A₂ is unsubstituted or is substituted with 1 to 2 groups independently selected from H, halo, halo-substituted C₁-C₆alkyl, C₁-C₆alkyl, deuterium-substituted C₁-C₆alkyl, nitrile, hydroxyl, C₁-C₆alkoxy and halo-substituted C₁-C₆alkoxy;
A₃ is an arylene or a heteroarylene, wherein the arylene and heteroarylene of A₃ is unsubstituted or is substituted with 1 to 2 groups independently selected from H, halo, halo-substituted C₁-C₆alkyl, C₁-C₆alkyl, deuterium-substituted C₁-C₆alkyl, nitrile, hydroxyl, C₁-C₆alkoxy and halo-substituted C₁-C₆alkoxy;
L₁ is —NR^{A}S(O)₁₋₂—* or —S(O)₁₋₂NR^{A}—*, where the * indicates the point of attachment to A₂;
L₂ is an alkylene, an alkoxylene, an alkylene oxide, a polyalkylene oxide, an aminoalkylene or a C₃-C₈cycloalkylene, wherein the alkylene, alkoxylene, alkylene oxide, polyalkylene oxide, aminoalkylene and C₃-C₈cycloalkylene of L₂ are unsubstituted or substituted with 1 to 3 groups independently selected from C₁-C₆alkyl, —OH, deuterium, —(CH₂)ₘC(═O)OR^{A}, C₁-C₆alkoxy, C₁-C₆alkyl substituted with 1 to 6 hydroxyl groups, deuterium-substituted C₁-C₆alkyl, and a spiro attached C₃-C₈cycloalkyl;
X_{A} is an optionally substituted divalent amino, an optionally substituted divalent amide, an optionally substituted heterocycloalkylene or —O—;
R^{A} is H or C₁-C₆alkyl,
and
m is 1, 2, 3, 4, 5, or 6.
   Embodiment 3. The compound of formula (I), or a pharmaceutically acceptable salt thereof,
wherein:
A₁ is a phenylene or a pyridylene, wherein the phenylene or pyridylene of A₁ is unsubstituted or is substituted with 1 to 2 groups independently selected from H, halo, halo-substituted C₁-C₆alkyl, C₁-C₆alkyl, deuterium-substituted C₁-C₆alkyl, nitrile, hydroxyl, C₁-C₆alkoxy and halo-substituted C₁-C₆alkoxy;
A₂ is a phenylene or a pyridylene, wherein the phenylene or pyridylene of A₂ is unsubstituted or is substituted with 1 to 2 groups independently selected from H, halo, halo-substituted C₁-C₆alkyl, C₁-C₆alkyl, deuterium-substituted C₁-C₆alkyl, nitrile, hydroxyl, C₁-C₆alkoxy and halo-substituted C₁-C₆alkoxy;
A₃ is a phenylene or a pyridylene, wherein the phenylene or pyridylene of A₃ is unsubstituted or is substituted with 1 to 2 groups independently selected from H, halo, halo-substituted C₁-C₆alkyl, C₁-C₆alkyl, deuterium-substituted C₁-C₆alkyl, nitrile, hydroxyl, C₁-C₆alkoxy and halo-substituted C₁-C₆alkoxy;
L₁ is —NR^{A}S(O)₁₋₂—* or —S(O)₁₋₂NR^{A}—*, where the * indicates the point of attachment to A₂;
L₂ is an alkylene, an alkoxylene, an alkylene oxide, a polyalkylene oxide, an aminoalkylene or a C₃-C₈cycloalkylene, wherein the alkylene, alkoxylene, alkylene oxide, polyalkylene oxide, aminoalkylene and C₃-C₈cycloalkylene of L_{z} are unsubstituted or substituted with 1 to 3 groups independently selected from C₁-C₆alkyl, —OH, deuterium, —(CH₂)ₘC(═O)OR^{A}, C₁-C₆alkoxy, C₁-C₆alkyl substituted with 1 to 6 hydroxyl groups, deuterium-substituted C₁-C₆alkyl, and a spiro attached C₃-C₈cycloalkyl;
X_{A} is an optionally substituted divalent amino, an optionally substituted divalent amide, an optionally substituted heterocycloalkylene or —O—;
R^{A} is H or C₁-C₆alkyl,
and
m is 1, 2, 3, 4, 5, or 6.
   Embodiment 4. The compound of formula (I), or a pharmaceutically acceptable salt thereof,
wherein:
A₁ is a pyridylene, wherein the pyridylene of A₁ is substituted with 1 to 2 groups independently selected from H, halo, halo-substituted C₁-C₆alkyl, C₁-C₆alkyl, deuterium-substituted C₁-C₆alkyl, nitrile, hydroxyl, C₁-C₆alkoxy and halo-substituted C₁-C₆alkoxy;
A₂ is a pyridylene, wherein the pyridylene of A₂ is unsubstituted;
A₃ is a phenylene or a pyridylene, wherein the phenylene or pyridylene of A₃ is unsubstituted or is substituted with 1 to 2 groups independently selected from H, halo, halo-substituted C₁-C₆alkyl, C₁-C₆alkyl, deuterium-substituted C₁-C₆alkyl, nitrile, hydroxyl, C₁-C₆alkoxy and halo-substituted C₁-C₆alkoxy;
L₁ is —NR^{A}S(O)₁₋₂—* or —S(O)₁₋₂NR^{A}—*, where the * indicates the point of attachment to A₂;
L₂ is an alkylene, an alkoxylene, an alkylene oxide, a polyalkylene oxide, an aminoalkylene or a C₃-C₈cycloalkylene, wherein the alkylene, alkoxylene, alkylene oxide, polyalkylene oxide, aminoalkylene and C₃-C₈cycloalkylene of L₂ are unsubstituted or substituted with 1 to 3 groups independently selected from C₁-C₆alkyl, —OH, deuterium, —(CH₂)ₘC(═O)OR^{A}, C₁-C₆alkoxy, C₁-C₆alkyl substituted with 1 to 6 hydroxyl groups, deuterium-substituted C₁-C₆alkyl, and a spiro attached C₃-C₈cycloalkyl;
X_{A} is an optionally substituted divalent amino, an optionally substituted divalent amide, an optionally substituted heterocycloalkylene or —O—;
R^{A} is H or C₁-C₆alkyl,
and
m is 1, 2, 3, 4, 5, or 6
   Embodiment 5. The compound of formula (I), or a pharmaceutically acceptable salt thereof, having the structure of formula (I-a), or a pharmaceutically acceptable salt thereof,

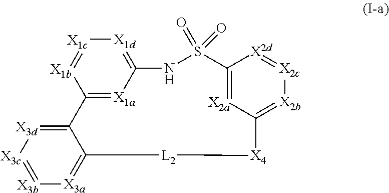

wherein:
X₁ₐ, X_{1b}, X_{1c} and X_{1d} are each independently selected from is CR¹ or N, wherein only 1 or 2 of X₁ₐ, X_{1b}, X_{1c} and X_{1d} can be N and the others are CR¹;
X₂ₐ, X_{2b}, X_{2c} and X_{2d} are each independently selected from is CR¹ or N, wherein only 1 or 2 of X₂ₐ, X_{2b}, X₂c and X_{2d} can be N and the others are CR¹;
X₃ₐ, X_{3b}, X_{3c} and X_{3d} are each independently selected from is CR² or N, wherein only 1 or 2 of X₃ₐ, X_{3b}, X₃c and X_{3d} can be N and the others are CR²;
X₄ is

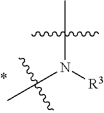

or —O—, wherein * indicates the point of attachment to L₂;
L₂ is (CR⁴R⁵)ₙ—, —O(CR⁴R⁵)ₙ—**, —NR⁷(CR⁴R⁵)ₙ—**, —(CR⁴R⁵)ₙO(CR⁶R¹⁰)ₚ—**, —(CR⁴R⁵)ₙ(CR⁶R¹⁸)—**, —(CR⁴R⁵)ₙ(CR⁸R⁹)ₚ(CR⁴R⁵)ₘ—, —(CR⁴R⁵)ₚNR⁷(CR⁶R¹⁰)ₙ—**, —(CR⁴R⁵)ₙO)ₜ(CR⁶R¹⁰)ₚ—**, or —O—C₃-C₈cycloalkylene-**, wherein ** indicates the point of attachment to X₄ at the point of attachment indicated by the * in X₄;
each R¹ is independently selected from H, halo, halo-substituted C₁-C₆alkyl, C₁-C₆alkyl, deuterium-substituted C₁-C₆alkyl, nitrile, hydroxyl, C₁-C₆alkoxy and halo-substituted C₁-C₆alkoxy;
each R² is independently selected from H, halo, nitrile, hydroxyl, halo-substituted C₁-C₆alkyl, C₁-C₆alkyl, deuterium-substituted C₁-C₆alkyl, hydroxy-substituted C₁-C₆alkyl, C₁-C₆alkoxy and halo-substituted C₁-C₆alkoxy;
R³ is H, —C₁-C₆alkyl, —(CR¹¹R¹²)_{y}R¹⁶, —(CR¹¹R¹²)_{y}C(═O)OR¹³, —((CR¹¹R¹²)_{y}O(CR¹⁴R¹⁵)_{z}C(═O)OR¹³, —((CR¹¹R¹²)_{y}O(CR¹⁴R¹⁵)_{z}OR¹³, —(CR¹¹R¹²)_{y}C(═O)R¹³, —(CR¹¹R¹²)_{y}OR¹³, —(CR¹¹R¹²)_{y}(CR⁸R⁹)_{z}C(═O)OR¹³, —(CR¹¹R¹²)_{y}(CR⁸R⁹)_{z}OR¹³, —(CR¹¹R¹²)_{y}(CR⁸R⁹)_{z}(CR¹⁴R¹⁵)_{q}OR¹³, —(CR¹¹R¹²)_{y}NR¹⁰(CR¹⁴R¹⁵)_{q}C(═O)OR¹³, —(CR¹¹R¹²)_{y}NR¹³R¹⁴, —(CR¹¹R¹²)_{y}R²⁰, —((CR¹¹R¹²)_{y}O)_{q}(CR¹⁴R¹⁵)_{z}C(═O)OR¹³, ((CR¹¹R¹²)_{y}O)_{q}(CR¹⁴R¹⁵)_{z}OR¹³,

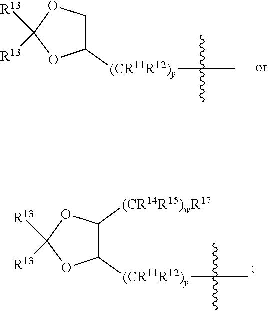

each R⁴ is independently selected from H, D, deuterium-substituted C₁-C₆alkyl and C₁. C₆alkyl;
each R⁵ is independently selected from H, D deuterium-substituted C₁-C₆alkyl and C₁-C₆alkyl;
each R⁶ is independently selected from H, D deuterium-substituted C₁-C₆alkyl and C₁-C₆alkyl;
each R⁷ is independently selected from H, and C₁-C₆alkyl;
each R⁸ and R⁹ are independently selected from H, D, deuterium-substituted C₁-C₆alkyl, C₁-C₆alkyl, or —OH;
or R⁸ and R⁹ together with carbon in CR⁸R⁹ form C₃-C₈cycloalkyl;
each R¹⁰ is independently selected from H, D and C₁-C₆alkyl;
each R¹¹ is independently selected from H, D and C₁-C₆alkyl;
each R¹² is independently selected from H, D, deuterium-substituted C₁-C₆alkyl and C₁-C₆alkyl;
each R¹³ is independently selected from H, and C₁-C₆alkyl;
each R¹⁴ is independently selected from H, D, deuterium-substituted C₁-C₆alkyl and C₁-C₆alkyl;
each R¹⁵ is independently selected from H, D, deuterium-substituted C₁-C₆alkyl and C₁-C₆alkyl;
R¹⁶ is a 4-6 membered heterocycloalkyl having 1-2 ring members independently selected from N, O, and S, wherein the 4-6 membered heterocycloalkyl is unsubstituted or substituted with 1-2 R¹⁷ groups;
each R¹⁷ is independently selected from C₁-C₆alkyl and hydroxyl;
R¹⁸ is H, C₁-C₆alkyl, —C(R⁴R⁵)ₘOR¹⁹, or —(CH₂)ₘC(═O)OR¹⁹;
each R¹⁹ is independently selected from H and C₁-C₆alkyl;
R²⁰ is

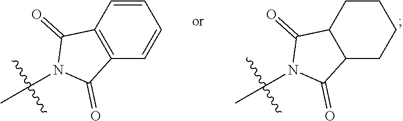 [SMILES: *N1C(=O)c2ccccc2C1=O ; *N1C(=O)C2CCCCC2C1=O]

each m is independently selected from 1, 2, 3, 4, 5, 6, 7, 8, 9 and 10;
each n is independently selected from 1, 2, 3, 4, 5, 6, 7, 8, 9 and 10;
each p is independently selected from 1, 2, 3, 4, 5, 6, 7, 8, 9 and 10;
each t is independently selected from 1, 2, 3, 4, 5, 6, 7, 8, 9 and 10.
each w is independently selected from 1, 2, 3, 4, 5, 6, 7, 8, 9 and 10;
each y is independently selected from 1, 2, 3, 4, 5, 6, 7, 8, 9 and 10;
each z is independently selected from 1, 2, 3, 4, 5, 6, 7, 8, 9 and 10;
and,
each q is independently selected from 1, 2, 3, 4, 5, 6, 7, 8, 9 and 10.
   Embodiment 6. The compound of Embodiment 5, having the structure of formula of Formula (I-b), or a pharmaceutically acceptable salt thereof,

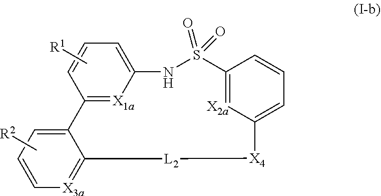

wherein X₁ₐ, X₂ₐ, X₃ₐ, X₄, L₂, R¹ and R² are as defined in Embodiment 5.
   Embodiment 7. The compound of Embodiment 5 or Embodiment 6, having the structure of formula of Formula (I-c) or Formula (I-d), or a pharmaceutically acceptable salt thereof,

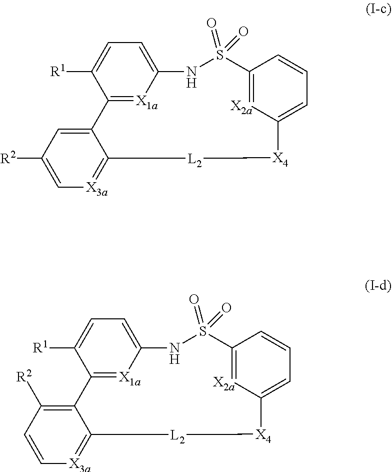

wherein L₂, R¹ and R² are as defined in Embodiment 5, X₁a is CH or N; X₂a is CH or N; and X₃ₐ is CH or N.
   Embodiment 8. The compound of any one of Embodiments 5 to 7, having the structure of Formula (I-e), Formula (I-f), Formula (I-g) or Formula (I-h), or a pharmaceutically acceptable salt thereof:

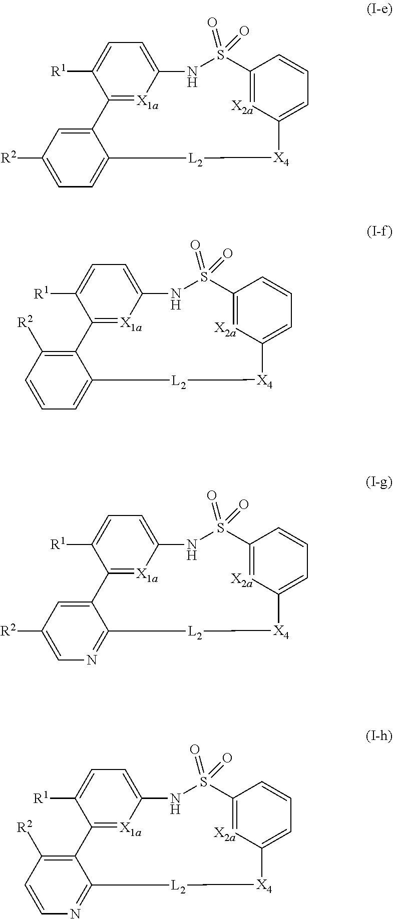

wherein L₂, R¹ and R² are as defined in Embodiment 5, X₁ₐ is CH or N; and X₂ₐ is CH or N.
   Embodiment 9. The compound of any one of Embodiments 5 to 8, having the structure of Formula (I-i), Formula (I-j), Formula (I-k) or Formula (I-l), or a pharmaceutically acceptable salt thereof,

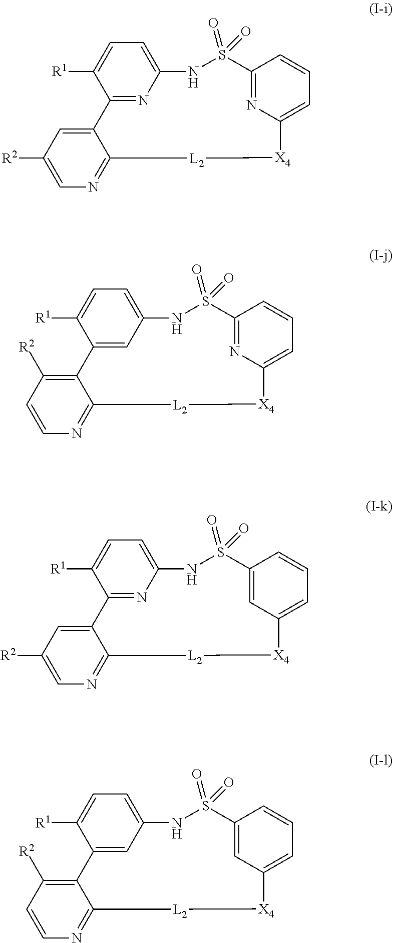

wherein L₂, R¹ and R² are as defined in Embodiment 5.
   Embodiment 10. The compound of any one of Embodiments 5 to 8, having the structure of Formula (I-m), Formula (I-n), Formula (I-o) or Formula (I-p), or a pharmaceutically acceptable salt thereof,

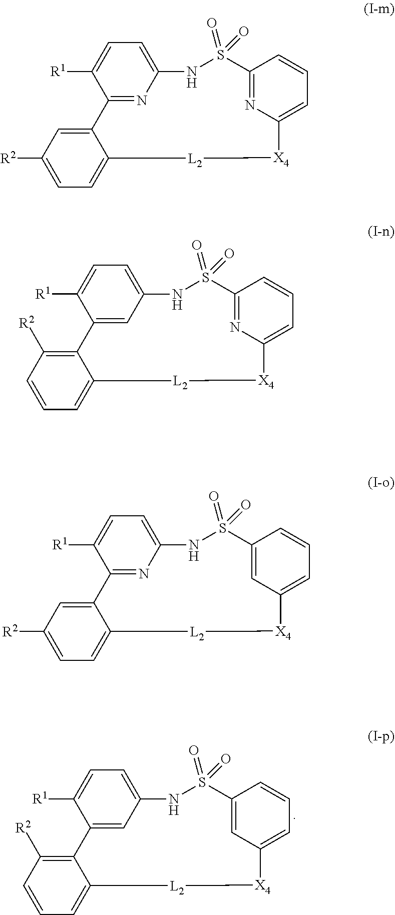

wherein L₂, R¹ and R² are as defined in Embodiment 5.
   Embodiment 11. The compound of any one of Embodiments 5 to 8, having the structure of Formula (I-i), or a pharmaceutically acceptable salt thereof,

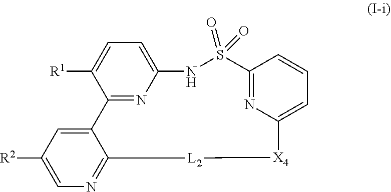

wherein L₂, R¹ and R² are as defined in Embodiment 5.
   Embodiment 12. The compound of any one of Embodiments 5 to 8, having the structure of Formula (I-m), or a pharmaceutically acceptable salt thereof,

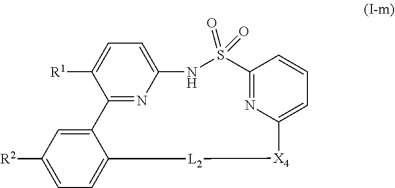

wherein L₂, R¹ and R² are as defined in Embodiment 5.
Embodiment 13. The compound of any one of Embodiments 5 to 12, or a pharmaceutically acceptable salt thereof, wherein:
   X₄ is

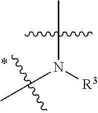

or —O—, wherein * indicates the point of attachment to L₂;
L₂ is —(CR⁴R⁵)ₙ—, —O(CR⁴R⁵)ₙ—**, —NR⁷(CR⁴R⁵)ₙ—**, —(CR⁴R⁵)ₙO(CR⁶R¹⁰)ₚ—**, —(CR⁴R⁵)ₙ(CR⁶R¹⁸)—**, —(CR⁴R⁵)ₙ(CR⁸R⁹)ₚ(CR⁴R⁵)ₘ—, —(CR⁴R⁵)ₚNR⁷(CR⁶R¹⁰)ₙ—**, or —O—C₃-C₈cycloalkylene-**, wherein ** indicates the point of attachment to X₄ at the point of attachment indicated by the * in X₄;
R¹ is H, halo, halo-substituted C₁-C₆alkyl, C₁-C₆alkyl, or C₁-C₆alkoxy;
R² is H, or halo;
R³ is H, —C₁-C₆alkyl, —(CR¹¹R¹²)_{y}R¹⁶, —(CR¹¹R¹²)_{y}C(═O)OR¹³, —((CR¹¹R¹²)_{y}O(CR¹⁴R¹⁵)_{z}C(═O)OR¹³, —((CR¹¹R¹²)_{y}O(CR¹⁴R¹⁵)_{z}OR¹³, —(CR¹¹R¹²)_{y}C(═O)R¹³, —(CR¹¹R¹²)_{y}OR¹³, —(CR¹¹R¹²)_{y}(CR⁸R⁹)_{z}C(═O)OR¹³, —(CR¹¹R¹²)_{y}(CR⁸R⁹)_{z}OR¹³, —(CR¹¹R¹²)_{y}(CR⁸R⁹)_{z}(CR¹⁴R¹⁵)_{q}OR¹³, —(CR¹¹R¹²)_{y}NR¹⁰(CR¹⁴R¹⁵)_{q}C(═O)OR¹³, —(CR¹¹R¹²)_{y}NR¹³R¹⁴, —(CR¹¹R¹²)_{y}R²⁰

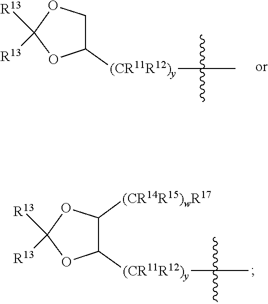

each R⁴ is H;
each R⁵ is H;
each R⁶ is H;
each R⁷ is independently selected from H, and C₁-C₆alkyl;
each R⁸ and R⁹ are independently selected from H, C₁-C₆alkyl, or —OH;
   or R⁸ and R⁹ together with carbon in CR⁸R⁹ form C₃-C₈cycloalkyl;
each R¹⁰ is H;
each R¹¹ is H;
each R¹² is H;
each R¹³ is independently selected from H, and C₁-C₆alkyl;
each R¹⁴ is H;
each R¹⁵ is H;
R¹⁶ is a 4-6 membered heterocycloalkyl having 1-2 ring members independently selected from N, or O, wherein said 4-6 membered heterocycloalkyl is unsubstituted or substituted with 1-2 R¹⁷ groups;
each R¹⁷ is independently selected from C₁-C₆alkyl and hydroxyl;
R¹⁸ is —C(R⁴R⁵)ₘOR¹⁹, or —(CH₂)ₘC(═O)OR¹⁹;
each R¹⁹ is H;
R²⁰ is

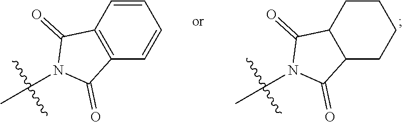 [SMILES: *N1C(=O)c2ccccc2C1=O ; *N1C(=O)C2CCCCC2C1=O]

each m is independently selected from 1, 2 and 3;
each n is independently selected from 1, 2, 3, 4, 5, 6, 7, 8, and 9;
each p is independently selected from 1, 2 and 3;
each w is independently selected from 1 and 2;
each y is independently selected from 1, 2, 3, 4 and 5;
each z is independently selected from 1, 2 and 3;
and,
each q is independently selected from 1 and 2.
Embodiment 14. The compound of any one of Embodiments 5 to 13, or a pharmaceutically acceptable salt thereof, wherein:
   X₄ is

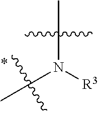

or —O—, wherein * indicates the point of attachment to L₂;
L₂ is —(CR⁴R⁵)ₙ—, —O(CR⁴R⁵)ₙ—**, or —(CR⁴R⁵)ₙO(CR⁶R¹⁰)ₚ—**, wherein ** indicates the point of attachment to X₄ at the point of attachment indicated by the * in X₄;
R¹ is halo or halo-substituted C₁-C₆alkyl;
R² is H, or halo;
R³ is —(CR¹¹R¹²)_{y}C(═O)OR¹³, —(CR¹¹R¹²)_{y}OR¹³, —(CR¹¹R¹²)_{y}(CR⁸R⁹)_{z}C(═O)OR¹³, or —(CR¹¹R¹²)_{y}(CR⁸R⁹)_{z}(CR¹⁴R¹⁵)_{q}OR¹³;
each R⁴ is H;
each R⁵ is H;
each R⁶ is H;
each R⁸ and R⁹ are independently selected from H or C₁-C₆alkyl;
or R⁸ and R⁹ together with carbon in CR⁸R⁹ form C₃-C₈cycloalkyl;
each R¹⁰ is H;
each R¹¹ is H;
each R¹² is H;
each R¹³ is independently selected from H and C₁-C₆alkyl;
each R¹⁴ is H;
each R¹⁵ is H;
each n is independently selected from 1, 2, 3, 4, 5, 6, 7, 8, and 9;
each p is independently selected from 1, 2 and 3;
each y is independently selected from 1, 2, 3, 4 and 5;
each z is independently selected from 1, 2 and 3;
and,
each q is independently selected from 1 and 2.
Embodiment 15. The compound of any one of Embodiments 5 to 14, or a pharmaceutically acceptable salt thereof, wherein:
   X₄ is

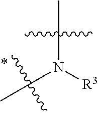

or —O—, wherein * indicates the point of attachment to L₂;
L₂ is —(CR⁴R⁵)ₙ—, —O(CR⁴R⁵)ₙ—**, or —(CR⁴R⁵)ₙO(CR⁶R¹⁰)ₚ wherein ** indicates the point of attachment to X₄ at the point of attachment indicated by the * in X₄;
R¹ is C₁, F or CF₃;
R² is H or F;
R³ is —(CR¹¹R¹²)_{y}C(═O)OR¹³, —(CR¹¹R¹²)_{y}OR¹³, —(CR¹¹R¹²)_{y}(CR⁸R⁹)_{z}C(═O)OR¹³, or —(CR¹¹R¹²)_{y}(CR⁸R⁹)_{z}(CR¹⁴R¹⁵)_{q}OR¹³;
each R⁴ is H;
each R⁵ is H;
each R⁶ is H;
each R⁸ and R⁹ are independently selected from H or methyl;
or R⁸ and R⁹ together with carbon in CR⁸R⁹ form a cyclopropyl;
each R¹⁰ is H;
each R¹¹ is H;
each R¹² is H;
each R¹³ is independently selected from H, methyl and ethyl;
each R¹⁴ is H;
each R¹⁵ is H;
each n is independently selected from 1, 2, 3, 4, 5, 6, 7, 8, and 9;
each p is independently selected from 1, 2 and 3;
each y is independently selected from 1, 2, 3, 4 and 5;
z is 1;
and,
q is 1.
Embodiment 16. The compound of any one of Embodiments 5 to 15, or a pharmaceutically acceptable salt thereof, wherein:
   X₄ is

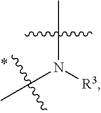

wherein * indicates the point of attachment to L₂;
L₂ is —(CR⁴R⁵)ₙ—;
R¹ is CF₃;
R² is H;
R³ is —(CR¹¹R¹²)_{y}C(═O)OR¹³;
each R⁴ is H;
each R⁵ is H;
each R¹¹ is H;
each R¹² is H;
each R¹³ is H;
n is 1, 2, 3, 4, 5, 6, 7, 8, or 9;
and
y is 2, 3 or 4.
   Embodiment 17. The compound of Embodiment 5, having the structure of formula of Formula (I-q), or a pharmaceutically acceptable salt thereof,

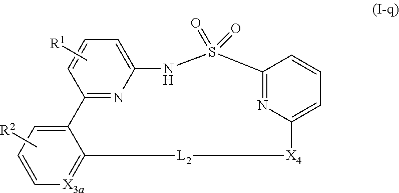

wherein X₃ₐ is CH or N; and X₄, L₂, R¹ and R² are as defined in Embodiment 13.
   Embodiment 18. The compound of Embodiment 17, wherein X₃a is CH or N; and X₄, L₂, R¹ and R² are as defined in Embodiment 14.
Embodiment 19. The compound of any one of Embodiments 5 to 18, or a pharmaceutically acceptable salt thereof, wherein:
   X₄ is

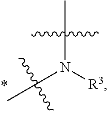

wherein * indicates the point of attachment to L₂.
Embodiment 20. The compound of any one of Embodiments 5 to 15 or Embodiments 17 to 18, or a pharmaceutically acceptable salt thereof, wherein X₄ is —O—.
Embodiment 21. The compound of any one of Embodiments 5 to 12 or Embodiments 17 to 18, or a pharmaceutically acceptable salt thereof, wherein L₂ is —(CR⁴R⁵)ₙ—, —O(CR⁴R⁵)ₙ—**, —NR⁷(CR⁴R⁵)ₙ—**, —(CR⁴R⁵)ₙO(CR⁶R¹⁰)ₚ—**, —(CR⁴R⁵)ₙ(CR⁶R¹⁸)ₙ—**, —(CR⁴R⁵)ₙ(CR⁸R⁹)ₚ(CR⁴R⁵)ₘ—, —(CR⁴R⁵)ₚNR⁷(CR⁶R¹⁰)ₙ—**, or —O—C₃-C₈cycloalkylene-**, wherein ** indicates the point of attachment to X₄ at the point of attachment indicated by the * in X₄.
Embodiment 22. The compound of any one of Embodiments 5 to 13 or Embodiments 17 to 20, or a pharmaceutically acceptable salt thereof, wherein L₂ is —(CR⁴R⁵)ₙ—, —O(CR⁴R⁵)ₙ—**, or —(CR⁴R⁵)ₙO(CR⁶R¹⁰)ₚ—**, wherein ** indicates the point of attachment to X₄ at the point of attachment indicated by the * in X₄.
Embodiment 23. The compound of any one of Embodiments 5 to 15 or Embodiments 17 to 20, or a pharmaceutically acceptable salt thereof, wherein L₂ is —(CR⁴R⁵)ₙ—.
Embodiment 24. The compound of any one of Embodiments 5 to 15 or Embodiments 17 to 20, or a pharmaceutically acceptable salt thereof, wherein L₂ is —O(CR⁴R⁵)ₙ—**, wherein ** indicates the point of attachment to X₄ at the point of attachment indicated by the * in X₄.
Embodiment 25. The compound of any one of Embodiments 5 to 15 or Embodiments 17 to 20, or a pharmaceutically acceptable salt thereof, wherein L₂ is —(CR⁴R⁵)ₙO(CR⁶R¹⁰)ₚ—**, wherein ** indicates the point of attachment to X₄ at the point of attachment indicated by the * in X₄.
Embodiment 26. The compound of any one of Embodiments 5 to 13 or Embodiments 17 to 25, or a pharmaceutically acceptable salt thereof, wherein R³ is H, —C₁-C₆alkyl, —(CR¹¹R¹²)_{y}R¹⁶, —(CR¹¹R¹²)_{y}C(═O)OR¹³, —((CR¹¹R¹²)_{y}O(CR¹⁴R¹⁵)_{z}C(═O)OR¹³, —((CR¹¹R¹²)_{y}O(CR¹⁴R¹⁵)_{z}OR¹³, —(CR¹¹R¹²)_{y}C(═O)R¹³, —(CR¹¹R¹²)_{y}OR¹³, —(CR¹¹R¹²)_{y}(CR⁸R⁹)_{z}C(═O)OR¹³, —(CR¹¹R¹²)_{y}(CR⁸R⁹)_{z}OR¹³, —(CR¹¹R¹²)_{y}(CR⁸R⁹)_{z}(CR¹⁴R¹⁵)_{q}OR¹³, —(CR¹¹R¹²)_{y}NR¹⁰ (CR¹⁴R¹⁵)_{q}C(═O)OR¹³, —(CR¹¹R¹²)_{y}NR¹³R¹⁴, —(CR¹¹R¹²)_{y}R²⁰,

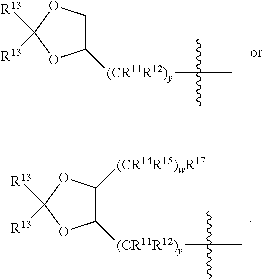

Embodiment 27. The compound of any one of Embodiments 5 to 13 or Embodiments 17 to 26, or a pharmaceutically acceptable salt thereof, wherein R³ is H or —C₁-C₆alkyl.
Embodiment 28. The compound of any one of Embodiments 5 to 13 or Embodiments 17 to 27, or a pharmaceutically acceptable salt thereof, wherein R³ is H, methyl or ethyl.
Embodiment 29. The compound of any one of Embodiments 5 to 13 or Embodiments 17 to 28, or a pharmaceutically acceptable salt thereof, wherein R³ is H.
Embodiment 30. The compound of any one of Embodiments 5 to 13 or Embodiments 17 to 28, or a pharmaceutically acceptable salt thereof, wherein R³ is methyl.
Embodiment 31. The compound of any one of Embodiments 5 to 13 or Embodiments 17 to 28, or a pharmaceutically acceptable salt thereof, wherein R³ is ethyl.
Embodiment 32. The compound of any one of Embodiments 5 to 13 or Embodiments 17 to 26, or a pharmaceutically acceptable salt thereof, wherein R³ is H, —(CR¹¹R¹²)_{y}C(═O)OR¹³, —((CR¹¹R¹²)_{y}O(CR¹⁴R¹⁵)_{z}C(═O)OR¹³, —((CR¹¹R²)_{y}O(CR¹⁴R¹⁵)_{z}OR¹³, —(CR¹¹R¹²)_{y}C(═O)R¹³. —(CR¹¹R¹²)_{y}OR¹³. —(CR¹¹R¹²)_{y}(CR⁸R⁹)_{z}C(═O)OR¹³, —(CR¹¹R¹²)_{y}(CR⁸R⁹)_{z}OR¹³, —(CR¹¹R¹²)_{y}(CR⁸R⁹)_{z}(CR¹⁴R¹⁵)_{q}OR¹³ or —(CR¹¹R¹²)_{y}NR¹⁰ (CR¹⁴R¹⁵)_{q}C(═O)OR¹³.
Embodiment 33. The compound of any one of Embodiments 5 to 15, Embodiments 17 to 26 or Embodiment 32, or a pharmaceutically acceptable salt thereof, wherein R³ is —(CR¹¹R¹²)_{y}C(═O)OR¹³, —(CR¹¹R¹²)_{y}OR¹³, —(CR¹¹R¹²)_{y}(CR⁸R⁹)_{z}C(═O)OR¹³ or —(CR¹¹R¹²)_{y}(CR⁸R⁹)_{z}(CR¹⁴R¹⁵)_{q}OR¹³.
Embodiment 34. The compound of any one of Embodiments 5 to 16, Embodiments 17 to 26 or Embodiments 32 to 33, or a pharmaceutically acceptable salt thereof, wherein R³ is —(CR¹¹R¹²)_{y}C(═O)OR¹³.
Embodiment 35. The compound of any one of Embodiments 5 to 15, Embodiments 17 to 26 or Embodiments 32 to 33, or a pharmaceutically acceptable salt thereof, wherein R³ is —(CR¹¹R¹²)_{y}OR¹³.
Embodiment 36. The compound of any one of Embodiments 5 to 15, Embodiments 17 to 26 or Embodiments 32 to 33, or a pharmaceutically acceptable salt thereof, wherein R³ is —(CR¹¹R¹²)_{y}(CR⁸R⁹)_{z}C(═O)OR¹³.
Embodiment 37. The compound of any one of Embodiments 5 to 15, Embodiments 17 to 26 or Embodiments 32 to 33, or a pharmaceutically acceptable salt thereof, wherein R³ is —(CR¹¹R¹²)_{y}(CR⁸R⁹)_{z}(CR¹⁴R¹⁵)_{q}OR¹³.
Embodiment 38. The compound of any one of Embodiments 5 to 12 or Embodiments 17 to 26, or a pharmaceutically acceptable salt thereof, wherein R³ is

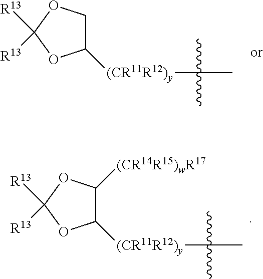

Embodiment 39. The compound of any one of Embodiments 5 to 13 or Embodiments 17 to 26, or a pharmaceutically acceptable salt thereof, wherein R³ is —(CR¹¹R¹²)_{y}R¹⁶ or —(CR¹¹R¹²)_{y}R²⁰.
Embodiment 40. The compound of any one of Embodiments 5 to 13, Embodiments 17 to 26 or Embodiment 39, or a pharmaceutically acceptable salt thereof, wherein:
   R¹⁶ is an unsubstituted 4-6 membered heterocycloalkyl having 1-2 ring members independently selected from N, or O.
Embodiment 41. The compound of any one of Embodiments 5 to 13, Embodiments 17 to 26 or Embodiments 39 to 40, or a pharmaceutically acceptable salt thereof, wherein R¹⁶ is an unsubstituted morpholinyl.
Embodiment 42. The compound of any one of Embodiments 5 to 13, Embodiments 17 to 26 or Embodiments 39 to 40, or a pharmaceutically acceptable salt thereof, wherein R¹⁶ is an unsubstituted pyrrolidinyl.
Embodiment 43. The compound of any one of Embodiments 5 to 13, Embodiments 17 to 26 or Embodiments 39 to 40, or a pharmaceutically acceptable salt thereof, wherein R¹⁶ is an unsubstituted piperazinyl.
Embodiment 44. The compound of any one of Embodiments 5 to 13, Embodiments 17 to 26 or Embodiment 39, or a pharmaceutically acceptable salt thereof, wherein:
   R¹⁶ is a 4-6 membered heterocycloalkyl having 1-2 ring members independently selected from N, O and S, substituted with 1-2 R¹⁷ groups.
Embodiment 45. The compound of any one of Embodiments 5 to 13, Embodiments 17 to 26, Embodiment 39 or Embodiment 44, or a pharmaceutically acceptable salt thereof, wherein: R¹⁶ is a azetidinyl substituted with a hydroxyl group.
Embodiment 46. The compound of any one of Embodiments 5 to 13, Embodiments 17 to 26, Embodiment 39 or Embodiment 44, or a pharmaceutically acceptable salt thereof, wherein: R¹⁶ is a pyrrolidinyl substituted with 1-2 hydroxyl groups.
Embodiment 47. The compound of any one of Embodiments 5 to 13, Embodiments 17 to 26, Embodiment 39 or Embodiment 44, or a pharmaceutically acceptable salt thereof, wherein: R¹⁶ is a piperazinyl substituted with a methyl group.
Embodiment 48. The compound of any one of Embodiments 5 to 13, Embodiments 17 to 26, or Embodiment 39, or a pharmaceutically acceptable salt thereof, wherein: R¹⁶ is morpholinyl, azetidinyl, pyrrolidinyl or piperazinyl, each of which is unsubstituted or substituted with 1-2 R¹⁷ groups.
Embodiment 49. The compound of any one of Embodiments 5 to 13, Embodiments 17 to 26, or Embodiment 39, or a pharmaceutically acceptable salt thereof, wherein: R¹⁶ is morpholinyl, azetidinyl, pyrrolidinyl or piperazinyl, each of which is unsubstituted or substituted with 1-2 groups independently selected from hydroxyl and methyl.
Embodiment 50. The compound of any one of Embodiments 5 to 13, Embodiments 17 to 26 or Embodiments 39, 44, or 48, or a pharmaceutically acceptable salt thereof, wherein each R¹⁷ is independently selected from C₁-C₆alkyl and hydroxyl.
Embodiment 51. The compound of any one of Embodiments 5 to 13, Embodiments 17 to 26 or Embodiments 39, 44, or 48, or a pharmaceutically acceptable salt thereof, wherein each R¹⁷ is independently selected from methyl, ethyl, propyl and hydroxyl.
Embodiment 52. The compound of any one of Embodiments 5 to 13, Embodiments 17 to 24 or Embodiments 39, 44, or 48, or a pharmaceutically acceptable salt thereof, wherein each R¹⁷ is independently selected from methyl and hydroxyl.
Embodiment 53. The compound of any one of Embodiments 5 to 13, Embodiments 17 to 26 or Embodiment 39, or a pharmaceutically acceptable salt thereof, wherein R²⁰ is

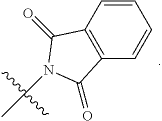 [SMILES: *N1C(=O)c2ccccc2C1=O]

Embodiment 54. The compound of any one of Embodiments 5 to 13, Embodiments 17 to 26 or Embodiment 39, or a pharmaceutically acceptable salt thereof, wherein R²⁰ is

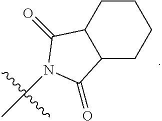 [SMILES: *N1C(=O)C2CCCCC2C1=O]

Embodiment 55. The compound of any one of Embodiments 5 to 13 or Embodiments 17 to 54, or a pharmaceutically acceptable salt thereof, wherein R¹⁸ is —C(R⁴R⁵)ₘOR¹⁹, or —(CH₂)ₘC(═O)OR¹⁹.
Embodiment 56. The compound of any one of Embodiments 5 to 13 or Embodiments 17 to 55, or a pharmaceutically acceptable salt thereof, wherein R¹⁸ is —C(R⁴R⁵)ₘOR¹⁹.
Embodiment 57. The compound of any one of Embodiments 5 to 13 or Embodiments 17 to 55, or a pharmaceutically acceptable salt thereof, wherein R¹⁸ is (CH₂)ₘC(═O)OR¹⁹.
Embodiment 58. The compound of any one of Embodiments 6 to 13 or Embodiments 17 to 57, or a pharmaceutically acceptable salt thereof, wherein R¹ is H, halo, halo-substituted C₁-C₆alkyl, C₁-C₆alkyl, or C₁-C₆alkoxy.
Embodiment 59. The compound of any one of Embodiments 6 to 13 or Embodiments 17 to 58, or a pharmaceutically acceptable salt thereof, wherein R¹ is H.
Embodiment 60. The compound of any one of Embodiments 6 to 14 or Embodiments 17 to 58, or a pharmaceutically acceptable salt thereof, wherein R¹ is halo.
Embodiment 61. The compound of any one of Embodiments 6 to 14, Embodiments 17 to 58 or Embodiment 60, or a pharmaceutically acceptable salt thereof, wherein R¹ is F.
Embodiment 62. The compound of any one Embodiments 6 to 14, Embodiments 17 to 58 or Embodiment 60, or a pharmaceutically acceptable salt thereof, wherein R¹ is C₁.
Embodiment 63. The compound of any one of Embodiments 6 to 14 or Embodiments 17 to 58, or a pharmaceutically acceptable salt thereof, wherein R¹ is halo-substituted C₁-C₆alkyl.
Embodiment 64. The compound of any one of Embodiments 6 to 14, Embodiments 17 to 58 or Embodiment 63, or a pharmaceutically acceptable salt thereof, wherein R¹ is CF₃.
Embodiment 65. The compound of any one of Embodiments 6 to 14, Embodiments 17 to 58 or Embodiment 63, or a pharmaceutically acceptable salt thereof, wherein R¹ is CHF₂.
Embodiment 66. The compound of any one of Embodiments 6 to 13 or Embodiments 17 to 58, or a pharmaceutically acceptable salt thereof, wherein R¹ is C₁-C₆alkyl.
Embodiment 67. The compound of any one of Embodiments 6 to 13, Embodiments 17 to 58 or Embodiment 66, or a pharmaceutically acceptable salt thereof, wherein R¹ is methyl.
Embodiment 68. The compound of any one of Embodiments 6 to 13 or Embodiments 17 to 58, or a pharmaceutically acceptable salt thereof, wherein R¹ is C₁-C₆alkoxy.
Embodiment 69. The compound of any one of Embodiments 6 to 13, Embodiments 17 to 58 or Embodiment 68, or a pharmaceutically acceptable salt thereof, wherein R¹ is methoxy.
Embodiment 70. The compound of Embodiment 5, or a pharmaceutically acceptable salt thereof, wherein each R¹ is independently selected from H, halo, halo-substituted C₁-C₆alkyl, C₁-C₆alkyl and C₁-C₆alkoxy.
Embodiment 71. The compound of Embodiment 5, or a pharmaceutically acceptable salt thereof, wherein each R¹ is independently selected from H and halo.
Embodiment 72. The compound of Embodiment 5, or a pharmaceutically acceptable salt thereof, wherein each R¹ is independently selected from H and F.
Embodiment 73. The compound of Embodiment 5, or a pharmaceutically acceptable salt thereof, wherein each R¹ is independently selected from H and C₁.
Embodiment 74. The compound of Embodiment 5, or a pharmaceutically acceptable salt thereof, wherein each R¹ is independently selected from H and halo-substituted C₁-C₆alkyl.
Embodiment 75. The compound of Embodiment 5, or a pharmaceutically acceptable salt thereof, wherein each R¹ is independently selected from H and —CF₃.
Embodiment 76. The compound of Embodiment 5, or a pharmaceutically acceptable salt thereof, wherein each R¹ is independently selected from H and —CHF₂.
Embodiment 77. The compound of Embodiment 5, or a pharmaceutically acceptable salt thereof, wherein each R¹ is independently selected from H and C₁-C₆alkyl.
Embodiment 78. The compound of Embodiment 5, a pharmaceutically acceptable salt thereof, wherein each R¹ is independently selected from H and methyl.
Embodiment 79. The compound of Embodiment 5, a pharmaceutically acceptable salt thereof, wherein each R¹ is independently selected from H and C₁-C₆alkoxy.
Embodiment 80. The compound of Embodiment 5, a pharmaceutically acceptable salt thereof, wherein each R¹ is independently selected from H and methoxy.
Embodiment 81. The compound of any one of Embodiments 6 to 14 or Embodiments 17 to 80, or a pharmaceutically acceptable salt thereof, wherein R² is H, or halo.
Embodiment 82. The compound of any one of Embodiments 6 to 14 or Embodiments 17 to 81, or a pharmaceutically acceptable salt thereof, wherein R² is halo.
Embodiment 83. The compound of any one of Embodiments 6 to 14 or Embodiments 17 to 82, or a pharmaceutically acceptable salt thereof, wherein R² is F.
Embodiment 84. The compound of any one of Embodiments 6 to 81, or a pharmaceutically acceptable salt thereof, wherein R² is H.
Embodiment 85. The compound of Embodiment 5, or a pharmaceutically acceptable salt thereof, wherein each R² is independently selected from H and halo.
Embodiment 86. The compound of Embodiment 5, or a pharmaceutically acceptable salt thereof, wherein each R² is independently selected from R² is H and F.
Embodiment 87. The compound of any one of Embodiments 5 to 86, or a pharmaceutically acceptable salt thereof, wherein each R⁴ is H.
Embodiment 88. The compound of any one of Embodiments 5 to 87, or a pharmaceutically acceptable salt thereof, wherein each R⁵ is H.
Embodiment 89. The compound of any one of Embodiments 5 to 88, or a pharmaceutically acceptable salt thereof, wherein each R⁶ is H.
Embodiment 90. The compound of any one of Embodiments 5 to 89, or a pharmaceutically acceptable salt thereof, wherein each R⁷ is independently selected from H and C₁-C₆alkyl.
Embodiment 91. The compound of any one of Embodiments 5 to 90, or a pharmaceutically acceptable salt thereof, wherein each R⁷ is independently selected from H and methyl.
Embodiment 92. The compound of any one of Embodiments 5 to 91, or a pharmaceutically acceptable salt thereof, wherein each R⁸ and R⁹ are independently selected from H, C₁-C₆alkyl, or —OH.
Embodiment 93. The compound of any one of Embodiments 5 to 92, or a pharmaceutically acceptable salt thereof, wherein each R⁸ and R⁹ are independently selected from H, methyl or —OH.
Embodiment 94. The compound of any one of Embodiments 5 to 91, or a pharmaceutically acceptable salt thereof, wherein R⁸ and R⁹ together with carbon in CR⁸R⁹ form a C₃. C₈cycloalkyl.
Embodiment 95. The compound of any one of Embodiments 5 to 91 or Embodiment 93, or a pharmaceutically acceptable salt thereof, wherein R⁸ and R⁹ together with carbon in CR⁸R⁹ form a cyclopropyl.
Embodiment 96. The compound of any one of Embodiments 5 to 95, or a pharmaceutically acceptable salt thereof, wherein each R¹⁰ is H; Embodiment 97. The compound of any one of Embodiments 5 to 96, or a pharmaceutically acceptable salt thereof, wherein each R¹¹ is H.
Embodiment 98. The compound of any one of Embodiments 5 to 97, or a pharmaceutically acceptable salt thereof, wherein each R¹² is H.
Embodiment 99. The compound of any one of Embodiments 5 to 98, or a pharmaceutically acceptable salt thereof, wherein each R¹³ is independently selected from H and C₁-C₆alkyl.
Embodiment 100. The compound of any one of Embodiments 5 to 98, or a pharmaceutically acceptable salt thereof, wherein each R¹³ is independently selected from H, methyl and ethyl.
Embodiment 101. The compound of any one of Embodiments 5 to 100, or a pharmaceutically acceptable salt thereof, wherein each R¹⁴ is H.
Embodiment 102. The compound of any one of Embodiments 5 to 100, or a pharmaceutically acceptable salt thereof, wherein each R¹⁵ is H.
Embodiment 103. The compound of any one of Embodiments 5 to 102, or a pharmaceutically acceptable salt thereof, wherein each R¹⁹ is H.
Embodiment 104. The compound of any one of Embodiments 5 to 102, or a pharmaceutically acceptable salt thereof, wherein each R¹⁹ is C₁-C₆alkyl.
Embodiment 105. The compound of any one of Embodiments 5 to 102, or a pharmaceutically acceptable salt thereof, wherein each R¹⁹ is methyl or ethyl.
Embodiment 106. The compound of any one of Embodiments 2 to 13 or Embodiments 17 to 105, or a pharmaceutically acceptable salt thereof, wherein each m is independently selected from 1, 2 and 3;
Embodiment 107. The compound of any one of Embodiments 5 to 13 or Embodiments 17 to 106, or a pharmaceutically acceptable salt thereof, wherein each w is independently selected from 1 and 2.
Embodiment 108. The compound of any one of Embodiments 5 to 14 or Embodiments 17 to 107, or a pharmaceutically acceptable salt thereof, wherein each z is independently selected from 1, 2 and 3.
Embodiment 109. The compound of any one of Embodiments 5 to 14 or Embodiments 17 to 108, or a pharmaceutically acceptable salt thereof, wherein each z is 1.
Embodiment 110. The compound of any one of Embodiments 5 to 15 or Embodiments 17 to 109, or a pharmaceutically acceptable salt thereof, wherein each q is independently selected from 1 and 2.
Embodiment 111. The compound of any one of Embodiments 5 to 15 or Embodiments 17 to 109, or a pharmaceutically acceptable salt thereof, wherein each q is 1.
Embodiment 112. The compound of any one of Embodiments 5 to 15 or Embodiments 17 to 111, or a pharmaceutically acceptable salt thereof, wherein each p is independently selected from 1, 2 and 3.
Embodiment 113. The compound of any one of Embodiments 5 to 112, or a pharmaceutically acceptable salt thereof, wherein each y is independently selected from 1, 2, 3, 4 and 5.
Embodiment 114. The compound of any one of Embodiments 5 to 112, or a pharmaceutically acceptable salt thereof, wherein each y is independently selected from 2, 3 and 4.
Embodiment 115. The compound of any one of Embodiments 5 to 114, or a pharmaceutically acceptable salt thereof, wherein each n is independently selected from 1, 2, 3, 4, 5, 6, 7, 8, and 9.
   Embodiment 116. The compound of any one of Embodiments 5 to 12 selected from
6-(2-morpholinoethyl)-2³-(trifluoromethyl)-4-thia-3,6-diaza-2,5(2,6)-dipyridina-1(1,2)-benzenacycloundecaphane 4,4-dioxide;
2³-(trifluoromethyl)-11-oxa-4-thia-3,6-diaza-2,5(2,6)-dipyridina-1(1,2)-benzenacycloundecaphane 4,4-dioxide;
2-(4,4-dioxido-2³-(trifluoromethyl)-1¹-oxa-4-thia-3,6-diaza-2,5(2,6)-dipyridina-1(1,2)-benzenacycloundecaphane-6-yl)acetic acid;
2³-(trifluoromethyl)-4-thia-3,6,1′-triaza-1(3,2),2,5(2,6)-tripyridinacycloundecaphane 4,4-dioxide;
2³-(trifluoromethyl)-1²-oxa-4-thia-3,6-diaza-1(3,2),2,5(2,6)-tripyridinacyclododecaphane 4,4-dioxide;
4-(1⁵-fluoro-4,4-dioxido-2³-(trifluoromethyl)-11-oxa-4-thia-3,6-diaza-2,5(2,6)-dipyridina-1(1,2)-benzenacycloundecaphane-6-yl)butanoic acid;
3-(2-(4,4-dioxido-2³-(trifluoromethyl)-4-thia-3,6-diaza-2,5(2,6)-dipyridina-1(1,2)-benzenacycloundecaphane-6-yl)ethoxy)propanoic acid;
4-(4,4-dioxido-2³-(trifluoromethyl)-4-thia-3,6-diaza-2,5(2,6)-dipyridina-1(1,2)-benzenacyclotridecaphane-6-yl)butanoic acid;
2³-(trifluoromethyl)-10-oxa-4-thia-3,6-diaza-2,5(2,6)-dipyridina-1(1,2)-benzenacycloundecaphane 4,4-dioxide;
4-(4,4-dioxido-2³-(trifluoromethyl)-4-thia-3,6-diaza-2,5(2,6)-dipyridina-1(1,2)-benzenacyclopentadecaphane-6-yl)butanoic acid;
6-(2-(3-hydroxypropoxy)ethyl)-2³-(trifluoromethyl)-4-thia-3,6-diaza-2,5(2,6)-dipyridina-1(1,2)-benzenacycloundecaphane 4,4-dioxide;
4-(4,4-dioxido-2³-(trifluoromethyl)-4-thia-3,6-diaza-2,5(2,6)-dipyridina-1(1,2)-benzenacycloundecaphane-6-yl)butanoic acid;
4-(1⁵-fluoro-4,4-dioxido-2³-(trifluoromethyl)-4-thia-3,6-diaza-2,5(2,6)-dipyridina-1(1,2)-benzenacyclododecaphane-6-yl)butanoic acid;
3-(4,4-dioxido-2³-(trifluoromethyl)-4-thia-3,6-diaza-2,5(2,6)-dipyridina-1(1,2)-benzenacyclotetradecaphane-6-yl)propanoic acid;
6-(4,4-dioxido-2³-(trifluoromethyl)-4-thia-3,6-diaza-2,5(2,6)-dipyridina-1(1,2)-benzenacycloundecaphane-6-yl)hexanoic acid:
4-(4,4-dioxido-2³-(trifluoromethyl)-4-thia-3,6-diaza-2,5(2,6)-dipyridina-1(1,2)-benzenacyclotetradecaphane-6-yl)butanoic acid;
3-(4,4-dioxido-2³-(trifluoromethyl)-4-thia-3,6-diaza-2,5(2,6)-dipyridina-1 (1,2)-benzenacyclotridecaphane-6-yl)propanoic acid;
4-(4,4-dioxido-2³-(trifluoromethyl)-10-oxa-4-thia-3,6-diaza-2,5(2,6)-dipyridina-1(1,2)-benzenacycloundecaphane-6-yl)butanoic acid;
4-(2³-methyl-4,4-dioxido-4-thia-3,6-diaza-2,5(2,6)-dipyridina-1(1,2)-benzenacycloundecaphane-6-yl)butanoic acid;
4-(4,4-dioxido-2³-(trifluoromethyl)-4-thia-3,6-diaza-2,5(2,6)-dipyridina-1(1,2)-benzenacycloundecaphane-6-yl)butanal;
2-(2-(4,4-dioxido-2³-(trifluoromethyl)-4-thia-3,6-diaza-2,5(2,6)-dipyridina-1(1,2)-benzenacycloundecaphane-6-yl)ethoxy)acetic acid;
3-(4,4-dioxido-2³-(trifluoromethyl)-4-thia-3,6-diaza-2,5(2,6)-dipyridina-1(1,2)-benzenacyclopentadecaphane-6-yl)propanoic acid;
4-(4,4-dioxido-2³-(trifluoromethyl)-4-thia-3,6-diaza-2,5(2,6)-dipyridina-1(1,2)-benzenacyclododecaphane-6-yl)butanoic acid;
5-(4,4-dioxido-2³-(trifluoromethyl)-4-thia-3,6-diaza-2,5(2,6)-dipyridina-1(1,2)-benzenacycloundecaphane-6-yl)pentanoic acid;
5-(4,4-dioxido-2³-(trifluoromethyl)-4-thia-3,6-diaza-2,5(2,6)-dipyridina-1(1,2)-benzenacyclododecaphane-6-yl)pentanoic acid;
4-(2³-chloro-4,4-dioxido-4-thia-3,6-diaza-2,5(2,6)-dipyridina-1(1,2)-benzenacycloundecaphane-6-yl)butanoic acid;
5-(4,4-dioxido-2³-(trifluoromethyl)-4-thia-3,6-diaza-2,5(2,6)-dipyridina-1(1,2)-benzenacyclodecaphane-6-yl)pentanoic acid;
3-(1⁵-fluoro-4,4-dioxido-2³-(trifluoromethyl)-11-oxa-4-thia-3,6-diaza-2,5(2,6)-dipyridina-1(1,2)-benzenacycloundecaphane-6-yl)propanoic acid;
6-(2³-chloro-4,4-dioxido-4-thia-3,6-diaza-2,5(2,6)-dipyridina-1(1,2)-benzenacycloundecaphane-6-yl)hexanoic acid;
4-(2³-methoxy-4,4-dioxido-4-thia-3,6-diaza-2,5(2,6)-dipyridina-1(1,2)-benzenacycloundecaphane-6-yl)butanoic acid;
6-(5-hydroxypentyl)-2³-(trifluoromethyl)-4-thia-3,6-diaza-2,5(2,6)-dipyridina-1(1,2)-benzenacycloundecaphane 4,4-dioxide;
6-(5-hydroxypentyl)-2³-(trifluoromethyl)-4-thia-3,6-diaza-2,5(2,6)-dipyridina-1(1,2)-benzenacyclododecaphane 4,4-dioxide;
3-(4,4-dioxido-2³-(trifluoromethyl)-4-thia-3,6-diaza-2,5(2,6)-dipyridina-1(1,2)-benzenacyclododecaphane-6-yl)propanoic acid;
6-(4-hydroxybutyl)-2³-(trifluoromethyl)-4-thia-3,6-diaza-2,5(2,6)-dipyridina-1(1,2)-benzenacyclododecaphane 4,4-dioxide;
6-(4-hydroxybutyl)-2³-methoxy-4-thia-3,6-diaza-2,5(2,6)-dipyridina-1(1,2)-benzenacycloundecaphane 4,4-dioxide;
6-ethyl-2³-(trifluoromethyl)-4-thia-3,6-diaza-2,5(2,6)-dipyridina-1(1,2)-benzenacycloundecaphane 4,4-dioxide;
6-(4-hydroxybutyl)-2³-methyl-4-thia-3,6-diaza-2,5(2,6)-dipyridina-1(1,2)-benzenacycloundecaphane 4,4-dioxide;
3-(1⁵-fluoro-4,4-dioxido-2³-(trifluoromethyl)-4-thia-3,6-diaza-2,5(2,6)-dipyridina-1(1,2)-benzenacyclododecaphane-6-yl)propanoic acid;
4-(1⁵-fluoro-4,4-dioxido-2³-(trifluoromethyl)-4-thia-3,6-diaza-2,5(2,6)-dipyridina-1(1,2)-benzenacycloundecaphane-6-yl)butanoic acid;
6-(3-hydroxypropyl)-2³-(trifluoromethyl)-4-thia-3,6-diaza-2,5(2,6)-dipyridina-1(1,2)-benzenacyclodecaphane 4,4-dioxide;
3-(2³-(difluoromethyl)-4,4-dioxido-4-thia-3,6-diaza-2,5(2,6)-dipyridina-1(1,2)-benzenacyclodecaphane-6-yl)propanoic acid;
6-(3-hydroxypropyl)-2³-(trifluoromethyl)-4-thia-3,6-diaza-2,5(2,6)-dipyridina-1(1,2)-benzenacyclododecaphane 4,4-dioxide;
4-(4,4-dioxido-2³-(trifluoromethyl)-9-oxa-4-thia-3,6-diaza-2,5(2,6)-dipyridina-1(1,2)-benzenacyclododecaphane-6-yl)butanoic acid;
3-(2³-chloro-4,4-dioxido-4-thia-3,6-diaza-2,5(2,6)-dipyridina-1(1,2)-benzenacycloundecaphane-6-yl)propanoic acid;
4-(4,4-dioxido-2³-(trifluoromethyl)-4-thia-3,6-diaza-2,5(2,6)-dipyridina-1(1,2)-benzenacyclodecaphane-6-yl)butanoic acid;
1⁵-fluoro-6-(3-hydroxypropyl)-2³-(trifluoromethyl)-11-oxa-4-thia-3,6-diaza-2,5(2,6)-dipyridina-1(1,2)-benzenacycloundecaphane 4,4-dioxide;
4-(2³-chloro-4,4-dioxido-4-thia-3,6-diaza-2,5(2,6)-dipyridina-1(1,2)-benzenacyclodecaphane-6-yl)butanoic acid;
1⁵-fluoro-6-(4-hydroxybutyl)-2³-(trifluoromethyl)-11-oxa-4-thia-3,6-diaza-2,5(2,6)-dipyridina-1(1,2)-benzenacycloundecaphane 4,4-dioxide;
3-(4,4-dioxido-2³-(trifluoromethyl)-4-thia-3,6-diaza-2,5(2,6)-dipyridina-1(1,2)-benzenacycloundecaphane-6-yl)propanoic acid;
2³-chloro-6-(3-hydroxypropyl)-4-thia-3,6-diaza-2,5(2,6)-dipyridina-1(1,2)-benzenacycloundecaphane 4,4-dioxide;
2³-(trifluoromethyl)-6,12-dioxa-4-thia-3-aza-1(3,2),2,5(2,6)-tripyridinacyclododecaphane 4,4-dioxide;
2³-chloro-4-thia-3,6,12-triaza-1(3,2),2.5(2,6)-tripyridinacyclododecaphane 4,4-dioxide;
6-(4-hydroxybutyl)-2³-(trifluoromethyl)-4-thia-3,6-diaza-2,5(2,6)-dipyridina-1(1,2)-benzenacycloundecaphane 4,4-dioxide;
3-(4,4-dioxido-4-thia-3,6-diaza-2,5(2,6)-dipyridina-1(1,2)-benzenacycloundecaphane-6-yl)propanoic acid;
1-((4,4-dioxido-2³-(trifluoromethyl)-4-thia-3,6-diaza-2,5(2,6)-dipyridina-1(1,2)-benzenacycloundecaphane-6-yl)methyl)cyclopropane-1-carboxylic acid;
1-((4,4-dioxido-2³-(trifluoromethyl)-9-oxa-4-thia-3,6-diaza-2,5(2,6)-dipyridina-1(1,2)-benzenacyclododecaphane-6-yl)methyl)cyclopropane-1-carboxylic acid;
3-(1⁵-fluoro-4,4-dioxido-2³-(trifluoromethyl)-4-thia-3,6-diaza-2,5(2,6)-dipyridina-1(1,2)-benzenacycloundecaphane-6-yl)-2,2-dimethylpropanoic acid;
3-(1⁵-fluoro-4,4-dioxido-2³-(trifluoromethyl)-4-thia-3,6-diaza-2,5(2,6)-dipyridina-1(1,2)-benzenacycloundecaphane-6-yl)propanoic acid;
6-(2-hydroxyethyl)-2³-(trifluoromethyl)-4-thia-3,6-diaza-2,5(2,6)-dipyridina-1(1,2)-benzenacyclodecaphane 4,4-dioxide;
6-(2-hydroxyethyl)-2³-(trifluoromethyl)-11-oxa-4-thia-3,6-diaza-2,5(2,6)-dipyridina-1(1,2)-benzenacycloundecaphane 4,4-dioxide;
3-(4,4-dioxido-2³-(trifluoromethyl)-9-oxa-4-thia-3,6-diaza-2,5(2,6)-dipyridina-1(1,2)-benzenacyclododecaphane-6-yl)propanoic acid;
2³-chloro-14-methyl-4-thia-3,6,12-triaza-1(3,2),2,5(2,6)-tripyridinacyclododecaphane 4,4-dioxide;
2³-(trifluoromethyl)-4-thia-3,6,12-triaza-1(3,2),2,5(2,6)-tripyridinacyclododecaphane 4,4-dioxide;
2³-(trifluoromethyl)-4-thia-3,6,13-triaza-1(3,2),2,5(2,6)-tripyridinacyclotridecaphane 4,4-dioxide;
3-(4,4-dioxido-2³-(trifluoromethyl)-4-thia-3,6-diaza-2,5(2,6)-dipyridina-1(1,2)-benzenacyclododecaphane-6-yl)-2,2-dimethylpropanoic acid;
1⁵-fluoro-6-(3-hydroxypropyl)-2³-(trifluoromethyl)-4-thia-3,6-diaza-2,5(2,6)-dipyridina-1(1,2)-benzenacycloundecaphane 4,4-dioxide;
6-(3-hydroxy-2,2-dimethylpropyl)-2³-(trifluoromethyl)-4-thia-3,6-diaza-2,5(2,6)-dipyridina-1(1,2)-benzenacycloundecaphane 4,4-dioxide;
3-(4,4-dioxido-2³-(trifluoromethyl)-4-thia-3,6-diaza-2,5(2,6)-dipyridina-1(1,2)-benzenacycloundecaphane-6-yl)-2,2-dimethylpropanoic acid;
6-(3-hydroxy-2,2-dimethylpropyl)-2³-(trifluoromethyl)-4-thia-3,6-diaza-2,5(2,6)-dipyridina-1(1,2)-benzenacyclododecaphane 4,4-dioxide;
6,13-dimethyl-2³-(trifluoromethyl)-4-thia-3,6,13-triaza-1(3,2),2,5(2,6)-tripyridinacyclotridecaphane 4,4-dioxide;
2³-chloro-12-oxa-4-thia-3,6-diaza-1(3,2),2,5(2.6)-tripyridinacyclododecaphane 4,4-dioxide;
6-(2-hydroxyethyl)-2³-(trifluoromethyl)-4-thia-3,6-diaza-2,5(2,6)-dipyridina-1(1,2)-benzenacycloundecaphane 4,4-dioxide;
6-(3-hydroxypropyl)-2³-(trifluoromethyl)-4-thia-3,6-diaza-2,5(2,6)-dipyridina-1(1,2)-benzenacycloundecaphane 4,4-dioxide;
3-(4,4-dioxido-2³-(trifluoromethyl)-6-oxa-4-thia-3-aza-2,5(2,6)-dipyridina-1(1,2)-benzenacycloundecaphane-7-yl)propanoic acid;
6,10-dimethyl-2³-(trifluoromethyl)-4-thia-3,6,10-triaza-1(3,2),2,5(2,6)-tripyridinacyclodecaphane 4,4-dioxide;
(4¹s,4⁵s)-1³-(trifluoromethyl)-3,5-dioxa-7-thia-8-aza-1,6(2,6),2(3,2)-tripyridina-4(1,5)-cyclooctanacyclooctaphane 7,7-dioxide;
6-(3-hydroxypropyl)-2³-(trifluoromethyl)-9-oxa-4-thia-3,6-diaza-2,5(2,6)-dipyridina-1(1,2)-benzenacyclododecaphane 4,4-dioxide;
1⁵-fluoro-2³-(trifluoromethyl)-4-thia-3,6-diaza-2,5(2,6)-dipyridina-1(1,2)-benzenacycloundecaphane 4,4-dioxide;
2³-(trifluoromethyl)-4-thia-3,6,10-triaza-1(3,2),2,5(2,6)-tripyridinacyclodecaphane 4,4-dioxide;
7-(3-hydroxypropyl)-2³-(trifluoromethyl)-6-oxa-4-thia-3-aza-2,5(2,6)-dipyridina-1(1,2)-benzenacycloundecaphane 4,4-dioxide;
ethyl 3-(4,4-dioxido-2³-(trifluoromethyl)-4-thia-3,6-diaza-2,5(2,6)-dipyridina-1(1,2)-benzenacyclododecaphane-6-yl)-2,2-dimethylpropanoate;
2³-(trifluoromethyl)-4-thia-3,6-diaza-2,5(2,6)-dipyridina-1(1,2)-benzenacyclodecaphane 4,4-dioxide;
2-(4,4-dioxido-2³-(trifluoromethyl)-4-thia-3,6-diaza-2,5(2,6)-dipyridina-1(1,2)-benzenacyclododecaphane-6-yl)acetic acid;
2³-(trifluoromethyl)-6-((2S,3S)-2,3,4-trihydroxybutyl)-4-thia-3,6-diaza-2,5(2,6)-dipyridina-1(1,2)-benzenacycloundecaphane 4,4-dioxide;
2³-chloro-6-oxa-4-thia-3-aza-2,5(2,6)-dipyridina-1(1,2)-benzenacyclododecaphane 4,4-dioxide;
2³-(trifluoromethyl)-4-thia-3,6-diaza-2,5(2,6)-dipyridina-1(1,2)-benzenacycloundecaphane 4,4-dioxide;
8-hydroxy-2³-(trifluoromethyl)-4-thia-3,6-diaza-2,5(2,6)-dipyridina-1(1,2)-benzenacyclodecaphane 4,4-dioxide;
6-(2-(piperazin-1-yl)ethyl)-2³-(trifluoromethyl)-4-thia-3,6-diaza-2,5(2,6)-dipyridina-1(1,2)-benzenacycloundecaphane 4,4-dioxide;
6-(2-(4-methylpiperazin-1-yl)ethyl)-2³-(trifluoromethyl)-4-thia-3,6-diaza-2,5(2,6)-dipyridina-1(1,2)-benzenacycloundecaphane 4,4-dioxide;
(2-(4,4-dioxido-2³-(trifluoromethyl)-4-thia-3,6-diaza-2,5(2,6)-dipyridina-1(1,2)-benzenacycloundecaphane-6-yl)ethyl)glycine;
6-(2-(3-hydroxyazetidin-1-yl)ethyl)-2³-(trifluoromethyl)-4-thia-3,6-diaza-2,5(2,6)-dipyridina-1(1,2)-benzenacycloundecaphane 4,4-dioxide;
²³-(trifluoromethyl)-9-oxa-4-thia-3,6-diaza-2,5(2,6)-dipyridina-1(1,2)-benzenacyclododecaphane 4,4-dioxide;
6-(2-((3S,4S)-3,4-dihydroxypyrrolidin-1-yl)ethyl)-2³-(trifluoromethyl)-4-thia-3,6-diaza-2,5(2,6)-dipyridina-1(1,2)-benzenacycloundecaphane 4,4-dioxide;
6-(((4S,5S)-5-(hydroxymethyl)-2,2-dimethyl-1,3-dioxolan-4-yl)methyl)-2³-(trifluoromethyl)-4-thia-3,6-diaza-2,5(2,6)-dipyridina-1(1,2)-benzenacycloundecaphane 4,4-dioxide;
2³-chloro-6-oxa-4-thia-3-aza-2,5(2,6)-dipyridina-1(1,2)-benzenacyclodecaphane 4,4-dioxide;
6-methyl-2³-(trifluoromethyl)-4-thia-3,6-diaza-2,5(2,6)-dipyridina-1(1,2)-benzenacyclododecaphane 4,4-dioxide;
2³-(trifluoromethyl)-4-thia-3,6-diaza-2,5(2,6)-dipyridina-1(1,2)-benzenacyclododecaphane 4,4-dioxide;
ethyl 2-(4,4-dioxido-2³-(trifluoromethyl)-11-oxa-4-thia-3,6-diaza-2,5(2,6)-dipyridina-1(1,2)-benzenacycloundecaphane-6-yl)acetate;
6-(2,3-dihydroxypropyl)-2³-(trifluoromethyl)-4-thia-3,6-diaza-2,5(2,6)-dipyridina-1(1,2)-benzenacycloundecaphane 4,4-dioxide;
6-(2-(pyrrolidin-1-yl)ethyl)-2³-(trifluoromethyl)-4-thia-3,6-diaza-2,5(2,6)-dipyridina-1(1,2)-benzenacycloundecaphane 4,4-dioxide;
methyl (2-(4,4-dioxido-2³-(trifluoromethyl)-4-thia-3,6-diaza-2,5(2,6)-dipyridina-1(1,2)-benzenacycloundecaphane-6-yl)ethyl)glycinate;
6-((2,2-dimethyl-1,3-dioxolan-4-yl)methyl)-2³-(trifluoromethyl)-4-thia-3,6-diaza-2,5(2,6)-dipyridina-1(1,2)-benzenacycloundecaphane 4,4-dioxide;
2³-chloro-6-oxa-4-thia-3-aza-2,5(2,6)-dipyridina-1(1,2)-benzenacycloundecaphane 4,4-dioxide;
6-(2-aminoethyl)-2³-(trifluoromethyl)-4-thia-3,6-diaza-2,5(2,6)-dipyridina-1(1,2)-benzenacyclodecaphane 4,4-dioxide;
2³-(trifluoromethyl)-4-thia-3,6,9-triaza-2,5(2,6)-dipyridina-1(1,2)-benzenacyclotridecaphane 4,4-dioxide;
2³-chloro-6-oxa-4-thia-3-aza-2,5(2,6)-dipyridina-1(1,2)-benzenacyclononaphane 4,4-dioxide;
8-hydroxy-2³-(trifluoromethyl)-4-thia-3,6-diaza-2,5(2,6)-dipyridina-1(1,2)-benzenacycloundecaphane 4,4-dioxide;
6-(2-aminoethyl)-2³-(difluoromethyl)-4-thia-3,6-diaza-2,5(2,6)-dipyridina-1(1,2)-benzenacycloundecaphane 4,4-dioxide;
2-(3-(4,4-dioxido-2³-(trifluoromethyl)-4-thia-3,6-diaza-2,5(2,6)-dipyridina-1(1,2)-benzenacycloundecaphane-6-yl)propyl)isoindoline-1,3-dione;
2-(3-(4,4-dioxido-2³-(trifluoromethyl)-4-thia-3,6-diaza-2,5(2,6)-dipyridina-1(1,2)-benzenacycloundecaphane-6-yl)propyl)hexahydro-1H-isoindole-1,3(2H)-dione;
methyl 2-(4,4-dioxido-2³-(trifluoromethyl)-4-thia-3,6-diaza-2,5(2,6)-dipyridina-1(1,2)-benzenacyclododecaphane-6-yl)acetate 6-(4-hydroxybutyl)-2³-(trifluoromethyl)-4-thia-3,6-diaza-2,5(2,6)-dipyridina-1(1,2)-benzenacyclotridecaphane 4,4-dioxide;
6-(5-hydroxypentyl)-2³-(trifluoromethyl)-4-thia-3,6-diaza-2,5(2,6)-dipyridina-1(1,2)-benzenacyclotridecaphane 4,4-dioxide;
6-(4-hydroxybutyl)-2³-(trifluoromethyl)-4-thia-3,6-diaza-2,5(2,6)-dipyridina-1(1,2)-benzenacyclopentadecaphane 4,4-dioxide;
6-(3-hydroxypropyl)-2³-(trifluoromethyl)-4-thia-3,6-diaza-2,5(2,6)-dipyridina-1(1,2)-benzenacyclotetradecaphane 4,4-dioxide;
6-(4-hydroxybutyl)-2³-(trifluoromethyl)-4-thia-3,6-diaza-2,5(2,6)-dipyridina-1(1,2)-benzenacyclotetradecaphane 4,4-dioxide;
6-(3-hydroxypropyl)-2³-(trifluoromethyl)-4-thia-3,6-diaza-2,5(2,6)-dipyridina-1(1,2)-benzenacyclopentadecaphane 4,4-dioxide;
2³-chloro-6-(4-hydroxybutyl)-4-thia-3,6-diaza-2,5(2,6)-dipyridina-1(1,2)-benzenacycloundecaphane 4,4-dioxide;
1⁵-fluoro-6-(4-hydroxybutyl)-2³-(trifluoromethyl)-4-thia-3,6-diaza-2,5(2,6)-dipyridina-1(1,2)-benzenacyclododecaphane 4,4-dioxide;
6-(6-hydroxyhexyl)-2³-(trifluoromethyl)-4-thia-3,6-diaza-2,5(2,6)-dipyridina-1(1,2)-benzenacycloundecaphane 4,4-dioxide;
6-(2-(2-hydroxyethoxy)ethyl)-2³-(trifluoromethyl)-4-thia-3,6-diaza-2,5(2,6)-dipyridina-1(1,2)-benzenacycloundecaphane 4,4-dioxide;
6-(5-hydroxypentyl)-2³-(trifluoromethyl)-4-thia-3,6-diaza-2,5(2,6)-dipyridina-1(1,2)-benzenacyclodecaphane 4,4-dioxide;
2³-chloro-6-(6-hydroxyhexyl)-4-thia-3,6-diaza-2,5(2,6)-dipyridina-1(1,2)-benzenacycloundecaphane 4,4-dioxide;
1⁵-fluoro-6-(3-hydroxypropyl)-2³-(trifluoromethyl)-4-thia-3,6-diaza-2,5(2,6)-dipyridina-1(1,2)-benzenacyclododecaphane 4,4-dioxide;
1⁵-fluoro-6-(4-hydroxybutyl)-2³-(trifluoromethyl)-4-thia-3,6-diaza-2,5(2,6)-dipyridina-1(1,2)-benzenacycloundecaphane 4,4-dioxide;
6-(4-hydroxybutyl)-2³-(trifluoromethyl)-9-oxa-4-thia-3,6-diaza-2,5(2,6)-dipyridina-1(1,2)-benzenacyclododecaphane 4,4-dioxide;
6-(4-hydroxybutyl)-2³-(trifluoromethyl)-4-thia-3,6-diaza-2,5(2,6)-dipyridina-1(1,2)-benzenacyclodecaphane 4,4-dioxide;
2³-chloro-6-(4-hydroxybutyl)-4-thia-3,6-diaza-2,5(2,6)-dipyridina-1(1,2)-benzenacyclodecaphane 4,4-dioxide;
6-(3-hydroxypropyl)-4-thia-3,6-diaza-2,5(2,6)-dipyridina-1(1,2)-benzenacycloundecaphane 4,4-dioxide;
6-((1-(hydroxymethyl)cyclopropyl)methyl)-2³-(trifluoromethyl)-4-thia-3,6-diaza-2,5(2,6)-dipyridina-1(1,2)-benzenacycloundecaphane 4,4-dioxide;
6-((1-(hydroxymethyl)cyclopropyl)methyl)-2³-(trifluoromethyl)-9-oxa-4-thia-3,6-diaza-2,5(2,6)-dipyridina-1(1,2)-benzenacyclododecaphane 4,4-dioxide;
1⁵-fluoro-6-(3-hydroxy-2,2-dimethylpropyl)-2³-(trifluoromethyl)-4-thia-3,6-diaza-2,5(2,6)-dipyridina-1(1,2)-benzenacycloundecaphane 4,4-dioxide;
3-(4,4-dioxido-2³-(trifluoromethyl)-4-thia-3,6-diaza-2,5(2,6)-dipyridina-1(1,2)-benzenacyclodecaphane-6-yl)propanoic acid;
5-(4,4-dioxido-2³-(trifluoromethyl)-4-thia-3,6-diaza-2,5(2,6)-dipyridina-1(1,2)-benzenacyclotridecaphane-6-yl)pentanoic acid;
6-(2-aminoethyl)-2³-(trifluoromethyl)-4-thia-3,6-diaza-2,5(2,6)-dipyridina-1(1,2)-benzenacycloundecaphane 4,4-dioxide, and
(R)-3-(4,4-dioxido-2³-(trifluoromethyl)-4-thia-3,6-diaza-2,5(2,6)-dipyridina-1(1,2)-benzenacycloundecaphane-7-yl)propanoic acid.
   Embodiment 117. The compound of any one of Embodiments 5 to 12, selected from:
4-(1⁵-fluoro-4,4-dioxido-2³-(trifluoromethyl)-11-oxa-4-thia-3,6-diaza-2,5(2,6)-dipyridina-1(1,2)-benzenacycloundecaphane-6-yl)butanoic acid;
4-(4,4-dioxido-2³-(trifluoromethyl)-4-thia-3,6-diaza-2,5(2,6)-dipyridina-1(1,2)-benzenacyclotridecaphane-6-yl)butanoic acid;
4-(4,4-dioxido-2³-(trifluoromethyl)-4-thia-3,6-diaza-2,5(2,6)-dipyridina-1(1,2)-benzenacyclopentadecaphane-6-yl)butanoic acid;
4-(4,4-dioxido-2³-(trifluoromethyl)-4-thia-3,6-diaza-2,5(2,6)-dipyridina-1(1,2)-benzenacycloundecaphane-6-yl)butanoic acid;
4-(1⁵-fluoro-4,4-dioxido-2³-(trifluoromethyl)-4-thia-3,6-diaza-2,5(2,6)-dipyridina-1(1,2)-benzenacyclododecaphane-6-yl)butanoic acid;
3-(4,4-dioxido-2³-(trifluoromethyl)-4-thia-3,6-diaza-2,5(2,6)-dipyridina-1(1,2)-benzenacyclotetradecaphane-6-yl)propanoic acid;
4-(4,4-dioxido-2³-(trifluoromethyl)-4-thia-3,6-diaza-2,5(2,6)-dipyridina-1(1,2)-benzenacyclotetradecaphane-6-yl)butanoic acid;
3-(4,4-dioxido-2³-(trifluoromethyl)-4-thia-3,6-diaza-2,5(2,6)-dipyridina-1(1,2)-benzenacyclotridecaphane-6-yl)propanoic acid;
4-(4,4-dioxido-2³-(trifluoromethyl)-10-oxa-4-thia-3,6-diaza-2,5(2,6)-dipyridina-1(1,2)-benzenacycloundecaphane-6-yl)butanoic acid;
3-(4,4-dioxido-2³-(trifluoromethyl)-4-thia-3,6-diaza-2,5(2,6)-dipyridina-1(1,2)-benzenacyclopentadecaphane-6-yl)propanoic acid;
4-(4,4-dioxido-2³-(trifluoromethyl)-4-thia-3,6-diaza-2,5(2,6)-dipyridina-1(1,2)-benzenacyclododecaphane-6-yl)butanoic acid;
5-(4,4-dioxido-2³-(trifluoromethyl)-4-thia-3,6-diaza-2,5(2,6)-dipyridina-1(1,2)-benzenacycloundecaphane-6-yl)pentanoic acid;
5-(4,4-dioxido-2³-(trifluoromethyl)-4-thia-3,6-diaza-2,5(2,6)-dipyridina-1(1,2)-benzenacyclododecaphane-6-yl)pentanoic acid;
4-(2³-chloro-4,4-dioxido-4-thia-3,6-diaza-2,5(2,6)-dipyridina-1(1,2)-benzenacycloundecaphane-6-yl)butanoic acid;
5-(4,4-dioxido-2³-(trifluoromethyl)-4-thia-3,6-diaza-2,5(2,6)-dipyridina-1(1,2)-benzenacyclodecaphane-6-yl)pentanoic acid;
6-(5-hydroxypentyl)-2³-(trifluoromethyl)-4-thia-3,6-diaza-2,5(2,6)-dipyridina-1(1,2)-benzenacyclododecaphane 4,4-dioxide;
3-(4,4-dioxido-2³-(trifluoromethyl)-4-thia-3,6-diaza-2,5(2,6)-dipyridina-1(1,2)-benzenacyclododecaphane-6-yl)propanoic acid.
6-(4-hydroxybutyl)-2³-(trifluoromethyl)-4-thia-3,6-diaza-2,5(2,6)-dipyridina-1(1,2)-benzenacyclododecaphane 4,4-dioxide;
3-(1⁵-fluoro-4,4-dioxido-2³-(trifluoromethyl)-4-thia-3,6-diaza-2,5(2,6)-dipyridina-1(1,2)-benzenacyclododecaphane-6-yl)propanoic acid;
4-(1⁵-fluoro-4,4-dioxido-2³-(trifluoromethyl)-4-thia-3,6-diaza-2,5(2,6)-dipyridina-1(1,2)-benzenacycloundecaphane-6-yl)butanoic acid;
4-(4,4-dioxido-2³-(trifluoromethyl)-9-oxa-4-thia-3,6-diaza-2,5(2,6)-dipyridina-1(1,2)-benzenacyclododecaphane-6-yl)butanoic acid;
1-((4,4-dioxido-2³-(trifluoromethyl)-9-oxa-4-thia-3,6-diaza-2,5(2,6)-dipyridina-1(1,2)-benzenacyclododecaphane-6-yl)methyl)cyclopropane-1-carboxylic acid;
3-(4,4-dioxido-2³-(trifluoromethyl)-4-thia-3,6-diaza-2,5(2,6)-dipyridina-1(1,2)-benzenacyclododecaphane-6-yl)-2,2-dimethylpropanoic acid;
1⁵-fluoro-6-(3-hydroxypropyl)-2³-(trifluoromethyl)-4-thia-3,6-diaza-2,5(2,6)-dipyridina-1(1,2)-benzenacycloundecaphane 4,4-dioxide;
3-(4,4-dioxido-2³-(trifluoromethyl)-4-thia-3,6-diaza-2,5(2,6)-dipyridina-1(1,2)-benzenacycloundecaphane-6-yl)-2,2-dimethylpropanoic acid;
6-(3-hydroxy-2,2-dimethylpropyl)-2³-(trifluoromethyl)-4-thia-3,6-diaza-2,5(2,6)-dipyridina-1(1,2)-benzenacyclododecaphane 4,4-dioxide;
6-(2-hydroxyethyl)-2³-(trifluoromethyl)-4-thia-3,6-diaza-2,5(2,6)-dipyridina-1(1,2)-benzenacycloundecaphane 4,4-dioxide;
6-(3-hydroxypropyl)-2³-(trifluoromethyl)-9-oxa-4-thia-3,6-diaza-2,5(2,6)-dipyridina-1(1,2)-benzenacyclododecaphane 4,4-dioxide;
ethyl 3-(4,4-dioxido-2³-(trifluoromethyl)-4-thia-3,6-diaza-2,5(2,6)-dipyridina-1(1,2)-benzenacyclododecaphane-6-yl)-2,2-dimethylpropanoate;
2³-chloro-6-oxa-4-thia-3-aza-2,5(2,6)-dipyridina-1(1,2)-benzenacyclododecaphane 4,4-dioxide, and
methyl 2-(4,4-dioxido-2³-(trifluoromethyl)-4-thia-3,6-diaza-2,5(2,6)-dipyridina-1(1,2)-benzenacyclododecaphane-6-yl)acetate.
   Embodiment 118. The compound of any one of Embodiments 5 to 12, selected from:
4-(4,4-dioxido-2³-(trifluoromethyl)-4-thia-3,6-diaza-2,5(2,6)-dipyridina-1(1,2)-benzenacyclotridecaphane-6-yl)butanoic acid;
4-(4,4-dioxido-2³-(trifluoromethyl)-4-thia-3,6-diaza-2,5(2,6)-dipyridina-1(1,2)-benzenacyclopentadecaphane-6-yl)butanoic acid;
4-(4,4-dioxido-2³-(trifluoromethyl)-4-thia-3,6-diaza-2,5(2,6)-dipyridina-1(1,2)-benzenacycloundecaphane-6-yl)butanoic acid;
3-(4,4-dioxido-2³-(trifluoromethyl)-4-thia-3,6-diaza-2,5(2,6)-dipyridina-1(1,2)-benzenacyclotetradecaphane-6-yl)propanoic acid;
4-(4,4-dioxido-2³-(trifluoromethyl)-4-thia-3,6-diaza-2,5(2,6)-dipyridina-1(1,2)-benzenacyclotetradecaphane-6-yl)butanoic acid;
3-(4,4-dioxido-2³-(trifluoromethyl)-4-thia-3,6-diaza-2,5(2,6)-dipyridina-1(1,2)-benzenacyclotridecaphane-6-yl)propanoic acid;
3-(4,4-dioxido-2³-(trifluoromethyl)-4-thia-3,6-diaza-2,5(2,6)-dipyridina-1(1,2)-benzenacyclopentadecaphane-6-yl)propanoic acid;
4-(4,4-dioxido-2³-(trifluoromethyl)-4-thia-3,6-diaza-2,5(2,6)-dipyridina-1(1,2)-benzenacyclododecaphane-6-yl)butanoic acid;
5-(4,4-dioxido-2³-(trifluoromethyl)-4-thia-3,6-diaza-2,5(2,6)-dipyridina-1(1,2)-benzenacycloundecaphane-6-yl)pentanoic acid;
5-(4,4-dioxido-2³-(trifluoromethyl)-4-thia-3,6-diaza-2,5(2,6)-dipyridina-1(1,2)-benzenacyclododecaphane-6-yl)pentanoic acid;
5-(4,4-dioxido-2³-(trifluoromethyl)-4-thia-3,6-diaza-2,5(2,6)-dipyridina-1(1,2)-benzenacyclodecaphane-6-yl)pentanoic acid, and
3-(4,4-dioxido-2³-(trifluoromethyl)-4-thia-3,6-diaza-2,5(2,6)-dipyridina-1(1,2)-benzenacyclododecaphane-6-yl)propanoic acid.
   Embodiment 119. The compound of any one of Embodiments 5 to 12, selected from:
4-(4,4-dioxido-2³-(trifluoromethyl)-4-thia-3,6-diaza-2,5(2,6)-dipyridina-1(1,2)-benzenacyclodecaphane-6-yl)butanoic acid;
4-(4,4-dioxido-2³-(trifluoromethyl)-4-thia-3,6-diaza-2,5(2,6)-dipyridina-1(1,2)-benzenacycloundecaphane-6-yl)butanoic acid:
4-(4,4-dioxido-2³-(trifluoromethyl)-4-thia-3,6-diaza-2,5(2,6)-dipyridina-1(1,2)-benzenacyclododecaphane-6-yl)butanoic acid;
4-(4,4-dioxido-2³-(trifluoromethyl)-4-thia-3,6-diaza-2,5(2,6)-dipyridina-1(1,2)-benzenacyclotridecaphane-6-yl)butanoic acid;
4-(4,4-dioxido-2³-(trifluoromethyl)-4-thia-3,6-diaza-2,5(2,6)-dipyridina-1(1,2)-benzenacyclotetradecaphane-6-yl)butanoic acid;
4-(4,4-dioxido-2³-(trifluoromethyl)-4-thia-3,6-diaza-2,5(2,6)-dipyridina-1(1,2)-benzenacyclopentadecaphane-6-yl)butanoic acid, and
6-(3-hydroxy-2,2-dimethylpropyl)-2³-(trifluoromethyl)-4-thia-3,6-diaza-2,5(2,6)-dipyridina-1(1,2)-benzenacyclododecaphane 4,4-dioxide.

Depending on the choice of the starting materials and procedures, the compounds can be present in the form of one of the possible stereoisomers or as mixtures thereof, for example as pure optical isomers, or as stereoisomer mixtures, such as racemates and diastereoisomer mixtures, depending on the number of asymmetric carbon atoms. The present invention is meant to include all such possible stereoisomers, including racemic mixtures, diasteriomeric mixtures and optically pure forms. Optically active (R)- and (S)-stereoisomers may be prepared using chiral synthons or chiral reagents, or resolved using conventional techniques. If the compound contains a double bond, the substituent may be E or Z configuration. If the compound contains a disubstituted cycloalkyl, the cycloalkyl substituent may have a cis- or trans-configuration. All tautomeric forms are also intended to be included.

As used herein, the terms “salt” or “salts” refers to an acid addition or base addition salt of a compound of the present invention. “Salts” include in particular “pharmaceutical acceptable salts”. The terms “pharmaceutically acceptable salt” or “pharmaceutically acceptable salts”, as used herein, refers to a salt or salts that retain the biological effectiveness and properties of the compounds of this invention and, which typically are not biologically or otherwise undesirable. In many cases, the compounds of the present invention are capable of forming acid and/or base salts by virtue of the presence of amino and/or carboxyl groups or groups similar thereto.

Pharmaceutically acceptable acid addition salts can be formed with inorganic acids and organic acids. The organic acid or inorganic acids used to form pharmaceutically acceptable acid addition salts of compounds of the present invention include, but are not limited to, acetic acid, adipic acid, ascorbic acid, aspartic acid, benzoic acid, benzenesulfonic acid, carbonic acid, camphor sulfonic acid, capric acid, chlorotheophyllinate, citric acid, ethanedisulfonic acid, fumaric acid, D-glycero-D-gulo-Heptonicacid, galactaric aid, galactaric acid/mucic acid, gluceptic acid, glucoheptonoic acid, gluconic acid, glucuronic acid, glutamatic acid, glutaric acid, glycolic acid, hippuric acid, hydrobromic acid, hydrochloric acid, hydroiodic acid, isethionic acid, lactic acid, lactobionic acid, lauryl sulfuric acid, malic acid, maleic acid, malonic acid, mandelic acid, mesylic acid, methanesulfonic acid, mucic acid, naphthoic acid, 1-hydroxy-2-naphthoic acid, naphthalenesulfonic acid, 2-naphthalenesulfonic acid, nicotinic acid, nitric acid, octadecanoic acid, oleaic acid, oxalic acid, palmitic acid, pamoic acid, phosphoric acid, polygalacturonic acid, propionic acid, sebacic acid, stearic acid, succinic acid, sulfosalicylic acid, sulfuric acid, tartaric acid, p-toluenesulfonic acid, trifluoroacetic acid and triphenylacetic acid.

Salt forms of the compounds of the present invention can be converted into the free compounds by treatment with a suitable basic agent.

Pharmaceutically acceptable acid addition salts of compounds of the present invention include, but are not limited to, a acetate, adipate, ascorbate, aspartate, benzoate, besylatye, benzenesulfonate, bicarbonate/carbonate, bisulfate/sulfate, bromide/hydrobromide, camphor sulfonate, camsylate, caprate, chloride/hydrochloride, chlorotheophyllinate, citrate, edisylate, ethanedisulfonate, fumarate, gluceptate, glucoheptonate, gluconate, glucuronate, glutamate, glutarate, glycolate, hippurate, hydroiodide/iodide, isethionate, lactate, lactobionate, laurylsulphate, malate, maleate, malonate, mandelate, mesylate, methanesulfonate, methylsulfate, mucate, naphthoate, napsylate, 2-napsylate, naphthalenesulfonate, 2-naphthalenesulfonate, nicotinate, nitrate, octadecanoate, oleate, oxalate, palmitate, pamoate, phosphate/hydrogen phosphate/dihydrogen phosphate, polygalacturonate, propionate, sebacate, stearate, succinate, sulfosalicylate, sulfate, tartrate, tosylate, p-toluenesulfonate, trifluoroacetate, trifenatate, triphenylacetete and xinafoate salt forms.

Pharmaceutically acceptable base addition salts can be formed with inorganic and organic bases. Organic bases used to form pharmaceutically acceptable base addition salts of compounds of the present invention include, but are not limited to, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine. Inorganic bases used to form pharmaceutically acceptable base addition salts of compounds of the present invention include, but are not limited to, sodium hydroxide, potassium hydroxide, ammonium hydroxide, ammonium salts and metals from columns I to XII of the periodic table. Pharmaceutically acceptable base addition salts of compounds of the present invention include, but are not limited to, sodium, potassium, ammonium, calcium, magnesium, iron, silver, zinc, and copper salts; particularly suitable salts include ammonium, potassium, sodium, calcium and magnesium salts.

In another aspect, the present invention provides 4-(4,4-dioxido-2³-(trifluoromethyl)-4-thia-3,6-diaza-2,5(2,6)-dipyridina-1(1,2)-benzenacyclodecaphane-6-yl)butanoic acid in sodium or potassium salt form.

In another aspect, the present invention provides 4-(4,4-dioxido-2³-(trifluoromethyl)-4-thia-3,6-diaza-2,5(2,6)-dipyridina-1(1,2)-benzenacycloundecaphane-6-yl)butanoic acid in sodium or potassium salt form.

In another aspect, the present invention provides 4-(4,4-dioxido-2³-(trifluoromethyl)-4-thia-3,6-diaza-2,5(2,6)-dipyridina-1(1,2)-benzenacyclododecaphane-6-yl)butanoic acid in sodium or potassium salt form.

In another aspect, the present invention provides 4-(4,4-dioxido-2³-(trifluoromethyl)-4-thia-3,6-diaza-2,5(2,6)-dipyridina-1(1,2)-benzenacyclotridecaphane-6-yl)butanoic acid in sodium or potassium salt form.

In another aspect, the present invention provides 4-(4,4-dioxido-2³-(trifluoromethyl)-4-thia-3,6-diaza-2,5(2,6)-dipyridina-1(1,2)-benzenacyclotetradecaphane-6-yl)butanoic acid in sodium or potassium salt form.

In another aspect, the present invention provides 4-(4,4-dioxido-2³-(trifluoromethyl)-4-thia-3,6-diaza-2,5(2,6)-dipyridina-1(1,2)-benzenacyclopentadecaphane-6-yl)butanoic acid in sodium or potassium salt form.

Any formula given herein is also intended to represent unlabeled forms as well as isotopically labeled forms of the compounds. Isotopically labeled compounds have structures depicted by the formulae given herein except that one or more atoms are replaced by an atom having a selected atomic mass or mass number. Isotopes that can be incorporated into compounds of the present invention include, for example, isotopes of hydrogen.

Further, incorporation of certain isotopes, particularly deuterium (i.e., ²H or D) may afford certain therapeutic advantages resulting from greater metabolic stability, for example increased in vivo half-life or reduced dosage requirements or an improvement in therapeutic index or tolerability. It is understood that deuterium in this context is regarded as a substituent of a compound of the present invention. The concentration of deuterium, may be defined by the isotopic enrichment factor. The term “isotopic enrichment factor” as used herein means the ratio between the isotopic abundance and the natural abundance of a specified isotope. If a substituent in a compound of this invention is denoted as being deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation). It should be understood that the term “isotopic enrichment factor” can be applied to any isotope in the same manner as described for deuterium.

Other examples of isotopes that can be incorporated into compounds of the present invention include isotopes of hydrogen, carbon, nitrogen, oxygen, phosphorous, fluorine, and chlorine, such as ³H, ¹¹C, ¹³C, ¹⁴C, ¹⁵N, ¹⁸F, ³¹P, ³²P, ³⁵S, ³⁶Cl, ¹²³I, ¹²⁴I, ¹²⁵I respectively. Accordingly it should be understood that the invention includes compounds that incorporate one or more of any of the aforementioned isotopes, including for example, radioactive isotopes, such as ³H and ¹⁴C, or those into which non-radioactive isotopes, such as ²H and ¹³C are present. Such isotopically labelled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F or labeled compound may be particularly desirable for PET or SPECT studies. Isotopically-labeled compounds of the present invention can generally be prepared by conventional techniques known to those skilled in the art or by processes analogous to those described in the accompanying Examples and Preparations using an appropriate isotopically-labeled reagents in place of the non-labeled reagent previously employed.

By way of example, compounds of the present invention can exist in a deuterated form as shown below:

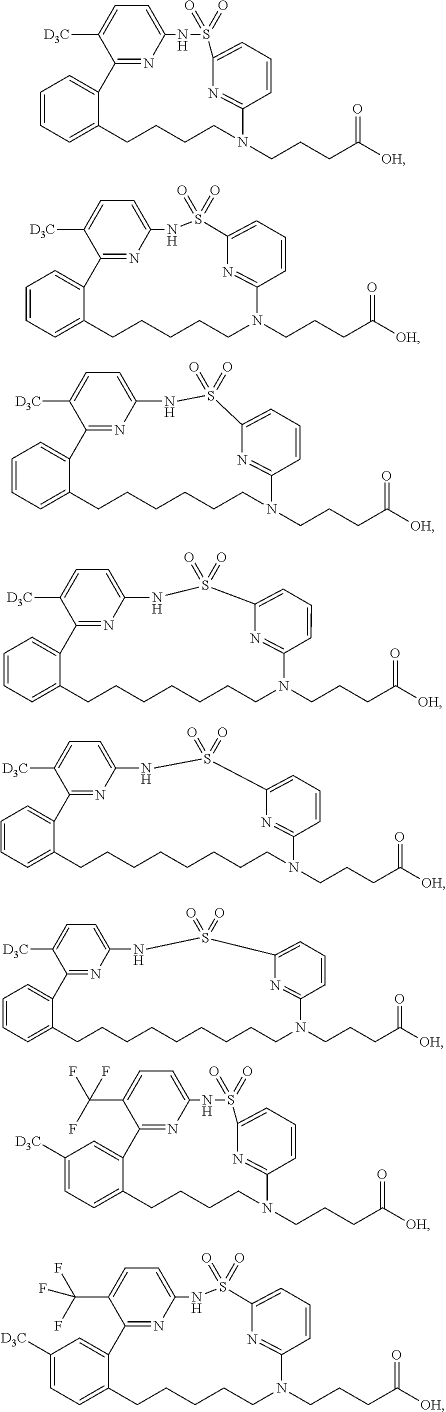

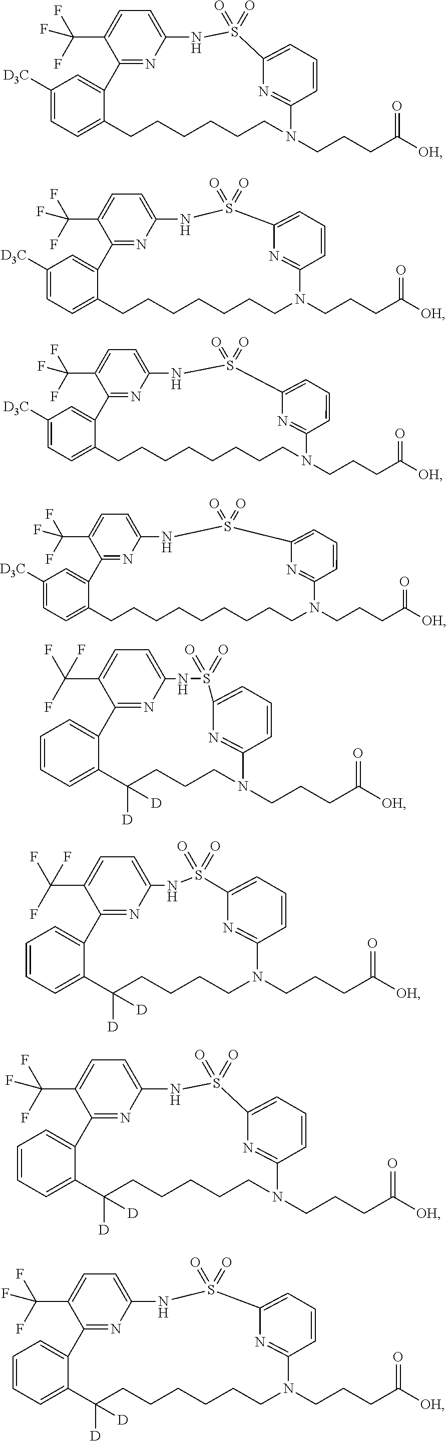

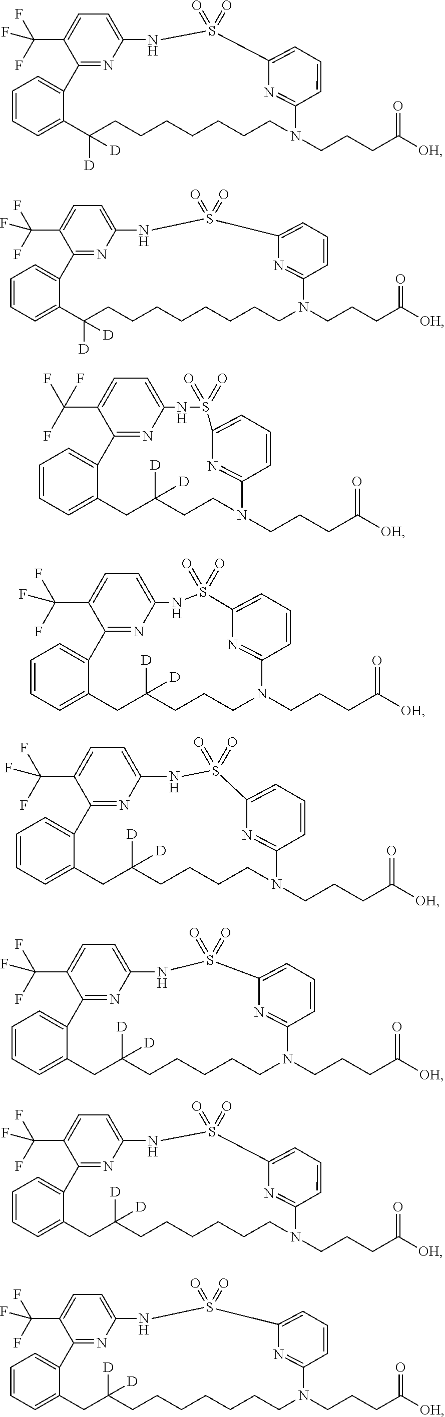

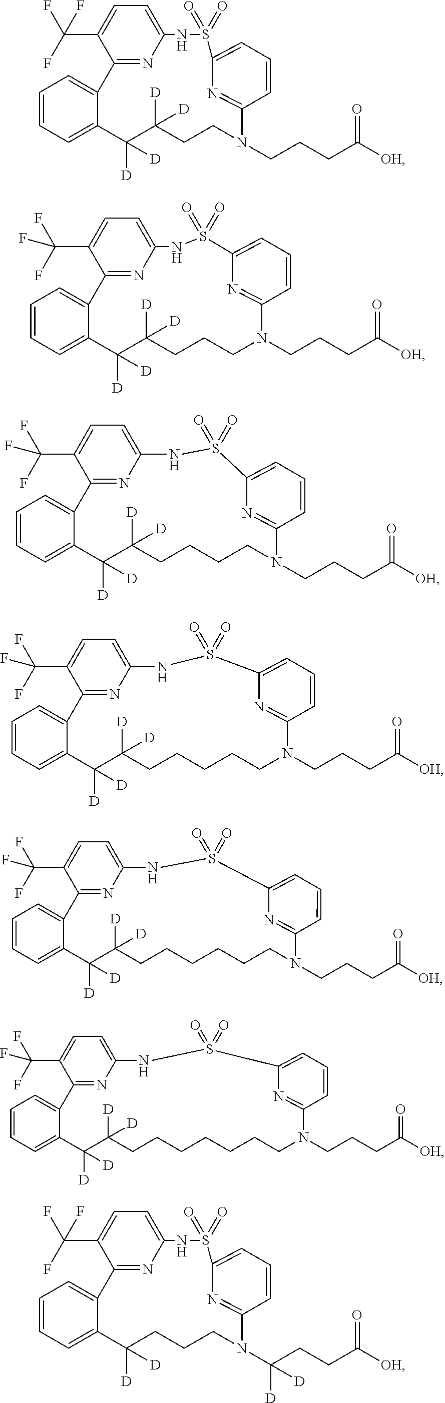

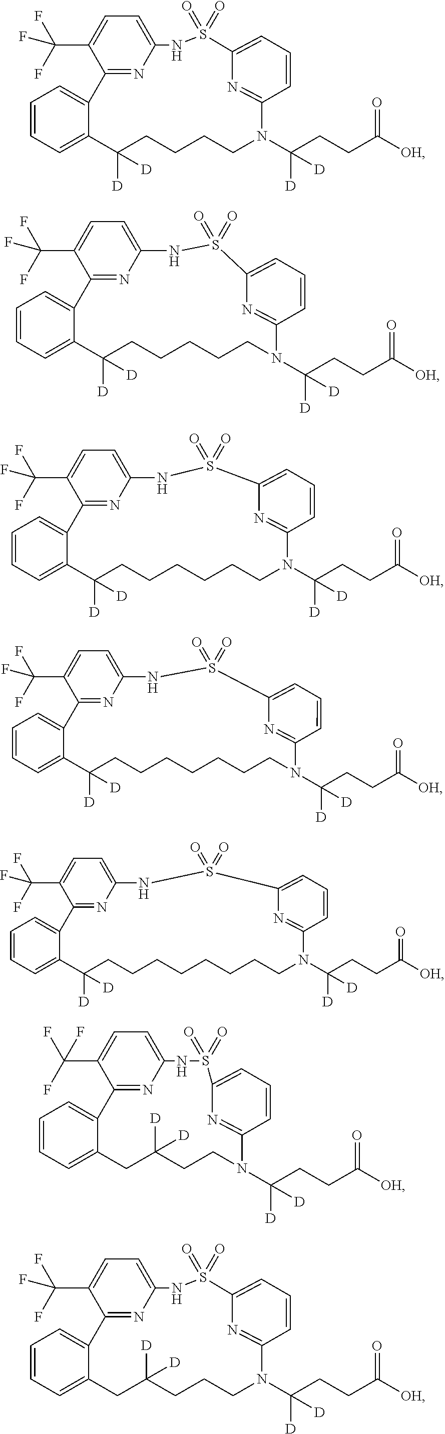

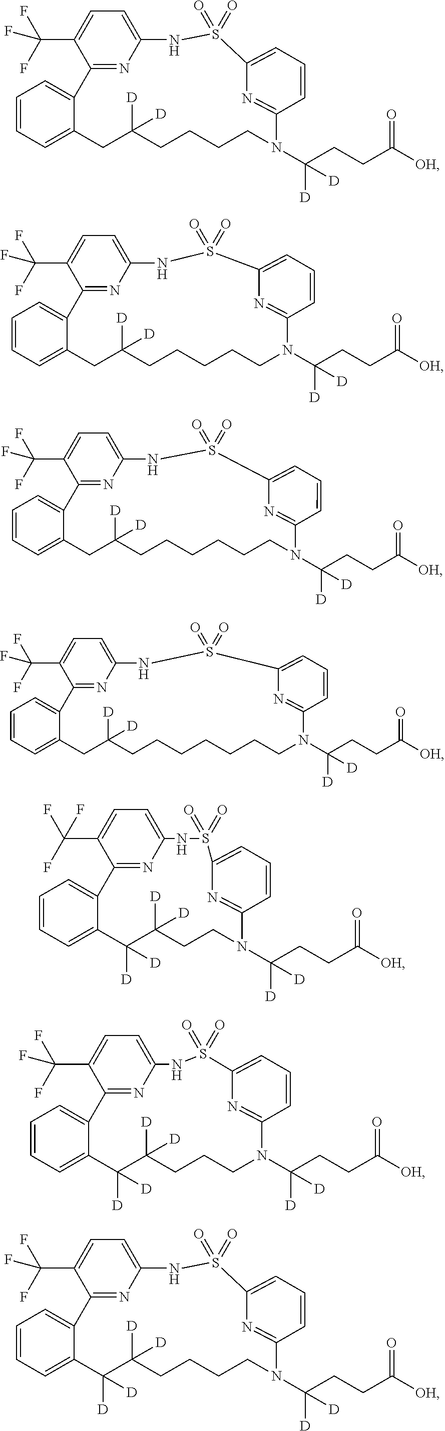

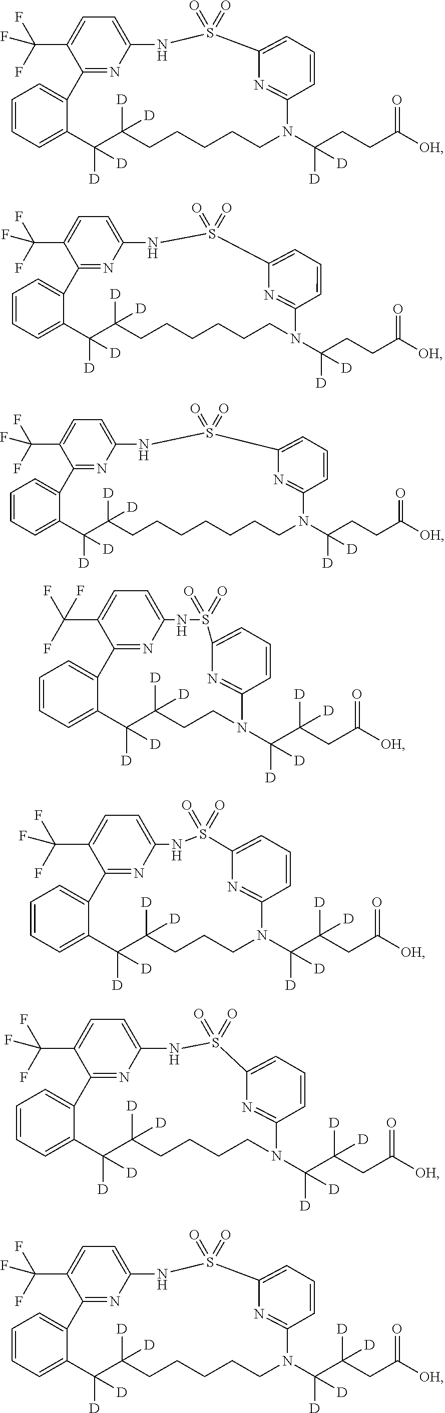

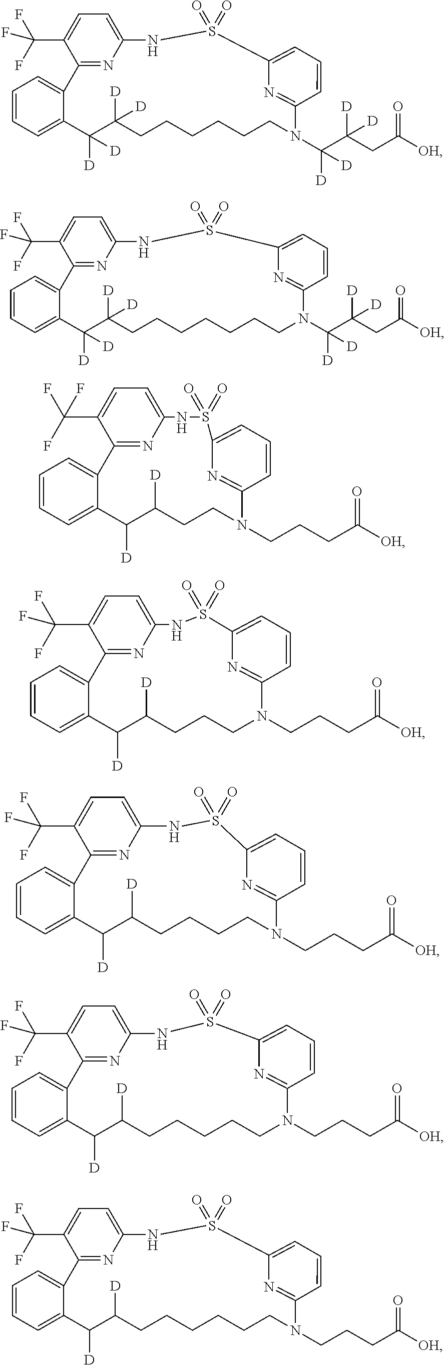

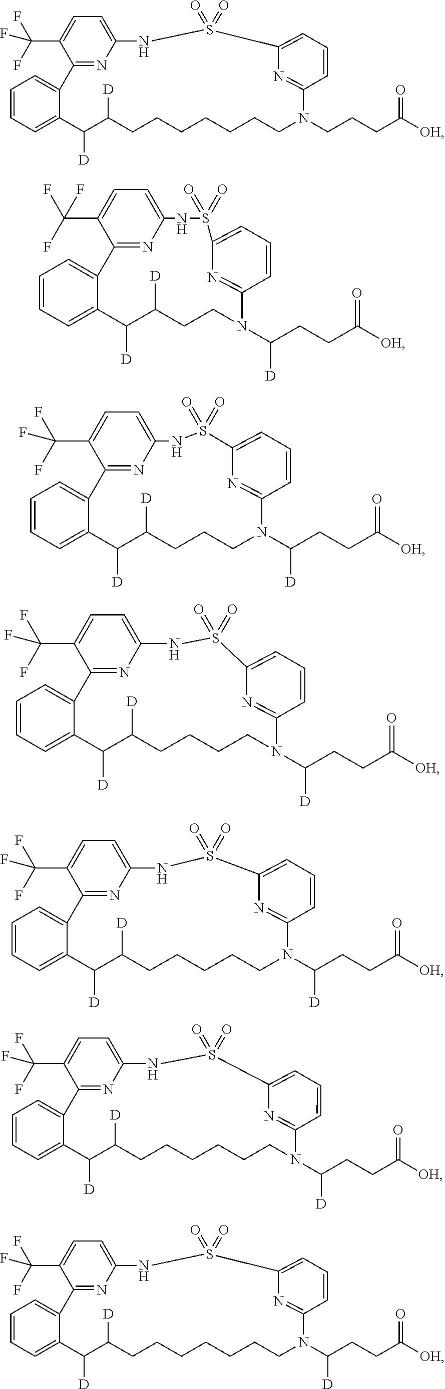

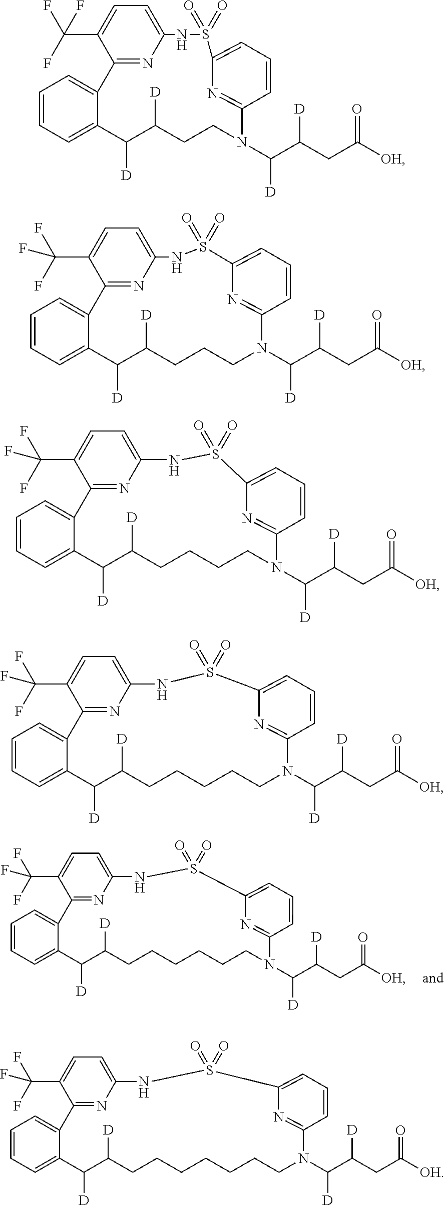

Any asymmetric atom (e.g., carbon or the like) of the compound(s) of the present invention can be present in racemic or enantiomerically enriched, for example the (R)-, (S)- or (R,S)-configuration. In certain embodiments, each asymmetric atom has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess in the (R)- or (S)-configuration. Substituents at atoms with unsaturated double bonds may, if possible, be present in cis-(Z)- or trans-(E)-form.

Accordingly, as used herein a compound of the present invention can be in the form of one of the possible isomers, rotamers, atropisomers, tautomers or mixtures thereof, for example, as substantially pure geometric (cis or trans) isomers, diastereomers, optical isomers (antipodes), racemates or mixtures thereof.

Any resulting mixtures of isomers can be separated on the basis of the physicochemical differences of the constituents, into the pure or substantially pure geometric or optical isomers, diastereomers, racemates, for example, by chromatography and/or fractional crystallization.

Any resulting racemates of final products or intermediates can be resolved into the optical antipodes by known methods, e.g., by separation of the diastereomeric salts thereof, obtained with an optically active acid or base, and liberating the optically active acidic or basic compound. In particular, a basic moiety may thus be employed to resolve the compounds of the present invention into their optical antipodes, e.g., by fractional crystallization of a salt formed with an optically active acid, e.g., tartaric acid, dibenzoyl tartaric acid, diacetyl tartaric acid, di-O,O′-p-toluoyl tartaric acid, mandelic acid, malic acid or camphor-10-sulfonic acid. Racemic products can also be resolved by chiral chromatography, e.g., high pressure liquid chromatography (HPLC) using a chiral adsorbent.

### Processes for Making Compounds of Invention

General procedures for preparing compounds of the present invention are described herein. In the reactions described, reactive functional groups, for example hydroxy, amino, imino or carboxy groups, where these are desired in the final product, may be protected to avoid their unwanted participation in the reactions. Within the scope of this text, only a readily removable group that is not a constituent of the particular desired end product of the compounds of the present invention is designated a “protecting group”, unless the context indicates otherwise. The protection of functional groups by such protecting groups, the protecting groups themselves, and their cleavage reactions are described for example in standard reference works, such as J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999.

Compounds of the present invention were made by processes described herein and as illustrated in the Examples. Non-limiting examples of synthetic schemes used to make compounds of the present invention are illustrated in Schemes 1-12 below.

Scheme 1 illustrates one embodiment for making compounds of the present invention, wherein amination of Intermediate (1a) with an amine comprising a terminal vinyl group gives intermediate (1 b) which comprises terminal vinyl groups. Catalytic ring closing metathesis (RCM) using a Ru-catalyst yields cyclic intermediate (1c), and subsequent hydrogenation give compounds of Formula (I-b).

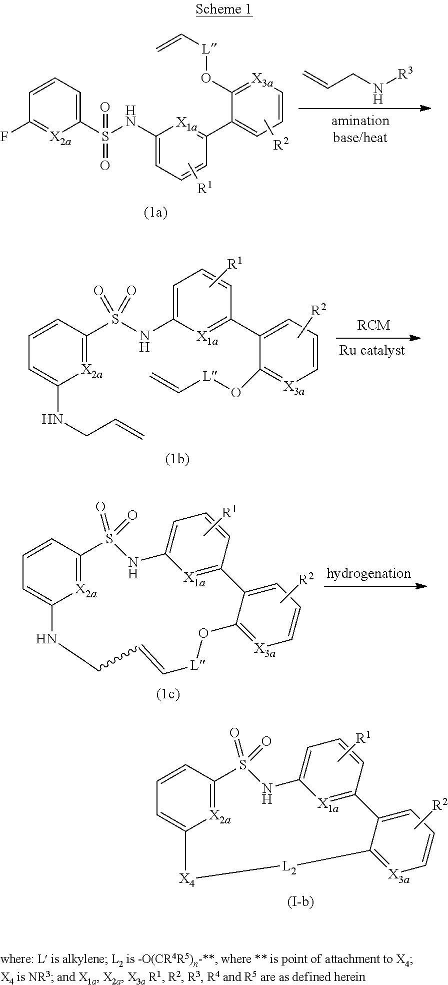

Scheme 2 illustrates another embodiment for making compounds of the present invention, wherein amination of Intermediate (2a) with a diamine yields compounds of Formula (I-b).

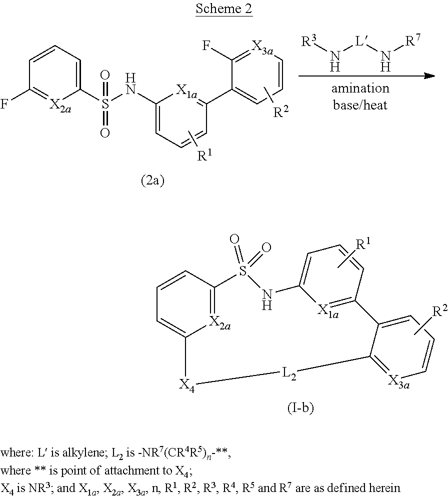

Scheme 3 illustrates another embodiment for making compounds of the present invention, wherein compounds of Formula (I-b) are obtained by ring closure upon ether formation via reaction of Intermediate (3a) with a diol.

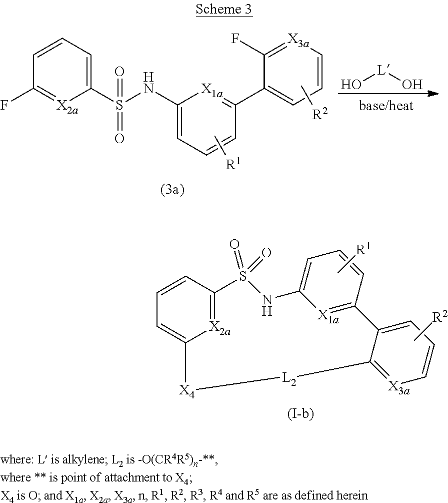

Scheme 4 illustrates another embodiment for making compounds of the present invention, wherein initial amination of Intermediate (4a) forms Intermediate (4b) which comprises a pendant alcohol group. Ring closure is achieved by intra-molecular ether formation thereby yielding compounds of Formula (I-b).

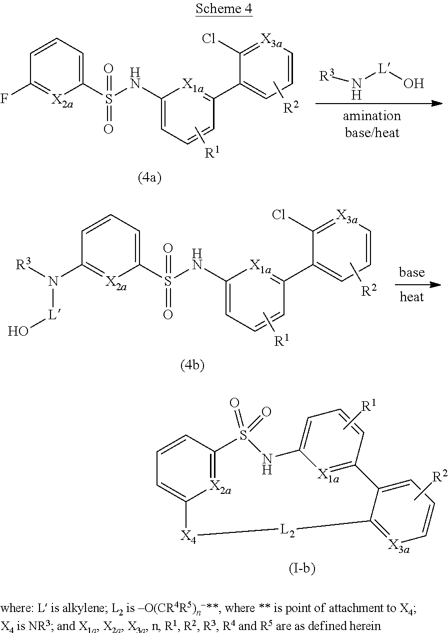

Scheme 5 illustrates another embodiment for making compounds of the present invention, wherein ring closure via amination of Intermediate (5a) yields compounds of Formula (I-b).

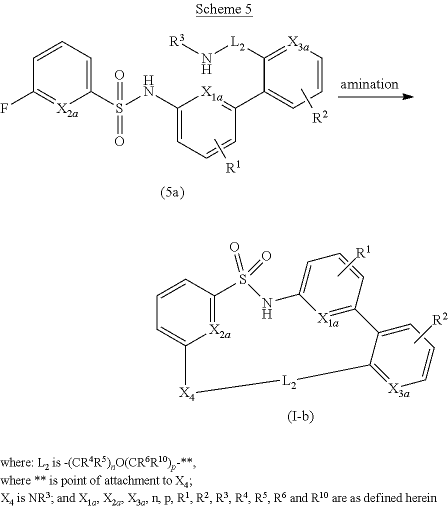

Scheme 6 illustrates another embodiment for making compounds of the present invention, wherein initial N-alkylation of Intermediate (6a) forms Intermediate (6b). Ring closure is achieved by intra-molecular amination thereby yielding Intermediate (6c) which is subsequently acidified to yield certain compounds of Formula (I-b).

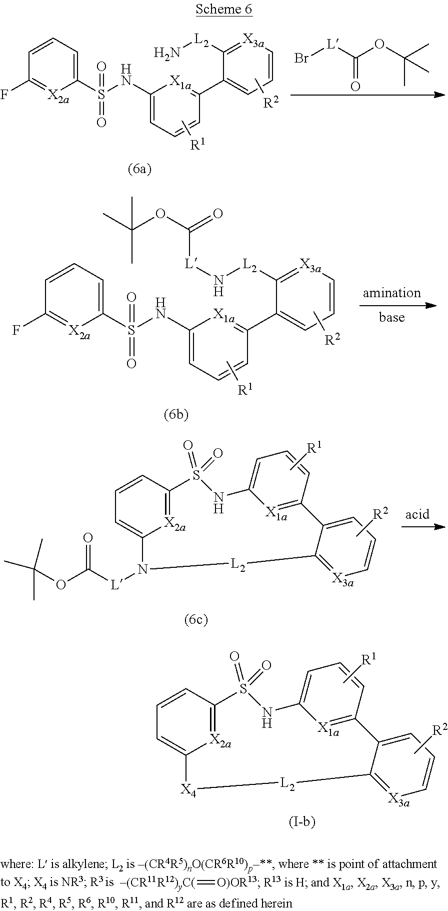

Scheme 7 illustrates another embodiment for making compounds of the present invention, wherein amination of Intermediate (7a) with an amine comprising a terminal vinyl group gives intermediate (7b) which comprises terminal vinyl groups. Catalytic ring closing metathesis (RCM) using a Ru-catalyst yields cyclic intermediate (7c), and subsequent hydrogenation gives compounds of Formula (I-b).

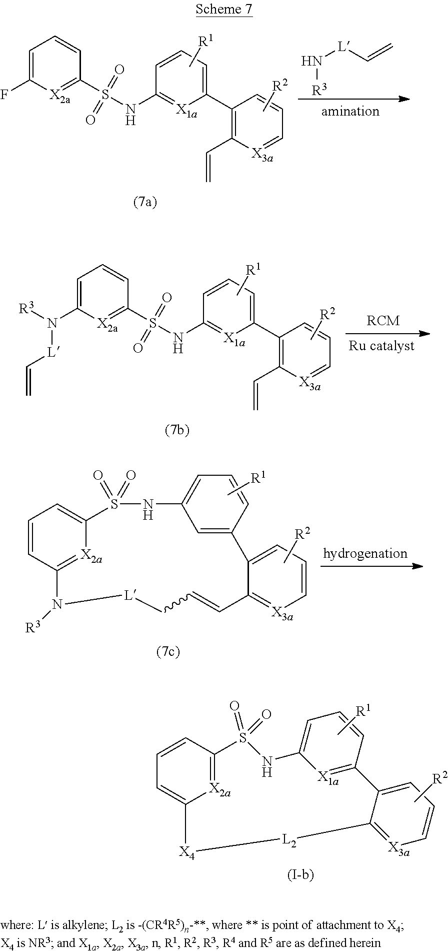

Compounds of the present invention and intermediates can also be converted into each other according to methods generally known to those skilled in the art. The following reaction schemes 8-12 illustrate general reactions used to transform certain compounds of the present invention to other compounds of the present invention.

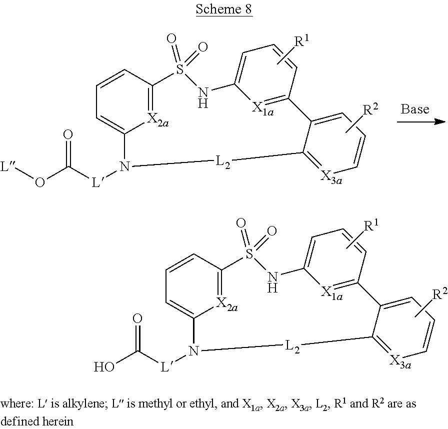

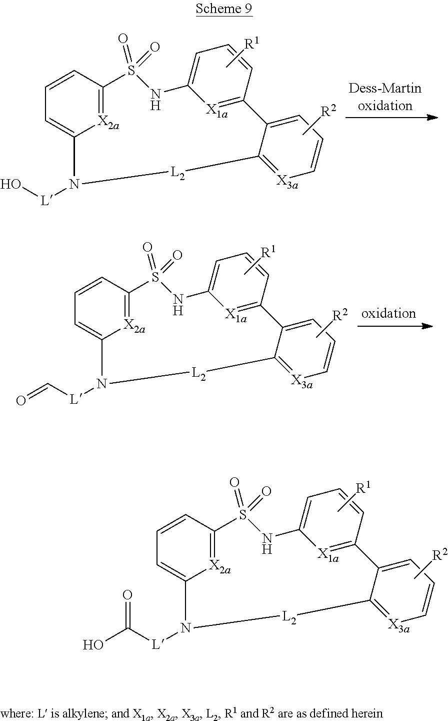

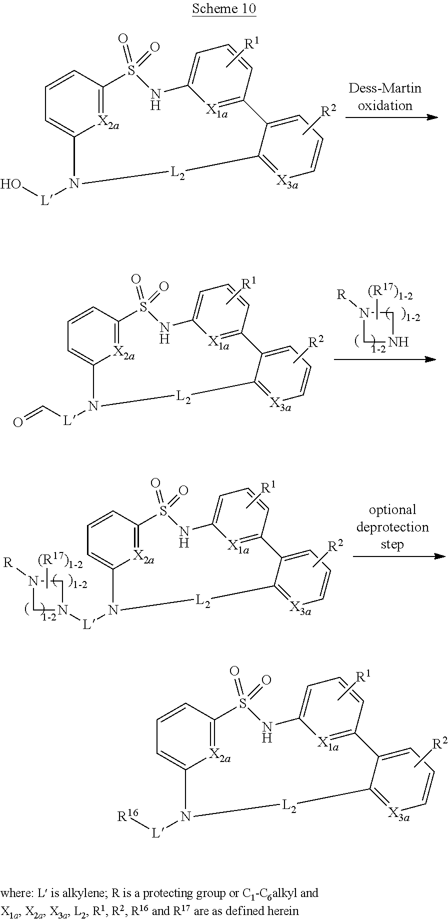

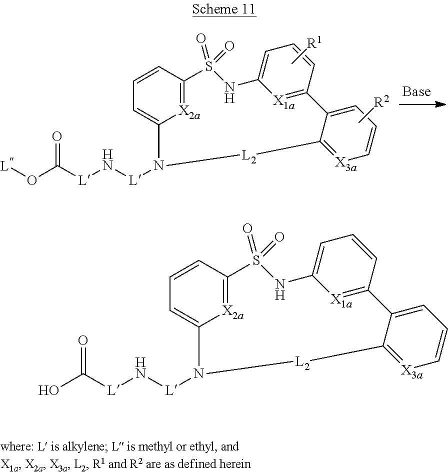

### EXAMPLES

The compounds of the present invention can be produced as shown in the following examples. The following examples are intended to illustrate the invention and are not to be construed as being limitations thereon. Temperatures are given in degrees Celsius. If not mentioned otherwise, all evaporations are performed under reduced pressure, typically between about 15 mm Hg and 100 mm Hg (=20-133 mbar). The structure of final products, intermediates and starting materials is confirmed by standard analytical methods, e.g., microanalysis and spectroscopic characteristics, e.g., MS, IR, NMR. Abbreviations used are those conventional in the art.

All starting materials, building blocks, reagents, acids, bases, dehydrating agents, solvents, and catalysts utilized to synthesis the compounds of the present invention are either commercially available or can be produced by organic synthesis methods known to one of ordinary skill in the art or can be produced by organic synthesis methods as described herein.

For illustrative purposes, the general reaction schemes depicted herein provide potential routes for synthesizing the compounds of the present invention as well as key intermediates. For a more detailed description of the individual reaction steps, see the Examples section below. Although specific starting materials and reagents are depicted in the schemes and discussed below, other starting materials and reagents can be easily substituted to provide a variety of derivatives and/or reaction conditions. In addition, many of the compounds prepared by the methods described below can be further modified in light of this disclosure using conventional chemistry well known to those skilled in the art.

### Abbreviations

Abbreviations used are those conventional in the art or the following:

| | |
|---|---|
| Ac: Acetyl | Min(s): minute(s) |
| AcOH, HOAc: acetic acid | Me: methyl |
| aq.: aqueous | m/z: mass to charge ratio |
| app. q: apparent quartet | Alloc: allyloxycarbonyl protecting |
| | group |
| Ar: aromatic | M and mM: molar and millimolar |
| ADME: absorption, | mg: milligram |
| distribution, metabolism and | |
| excretion | |
| BPR: backpressure regulator | EDCI: 1-ethyl-3-(3- |
| | dimethylaminopropyl)carbodiimide |
| br: broad | BOP: (Benzotriazol-1- |
| | yloxy)tris(dimethylamino) |
| | phosphonium |
| | hexaflurorophosphate |
| DCC: | μL, mL and L: microliter, milliliter |
| dicyclohexylcarbodiimide | and liter |
| PyBOP: (Benzotriazol-1- | N: equivalent per liter |
| yloxy) | |
| tripyrrolidinophosphonium | |
| hexaflurorophosphate | |
| calc: calculated | n-BuLi: n-butyllitium |
| d: doublet; dd: doublet of | NMR: nuclear magnetic resonance |
| doublets | |
| DCM: dichloromethane | o/n: over night |
| Diox: Dioxane | PFA: pertluoroalkoxy (fluoropolymer) |
| DMF: N,N- | ppm: parts per million |
| dimethylformamide | |
| DMSO: dimethylsulfoxide | Ph: phenyl |
| DIEA or DIPEA: N,N- | q: quartet |
| diisopropylethylamine | |
| dppp: 1,3- | rt: room temperature |
| bis(diphenylphosphino) | |
| propane | |
| ESI-MS: electrospray | rpm: revolutions per minute |
| ionization mass spectrometry | |
| Et and EtOAc: ethyl and | s: singlet |
| ethyl acetate | |
| HATU: O-(7- | SFC: supercritical fluid |
| azobenzotriazol- | chromatography |
| 1-yl)-1,1,3,3- | |
| tetramethyluroniumhexa- | |
| fluorophosphate | |
| HOAt: 1-hydroxy-7- | t: triplet |
| azabenzotriazole | |
| HPLC: high pressure liquid | TEA: triethylamine |
| chromatography | |
| h, hr: hour(s) | THF: tetrahydrofuran |
| HRMS: high resolution mass | 2-MeTHF: 2-methyltetrahydrofuran |
| spectrometry | |
| LC and LCMS: liquid | TFA: trifluoroacetic acid |
| chromatography and liquid | |
| chromatography-mass | |
| spectrometry | |
| NMU: N-nitroso-N- | HEK293: Human Embryonic |
| methylurea | Kidney 293 cells |
| MeOH: methanol | DMEM: Dulbecco's modified |
| | eagle medium |
| HEPES: 4-(2-hydroxyethyl)- | wt: weight |
| 1-piperazineethanesulfonic | |
| acid | |
| EGTA: ethylene glycol | TBME: tert-butyl methy ether |
| tetraacetic acid | |
| PBS: Phosphate Buffered | TFAA: Trifluoroacetic acid |
| Saline, pH7.4 | |
| MS: mass | UHP: urea-hydrogen peroxide |
| m: multiplet | Isco, ISCO: Flash chromatography |
| | cartridge containing |
| | silica gel provided by |
| | Teledyne Isco |
| NMP: N- | HMDS: hexamethyldisilazane |
| methylpyrrolidinone | |
| LHMDS or | Grubbs II: (1,3-Bis(2,4,6- |
| LiHMDS: Lithium | trimethylphenyl)-2- |
| hexamethyldilazane | imidazolidinylidene)dichloro |
| | (phenylmethylene) |
| | (tricyclohexylphosphine)ruthenium |
| mCPBA: m- | Ts: Toluenesulfonyl or Tosyl |
| chloroperbenzoic acid | |
| TBAF: tetrabutylammonium | DCE: Dichlorethane |
| fluoride | |
| Boc: t-Butoxycarbonyl | tBuOH: tert-butanol |
| ACN: Acetonitrile | |

Analytical Methods

ESI-MS data (also reported herein as simply MS) were recorded using Waters System (Acquity UPLC and a Micromass ZQ mass spectrometer); all masses reported are the m/z of the protonated parent ions unless recorded otherwise.

LC/MS:

The sample is dissolved in suitable solvent such as MeCN, DMSO or MeOH and is injected directly into the column using an automated sample handler. The analysis is performed using one of the following methods:

HPLC Conditions:

Condition 1: Agilent 1200 Series HPLC system:
Agilent Binary Gradient Manager with Degasser
Agilent Diode Array Detector
Agilent 6140 Quadrupole LC/MS
SoftA ELS Detector

HPLC column: Waters Acquity HSS T3 C18 1.8 um, 2.1×50 mm

Flow rate: 0.9 mL/min

Temperature: 60° C. (column temp)

Mobile phase compositions: A: 0.05% trifluoroacetic acid in water.
B: 0.035% trifluoroacetic acid in acetonitrile.

Gradient:

| Time (min) | Flow (mL/min) | % A | % B |
|---|---|---|---|
| 0 | 0.9 | 90 | 10 |
| 0.15 | 0.9 | 90 | 10 |
| 1.50 | 0.9 | 0 | 100 |
| 1.95 | 0.9 | 0 | 100 |
| 2 | 0.9 | 90 | 10 |
| 2.25 | 0.9 | 90 | 10 |

### SYNTHESIS OF INTERMEDIATES

### Synthesis of intermediate N-(2-morpholinoethyl)pent-4-en-1-amine (int-a1)

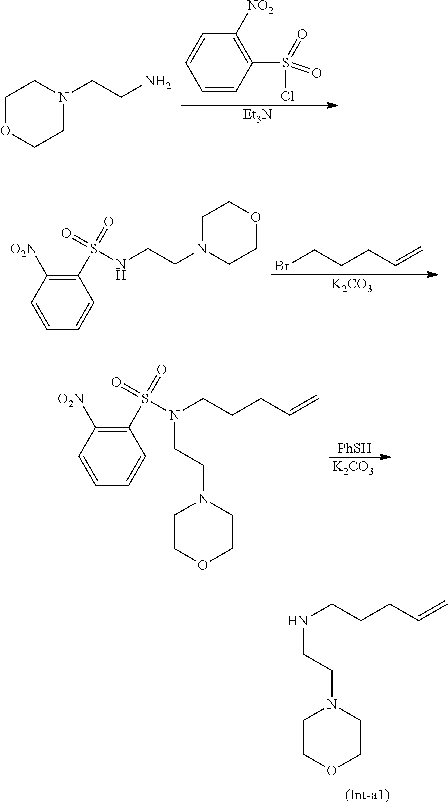

### Step 1. Synthesis of N-(2-morpholinoethyl)-2-nitrobenzenesulfonamide

To a cold (0° C.) solution of 2-morpholinoethanamine (0.651 g, 5 mmol) and Et₃N (2.1 mL, 15 mmol) in DCM (20 mL) was added 2-nitrobenzene-1-sulfonyl chloride (1.551 g, 7 mmol) and stirred overnight at room temperature. LCMS indicated the reaction was complete. The reaction solution was washed with water, brine, and dried over Na₂SO₄. The organic phase was filtered, concentrated and purified on a 40 g Gold ISCO column (EtOAc/Heptane 0-100%) to give the title compound (1.536 g, 97%) as a tan crystal. LCMS (Condition 1): m/z 316.2 [M+H]⁺, 0.41 min. ¹H NMR (400 MHz, DMSO-d₆) δ 8.05 (m, 1H), 7.98 (m, 1H), 7.87 (m, 3H), 3.43 (m, 4H), 3.04 (q, J=6.3 Hz, 2H), 2.31 (t, J=6.5 Hz, 2H), 2.23 (m, 4H).

### Step 2. Synthesis of N-(2-morpholinoethyl)-2-nitro-N-(pent-4-en-1-yl)benzenesulfonamide

A mixture of N-(2-morpholinoethyl)-2-nitrobenzenesulfonamide (631 mg, 2 mmol), 5-bromopent-1-ene (332 μL, 2.8 mmol), and K₂CO₃ (553 mg, 4.00 mmol) in DMF (8 mL) was stirred at 100° C. for 2 h. LCMS indicated that the reaction was complete. Then water was added to cold reaction solution, extracted with EtOAc, washed with water (×3), brine, and dried over Na₂SO₄. The combined extracts were filtered, concentrated and purified on 24 g Gold ISCO column (EtOAc/Heptane 0-100%) to give the title compound (771 mg, 1.95 mmol, 98% yield) as a colorless oil. LCMS (Condition 1): m/z 384.2 [M+H]⁺, 1.19 min. ¹H NMR (400 MHz, DMSO-d₆) δ 8.07 (m, 1H), 7.97 (m, 1H), 7.86 (m, 2H), 5.77 (ddt, J=6.6, 10.2, 16.8 Hz, 1H), 4.98 (m, 2H), 3.48 (m, 4H), 3.38 (t, J=6.6 Hz, 2H), 3.29 (m, 2H), 2.41 (t, J=6.6 Hz, 2H), 2.33 (m, 4H), 1.98 (m, 2H), 1.60 (p, J=7.5 Hz, 2H).

### Step 3. Synthesis of N-(2-morpholinoethyl)pent-4-en-1-amine (int-a1)

A mixture of N-(2-morpholinoethyl)-2-nitro-N-(pent-4-en-1-yl)benzenesulfonamide (748 mg, 1.95 mmol), K₂CO₃ (809 mg, 5.85 mmol), and thiophenol (0.24 mL, 2.34 mmol) in acetonitrile (8 mL) was stirred at room temperature for two days. LC-MS indicated the reaction was complete. The reaction solution was filtered, concentrated, and purified on an ISCO column (MeOH/DCM 0-10%) to give the title compound (344 mg, 89% yield) as an oil. LCMS (Condition 1): m/z 199.3 [M+H]⁺, 0.23 min. ¹H NMR (400 MHz, DMSO-d₆) 5.81 (ddt, J=6.6, 10.2, 16.9 Hz, 1H), 5.01 (m, 1H), 4.94 (ddt, J=1.2, 2.3, 10.2 Hz, 1H), 3.56 (m, 4H), 2.57 (t, J=6.5 Hz, 2H), 2.49 (m, 2H), 2.35 (m, 6H), 2.04 (m, 2H), 1.57 (s, 1H), 1.47 (m, 2H).

### Synthesis of intermediate 3-(2-(pent-4-en-1-ylamino)ethoxy)propan-1-ol (int-a2)

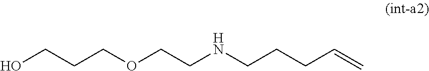

Synthesized using the procedure used for intermediate (int-a1) except in step 1 where 2-morpholinoethanamine was replaced with 3-(2-aminoethoxy)propan-1-ol. LCMS (Condition 1): m/z 188.2 [M+H]⁺, 0.28 min. ¹H NMR (400 MHz, DMSO-d₆) δ 5.80 (ddt, J=16.9, 10.2, 6.6 Hz, 1H), 5.01 (m, 1H), 4.94 (ddt, J=10.2, 2.3, 1.2 Hz, 1H), 3.41 (m, 7H), 2.65 (t, J=5.7 Hz, 2H), 2.52 (m, 2H), 2.02 (m, 2H), 1.63 (p, J=6.4 Hz, 2H), 1.48 (p, J=7.3 Hz, 2H).

### Synthesis of Intermediate 6-(pent-4-en-1-ylamino)hexan-1-ol (int-a3)

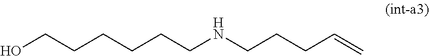

Synthesized using the procedure used for intermediate (int-a1) except in step 1 where 2-morpholinoethanamine was replaced with 6-aminohexan-1-ol. LCMS (Condition 1): m/z 186.3 [M+H]⁺, 1.09 min. ¹H NMR (400 MHz, DMSO-d₆) δ 5.80 (ddt, J=16.9, 10.2, 6.6 Hz, 1H), 5.00 (dq, J=17.2, 1.7 Hz, 1H), 4.93 (ddt, J=10.2, 2.4, 1.2 Hz, 1H), 4.31 (s, 1H), 3.37 (t, J=6.5 Hz, 2H), 2.46 (m, 4H), 2.03 (m, 2H), 1.46 (p, J=7.3 Hz, 2H), 1.38 (m, 4H), 1.26 (m, 4H).

### Synthesis of intermediate 2-(2-(pent-4-en-1-ylamino)ethoxy)ethan-1-ol (int-a4)

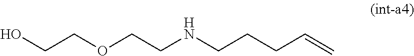

Synthesized using the procedure used for intermediate (int-a1) except in step 1 where 2-morpholinoethanamine was replaced with 2-(2-aminoethoxy)ethan-1-ol. LCMS (Condition 1): m/z 174.2 [M+H]⁺, 0.38 min. ¹H NMR (400 MHz, DMSO-d₆) δ 5.81 (ddt, J=16.9, 10.2, 6.6 Hz, 1H), 5.01 (m, 1H), 4.94 (ddt, J=10.1, 2.3, 1.2 Hz, 1H), 3.49 (m, 2H), 3.45 (t, J=5.7 Hz, 2H), 3.40 (m, 2H), 2.65 (t, J=5.7 Hz, 2H), 2.52 (m, 2H), 2.04 (m, 2H), 1.48 (p, J=7.3 Hz, 2H).

### Synthesis of intermediate 5-(but-3-en-1-ylamino)pentan-1-ol (int-a5)

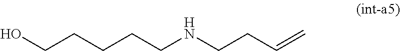

Synthesized using the procedure used for intermediate (int-a1) except in step 1 where 2-morpholinoethanamine was replaced with 5-aminopentan-1-ol, and in step 2 where 5-bromopent-1-ene was replaced with 4-bromobut-1-ene. LCMS (Condition 1): m/z 158.2 [M+H]⁺, 0.32 min. ¹H NMR (400 MHz, DMSO-d₆) δ 5.80 (ddt, J=17.0, 10.2, 6.8 Hz, 1H), 5.03 (dq, J=17.2, 1.6 Hz, 1H), 4.97 (ddt, J=10.2, 2.3, 1.2 Hz, 1H), 4.33 (s, 1H), 3.37 (t, J=6.5 Hz, 2H), 2.55 (t, J=7.2 Hz, 2H), 2.47 (m, 2H), 2.15 (qt, J=7.1, 1.4 Hz, 2H), 1.38 (m, 4H), 1.29 (m, 2H).

### Synthesis of intermediate 4-((2-(allyloxy)ethyl)amino)butan-1-ol (int-a6)

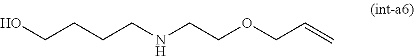

Synthesized using the procedure used for intermediate (int-a1) except in step 1 where 2-morpholinoethanamine was replaced with 4-aminobutan-1-ol, and in step 2 where 5-bromopent-1-ene was replaced with 3-(2-bromoethoxy)prop-1-ene. LCMS (Condition 1): m/z 174.3 [M+H]⁺, 0.32 min. ¹H NMR (400 MHz, DMSO-d₆) δ 5.88 (ddt, J=17.3, 10.6, 5.4 Hz, 1H), 5.24 (dq, J=17.3, 1.8 Hz, 1H), 5.13 (m, 1H), 3.92 (dt, J=5.3, 1.6 Hz, 2H), 3.42 (t, J=5.8 Hz, 2H), 3.37 (m, 2H), 2.64 (t, J=5.8 Hz, 2H), 2.48 (m, 2H), 1.41 (m, 4H).

### Synthesis of intermediate 4-(but-3-en-1-ylamino)butan-1-ol (int-a7)

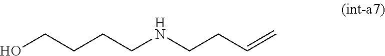

Synthesized using the procedure used for intermediate (int-a1) except in step 1 where 2-morpholinoethanamine was replaced with 4-aminobutan-1-ol, and in step 2 where 5-bromopent-1-ene was replaced with 4-bromobut-1-ene. LCMS (Condition 1): m/z 144.2 [M+H]⁺, 0.39 min. ¹H NMR (400 MHz, DMSO-d₆) δ 5.80 (ddt, J=17.1, 10.2, 6.8 Hz, 1H), 5.03 (m, 1H), 4.97 (ddt, J=10.2, 2.4, 1.2 Hz, 1H), 3.37 (m, 2H), 2.54 (m, 2H), 2.46 (m, 2H), 2.14 (qt, J=7.1, 1.4 Hz, 2H), 1.41 (m, 4H).

### Synthesis of intermediate (1-((pent-4-en-1-ylamino)methyl)cyclopropyl)methanol (int-a8)

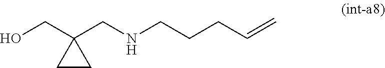

Synthesized using the procedure used for intermediate (int-a1) except in step 1 where 2-morpholinoethanamine was replaced with (1-(aminomethyl)cyclopropyl)methanol. LCMS (Condition 1): m/z 170.3 [M+H]⁺, 0.32 min. ¹H NMR (400 MHz, DMSO-d₆) δ 5.80 (ddt, J=6.6, 10.2, 16.9 Hz, 1H), 5.01 (ddt, J=1.6, 2.1, 17.2 Hz, 1H), 4.95 (ddt, J=1.2, 2.3, 10.2 Hz, 1H), 4.22 (bs, 2H), 3.32 (s, 2H), 2.56 (m, 4H), 2.03 (m, 2H), 1.51 (p, J=7.4 Hz, 2H), 0.34 (m, 4H).

### Synthesis of intermediate 3-((2-(allyloxy)ethyl)amino)propan-1-ol (int-a9)

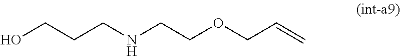

Synthesized using the procedure used for intermediate (int-a1) except in step 1 where 2-morpholinoethanamine was replaced with 3-aminopropan-1-ol, and in step 2 where 5-bromopent-1-ene was replaced with 3-(2-bromoethoxy)prop-1-ene. LCMS (Condition 1): m/z 160.2 [M+H]⁺, 0.28 min. ¹H NMR (400 MHz, DMSO-d₆) δ 5.88 (ddt, J=5.3, 10.6, 17.3 Hz, 1H), 5.24 (dq, J=1.8, 17.3 Hz, 1H), 5.13 (m, 1H), 3.92 (dt, J=1.5, 5.3 Hz, 2H), 3.43 (m, 4H), 2.64 (t, J=5.7 Hz, 2H), 2.56 (t, J=6.8 Hz, 2H), 1.53 (p, J=6.6 Hz, 2H).

### Synthesis of intermediate N-((2,2-dimethyl-1,3-dioxolan-4-yl)methyl)pent-4-en-1-amine (int-a10)

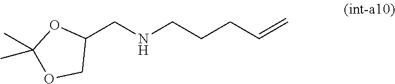

Synthesized using the procedure used for intermediate (int-a1) except in step 1 where 2-morpholinoethanamine was replaced with (2,2-dimethyl-1,3-dioxolan-4-yl)methanamine. LCMS (Condition 1): m/z 160.2 [M+H]⁺, 0.28 min. ¹H NMR (400 MHz, DMSO-d₆) δ 5.80 (ddt, J=6.6, 10.2, 16.9 Hz, 1H), 5.00 (m, 1H), 4.93 (ddt, J=1.2, 2.3, 10.2 Hz, 1H), 4.08 (p, J=6.1 Hz, 1H), 3.95 (dd, J=6.3, 8.0 Hz, 1H), 3.58 (dd, J=6.5, 8.0 Hz, 1H), 2.62 (dd, J=5.9, 11.9 Hz, 1H), 2.52 (m, 3H), 2.02 (m, 2H), 1.56 (s, 1H), 1.46 (p, J=7.4 Hz, 2H), 1.30 (s, 3H), 1.25 (s, 3H).

### Synthesis of intermediate (1-(((2-(allyloxy)ethyl)amino)methyl)cyclopropyl)methanol (int-a11)

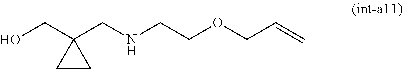

Synthesized using the procedure used for intermediate (int-a1) except in step 1 where 2-morpholinoethanamine was replaced with (1-(aminomethyl)cyclopropyl)methanol, and in step 2 where 5-bromopent-1-ene was replaced with 3-(2-bromoethoxy)prop-1-ene. LCMS (Condition 1): m/z 186.3 [M+H]⁺, 0.31 min. ¹H NMR (400 MHz, DMSO-d₆) δ 5.88 (ddt, J=5.3, 10.5, 17.3 Hz, 1H), 5.24 (dq, J=1.7, 17.3 Hz, 1H), 5.13 (ddt, J=1.4, 2.1, 10.5 Hz, 1H), 3.93 (dt, J=1.5, 5.3 Hz, 2H), 3.42 (t, J=5.7 Hz, 2H), 3.30 (s, 2H), 2.65 (t, J=5.7 Hz, 2H), 2.51 (s, 2H), 0.31 (m, 2H), 0.27 (m, 2H).

### Synthesis of intermediate 5-(hept-6-en-1-ylamino)pentan-1-ol (int-a12)

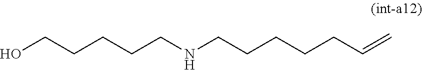

Synthesized using the procedure used for intermediate (int-a1) except where 5-bromopent-1-ene was replaced with 4-bromobut-1-ene and 2-aminoethanol was replaced with tert-butyl (2-aminoethyl)carbamate. LCMS (Condition 1): : m/z 200.3 [M+H]+. 1.18 min. ¹H NMR (400 MHz, DMSO-d₆) δ 5.79 (ddt, J=16.9, 10.2, 6.7 Hz, 1H), 5.00 (m, 1H), 4.93 (ddt, J=10.2, 2.3, 1.2 Hz, 1H), 3.37 (t, J=6.5 Hz, 2H), 2.48 (m, 4H), 2.00 (m, 2H), 1.32 (m, 12H).

### Synthesis of intermediate ((4S,5S)-2,2-dimethyl-5-((pent-4-en-1-ylamino)methyl)-1,3-dioxolan-4-yl)methanol (int-a13)

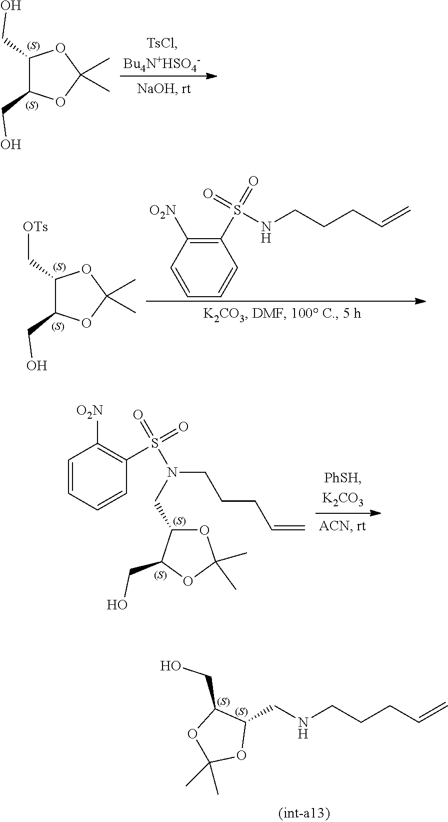

### Step 1. Synthesis of ((4S,5S)-5-(hydroxymethyl)-2,2-dimethyl-1,3-dioxolan-4-yl)methyl 4-methylbenzenesulfonate

To a solution of ((4S,5S)-2,2-dimethyl-1,3-dioxolane-4,5-diyl)dimethanol (1.622 g, 10 mmol), tetrabutylammonium hydrogen sulfate (0.352 g, 1.037 mmol) and DCM (41 mL) was added a solution of NaOH (0.44 g, 11 mmol) in water (2.87 mL) and followed by a solution of TsCl (2.1 g, 11 mmol) in DCM (10 mL). The resulting reaction solution was stirred at room temperature for 70 min. LCMS indicated that the reaction was complete. The reaction solution was washed with water, saturated NaHCO₃ solution, brine, dried over Na₂SO₄ and filtered. The filtrate was concentrated and purified on an ISCO column (EtOAc/heptane 0-50%) to give the title compound (2.805 g, 89% yield) as an oil. LCMS (Condition 1): m/z 317.2 [M+H]⁺, 1.30 min. ¹H NMR (400 MHz, DMSO-d₆) δ 7.79 (m, 2H), 7.49 (m, 2H), 4.89 (s, 1H), 4.20 (dd, J=2.7, 10.8 Hz, 1H), 4.05 (m, 1H), 3.96 (ddd, J=2.6, 6.3, 8.1 Hz, 1H), 3.75 (dt, J=5.1, 8.0 Hz, 1H), 3.48 (dd, J=4.8, 11.4 Hz, 1H), 3.43 (dd, J=5.3, 11.4 Hz, 1H), 2.43 (s, 3H), 1.27 (s, 3H), 1.23 (s, 3H).

### Step 2. Synthesis of N-(((4S,5S)-5-(hydroxymethyl)-2,2-dimethyl-1,3-dioxolan-4-yl)methyl)-2-nitro-N-(pent-4-en-1-yl)benzenesulfonamide

A mixture of 2-nitro-N-(pent-4-en-1-yl)benzenesulfonamide (0.811 g, 3 mmol), ((4S,5S)-5-(hydroxymethyl)-2,2-dimethyl-1,3-dioxolan-4-yl)methyl 4-methylbenzenesulfonate (1.234 g, 3.9 mmol), and K₂CO₃ (1.244 g, 9 mmol) in DMF (10 mL) was stirred at 100° C. for 5 h. LCMS indicated that the reaction was complete. Water was added to the cold reaction mixture, extracted with EtOAc. The combined extracts were washed with water (3×), brine, dried over Na₂SO₄ and filtered. The filtrate was concentrated and purified on an ISCO column (EtOAs/heptane 0-100%) to give the title compound (459 mg, 37% yield) as a tan oil. LCMS (Condition 1): m/z 415.3 [M+H]⁺, 1.47 min, ¹H NMR (400 MHz, DMSO-d₆) δ 8.04 (dd, J=1.6, 7.6 Hz, 1H), 7.97 (dd, J=1.4, 7.8 Hz, 1H), 7.88 (td, J=1.6, 7.6 Hz, 1H), 7.84 (td, J=1.6, 7.6 Hz, 1H), 5.74 (ddt, J=6.5, 10.2, 16.8 Hz, 1H), 4.96 (m, 2H), 4.89 (t, J=5.4 Hz, 1H), 3.91 (td, J=2.7, 8.1 Hz, 1H), 3.70 (dt, J=4.8, 8.1 Hz, 1H), 3.58 (dd, J=2.6, 15.1 Hz, 1H), 3.49 (t, J=5.3 Hz, 2H), 3.38 (m, 2H), 3.28 (m, 1H), 1.95 (q, J=6.9 Hz, 2H), 1.60 (p, J=7.6 Hz, 2H), 1.26 (s, 3H), 1.25 (s, 3H).

### Step 3. Synthesis of ((4S,5S)-2,2-dimethyl-5-((pent-4-en-1-ylamino)methyl)-1,3-dioxolan-4-yl)methanol (int-a13)

A mixture of N-(((4S,5S)-5-(hydroxymethyl)-2,2-dimethyl-1,3-dioxolan-4-yl)methyl)-2-nitro-N-(pent-4-en-1-yl)benzenesulfonamide (458 mg, 1.11 mmol), K₂CO₃ (458 mg, 3.32 mmol), and thiophenol (0.148 mL, 1.437 mmol) in ACN (4.5 mL) was stirred overnight at room temperature. LCMS indicated the reaction was complete. The reaction was cooled to room temperature, the precipitate was filtered, washed with ACN. The filtrate was concentrated, and purified on an ISCO column (MeOH/DCM 0-10%) to give the title compound (233 mg, 92% yield) as a colorless oil. LCMS (Condition 1): m/z 230.3 [M+H]⁺, 0.78 min. ¹H NMR (400 MHz, DMSO-d₆) 5.80 (ddt, J=6.6, 10.2, 16.9 Hz, 1H), 5.01 (m, 1H), 4.94 (ddt, J=1.2, 2.3, 10.2 Hz, 1H), 3.77 (dt, J=5.7, 8.0 Hz, 1H), 3.68 (ddd, J=4.8, 5.7, 8.0 Hz, 1H), 3.51 (dd, J=4.7, 11.2 Hz, 1H), 3.45 (dd, J=5.7, 11.2 Hz, 1H), 2.72 (dd, J=5.6, 12.0 Hz, 1H), 2.62 (dd, J=5.8, 12.0 Hz, 1H), 2.45-2.58 (m, 2H), 2.03 (m, 2H), 1.47 (p, J=7.2 Hz, 2H), 1.30 (s, 3H), 1.29 (s, 3H).

### Synthesis of intermediate ethyl 2-(allylamino)acetate (int-a14)

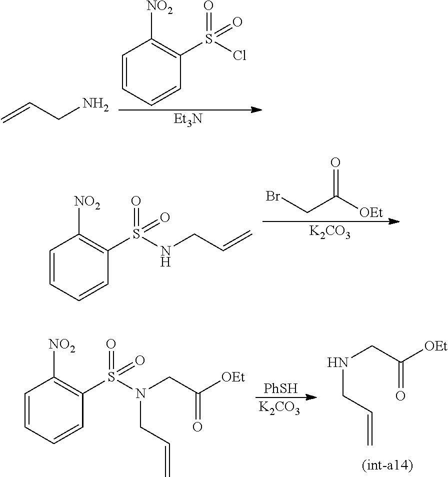

### Step 1. Synthesis of N-allyl-2-nitrobenzenesulfonamide

To a mixture of allylamine (5.00 mL, 66.6 mmol) and triethylamine (18.58 mL, 133 mmol) in anhydrous DCM (100 mL) was added in portion 2-nitrobenzene-1-sulfonyl chloride (14.77 g, 66.6 mmol) at 0° C. The resulting mixture was stirred overnight at room temperature. LCMS indicated that the reaction was complete. The mixture was diluted with water and the pH adjusted to 8-9 with 1 N HCl (50 mL). The layers were separated and the organic layer was dried over MgSO₄, filtered, and concentrated to obtain the title compound (15 g, 55.7 mmol, 84% yield) without further purification as a brown oil. LCMS (Condition 1): m/z 243.2 [M+H]⁺, 1.18 min.

### Step 2. Synthesis of ethyl 2-(N-allyl-2-nitrophenylsulfonamido)acetate

A mixture of N-allyl-2-nitrobenzenesulfonamide (2.00 g, 8.26 mmol), K₂CO₃ (2.28 g, 16.50 mmol), and ethyl 2-bromoacetate (1.10 mL, 9.91 mmol) in DMF (15 mL) was stirred at room temperature for 4 days to achieve orange suspension. The mixture was diluted with EtOAc (50 mL) and saturated aqueous NH₄Cl. The organic layer was washed with water (3×) and brine, dried over MgSO₄, filtered, concentrated and purified by an ISCO column (EtOAc/heptane 0-100%) to afford the title compound (2.5 g, 7.23 mmol, 88% yield) as a light yellow oil. LCMS (Condition 1): m/z 329.2 [M+H]⁺, 1.45 min.

### Step 3 Synthesis of ethyl 2-(allylamino)acetate (int-a14)

To a mixture of ethyl 2-(N-allyl-2-nitrophenylsulfonamido)acetate (2.5 g, 7.61 mmol), and K₂CO₃ (3.16 g, 22.84 mmol) in ACN (50 mL) was added benzenethiol (0.784 mL, 7.61 mmol) and the mixture was stirred overnight at room temperature to achieve a yellow suspension. The mixture was diluted with DCM (50 mL) and then filtered. The filtrate was concentrated and purified by an ISCO column (100% EtOAc; fractions were checked by LCMS and by KMnO₄ stain) to afford the title compound (580 mg, 4.05 mmol, 53% yield) as an oil. LCMS (Condition 1): m/z 144.2 [M+H]⁺, 0.24 min.

### Synthesis of intermediate 2-(allylamino)ethan-1-ol (int-a15)

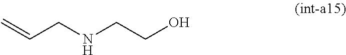

Synthesized using the procedure used for intermediate (int-a14) except in step 2 where ethyl 2-bromoacetate was replaced with 2-bromoethan-1-ol. LCMS (Condition 1): m/z 102.2 [M+H]⁺, 0.22 min.

### Synthesis of intermediate benzyl (2-(pent-4-en-1-ylamino)ethyl)carbamate (int-a16)

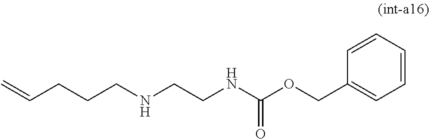 [SMILES: C=CCCCNCCNC(=O)OCc1ccccc1]

Synthesized using the procedure used for intermediate (int-a14) except in step 1 where allylamine was replaced with pent-4-en-1-amine, and in step 2 where ethyl 2-bromoacetate was replaced with benzyl (2-bromoethyl)carbamate. LCMS (Condition 1): m/z 263.3 [M+H]⁺, 1.12 min.

### Synthesis of intermediate 3-(hex-5-en-1-ylamino)propan-1-ol (int-a17)

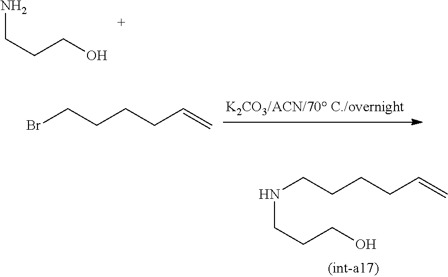

A mixture of 3-aminopropan-1-ol (1.13 g, 15.00 mmol), 6-bromohex-1-ene (1.63 g, 10.00 mmol) and K₂CO₃ (2.76 g, 20.00 mmol) in ACN (75 mL) was stirred overnight at 70° C. LCMS indicated that the reaction was almost complete, with two products being formed (TLC indicated that the desired mono-alkylation product was the major product formed). Then the reaction was filtered through a celite pad, the filtrate was concentrated and the residue was subjected to an ISCO purification (MeOH/DCM 0-10%) to get the desired title compound (1.03 g, 65% yield). The di-alkylation by-product was not collected. LCMS (Condition 1): m/z 158.2 [M+1]+, 0.49 min. ¹H NMR (400 MHz, Methanol-d₄) δ 5.83 (ddt, J=16.9, 10.2, 6.7 Hz, 1H), 5.05 (dq, J=17.1, 1.6 Hz, 1H), 4.99 (ddt, J=10.2, 2.2, 1.2 Hz, 1H), 3.72-3.64 (m, 2H), 3.16-3.08 (m, 2H), 3.05-2.95 (m, 2H), 2.17-2.08 (m, 2H), 1.95-1.84 (m, 2H), 1.77-1.64 (m, 2H), 1.55-1.44 (m, 2H).

### Synthesis of intermediate methyl 2-(hex-5-en-1-ylamino)-2-methylpropanoate (int-a18)

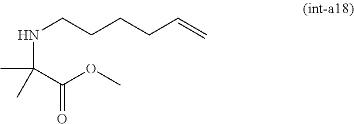

Synthesized using the procedure used for intermediate (int-a17) except where 3-aminopropan-1-ol was replaced with methyl 2-amino-2-methylpropanoate. LCMS (Condition 1): m/z 200.3 [M+H]⁺, 0.30 min.

### Synthesis of intermediate methyl hex-5-en-1-ylglycinate (int-a19)

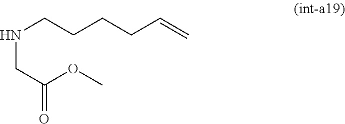

Synthesized using the procedure used for intermediate (int-a17) except where 3-aminopropan-1-ol was replaced with methyl glycinate. LCMS (Condition 1): m/z 172.2 [M+H]⁺, 0.27 min.

### Synthesis of intermediate ethyl 3-(hex-5-en-1-ylamino)-2,2-dimethylpropanoate (int-a20)

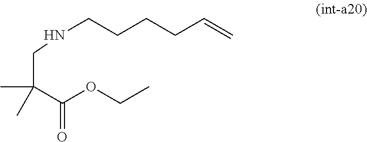 [SMILES: C=CCCCCNCC(C)(C)C(=O)OCC]

Synthesized using the procedure used for intermediate (int-a17) except where 3-aminopropan-1-ol was replaced with ethyl 3-amino-2,2-dimethylpropanoate. LCMS (Condition 1): m/z 228.3 [M+H]⁺, 1.04 min.

### Synthesis of intermediate ethyl pent-4-en-1-ylglycinate (int-a21)

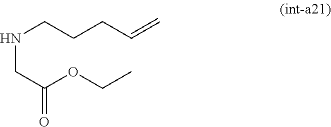

Synthesized using the procedure used for intermediate (int-a17) except where 3-aminopropan-1-ol was replaced with ethyl glycinate and 6-bromohex-1-ene was replaced with 5-bromopent-1-ene. LCMS (Condition 1): m/z 172.2 [M+H]⁺, 0.50 min.

### Synthesis of intermediate ethyl (2-(allyloxy)ethyl)glycinate (int-a22)

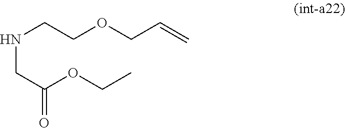

Synthesized using the procedure used for intermediate (int-a17) except where 3-aminopropan-1-ol was replaced with ethyl glycinate and 6-bromohex-1-ene was replaced with 3-(2-bromoethoxy)prop-1-ene. LCMS (Condition 1): m/z 188.2 [M+H]⁺, 0.32 min.

### Synthesis of intermediate ethyl 4-(pent-4-en-1-ylamino)butanoate (int-a23)

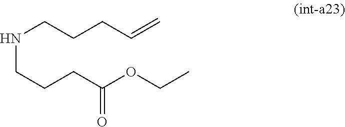

Synthesized using the procedure used for intermediate (int-a17) except where 3-aminopropan-1-ol was replaced with ethyl 4-aminobutanoate and 6-bromohex-1-ene was replaced with 5-bromopent-1-ene. LCMS (Condition 1): m/z 200.3 [M+H]⁺, 0.96 min.

### Synthesis of intermediate 3-(pent-4-en-1-ylamino)propan-1-ol (int-a24)

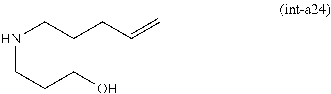

Synthesized using the procedure used for intermediate (int-a17) except where 6-bromohex-1-ene was replaced with 5-bromopent-1-ene. LCMS (Condition 1): m/z 200.3 [M+H]⁺, 0.96 min.

### Synthesis of intermediate 2,2-dimethyl-3-(pent-4-en-1-ylamino)propan-1-ol (int-a25)

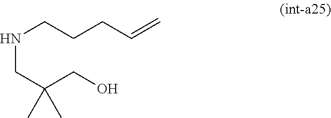

Synthesized using the procedure used for intermediate (int-a17) except where 3-aminopropan-1-ol was replaced with 3-amino-2,2-dimethylpropan-1-ol and 6-bromohex-1-ene was replaced with 5-bromopent-1-ene. LCMS (Condition 1): m/z 172.3 [M+H]⁺, 0.87 min.

### Synthesis of intermediate 3-(hex-5-en-1-ylamino)-2,2-dimethylpropan-1-ol (int-a26)

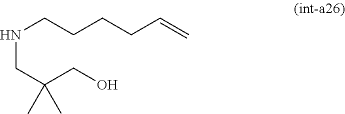

Synthesized using the procedure used for intermediate (int-a17) except where 3-aminopropan-1-ol was replaced with 3-amino-2,2-dimethylpropan-1-ol. LCMS (Condition 1): m/z 186.3 [M+H]⁺, 0.49 min.

### Synthesis of intermediate (3-((pent-4-en-1-ylamino)methyl)oxetan-3-yl)methanol (int-a27)

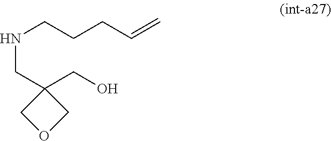

Synthesized using the procedure used for intermediate (int-a17) except where 3-aminopropan-1-ol was replaced with (3-(aminomethyl)oxetan-3-yl)methanol and 6-bromohex-1-ene was replaced with 5-bromopent-1-ene. LCMS (Condition 1): m/z 186.2 [M+H]⁺, 0.35 min.

### Synthesis of intermediate 3-(but-3-en-1-ylamino)propan-1-ol (int-a28)

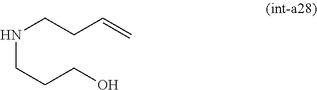

Synthesized using the procedure used for intermediate (int-a17) except where 6-bromohex-1-ene was replaced with 4-bromobut-1-ene. LCMS (Condition 1): m/z 130.2 [M+H]⁺, 0.33 min.

### Synthesis of intermediate 4-(pent-4-en-1-ylamino)butan-1-ol (int-a29)

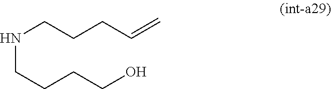

Synthesized using the procedure used for intermediate (int-a17) except where 6-bromohex-1-ene was replaced with 5-bromopent-1-ene. LCMS (Condition 1): m/z 158.3 [M+H]⁺, 0.50 min.

### Synthesis of intermediate 3-(allylamino)propan-1-ol (int-a30)

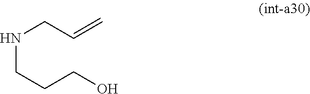

Synthesized using the procedure used for intermediate (int-a17) except where 6-bromohex-1-ene was replaced with 3-bromoprop-1-ene. LCMS (Condition 1): m/z 116.2 [M+H]⁺, 0.37 min.

### Synthesis of intermediate 4-(allylamino)butan-1-ol (int-a31)

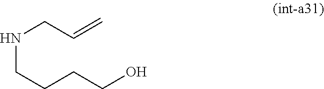

Synthesized using the procedure used for intermediate (int-a17) except where 3-aminopropan-1-ol was replaced with 4-aminobutan-1-ol and 6-bromohex-1-ene was replaced with 3-bromoprop-1-ene. LCMS (Condition 1): m/z 130.2 [M+H]⁺, 0.22 min.

### Synthesis of intermediate N-ethylpent-4-en-1-amine (int-a32)

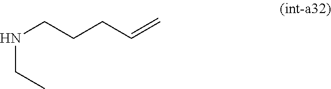

Synthesized using the procedure used for intermediate (int-a17) except where 3-aminopropan-1-ol was replaced with ethanamine and 6-bromohex-1-ene was replaced with 5-bromopent-1-ene. LCMS (Condition 1): m/z 114.2 [M+H]⁺, 0.38 min.

### Synthesis of intermediate 5-(pent-4-en-1-ylamino)pentan-1-ol (int-a33)

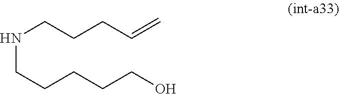

Synthesized using the procedure used for intermediate (int-a17) except where 3-aminopropan-1-ol was replaced with 5-aminopentan-1-ol and 6-bromohex-1-ene was replaced with 5-bromopent-1-ene. LCMS (Condition 1): m/z 172.2 [M+H]⁺, 0.71 min.

### Synthesis of intermediate 5-(hex-5-en-1-ylamino)pentan-1-ol (int-a34)

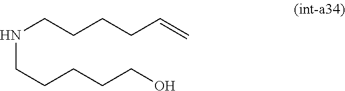

Synthesized using the procedure used for intermediate (int-a17) except where 3-aminopropan-1-ol was replaced with 5-aminopentan-1-ol. LCMS (Condition 1): m/z 186.2 [M+H]⁺, 0.57 min.

### Synthesis of intermediate 4-(hex-5-en-1-ylamino)butan-1-ol (int-a35)

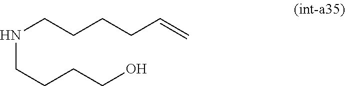

Synthesized using the procedure used for intermediate (int-a17) except where 3-aminopropan-1-ol was replaced with 4-aminobutan-1-ol. LCMS (Condition 1): m/z 172.2 [M+H]⁺, 0.44 min.

### Synthesis of intermediate 4-(hept-6-en-1-ylamino)butan-1-ol (int-a36)

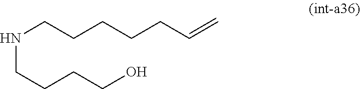

Synthesized using the procedure used for intermediate (int-a17) except where 3-aminopropan-1-ol was replaced with 4-aminobutan-1-ol and 6-bromohex-1-ene was replaced with 7-bromohept-1-ene. LCMS (Condition 1): m/z 186.3 [M+H]⁺, 1.04 min.

### Synthesis of intermediate 4-(oct-7-en-1-ylamino)butan-1-ol (int-a37)

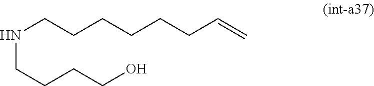

Synthesized using the procedure used for intermediate (int-a17) except where 3-aminopropan-1-ol was replaced with 4-aminobutan-1-ol and 6-bromohex-1-ene was replaced with 8-bromooct-1-ene. LCMS (Condition 1): m/z 200.3 [M+H]⁺, 1.24 min.

### Synthesis of intermediate 4-(non-8-en-1-ylamino)butan-1-ol (int-a38)

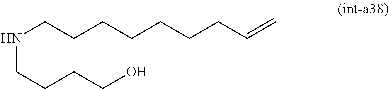

Synthesized using the procedure used for intermediate (int-a17) except where 3-aminopropan-1-ol was replaced with 4-aminobutan-1-ol and 6-bromohex-1-ene was replaced with 9-bromonon-1-ene. LCMS (Condition 1): m/z 214.3 [M+H]⁺, 1.30 min.

### Synthesis of intermediate 3-(hept-6-en-1-ylamino)propan-1-ol (int-a39)

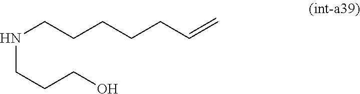

Synthesized using the procedure used for intermediate (int-a17) except where 6-bromohex-1-ene was replaced with 7-bromohept-1-ene. LCMS (Condition 1): m/z 172.2 [M+H]⁺, 0.59 min.

### Synthesis of intermediate 3-(oct-7-en-1-ylamino)propan-1-ol (int-a40)

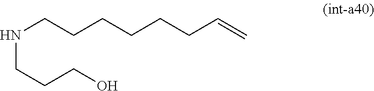

Synthesized using the procedure used for intermediate (int-a17) except where 6-bromohex-1-ene was replaced with 8-bromooct-1-ene. LCMS (Condition 1): m/z 186.3 [M+H]⁺, 1.13 min.

### Synthesis of intermediate 3-(non-8-en-1-ylamino)propan-1-ol (int-a41)

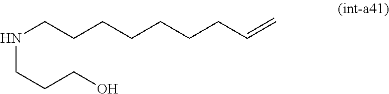

Synthesized using the procedure used for intermediate (int-a17) except where 6-bromohex-1-ene was replaced with 9-bromonon-1-ene. LCMS (Condition 1): m/z 200.3 [M+H]⁺, 1.27 min.

### Synthesis of intermediate 2-(pent-4-en-1-ylamino)ethan-1-ol (int-a42)

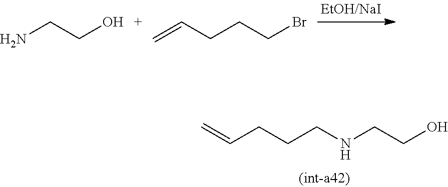

To a solution of 5-bromopent-1-ene (2.0 mL, 16.80 mmol) and 2-aminoethanol (5.0 mL, 83 mmol) in EtOH (40 mL) was added NaI (0.25 g, 1.68 mmol). The reaction mixture was stirred at room temperature for 4 days then heated under reflux for 1 h. The mixture was then cooled to room temperature and evaporated in vacuo. The residue was partitioned between saturated aqueous NH₄Cl solution and EtOAc (contain mainly dialkylation product). The aqueous layer was made basic with 40% sodium hydroxide (3 mL) to pH ˜10 and extracted with EtOAc (3×). The combined organic layers were dried over MgSO₄ and concentrated to afford the title compound (1.60 g, 12.38 mmol, 74% yield) as a yellow brown oil. LCMS (Condition 1): m/z 130.2 [M+H]⁺, 0.70 min.

### Synthesis of intermediate tert-butyl (2-(but-3-en-1-ylamino)ethyl)carbamate (int-a43)

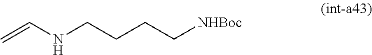

Synthesized using the procedure used for intermediate (int-a42) except where 5-bromopent-1-ene was replaced with 4-bromobut-1-ene and 2-aminoethanol was replaced with tert-butyl (2-aminoethyl)carbamate. LCMS (Condition 1): m/z 215.2 [M+H]⁺, 1.18 min.

### Synthesis of (R)-5-(but-3-en-1-yl)pyrrolidin-2-one (int-a44)

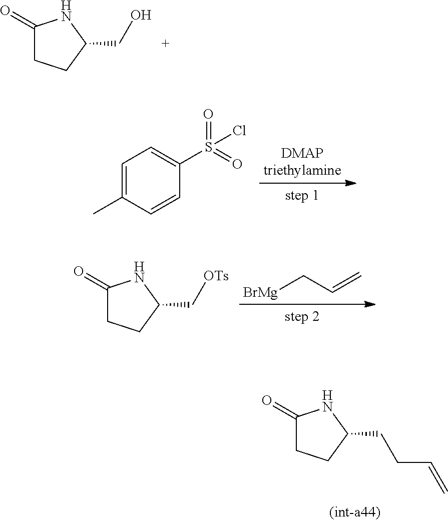

Step 1. To a solution of (S)-5-(hydroxymethyl)pyrrolidin-2-one (5.15 g, 44.7 mmol) in DCM (100 mL) was added 4-methylbenzene-1-sulfonyl chloride (10.23 g, 53.7 mmol), triethylamine (9.05 g, 89 mmol) and DMAP (0.546 g, 4.47 mmol). The clear solution was stirred at rt overnight and the reaction was stopped and diluted with DCM (100 mL). Water (250 mL) was then added, followed by conc. HCl (4 mL). The aqueous phase was separated and extracted with DCM (2×50 mL). DCM was combined and dried over Na₂SO₄. Silica gel (12 g) was added to the solution and the solvent was evaporated. The silica gel was transferred to a short column and eluted with (MeOH:EtOAc/1:20) to give (S)-(5-oxopyrrolidin-2-yl)methyl 4-methylbenzenesulfonate as a white solid: MS 270.1 [M+H]+, rt=0.96 min

Step 2. Allylmagnesium bromide (74.3 mL, 74.3 mmol) in ether was added to a solution of (S)-(5-oxopyrrolidin-2-yl)methyl 4-methylbenzenesulfonate (4 g, 14.85 mmol) in THF (150 mL) over a period of 30 min at 0° C. The reaction was then stirred at 70° C. for 3 h and the reaction was then quenched with careful addition of saturated NH₄Cl (150 mL). The aqueous phase was separated and extracted with DCM (4×50 mL). The organic phases were combined, dried over Na₂SO₄ and evaporated. The resulting liquid was purified by flash chromatography (EtOAc:heptane/4:1) with addition of 0.5% TEA to give (R)-5-(but-3-en-1-yl)pyrrolidin-2-one (int-a44). MS 140.1 [M+H]+, rt=0.82 min; 1H NMR (500 MHz, Chloroform-d) δ 5.82 (ddt, J=17.0, 10.2, 6.7 Hz, 1H), 5.73 (s, 1H), 5.08 (dq, J=17.1, 1.6 Hz, 1H), 5.06-4.99 (m, 1H), 3.68 (p, J=6.6 Hz, 1H), 2.43-2.24 (m, 3H), 2.14 (m, 2H), 1.81-1.71 (m, 1H), 1.71-1.61 (m, 2H).

### Synthetic Procedure for Intermediate 6-fluoro-N-(6-(5-fluoro-2-vinylphenyl)-5-(trifluoromethyl)pyridin-2-yl)pyridine-2-sulfonamide (int-b1)

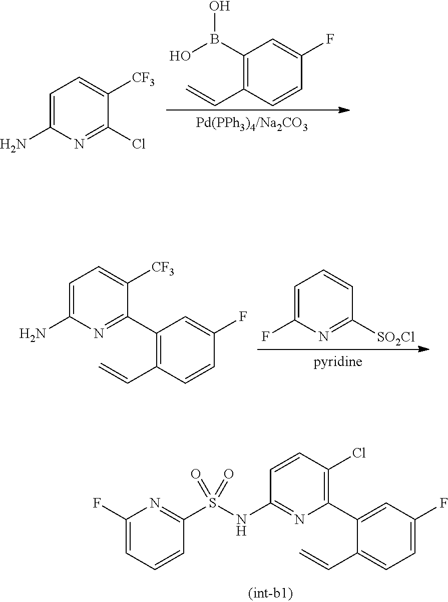

### Step 1. 6-(5-fluoro-2-vinylphenyl)-5-(trifluoromethyl)pyridin-2-amine

6-Chloro-5-(trifluoromethyl)pyridin-2-amine (5 g, 25.4 mmol) and (5-fluoro-2-vinylphenyl)boronic acid (5.28 g, 31.8 mmol) were dissolved in dioxane (60 mL) and water (9 mL) and treated with sodium carbonate (10.78 g, 102 mmol). The mixture was degassed using argon. Tetrakis(triphenylphosphino)palladium(0) (2.94 g, 2.54 mmol) was added and the mixture was degassed again. The mixture was stirred at 115° C. for 18 h. LCMS showed the reaction was complete. The mixture was cooled and filtered. The solids were washed with more dioxane (25 mL). The combined filtrate was dried over Na₂SO₄, filtered and concentrated to yield a reddish oil. The crude product was purified by silica gel chromatography (330 g ISCO column) (EtOAc/heptane 10-40%) to give the title compound as a yellow solid (6.10 g, 21.61 mmol, 85% yield). LCMS (Condition 1): m/z 283.2 [M+H]⁺, 1.28 min. ¹H NMR (400 MHz, Chloroform-d) δ 7.77 (d, J=8.7 Hz, 1H), 7.62 (dd, J=5.6, 8.7 Hz, 1H), 7.09 (td, J=2.7, 8.5 Hz, 1H), 6.94 (dd, J=2.7, 8.9 Hz, 1H), 6.54 (d, J=8.7 Hz, 1H), 6.32 (dd, J=11.0, 17.5 Hz, 1H), 5.59 (d, J=17.4 Hz, 1H), 5.12 (d, J=11.0 Hz, 1H), 4.88 (s, 2H). ¹⁹F NMR (376 MHz, Chloroform-d) δ-57.67 (s, 3F), −114.93 (s, 1F).

### Step 2. 6-fluoro-N-(6-(5-fluoro-2-vinylphenyl)-5-(trifluoromethyl)pyridin-2-yl)pyridine-2-sulfonamide (int-b1)

6-(5-Fluoro-2-vinylphenyl)-5-(trifluoromethyl)pyridin-2-amine (6.1 g, 21.61 mmol) was dissolved in pyridine (30 mL) and treated with 6-fluoropyridine-2-sulfonyl chloride (5.50 g, 28.1 mmol). The resulting red solution was stirred at 20° C. for 2 days. LCMS showed the reaction was complete. The reaction mixture was diluted with EtOAc (200 mL) and 1 N HCl (50 mL). It was extracted with EtOAc (2×100 mL). The combined extracts were dried over Na₂SO₄ and concentrated. The oil obtained was treated with toluene (70 mL) and concentrated to yield a brown paste. The residue was taken up in DCM (30 mL). A white precipitate (desired product) formed. It was collected by filtration, washed with small amount of DCM and air-dried. The DCM filtrates were combined and purified by silica gel chromatography (330 g ISCO column) (EtOAc/heptane 10-40%). The desired material thus obtained was combined with the precipitated product obtained before. The title compound was obtained as an off-white powder (8.63 g, 19.52 mmol, 90%). Also, a mixture of the desired product and a by-product 6-fluoro-N-(6-(5-fluoro-2-vinylphenyl)-5-(trifluoromethyl)pyridin-2-yl)-N-((6-fluoropyridin-2-yl)sulfonyl)pyridine-2-sulfonamide was also obtained (1.2 g, 30% desired product and 70% by-product based on ¹H NMR). LCMS (Condition 1): m/z 441.9 [M+H]⁺, 1.68 min. ¹H NMR (500 MHz, DMSO-d₆) δ 12.19 (s, 1H), 8.20 (d, J=8.9 Hz, 1H), 8.11 (q, J=7.7 Hz, 1H), 7.83 (dd, J=1.7, 7.5 Hz, 1H), 7.71 (dd, J=5.7, 8.8 Hz, 1H), 7.48 (dd, J=1.9, 8.3 Hz, 1H), 7.29 (td, J=2.8, 8.5 Hz, 2H), 6.95 (d, J=8.7 Hz, 1H), 5.81 (dd, J=11.1, 17.4 Hz, 1H), 5.55 (d, J=17.4 Hz, 1H), 4.99 (d, J=11.1 Hz, 1H). ¹⁹F NMR (471 MHz, DMSO-d₆) δ−57.13 (s, 3F), −66.34 (s, 1F), −114.95 (s, 1F).

### Synthesis of intermediate 6-fluoro-N-(5-(trifluoromethyl)-6-(2-vinylphenyl)pyridin-2-yl)pyridine-2-sulfonamide (int-b2)

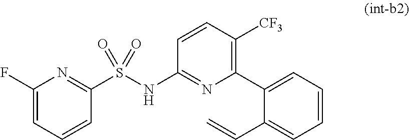 [SMILES: C=Cc1ccccc1-c1nc(NS(=O)(=O)c2cccc(F)n2)ccc1C(F)(F)F]

Synthesized using the procedure used for intermediate (int-b1) except in step 1 where 5-fluoro-2-vinylphenyl)boronic acid was replaced with (2-vinylphenyl)boronic acid. LCMS (Condition 1): m/z 424.1 [M+H]1, 1.37 min.

### Synthesis of intermediate N-(5-chloro-6-(2-vinylphenyl)pyridin-2-yl)-6-fluoropyridine-2-sulfonamide (int-b3)

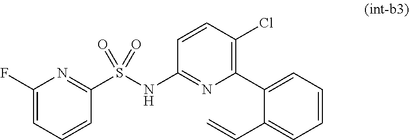 [SMILES: C=Cc1ccccc1-c1nc(NS(=O)(=O)c2cccc(F)n2)ccc1Cl]

Synthesized using the procedure used for intermediate (int-b1) except in step 1 where 5-fluoro-2-vinylphenyl)boronic acid was replaced with (2-vinylphenyl)boronic acid and 6-Chloro-5-(trifluoromethyl)pyridin-2-amine was replaced with (5,6-dichloropyridin-2-amine. LCMS (Condition 1): m/z 390.1 [M+H]⁺, 1.55 min.

### Synthesis of intermediate 6-fluoro-N-(5-methyl-6-(2-vinylphenyl)pyridin-2-yl)pyridine-2-sulfonamide (int-b4)

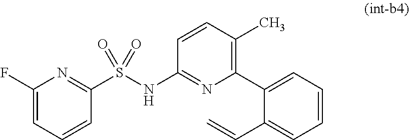 [SMILES: C=Cc1ccccc1-c1nc(NS(=O)(=O)c2cccc(F)n2)ccc1C]

Synthesized using the procedure used for intermediate (int-b1) except in step 1 where 5-fluoro-2-vinylphenyl)boronic acid was replaced with (2-vinylphenyl)boronic acid and 6-Chloro-5-(trifluoromethyl)pyridin-2-amine was replaced with 6-chloro-5-methylpyridin-2-amine. LCMS (Condition 1): m/z 390.1 [M+H]⁺, 1.55 min.

### Synthesis of intermediate 6-fluoro-N-(5-methoxy-6-(2-vinylphenyl)pyridin-2-yl)pyridine-2-sulfonamide (int-b5)

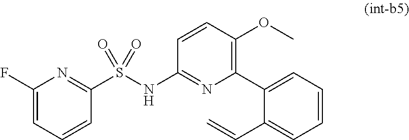 [SMILES: C=Cc1ccccc1-c1nc(NS(=O)(=O)c2cccc(F)n2)ccc1OC]

Synthesized using the procedure used for intermediate (int-b1) except in step 1 where 5-fluoro-2-vinylphenyl)boronic acid was replaced with (2-vinylphenyl)boronic acid and 6-Chloro-5-(trifluoromethyl)pyridin-2-amine was replaced with 6-chloro-5-methoxypyridin-2-amine. LCMS (Condition 1): m/z 386.2 [M+H]⁺, 1.52 min.

### Synthesis of intermediate 6-fluoro-N-(6-(2-vinylphenyl)pyridin-2-yl)pyridine-2-sulfonamide (int-b6)

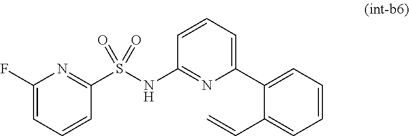 [SMILES: C=Cc1ccccc1-c1cccc(NS(=O)(=O)c2cccc(F)n2)n1]

Synthesized using the procedure used for intermediate (int-b1) except in step 1 where 5-fluoro-2-vinylphenyl)boronic acid was replaced with (2-vinylphenyl)boronic acid and 6-Chloro-5-(trifluoromethyl)pyridin-2-amine was replaced with 6-chloropyridin-2-amine. LCMS (Condition 1): m/z 356.1 [M+H]⁺1, 1.66 min.

### Synthesis of intermediate N-(6-(2-(allyloxy)phenyl)-5-(trifluoromethyl)pyridin-2-yl)-6-fluoropyridine-2-sulfonamide (int-b7)

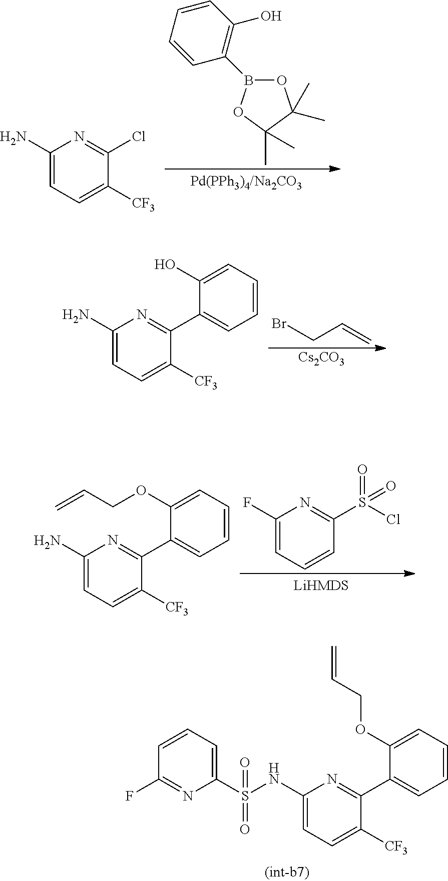

### Step 1. 2-(6-amino-3-(trifluoromethyl)pyridin-2-yl)phenol

In a 150 mL pressure flask containing a mixture of 6-chloro-5-(trifluoromethyl)pyridin-2-amine (1.50 g, 7.63 mmol), 2-(4,4,5,5-tetramethyl-1,3,2-dioxaborolan-2-yl)phenol (2.10 g, 9.16 mmol) and Na₂CO₃ (0.081 g, 0.76 mmol) in dioxane (4 mL) and water (1 mL) was evacuated and backfilled with nitrogen (2×). Pd(Ph₃P)₄ (0.25 g, 0.22 mmol) was added and the resulting mixture was heated in an oil bath at 120° C. for 4 h. The mixture was cooled to room temperature and then extracted with EtOAc (2×) and washed with water and brine. The combined extracts were dried over MgSO₄, filtered, and concentrated. The residue was purified by an ISCO column (EtOAc/heptane 0-100%) to afford the title compound (1.60 g, 6.29 mmol, 82% yield) as a white solid. LCMS (Condition 1): m/z 255.2 [M+H]⁺, 0.98 min.

### Step 2. Synthesis of 6-(2-(allyloxy)phenyl)-5-(trifluoromethyl)pyridin-2-amine

A mixture of 2-(6-amino-3-(trifluoromethyl)pyridin-2-yl)phenol (0.87 g, 3.42 mmol), Cs₂CO₃ (3.35 g, 10.27 mmol) and allyl bromide (0.36 mL, 4.11 mmol) in ACN (3 mL) was stirred at 60° C. for 3 h. The reaction mixture was quenched with water and acidified with 1 N HCl (10 mL). The mixture was extracted with EtOAc (2×). The organic layers were combined, dried (MgSO₄), filtered, and concentrated. The crude product was purified by an ISCO column (EtOAc/heptane 0-100%) to afford the title compound (530 mg, 1.80 mmol, 53% yield) as a white solid. LCMS (Condition 1): m/z 295.2 [M+H]⁺, 1.25 min.

### Step 3. N-(6-(2-(allyloxy)phenyl)-5-(trifluoromethyl)pyridin-2-yl)-6-fluoropyridine-2-sulfonamide (int-b7)

To a solution of 6-(2-(allyloxy)phenyl)-5-(trifluoromethyl)pyridin-2-amine (530 mg, 1.8 mmol) in THE (15 mL) was added dropwise 1 M LHMDS in THE (0.170 mL, 0.170 mmol) at 0° C. to achieve an orange solution. After 5 min, 6-fluoropyridine-2-sulfonyl chloride (0.35 mL, 2.7 mmol) in 2 mL of THE was added dropwise at 0° C. The reaction mixture was quenched with water and then acidified with 1 N HCl (5 mL). The mixture was extracted with EtOAc (2×). The organic layers were combined, dried over MgSO₄, filtered, and concentrated. The crude product was purified by an ISCO column (EtOAc/heptane 0-100%) to afford the title compound (480 mg, 1.1 mmol, 59% yield) as a brown solid. LCMS (Condition 1): m/z 454.2 [M+H]1, 1.54 min.

### Synthesis of intermediate N-(6-(2-(allyloxy)-5-fluorophenyl)-5-(trifluoromethyl)pyridin-2-yl)-6-fluoropyridine-2-sulfonamide (int-b8)

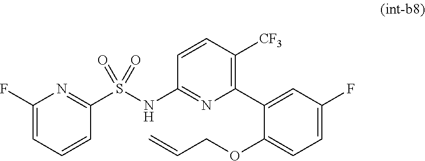 [SMILES: C=CCOc1ccc(F)cc1-c1nc(NS(=O)(=O)c2cccc(F)n2)ccc1C(F)(F)F]

Synthesized using the procedure used for intermediate (int-b7) except in step 1 where 2-(4,4,5,5-tetramethyl-1,3,2-dioxaborolan-2-yl)phenol was replaced with 4-fluoro-2-(4,4,5,5-tetramethyl-1,3,2-dioxaborolan-2-yl)phenol. LCMS (Condition 1): m/z 472.1 [M+H]⁺, 1.81 min.

### Synthesis of intermediate N-(5-chloro-6-(2-vinylphenyl)pyridin-2-yl)-6-(pent-4-en-1-yloxy)pyridine-2-sulfonamide (int-b9)

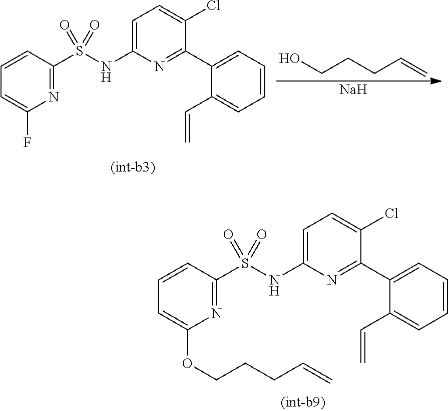

To a solution of N-(5-chloro-6-(2-vinylphenyl)pyridin-2-yl)-6-fluoropyridine-2-sulfonamide (int-b3) (59 mg, 0.15 mmol) and pent-4-en-1-ol (39 mg, 0.45 mmol) in DMF (2 mL) was added NaH (36 mg, 1.5 mmol) under nitrogen atmosphere. The mixture was stirred at room temperature for 1 h, and quenched with water. The reaction was acidified with a 10% citric acid solution, extracted with EtOAc, washed with water, NaHCO₃, brine (2×) and dried over Na₂SO₄. The product was purified on an ISCO silica gel column (EtOAc/heptane 0-20%) to get the title compound (53 mg, 75% yield) as an oil. LCMS (Condition 1): m/z 456.2 [M+H]⁺, 1.80 min, ¹H NMR (400 MHz, DMSO-d₆) 11.38 (s, 1H), 7.95 (d, J=8.8 Hz, 1H), 7.90 (dd, J=7.4, 8.3 Hz, 1H), 7.69 (d, J=7.9 Hz, 1H), 7.52 (d, J=7.3 Hz, 1H), 7.42 (m, 1H), 7.32 (m, 2H), 7.08 (d, J=8.3 Hz, 1H), 6.98 (d, J=7.3 Hz, 1H), 6.12 (dd, J=11.0, 17.6 Hz, 1H), 5.76 (ddt, J=6.6, 10.3, 16.9 Hz, 1H), 5.66 (m, 1H), 5.07 (d, J=11.2 Hz, 1H), 4.95 (m, 2H), 4.06 (t, J=6.6 Hz, 2H), 2.05 (m, 2H), 1.67 (m, 2H).

### Synthesis of intermediate 6-fluoro-N-(2′-fluoro-3-(trifluoromethyl)-[2,3′-bipyridin]-6-yl)pyridine-2-sulfonamide (int-b10)

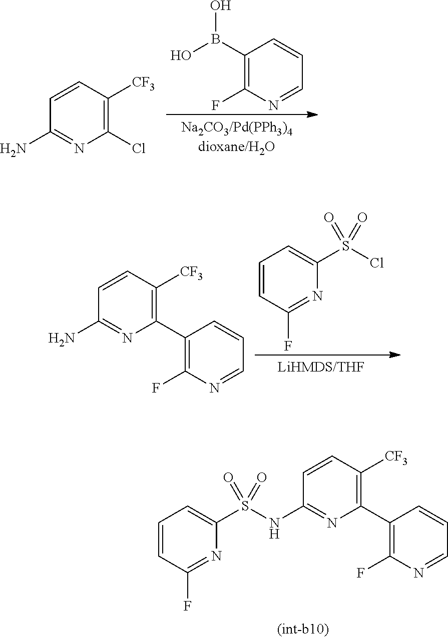

### Step 1. Synthesis of 2′-fluoro-3-(trifluoromethyl)-[2,3′-bipyridin]-6-amine

In a flask with an attached reflux condenser, 6-chloro-5-(trifluoromethyl)pyridin-2-amine (4.00 g, 20.4 mmol), 2-fluoropyridine-3-boronic acid (4.30 g, 30.5 mmol), Na₂CO₃ (6.47 g, 61.1 mmol), and Pd(Ph₃P)₄ (2.35 g, 2.04 mmol) were taken up in a mixture of dioxane (100 mL) and H₂O (16 mL). The mixture was subsequently sparged with argon and heated to 120° C. for 3 days. The crude reaction material was evaporated directly on silica gel and purified by flash column chromatography (80 g silica gel column, 0-8% MeOH/DCM, dry compound loading) to give the title compound (3.52 g, 13.7 mmol, 67% yield) as a yellow solid. LCMS (Condition 1): m/z 258.1 [M+H]⁺, 1.26 min.

### Step 2. Synthesis of 6-fluoro-N-(2′-fluoro-3-(trifluoromethyl)-[2,3′-bipyridin]-6-yl)pyridine-2-sulfonamide

In a flask, 2′-fluoro-3-(trifluoromethyl)-[2,3′-bipyridin]-6-amine (3.52 g, 13.7 mmol) was taken up in THE (100 mL) and the resulting solution was cooled to 0° C. To the solution was added 1 M LiHMDS in THE (27.4 mL, 27.4 mmol), then a solution of 6-fluoropyridine-2-sulfonyl chloride (4.01 g, 20.5 mmol) that had been dissolved in THE (10 mL). The reaction was stirred for 1 hr then quenched with 1 M HCl and extracted into EtOAc (×3). The organics were then washed with water and brine, dried over MgSO₄, and concentrated in vacuo. The crude material was evaporated on silica gel and purified by flash column chromatography (80 g silica gel column, 0-80% EtOAc/heptanes, dry compound loading) to give the title compound (2.49 g, 5.98 mmol, 44% yield) as a light tan solid. LCMS (Condition 1): m/z 417.0 [M+H]⁺, 1.49 min. ¹H NMR (600 MHz, DMSO-d₆) δ 12.28 (s, 1H), 8.39-8.32 (m, 1H), 8.24 (d, J=8.9 Hz, 1H), 8.20-8.14 (m, 1H), 7.88 (dd, J=7.5, 1.6 Hz, 1H), 7.80-7.74 (m, 1H), 7.52 (dd, J=8.3, 1.8 Hz, 1H), 7.48-7.43 (m, 1H), 7.28 (d, J=8.7 Hz, 1H).

### Synthesis of intermediate N-(2′,3-dichloro-[2,3′-bipyridin]-6-yl)-6-fluoropyridine-2-sulfonamide (int-b11)

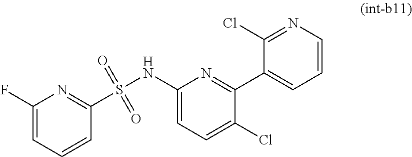 [SMILES: O=S(=O)(Nc1ccc(Cl)c(-c2cccnc2Cl)n1)c1cccc(F)n1]

Synthesized using the procedure used for intermediate (int-b10) except in step 1 where 6-chloro-5-(trifluoromethyl)pyridin-2-amine was replaced with 6-bromo-5-chloropyridin-2-ylamine and 2-fluoropyridine-3-boronic acid was replaced with 2-chloropyridine-3-boronic acid. LCMS (Condition 1): LCMS (Condition 1): m/z 398.9 [M+H]⁺, 1.46 min.

### Synthesis of intermediate N-(2′,3-dichloro-4′-methyl-[2,3′-bipyridin]-6-yl)-6-fluoropyridine-2-sulfonamide (int-b12)

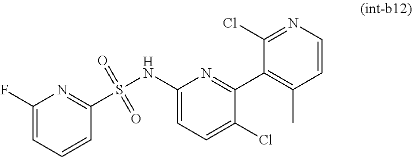 [SMILES: Cc1ccnc(Cl)c1-c1nc(NS(=O)(=O)c2cccc(F)n2)ccc1Cl]

Synthesized using the procedure used for intermediate (int-b10) except in step 1 where 6-chloro-5-(trifluoromethyl)pyridin-2-amine was replaced with 6-bromo-5-chloropyridin-2-ylamine and 2-fluoropyridine-3-boronic acid was replaced with 2-chloro-4-methylpyridine-3-boronic acid. LCMS (Condition 1): m/z 412.9 [M+H]⁺, 1.59 min

### Synthesis of intermediate N-(6-(2-((3-aminopropoxy)methyl)phenyl)-5-(trifluoromethyl)pyridin-2-yl)-6-fluoropyridine-2-sulfonamide (int-b13)

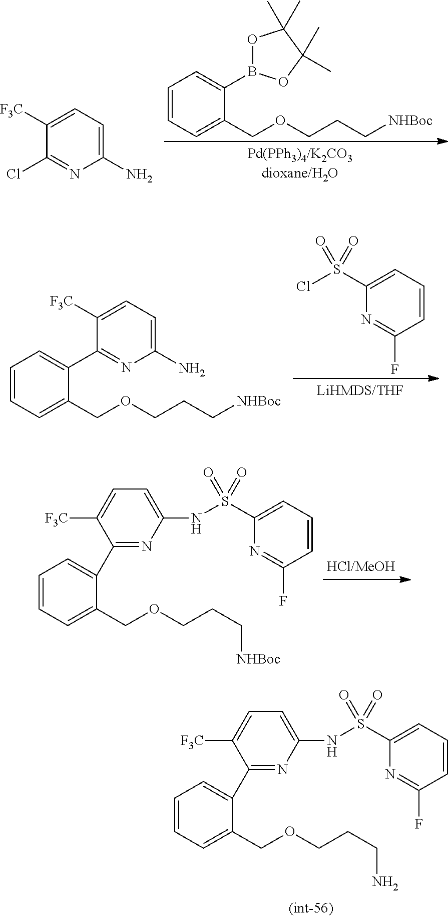 [SMILES: Nc1ccc(C(F)(F)F)c(Cl)n1 ; CC(C)(C)OC(=O)NCCCOCc1ccccc1B1OC(C)(C)C(C)(C)O1 ; CC(C)(C)OC(=O)NCCCOCc1ccccc1-c1nc(N)ccc1C(F)(F)F ; O=S(=O)(Cl)c1cccc(F)n1 ; CC(C)(C)OC(=O)NCCCOCc1ccccc1-c1nc(NS(=O)(=O)c2cccc(F)n2)ccc1C(F)(F)F ; NCCCOCc1ccccc1-c1nc(NS(=O)(=O)c2cccc(F)n2)ccc1C(F)(F)F]

### Step 1. Synthesis of tert-butyl (3-((2-(6-amino-3-(trifluoromethyl)pyridin-2-yl)benzyl)oxy)propyl)carbamate

6-chloro-5-(trifluoromethyl)pyridin-2-amine (80 mg, 0.20 mmol), tert-butyl (3-((2-(4,4,5,5-tetramethyl-1,3,2-dioxaborolan-2-yl)benzyl)oxy)propyl)carbamate (175 mg, 0.45) and K₂CO₃ (113 mg, 0.81 mmol) were suspended in Dioxane/water (3:1, 4 mL) in a microwave vial and flushed with N₂ for 2 min. Pd(PPh₃)₄(24 mg, 0.02 mmol) was added and flushed with N₂ for 2 min and closed the vial. The reaction was then microwaved at 135° C. for 45 min. It was filtered and washed with EtOAc. The filtrate was washed with brine and then with water, dried over Na₂SO₄, filtered and concentrated. It was then subjected to flash column chromatography (ISCO, 24 g column, EtOAc/heptane 0-60%) to give the title compound (120 mg, 0.14 mmol, 70% yield). LC-MS (Condition 1): m/z 426.2 [M+H]⁺, 1.98 min.

### Step 2. Synthesis of tert-butyl (3-((2-(6-((6-fluoropyridine)-2-sulfonamido)-3-(trifluoromethyl)pyridin-2-yl)benzyl)oxy)propyl)carbamate

tert-Butyl (3-((2-(6-amino-3-(trifluoromethyl)pyridin-2-yl)benzyl)oxy)propyl)carbamate (100 mg, 0.24 mmol) was suspended in THE (5 mL) and cooled at 0° C. under N₂ atmosphere. LiHMDS (79 mg, 0.47 mmol) was added in dropwise fashion. Continued stirring for 30 minutes and 6-fluoropyridine-2-sulfonyl chloride (40 mg, 0.21 mmol) in THE (1 mL) was added in dropwise, the reaction mixture was brought to room temperature and stirred for 3 h. Poured onto cold saturated NH₄Cl solution and worked up with EtOAc. Dried over Na₂SO₄ and concentrated. It was purified by flash column chromatography (ISCO, 12 g column, EtOAc/heptane 0-10%) to give the title compound as a yellow solid (76 mg, 0.12 mmol, 50% yield). LCMS (Condition 1): m/z 585.3 [M+H]⁺, 1.78 min. ¹H NMR (400 MHz, DMSO-d₆) δ 12.17 (s, 1H), 8.18 (d, J=8.9 Hz, 1H), 8.09 (q, J=7.8, 1H), 7.84 (dd, J=7.3, 2.0 Hz, 1H), 7.52-7.38 (m, 3H), 7.30 (ddd, J=7.6, 5.1, 3.7 Hz, 1H), 7.27-7.20 (m, 1H), 7.04 (d, J=7.5 Hz, 1H), 6.70 (t, J=5.6 Hz, 1H), 3.95 (s, 2H), 3.09 (dt, J=21.7, 5.0 Hz, 2H), 2.85 (q, J=6.6 Hz, 2H), 1.54-1.39 (m, 2H), 1.35 (s, 9H).

### Step 3. Synthesis of N-(6-(2-((3-aminopropoxy)methyl)phenyl)-5-(trifluoromethyl)pyridin-2-yl)-6-fluoropyridine-2-sulfonamide (int-b13)

tert-Butyl(3-((2-(6-((6-fluoropyridine)-2-sulfonamido)-3-(trifluoromethyl)pyridin-2-yl)benzyl)oxy)-propyl)carbamate (55 mg, 0.09 mmol) was stirred in HCl in MeOH (3.0 M, 1.0 mL, 3.0 mmol) in a vial at 55° C. for 3 h. The solvent was removed, the residue was dried and washed with diethyl ether to give the crude title compound (43 mg, 94% yield) as a brown solid. LCMS (Condition 1): m/z 485.2 [M+H]⁺, 1.39 min. ¹H NMR (400 MHz, DMSO-d₆) δ 12.27 (s, 1H), 8.18 (d, J=8.8 Hz, 1H), 8.14-7.97 (m, 3H), 7.83 (dd, J=7.4, 2.1 Hz, 1H), 7.49 (dd, J=8.2, 2.2 Hz, 1H), 7.46-7.39 (m, 2H), 7.36-7.19 (m, 2H), 7.02 (d, J=7.5 Hz, 1H), 4.00 (s, 2H), 3.29-2.98 (m, 2H), 2.82-2.59 (m, 2H), 1.85-1.49 (m, 2H).

### Synthesis of 6-bromo-N-(5-(trifluoromethyl)-6-(2-vinylphenyl)pyridin-2-yl)pyridine-2-sulfonamide (int-b14)

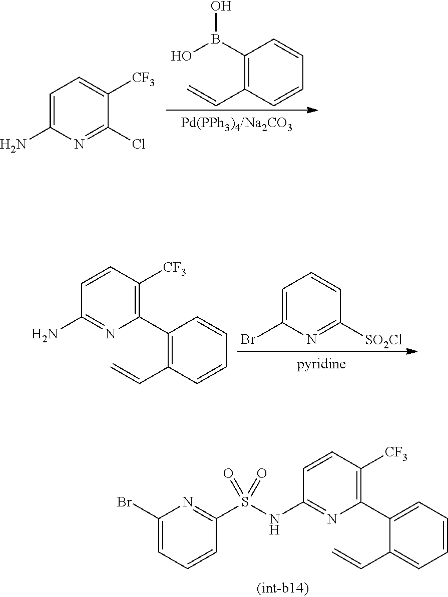

Synthesized using the procedure used for intermediate (int-b1) except in step 1 where 5-fluoro-2-vinylphenyl)boronic acid was replaced with 2-vinylphenyl)boronic acid and in step 2 where 6-fluoropyridine-2-sulfonyl chloride was replaced with 6-bromopyridine-2-sulfonyl chloride. LCMS (Condition 1): m/z 483.9 [M+H]⁺, 1.41 min.

### SYNTHESIS OF EXEMPLARY COMPOUNDS

### Example 1: Synthesis of 2³-(trifluoromethyl)-11-oxa-4-thia-3,6-diaza-2,5(2,6)-dipyridina-1(1,2)-benzenacycloundecaphane 4,4-dioxide (1)

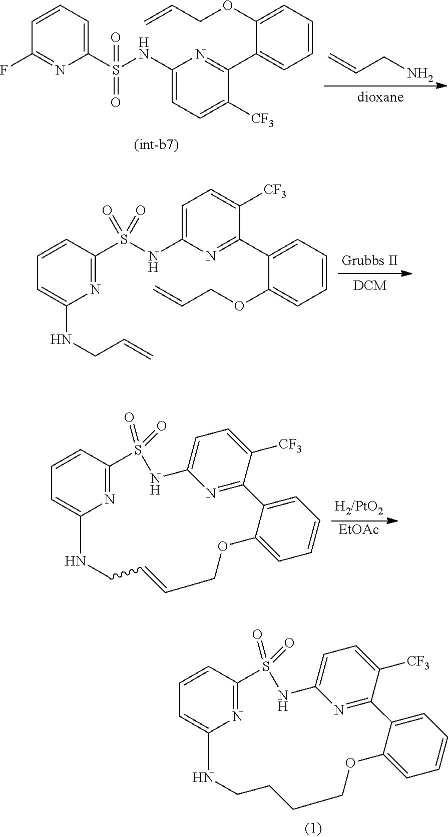

### Step 1. Synthesis of 6-(allylamino)-N-(6-(2-(allyloxy)phenyl)-5-(trifluoromethyl)pyridin-2-yl)pyridine-2-sulfonamide

A mixture containing N-(6-(2-(allyloxy)phenyl)-5-(trifluoromethyl)pyridin-2-yl)-6-fluoropyridine-2-sulfonamide (int-b7) (100 mg, 0.22 mmol), and prop-2-en-1-amine (63.0 mg, 1.10 mmol) in dioxane (5 mL) was heated at 12000 for 16 h. The mixture was diluted with water and 1 N aqueous HCl (2 mL) and then extracted with EtOAc. The organic layer was collected, dried over MgSO₄, filtered and concentrated. The residue was purified by an ISCO column (EtOAc/heptane 0-100%) to afford the title compound as a glassy solid. LCMS (Condition 1): m/z 491.2 [M+H]⁺, 1.62 mmi.

### Step 2. Synthesis of 23-(trifluoromethyl)-11-oxa-4-thia-3,6-diaza-2,5(2,6)-dipyridina-1(1,2)-benzenacycloundecaphan-8-ene 4,4-dioxide

To a solution of 6-(allylamino)-N-(6-(2-(allyloxy)phenyl)-5-(trifluoromethyl)pyridin-2-yl)pyridine-2-sulfonamide (80 mg, 0.163 mmol) in DCM (50 ml) was added Grubbs II catalyst ((1,3-Bis(2,4,6-trimethylphenyl)-2-imidazolidinylidene)dichloro(phenylmethylene)(tricyclohexylphosphine)ruthenium) (40 mg, 0.047 mmol) under nitrogen atmosphere. The mixture was heated at 50° C. in an oil bath for 2 h, then continued stirring at room temperature for 5 days. The mixture was concentrated in vacuo and the residue was purified by an ISCO column (EtOAc/heptane 0-100%) to afford the title compound as a brown solid. LCMS (Condition 1): m/z 463.2 [M+H]⁺, 1.53 min.

### Step 3. Synthesis of 2³-(trifluoromethyl)-11-oxa-4-thia-3,6-diaza-2,5(2,6)-dipyridna-(1,2)-benzenacycloundecaphane 4,4-dioxide (1)

To a solution of 2³-(trifluoromethyl)-11-oxa-4-thia-3,6-diaza-2,5(2,6)-dipyridina-1 (1,2)-benzenacycloundecaphan-8-ene 4,4-dioxide (55 mg, 0.119 mmol) in EtOAc (15 mL), was hydrogenated over PtO₂ hydrate (25 mg, 0.110 mmol) under hydrogen atmosphere at room temperature for 1.5 h. The mixture was filtered through Celite, rinsed with EtOAc, and the filtrate was concentrated in vacuo. The residue was purified by an ISCO column (EtOAc/heptane 0-100%) to afford the title compound as a white solid. LCMS (Condition 1): m/z 465.2 [M+H]⁺, 1.58 min ¹H NMR (400 MHz, DMSO-d₆) δ 11.32 (s, 1H), 8.09 (d, J=8.9 Hz, 1H), 7.56-7.45 (m, 2H), 7.40 (t, J=7.8 Hz, 1H), 7.17 (d, J=6.9 Hz, 1H), 7.13-6.94 (m, 4H), 6.62 (d, J=8.5 Hz, 1H), 4.00-3.82 (m, 2H), 3.02-2.89 (m, 1H), 2.87-2.73 (m, 1H), 1.77-1.61 (m, 1H), 1.62-1.44 (m, 2H), 1.42-1.26 (m, 1H).

### Example 2: Synthesis of 6-(2-hydroxyethyl)-2³-(trifluoromethyl)-11-oxa-4-thia-3,6-diaza-2,5(2,6)-dipyridina-1(1,2)-benzenacycloundecaphane 4,4-dioxide (2)

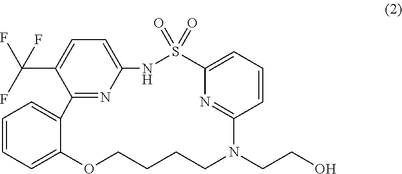 [SMILES: O=S1(=O)Nc2ccc(C(F)(F)F)c(n2)-c2ccccc2OCCCCN(CCO)c2cccc1n2]

6-(2-hydroxyethyl)-2³-(trifluoromethyl)-11-oxa-4-thia-3,6-diaza-2,5(2,6)-dipyridina-1(1,2)-benzenacycloundecaphane 4,4-dioxide (2) was synthesized using the procedure described in Example 1, except in step 1 where prop-2-en-1-amine was replaced with 2-(allylamino)ethan-1-ol (int-a15). LCMS (Condition 1): m/z 509.2 [M+H]⁺, 1.52 min. ¹H NMR (400 MHz, DMSO-d₆) δ 11.60 (s, 1H), 8.03 (d, J=9.0 Hz, 1H), 7.67 (dd, J=8.6, 7.3 Hz, 1H), 7.40 (t, J=7.7 Hz, 1H), 7.24 (t, J=7.6 Hz, 2H), 7.16 (d, J=6.2 Hz, 1H), 7.05 (d, J=8.3 Hz, 1H), 6.98 (t, J=7.4 Hz, 1H), 6.90 (d, J=8.7 Hz, 1H), 4.77 (t, J=5.2 Hz, 1H), 4.00 (s, 2H), 3.86 (s, 1H), 3.55-3.49 (m, 2H), 3.30-3.19 (m, 1H), 3.10-2.95 (m, 1H), 1.78 (s, 1H), 1.50 (s, 2H), 1.32-1.10 (m, 1H), 0.89-0.83 (m, 1H).

### Example 3: Synthesis of ethyl 2-(4,4-dioxido-2³-(trifluoromethyl)-11-oxa-4-thia-3,6-diaza-2,5(2,6)-dipyridina-1(1,2)-benzenacycloundecaphane-6-yl)acetate (3)

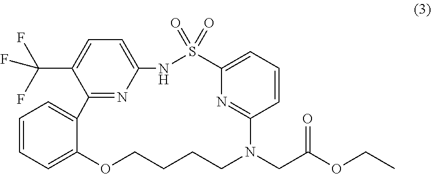 [SMILES: CCOC(=O)CN1CCCCOc2ccccc2-c2nc(ccc2C(F)(F)F)NS(=O)(=O)c2cccc1n2]

Ethyl 2-(4,4-dioxido-2³-(trifluoromethyl)-11-oxa-4-thia-3,6-diaza-2,5(2,6)-dipyridina-1(1,2)-benzenacycloundecaphane-6-yl)acetate (3) was synthesized using the procedure described in Example 1, except in step 1 where prop-2-en-1-amine was replaced with ethyl 2-(allylamino)acetate (int-a14). LCMS (Condition 1): m/z 551.2 [M+H]⁺, 1.65 min.

### Example 4: Synthesis of 2-(4,4-dioxido-2³-(trifluoromethyl)-1¹-oxa-4-thia-3,6-diaza-2,5(2,6)-dipyridina-1(1,2)-benzenacycloundecaphane-6-yl)acetic acid (4)

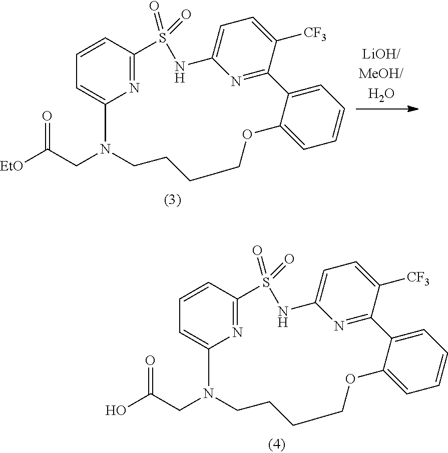 [SMILES: CCOC(=O)CN1CCCCOc2ccccc2-c2nc(ccc2C(F)(F)F)NS(=O)(=O)c2cccc1n2 ; O=C(O)CN1CCCCOc2ccccc2-c2nc(ccc2C(F)(F)F)NS(=O)(=O)c2cccc1n2]

A mixture of ethyl 2-(4,4-dioxido-2³-(trifluoromethyl)-11-oxa-4-thia-3,6-diaza-2,5(2,6)-dipyridina-1(1,2)-benzenacycloundecaphane-6-yl)acetate (3) (10 mg, 0.018 mmol) and LiOH (2.2 mg, 0.091 mmol) in MeOH (1.5 mL) and water (0.5 mL) was stirred overnight at room temperature. The mixture was concentrated in vacuo to remove most of MeOH. The remaining aqueous mixture was treated with 1N HCl (0.5 mL) to achieve a milky suspension. The mixture was then extracted with DCM, dried over MgSO₄, filtered, concentrated and dried under high vacuum with heating at 70° C. for 1 h to afford 2-(4,4-dioxido-2³-(trifluoromethyl)-11-oxa-4-thia-3,6-diaza-2,5(2,6)-dipyridina-1(1,2)-benzenacycloundecaphane-6-yl)acetic acid (4) as a white solid. LCMS 9 (Condition 1): m/z 523.2 [M+H]⁺, 1.50 min. ¹H NMR (400 MHz, DMSO-d₆) δ 12.75 (s, 1H), 11.65 (s, 1H), 8.03 (d, J=8.9 Hz, 1H), 7.70 (dd, J=8.5, 7.4 Hz, 1H), 7.40 (t, J=7.8 Hz, 1H), 7.32 (d, J=7.2 Hz, 1H), 7.24 (d, J=8.9 Hz, 1H), 7.17 (d, J=8.0 Hz, 1H), 7.05 (d, J=8.3 Hz, 1H), 6.98 (t, J=7.5 Hz, 1H), 6.69 (d, J=8.4 Hz, 1H), 4.24-4.19 (m, 1H), 4.07-4.02 (m, 2H), 3.84 (s, 1H), 2.97 (s, 1H), 1.82 (s, 1H), 1.51 (s, 2H), 1.24 (s, 2H).

### Example 5: Synthesis of 2³-(trifluoromethyl)-4-thia-3,6,11-triaza-1(3,2),2,5(2,6)-tripyridinacycloundecaphane 4,4-dioxide (5)

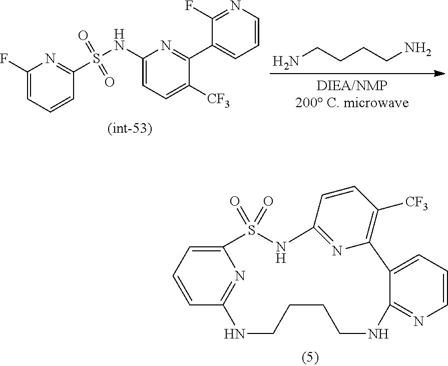

In a microwave vial, 6-fluoro-N-(2′-fluoro-3-(trifluoromethyl)-[2,3′-bipyridin]-6-yl)pyridine-2-sulfonamide (int-b10) (13.1 mg, 0.031 mmol) and 1,4-diaminobutane (2.8 mg, 0.031 mmol) were taken up in NMP (3 mL) and then DIEA (16 μL, 0.094 mmol) was added and the reaction was heated in the microwave to 200° C. for 1 h. The crude reaction material was evaporated on silica gel and purified by flash column chromatography (ISCO, 4 g RediSep Rf Gold column, EtOAc/heptane 0-60%, dry load) to give the product 2³-(trifluoromethyl)-4-thia-3,6,11-triaza-1(3,2),2,5(2,6)-tripyridinacycloundecaphane 4,4-dioxide (5) as an off white solid. LCMS (Condition 1): m/z 465.1 [M+H]⁺, 1.28 min. ¹H NMR (400 MHz, DMSO-d₆) δ 11.79 (s, 1H), 8.18 (d, J=8.9 Hz, 1H), 8.10 (dd, J=4.9, 1.7 Hz, 1H), 7.54-7.50 (m, 1H), 7.39-7.35 (m, 3H), 7.18 (d, J=7.1 Hz, 1H), 6.63 (dd, J=7.3, 5.1 Hz, 2H), 5.62 (s, 1H), 3.26-3.24 (m, 4H), 1.54 (s, 4H).

### Example 6: Synthesis of 2³-(trifluoromethyl)-4-thia-3,6,12-triaza-1(3,2),2,5(2,6)-tripyridinacyclododecaphane 4,4-dioxide (6)

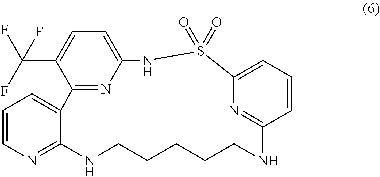 [SMILES: O=S1(=O)Nc2ccc(C(F)(F)F)c(n2)-c2cccnc2NCCCCCNc2cccc1n2]

2³-(trifluoromethyl)-4-thia-3,6,12-triaza-1(3,2),2,5(2,6)-tripyridinacyclododecaphane 4,4-dioxide (6) was synthesized using the procedure described in Example 5, except 1,4-diaminobutane was replaced with 1,5-diaminopentane. LCMS (Condition 1): m/z 479.1 [M+H]⁺, 1.26 min. ¹H NMR (400 MHz, DMSO-d₆) δ 11.70 (s, 1H), 8.26 (d, J=9.0 Hz, 1H), 8.00 (dd, J=6.1, 1.5 Hz, 1H), 7.74 (s, 2H), 7.62 (d, J=8.2 Hz, 1H), 7.49 (dd, J=8.4, 7.3 Hz, 1H), 7.08 (d, J=6.7 Hz, 2H), 6.92 (s, 1H), 6.60 (d, J=8.4 Hz, 1H), 3.51 (s, 2H), 3.31 (s, 2H), 1.49 (s, 2H), 1.37-1.36 (m, 2H), 1.22-1.21 (m, 2H).

### Example 7: Synthesis of 2³-(trifluoromethyl)-4-thia-3,6,13-triaza-1(3,2),2,5(2,6)-tripyridinacyclotridecaphane 4,4-dioxide (7)

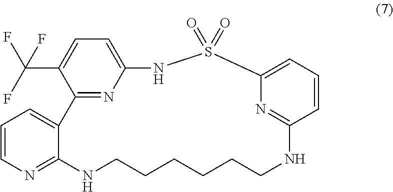 [SMILES: O=S1(=O)Nc2ccc(C(F)(F)F)c(n2)-c2cccnc2NCCCCCCNc2cccc1n2]

2³-(trifluoromethyl)-4-thia-3,6,13-triaza-1(3,2),2,5(2,6)-tripyridinacyclotridecaphane 4,4-dioxide (7) was synthesized using the procedure described in Example 5, except 1,4-diaminobutane was replaced with 1,6-diaminohexane. LCMS (Condition 1): m/z 493.2 [M+H]⁺, 1.35 min. ¹H NMR (400 MHz, DMSO-d₆) δ 11.88 (s, 1H), 8.21 (d, J=9.0 Hz, 1H), 8.04 (dd, J=5.9, 1.6 Hz, 1H), 7.67 (s, 1H), 7.55 (dd, J=8.5, 7.2 Hz, 1H), 7.37 (d, J=9.0 Hz, 1H), 7.22 (d, J=7.1 Hz, 1H), 7.12 (d, J=5.0 Hz, 1H), 6.88 (s, 1H), 6.67 (d, J=8.5 Hz, 1H), 3.29 (s, 2H), 3.21 (s, 2H), 1.53 (s, 4H), 1.31-1.22 (m, 4H).

### Example 8: Synthesis of 6,13-dimethyl-2³-(trifluoromethyl)-4-thia-3,6,13-triaza-1(3,2),2,5(2,6)-tripyridinacyclotridecaphane 4,4-dioxide (8)

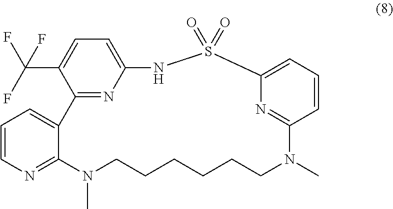 [SMILES: CN1CCCCCCN(C)c2ncccc2-c2nc(ccc2C(F)(F)F)NS(=O)(=O)c2cccc1n2]

6,13-dimethyl-2³-(trifluoromethyl)-4-thia-3,6,13-triaza-1(3,2),2,5(2,6)-tripyridinacyclotridecaphane 4,4-dioxide (8) was synthesized using the procedure described in Example 5, except 1,4-diaminobutane was replaced with N¹,N6-dimethylhexane-1,6-diamine. LCMS (Condition 1): m/z 521.2 [M+H]⁺, 1.44 min. ¹H NMR (400 MHz, DMSO-d₆) δ 11.73 (s, 1H), 8.20 (dd, J=4.8, 1.8 Hz, 1H), 8.15 (d, J=8.9 Hz, 1H), 7.68 (dd, J=8.6, 7.3 Hz, 1H), 7.52-7.36 (m, 2H), 7.14 (d, J=7.2 Hz, 1H), 6.88 (dd, J=7.4, 4.9 Hz, 1H), 6.81 (d, J=8.7 Hz, 1H), 3.95 (s, 1H), 3.38-3.37 (m, 1H), 2.89 (s, 3H), 2.66 (s, 4H), 2.39-2.25 (m, 1H), 1.47 (s, 1H), 1.33 (s, 1H), 1.10 (s, 1H), 0.76 (s, 5H).

### Example 9: Synthesis of 6,10-dimethyl-2³-(trifluoromethyl)-4-thia-3,6,10-triaza-1(3,2),2,5(2,6)-tripyridinacyclodecaphane 4,4-dioxide (9)

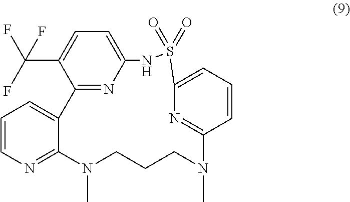 [SMILES: CN1CCCN(C)c2ncccc2-c2nc(ccc2C(F)(F)F)NS(=O)(=O)c2cccc1n2]

6,10-dimethyl-2³-(trifluoromethyl)-4-thia-3,6,10-triaza-1(3,2),2,5(2,6)-tripyridinacyclodecaphane 4,4-dioxide (9) was synthesized using the procedure described in Example 5, except 1,4-diaminobutane was replaced with N¹,N3-dimethylpropane-1,3-diamine. LCMS (Condition 1): m/z 479.1 [M+H]⁺, 1.35 min. ¹H NMR (400 MHz, Methanol-d₄) δ 8.12 (dd, J=5.0, 1.9 Hz, 1H), 7.65-7.52 (m, 2H), 7.41 (dd, J=7.3, 1.9 Hz, 1H), 7.20-7.10 (m, 2H), 6.85 (dd, J=7.3, 5.0 Hz, 1H), 6.54 (d, J=8.4 Hz, 1H), 3.55-3.48 (m, 1H), 3.26-3.15 (m, 1H), 3.00-2.95 (m, 1H), 2.92 (s, 3H), 2.83 (s, 3H), 2.63-2.51 (m, 1H), 1.73-1.69 (m, 1H), 0.80-0.76 (m, 1H).

### Example 10: Synthesis of 2³-(trifluoromethyl)-4-thia-3,6,10-triaza-1(3,2),2,5(2,6)-tripyridinacyclodecaphane 4,4-dioxide (10)

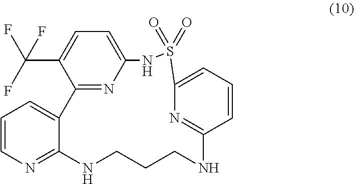 [SMILES: O=S1(=O)Nc2ccc(C(F)(F)F)c(n2)-c2cccnc2NCCCNc2cccc1n2]

2³-(trifluoromethyl)-4-thia-3,6,10-triaza-1(3,2),2,5(2,6)-tripyridinacyclodecaphane 4,4-dioxide (10) was synthesized using the procedure described in Example 5, except 1,4-diaminobutane was replaced with 1,3-diaminopropane. LCMS (Condition 1): m/z 451.1 [M+H]⁺, 1.19 min. ¹H NMR (400 MHz, DMSO-d₆) δ 11.33 (s, 1H), 8.27 (d, J=8.8 Hz, 1H), 8.06 (dd, J=6.0, 1.5 Hz, 1H), 7.80 (s, 1H), 7.56-7.44 (m, 2H), 7.02 (d, J=7.2 Hz, 2H), 6.91 (s, 1H), 6.63 (d, J=8.5 Hz, 1H), 3.44 (s, 4H), 1.73 (s, 2H).

### Example 11: Synthesis of 2³-(trifluoromethyl)-10-oxa-4-thia-3,6-diaza-2,5(2,6)-dipyridina-1(1,2)-benzenacycloundecaphane 4,4-dioxide (11)

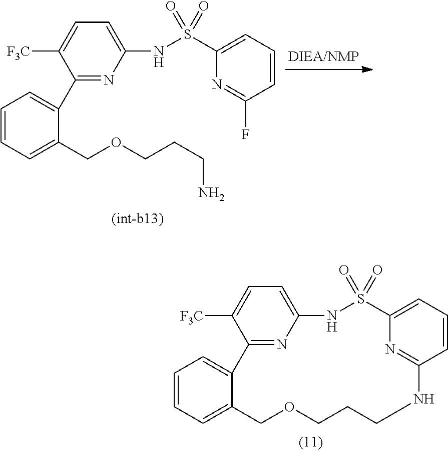 [SMILES: NCCCOCc1ccccc1-c1nc(NS(=O)(=O)c2cccc(F)n2)ccc1C(F)(F)F ; O=S1(=O)Nc2ccc(C(F)(F)F)c(n2)-c2ccccc2COCCCNc2cccc1n2]

N-(6-(2-((3-aminopropoxy)methyl)phenyl)-5-(trifluoromethyl)pyridin-2-yl)-6-fluoropyridine-2-sulfonamide (int-b13) (31 mg, 0.07 mmol) was dissolved in NMP (2 mL) in a vial and DIEA (0.27 mL, 1.54 mmol) was added. The vial was closed after flushing with nitrogen and stirred at 135° C. for 16 h. Poured onto water and extracted with EtOAc. Dried over Na₂SO₄, filtered and concentrated. It was purified by flash column chromatography (ISCO, 12 g column, MeOH/DCM 0-10%, dry load) to give 2³-(trifluoromethyl)-10-oxa-4-thia-3,6-diaza-2,5(2,6)-dipyridina-1(1,2)-benzenacycloundecaphane 4,4-dioxide (11) as a white solid. LCMS (Condition 1): m/z 465.2 [M+H]+. 1.74 min. ¹H NMR (400 MHz, DMSO-d₆) δ 12.27 (s, 1H), 8.18 (d, J=8.8 Hz, 1H), 8.15-7.97 (m, 2H), 7.83 (dd, J=7.4, 2.1 Hz, 1H), 7.49 (dd, J=8.2, 2.2 Hz, 1H), 7.43-7.37 (m, 2H), 7.35-7.19 (m, 2H), 7.02 (d, J=7.5 Hz, 1H), 4.00 (s, 2H), 3.29-3.01 (m, 2H), 2.80-2.54 (m, 2H), 1.82-1.50 (m, 2H).

### Example 12: Synthesis of 6-(5-hydroxypentyl)-2³-(trifluoromethyl)-4-thia-3,6-diaza-2,5(2,6)-dipyridina-1(1,2)-benzenacyclododecaphane 4,4-dioxide (12)

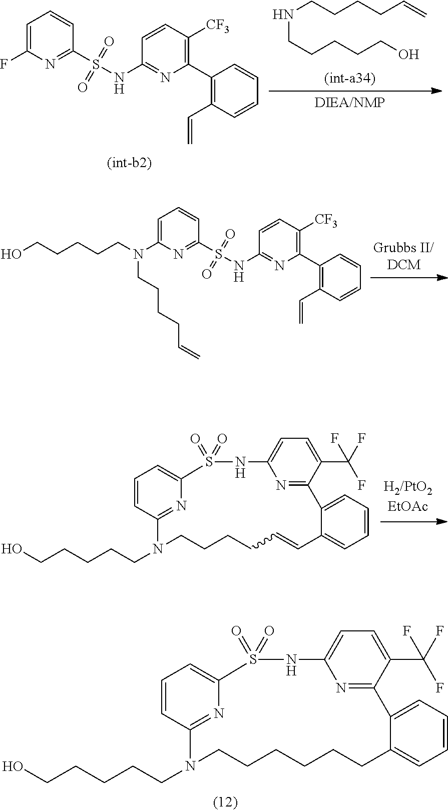 [SMILES: C=Cc1ccccc1-c1nc(NS(=O)(=O)c2cccc(F)n2)ccc1C(F)(F)F ; C=CCCCCNCCCCCO ; C=CCCCCN(CCCCCO)c1cccc(S(=O)(=O)Nc2ccc(C(F)(F)F)c(-c3ccccc3C=C)n2)n1 ; O=S1(=O)Nc2ccc(C(F)(F)F)c(n2)-c2ccccc2C=CCCCCN(CCCCCO)c2cccc1n2 ; O=S1(=O)Nc2ccc(C(F)(F)F)c(n2)-c2ccccc2CCCCCCN(CCCCCO)c2cccc1n2]

### Step 1. Synthesis of 6-(hex-5-en-1-yl(5-hydroxypentyl)amino)-N-(5-(trifluoromethyl)-6-(2-vinylphenyl)pyridin-2-yl)pyridine-2-sulfonamide

In a sealed tube, the mixture of 5-(hex-5-en-1-ylamino)pentan-1-ol (int-a34) (263 mg, 1.41 mmol), 6-fluoro-N-(5-(trifluoromethyl)-6-(2-vinylphenyl)pyridin-2-yl)pyridine-2-sulfonamide (int-b2) (200 mg, 0.47 mmol) and DIEA (0.33 mL, 1.88 mmol) in NMP (3 mL) was stirred overnight at 150° C., LCMS indicated the reaction was almost complete. The reaction mixture was poured into 200 mL of EtOAc in a 500-mL separation funnel, acidified with 10% citric acid, then washed with water (20 mL×4) and brine (20 mL). The organic phase was dried over Na₂SO₄, filtered and concentrated, the residue was then subjected to ISCO purification (40 g column, EtOAc/heptane 0-100%) to get the title compound (225 mg, 0.38 mmol, 81% yield) as a slightly brown solid. LCMS (Condition 1): m/z 589.2 [M+1]*, 1.93 min.

### Step 2. 6-(5-hydroxypentyl)-2³-(trifluoromethyl)-4-thia-3,6-diaza-2,5(2,6)-dipyridina-1(1,2)-benzenacyclododecaphan-11-ene 4,4-dioxide

In a 500-mL flask with a solution of 6-(hex-5-en-1-yl(5-hydroxypentyl)amino)-N-(5-(trifluoromethyl)-6-(2-vinylphenyl)pyridin-2-yl)pyridine-2-sulfonamide (220 mg, 0.37 mmol) in 200 mL of DCM (or DCE) was purged with argon. Grubbs II (64 mg, 0.075 mmol) catalyst was added and purged with argon again. The mixture was stirred overnight at 55° C. (or overnight in DCE at 70-75° C.). LC-MS indicated the reaction was complete. The reaction was concentrated and then the residue was subjected to ISCO purification (80 g column, EtOAc/hexane 0-100%) to give the title compound (194 mg, 0.35 mmol, 94% yield) (a mixture of cis-& trans-product but mainly trans-product) as a white solid. LC-MS: m/z 561.2 [M+1]⁺, 1.88 min.

### Step 3. 6-(5-hydroxypentyl)-2³-(trifluoromethyl)-4-thia-3,6-diaza-2,5(2,6)-dipyridina-1(1,2)-benzenacyclododecaphane 4,4-dioxide (12)

The mixture of 6-(5-hydroxypentyl)-2³-(trifluoromethyl)-4-thia-3,6-diaza-2,5(2,6)-dipyridina-1(1,2)-benzenacyclododecaphan-11-ene 4,4-dioxide (a mixture of cis-& trans-product but mainly trans-product) (160 mg, 0.285 mmol) and PtO₂ (65 mg, 0.285 mmol) in EtOAc (24 mL) was stirred overnight under hydrogen at room temperature, LC-MS indicated the reaction was complete. The reaction mixture was filtered through a layer of celite to remove platinum and the filtrate was then concentrated. The residue was subjected to ISCO purification (40 g column, EtOAc in heptane 0-100%) to get the title compound was obtained. LC-MS (Condition 1): m/z 563.2 [M+1]⁺, 1.92 min. ¹H NMR (400 MHz, DMSO-d₆)¹H NMR (400 MHz, DMSO-d6) δ 11.67 (s, 1H), 8.16 (d, J=8.9 Hz, 1H), 7.69 (dd, J=8.7, 7.2 Hz, 1H), 7.51-7.37 (m, 1H), 7.34 (td, J=7.4, 1.5 Hz, 1H), 7.29-7.20 (m, 2H), 7.20-7.10 (m, 2H), 6.84 (d, J=8.7 Hz, 1H), 4.38 (t, J=5.1 Hz, 1H), 3.72-3.49 (m, 1H), 3.43-3.35 (m, 3H), 3.33-3.11 (m, 2H), 2.71-2.54 (m, 1H), 2.14-1.91 (m, 1H), 1.55-1.17 (m, 9H), 1.17-0.98 (m, 3H), 0.96-0.82 (m, 1H), 0.82-0.67 (m, 1H).

### Example 13: Synthesis of 6-(2-morpholinoethyl)-2³-(trifluoromethyl)-4-thia-3,6-diaza-2,5(2,6)-dipyridina-1(1,2)-benzenacycloundecaphane 4,4-dioxide (13)

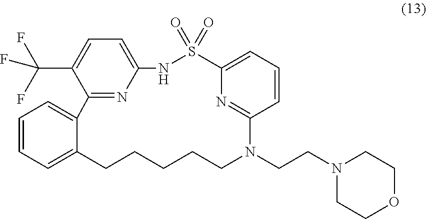 [SMILES: O=S1(=O)Nc2ccc(C(F)(F)F)c(n2)-c2ccccc2CCCCCN(CCN2CCOCC2)c2cccc1n2]

6-(2-morpholinoethyl)-2³-(trifluoromethyl)-4-thia-3,6-diaza-2,5(2,6)-dipyridina-1(1,2)-benzenacycloundecaphane 4,4-dioxide (13) was synthesized using the procedure described in Example 12 except 5-(hex-5-en-1-ylamino)pentan-1-ol (int-a34) was replaced with N-(2-morpholinoethyl)pent-4-en-1-amine (int-a1). LCMS (Condition 1): m/z 576.3 [M+H]⁺, 1.26 min. ¹H NMR (400 MHz, DMSO-d₆) δ 11.77 (s, 1H), 8.09 (d, J=9.0 Hz, 1H), 7.72 (dd, J=7.3, 8.7 Hz, 1H), 7.36 (td, J=1.4, 7.5 Hz, 1H), 7.29 (m, 1H), 7.23 (m, 2H), 7.19 (d, J=8.8 Hz, 1H), 7.14 (d, J=7.5 Hz, 1H), 6.84 (d, J=8.8 Hz, 1H), 3.95 (m, 1H), 3.53 (m, 4H), 3.38 (m, 3H), 2.89 (m, 1H), 2.48 (m, 1H), 2.40 (m, 6H), 1.90 (m, 1H), 1.72 (m, 1H), 1.35 (m, 2H), 1.17 (m, 1H), 1.03 (m, 1H). ¹⁹F NMR (376 MHz, DMSO-d6) δ −56.88 (s).

### Example 14: Synthesis of 6-(2-(3-hydroxypropoxy)ethyl)-2³-(trifluoromethyl)-4-thia-3,6-diaza-2.5(2.6)-dipyridina-1(1.2)-benzenacycloundecaphane 4,4-dioxide (14)

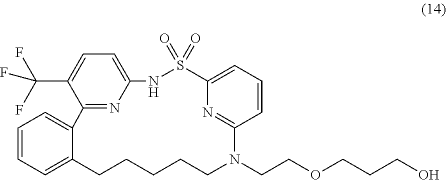 [SMILES: O=S1(=O)Nc2ccc(C(F)(F)F)c(n2)-c2ccccc2CCCCCN(CCOCCCO)c2cccc1n2]

6-(2-(3-hydroxypropoxy)ethyl)-2³-(trifluoromethyl)-4-thia-3,6-diaza-2,5(2,6)-dipyridina-1(1,2)-benzenacycloundecaphane 4,4-dioxide (14) was synthesized using the procedure described in Example 12 except 5-(hex-5-en-1-ylamino)pentan-1-ol (int-a34) was replaced with 3-(2-(pent-4-en-1-ylamino)ethoxy)propan-1-ol (int-a2). LCMS (Condition 1): m/z 565.2 [M+H]⁺, 1.87 min. ¹H NMR (400 MHz, DMSO-d₆) δ 11.79 (s, 1H), 8.09 (d, J=9.0 Hz, 1H), 7.70 (dd, J=8.7, 7.3 Hz, 1H), 7.35 (m, 1H), 7.28 (m, 1H), 7.23 (m, 2H), 7.18 (d, J=8.8 Hz, 1H), 7.14 (d, J=7.5 Hz, 1H), 6.90 (d, J=8.8 Hz, 1H), 4.35 (t, J=5.2 Hz, 1H), 3.94 (m, 1H), 3.42 (m, 8H), 2.91 (m, 1H), 2.39 (m, 1H), 1.91 (m, 1H), 1.73 (m, 1H), 1.58 (p, J=6.4 Hz, 2H), 1.32 (m, 2H), 1.10 (m, 3H). ¹⁹F NMR (376 MHz, DMSO-d6) δ −56.86 (s).

### Example 15: Synthesis of 6-(5-hydroxypentyl)-2³-(trifluoromethyl)-4-thia-3,6-diaza-2,5(2,6)-dipyridina-1(1,2)-benzenacycloundecaphane 4,4-dioxide (15)

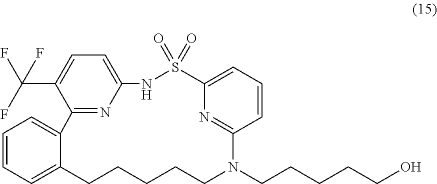 [SMILES: O=S1(=O)Nc2ccc(C(F)(F)F)c(n2)-c2ccccc2CCCCCN(CCCCCO)c2cccc1n2]

6-(5-hydroxypentyl)-2³-(trifluoromethyl)-4-thia-3,6-diaza-2,5(2,6)-dipyridina-1(1,2)-benzenacycloundecaphane 4,4-dioxide (15) was synthesized using the procedure described in Example 12 except 5-(hex-5-en-1-ylamino)pentan-1-ol (int-a34) was replaced with 5-(pent-4-en-1-ylamino)pentan-1-ol (int-a33). LC-MS (Condition 1): m/z 549.2 [M+1]⁺, 1.94 min. ¹H NMR (400 MHz, DMSO-d₆) δ 11.77 (s, 1H), 8.10 (d, J=8.9 Hz, 1H), 7.71 (dd, J=8.7, 7.2 Hz, 1H), 7.36 (td, J=7.4, 1.4 Hz, 1H), 7.31-7.26 (m, 1H), 7.26-7.17 (m, 3H), 7.14 (d, J=7.6 Hz, 1H), 6.81 (d, J=8.7 Hz, 1H), 4.36 (t, J=5.1 Hz, 1H), 4.04-3.86 (m, 1H), 3.40-3.34 (m, 3H), 3.33-3.12 (m, 2H), 2.95-2.79 (m, 1H), 2.46-2.34 (m, 1H), 1.96-1.81 (m, 1H), 1.79-1.62 (m, 1H), 1.56-1.36 (m, 4H), 1.36-1.21 (m, 3H), 1.21-1.08 (m, 2H), 1.08-0.92 (m, 1H).

### Example 16: Synthesis of 6-(4-hydroxybutyl)-2³-(trifluoromethyl)-4-thia-3,6-diaza-2,5(2,6)-dipyridina-1(1,2)-benzenacyclododecaphane 4,4-dioxide (16)

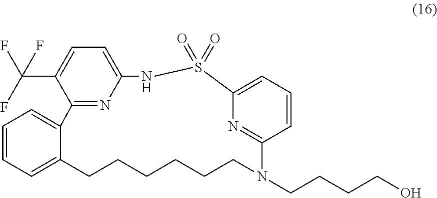 [SMILES: O=S1(=O)Nc2ccc(C(F)(F)F)c(n2)-c2ccccc2CCCCCCN(CCCCO)c2cccc1n2]

6-(4-hydroxybutyl)-2³-(trifluoromethyl)-4-thia-3,6-diaza-2,5(2,6)-dipyridina-1(1,2)-benzenacyclododecaphane 4,4-dioxide (16) was synthesized using the procedure described in Example 12 except 5-(hex-5-en-1-ylamino)pentan-1-ol (int-a34) was replaced with 4-(hex-5-en-1-ylamino)butan-1-ol (int-a35). LC-MS (Condition 1): m/z 549.2 [M+1]⁺, 1.87 min. ¹H NMR (400 MHz, DMSO-d₆) δ 11.67 (s, 1H), 8.16 (d, J=8.9 Hz, 1H), 7.70 (dd, J=8.8, 7.2 Hz, 1H), 7.51-7.37 (m, 1H), 7.34 (td, J=7.4, 1.5 Hz, 1H), 7.30-7.20 (m, 2H), 7.20-7.10 (m, 2H), 6.85 (d, J=8.7 Hz, 1H), 4.44 (t, J=5.1 Hz, 1H), 3.70-3.50 (m, 1H), 3.45-3.34 (m, 3H), 3.32-3.11 (m, 2H), 2.68-2.54 (m, 1H), 2.12-1.92 (m, 1H), 1.58-1.17 (m, 8H), 1.16-1.00 (m, 2H), 0.96-0.82 (m, 1H), 0.82-0.68 (m, 1H).

### Example 17: Synthesis of 6-(4-hydroxybutyl)-2³-methoxy-4-thia-3,6-diaza-2,5(2,6)-dipyridina-1(1,2)-benzenacycloundecaphane 4,4-dioxide (17)

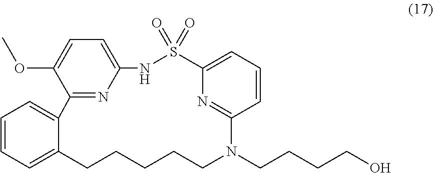 [SMILES: COc1ccc2nc1-c1ccccc1CCCCCN(CCCCO)c1cccc(n1)S(=O)(=O)N2]

6-(4-hydroxybutyl)-2³-methoxy-4-thia-3,6-diaza-2,5(2,6)-dipyridina-1(1,2)-benzenacycloundecaphane 4,4-dioxide (17) was synthesized using the procedure described in Example 12 except 6-fluoro-N-(5-(trifluoromethyl)-6-(2-vinylphenyl)pyridin-2-yl)pyridine-2-sulfonamide (int-b2) was replaced with 6-fluoro-N-(5-methoxy-6-(2-vinylphenyl)pyridin-2-yl)pyridine-2-sulfonamide (int-b5) and 5-(hex-5-en-1-ylamino)pentan-1-ol (int-a34) was replaced with 4-(pent-4-en-1-ylamino)butan-1-ol (int-a29). LC-MS (Condition 1): m/z 497.3 [M+1]⁺, 1.70 min. ¹H NMR (400 MHz, Methanol-d₄) δ 7.63 (ddd, J=8.7, 7.2, 2.4 Hz, 1H), 7.43 (dd, J=9.0, 2.5 Hz, 1H), 7.35-7.27 (m, 1H), 7.28-7.17 (m, 4H), 7.15 (dt, J=7.6, 1.9 Hz, 1H), 6.74 (ddd, J=8.8, 1.6, 0.6 Hz, 1H), 3.70 (s, 3H), 3.45-3.38 (m, 2H), 2.42 (t, J=7.3 Hz, 2H), 2.33 (t, J=6.9 Hz, 2H), 1.80-1.74 (m, 2H), 1.68-1.38 (m, 5H), 1.27 (t, J=7.7 Hz, 3H), 1.09-0.90 (m, 2H).

### Example 18: Synthesis of 6-ethyl-2³-(trifluoromethyl)-4-thia-3,6-diaza-2,5(2,6)-dipyridina-1(1,2)-benzenacycloundecaphane 4,4-dioxide (18)

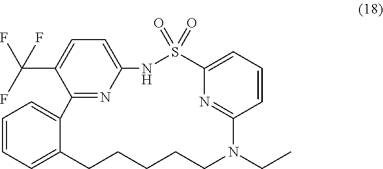 [SMILES: CCN1CCCCCc2ccccc2-c2nc(ccc2C(F)(F)F)NS(=O)(=O)c2cccc1n2]

6-ethyl-2³-(trifluoromethyl)-4-thia-3,6-diaza-2,5(2,6)-dipyridina-1(1,2)-benzenacycloundecaphane 4,4-dioxide (18) was synthesized using the procedure described in Example 12 except 5-(hex-5-en-1-ylamino)pentan-1-ol (int-a34) was replaced with N-ethylpent-4-en-1-amine (int-a32). LC-MS (Condition 1): m/z 491.2 [M+1]⁺, 1.98 min. ¹H NMR (400 MHz, DMSO-d₆) δ 11.77 (s, 1H), 8.10 (d, J=8.9 Hz, 1H), 7.71 (dd, J=8.7, 7.2 Hz, 1H), 7.40-7.32 (m, 1H), 7.31-7.26 (m, 1H), 7.26-7.17 (m, 3H), 7.14 (d, J=7.6 Hz, 1H), 6.83 (d, J=8.7 Hz, 1H), 4.04-3.85 (m, 1H), 3.43-3.22 (m, 3H), 2.93-2.79 (m, 1H), 2.46-2.34 (m, 1H), 1.94-1.81 (m, 1H), 1.78-1.62 (m, 1H), 1.42-1.22 (m, 2H), 1.20-1.09 (m, 2H), 1.04 (t, J=7.0 Hz, 3H).

### Example 19: Synthesis of 6-(4-hydroxybutyl)-2³-methyl-4-thia-3,6-diaza-2,5(2,6)-dipyridina-1(1,2)-benzenacycloundecaphane 4,4-dioxide (19)

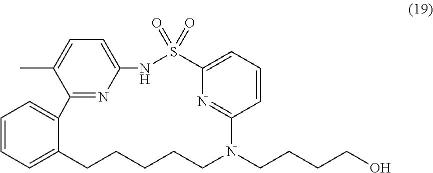 [SMILES: Cc1ccc2nc1-c1ccccc1CCCCCN(CCCCO)c1cccc(n1)S(=O)(=O)N2]

6-(4-hydroxybutyl)-2³-methyl-4-thia-3,6-diaza-2,5(2,6)-dipyridina-1(1,2)-benzenacycloundecaphane 4,4-dioxide (19) was synthesized using the procedure described in Example 12 except 6-fluoro-N-(5-(trifluoromethyl)-6-(2-vinylphenyl)pyridin-2-yl)pyridine-2-sulfonamide (int-b2) was replaced with 6-fluoro-N-(5-methyl-6-(2-vinylphenyl)pyridin-2-yl)pyridine-2-sulfonamide (int-b4) and 5-(hex-5-en-1-ylamino)pentan-1-ol (int-a34) was replaced with 4-(pent-4-en-1-ylamino)butan-1-ol (int-a29). LCMS (Condition 1): m/z 481.2 [M+H]⁺, 1.27 min; ¹H NMR (400 MHz, Chloroform-d) δ 7.56-7.48 (m, 2H), 7.47-7.42 (m, 1H), 7.39-7.33 (m, 1H), 7.30-7.28 (m, 1H), 7.26-7.25 (m, 1H), 7.16-7.10 (m, 1H), 6.54 (d, J=8.7 Hz, 2H), 3.67 (t, J=5.8 Hz, 4H), 3.25 (t, J=7.3 Hz, 4H), 2.51 (s, 1H), 2.05 (s, 3H), 1.65-1.56 (m, 2H), 1.36 (br s, 6H), 1.12 (m, 3H).

### Example 20: Synthesis of 6-(3-hydroxypropyl)-2³-(trifluoromethyl)-4-thia-3,6-diaza-2,5(2,6)-dipyridina-1(1,2)-benzenacyclodecaphane 4,4-dioxide (20)

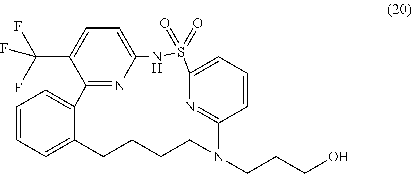 [SMILES: O=S1(=O)Nc2ccc(C(F)(F)F)c(n2)-c2ccccc2CCCCN(CCCO)c2cccc1n2]

6-(3-hydroxypropyl)-2³-(trifluoromethyl)-4-thia-3,6-diaza-2,5(2,6)-dipyridina-1(1,2)-benzenacyclodecaphane 4,4-dioxide (20) was synthesized using the procedure described in Example 12 except 5-(hex-5-en-1-ylamino)pentan-1-ol (int-a34) was replaced with 3-(but-3-en-1-ylamino)propan-1-ol (int-a28). LC-MS (Condition 1): m/z 507.2 [M+1]⁺, 1.81 min. ¹H NMR (400 MHz, DMSO-d₆) δ 11.67 (s, 1H), 8.15 (d, J=8.8 Hz, 1H), 7.71 (dd, J=8.7, 7.3 Hz, 1H), 7.39-7.30 (m, 2H), 7.30-7.20 (m, 3H), 7.16 (d, J=7.1 Hz, 1H), 6.83 (d, J=8.6 Hz, 1H), 4.56 (t, J=4.9 Hz, 1H), 4.00-3.82 (m, 1H), 3.45-3.34 (m, 3H), 3.32-3.20 (m, 2H), 2.93-2.80 (m, 1H), 2.60-2.51 (m, 1H), 1.81-1.69 (m, 1H), 1.69-1.53 (m, 2H), 1.39-1.17 (m, 2H), 0.75-0.57 (m, 1H).

### Example 21: Synthesis of 6-(3-hydroxypropyl)-2³-(trifluoromethyl)-4-thia-3,6-diaza-2,5(2,6)-dipyridina-1(1,2)-benzenacyclododecaphane 4,4-dioxide (21)

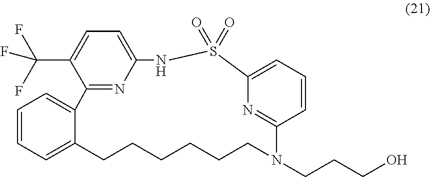 [SMILES: O=S1(=O)Nc2ccc(C(F)(F)F)c(n2)-c2ccccc2CCCCCCN(CCCO)c2cccc1n2]

6-(3-hydroxypropyl)-2³-(trifluoromethyl)-4-thia-3,6-diaza-2,5(2,6)-dipyridina-1(1,2)-benzenacyclododecaphane 4,4-dioxide_(21) was synthesized using the procedure described in Example 12 except 5-(hex-5-en-1-ylamino)pentan-1-ol (int-a34) was replaced with 3-(hex-5-en-1-ylamino)propan-1-ol (int-a17). LC-MS (Condition 1): m/z 535.2 [M+1]⁺, 1.83 min. ¹H NMR (400 MHz, DMSO-d₆) δ 11.68 (s, 1H), 8.17 (d, J=8.9 Hz, 1H), 7.70 (dd, J=8.7, 7.2 Hz, 1H), 7.51-7.38 (m, 1H), 7.34 (td, J=7.5, 1.5 Hz, 1H), 7.29-7.20 (m, 2H), 7.20-7.11 (m, 2H), 6.87 (d, J=8.8 Hz, 1H), 4.56 (t, J=4.9 Hz, 1H), 3.69-3.51 (m, 1H), 3.47-3.35 (m, 4H), 3.32-3.17 (m, 1H), 2.66-2.55 (m, 1H), 2.12-1.94 (m, 1H), 1.70-1.55 (m, 2H), 1.44-0.97 (m, 6H), 0.96-0.68 (m, 2H).

### Example 22: Synthesis of 1⁵-fluoro-6-(3-hydroxypropyl)-2³-(trifluoromethyl)-11-oxa-4-thia-3,6-diaza-2,5(2,6)-dipyridina-1(1,2)-benzenacycloundecaphane 4,4-dioxide (22)

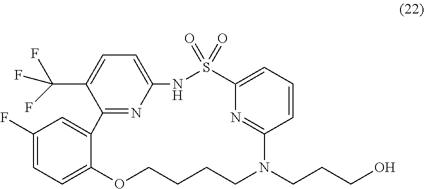 [SMILES: O=S1(=O)Nc2ccc(C(F)(F)F)c(n2)-c2cc(F)ccc2OCCCCN(CCCO)c2cccc1n2]

1⁵-fluoro-6-(3-hydroxypropyl)-2³-(trifluoromethyl)-11-oxa-4-thia-3,6-diaza-2,5(2,6)-dipyridina-1(1,2)-benzenacycloundecaphane 4,4-dioxide (22) was synthesized using the procedure described in Example 12 except 6-fluoro-N-(5-(trifluoromethyl)-6-(2-vinylphenyl)pyridin-2-yl)pyridine-2-sulfonamide (int-b2) was replaced with N-(6-(2-(allyloxy)-5-fluorophenyl)-5-(trifluoromethyl)pyridin-2-yl)-6-fluoropyridine-2-sulfonamide (int-b8) and 5-(hex-5-en-1-ylamino)pentan-1-ol (int-a34) was replaced with 3-(but-3-en-1-ylamino)propan-1-ol (int-a28). LC-MS (Condition 1): m/z 541.15 [M+1]⁺, 1.71 min. ¹H NMR (400 MHz, DMSO-d₆) δ 11.66 (s, 1H), 8.06 (d, J=8.9 Hz, 1H), 7.69 (dd, J=8.7, 7.2 Hz, 1H), 7.32-7.19 (m, 3H), 7.14-7.01 (m, 2H), 6.85 (d, J=8.8 Hz, 1H), 4.57 (t, J=4.9 Hz, 1H), 4.04-3.77 (m, 3H), 3.50-3.38 (m, 3H), 3.29-3.19 (m, 1H), 3.03-2.90 (m, 1H), 1.77-1.55 (m, 3H), 1.54-1.35 (m, 2H), 1.27-1.11 (m, 1H).

### Example 23: Synthesis of 1⁵-fluoro-6-(4-hydroxybutyl)-2³-(trifluoromethyl)-11-oxa-4-thia-3,6-diaza-2.5(2.6)-dipyridina-1(1.2)-benzenacycloundecaphane 4,4-dioxide (23)

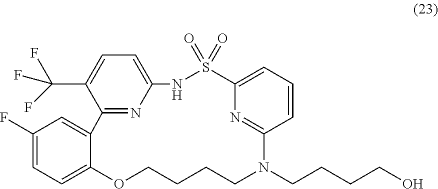 [SMILES: O=S1(=O)Nc2ccc(C(F)(F)F)c(n2)-c2cc(F)ccc2OCCCCN(CCCCO)c2cccc1n2]

1⁵-fluoro-6-(4-hydroxybutyl)-2³-(trifluoromethyl)-11-oxa-4-thia-3,6-diaza-2,5(2,6)-dipyridina-1(1,2)-benzenacycloundecaphane 4,4-dioxide (23) was synthesized using the procedure described in Example 12 except 6-fluoro-N-(5-(trifluoromethyl)-6-(2-vinylphenyl)pyridin-2-yl)pyridine-2-sulfonamide (int-b2) was replaced with N-(6-(2-(allyloxy)-5-fluorophenyl)-5-(trifluoromethyl)pyridin-2-yl)-6-fluoropyridine-2-sulfonamide (int-b8) and 5-(hex-5-en-1-ylamino)pentan-1-ol (int-a34) was replaced with 4-(but-3-en-1-ylamino)butan-1-ol (int-a7). LC-MS (Condition 1): m/z 555.2 [M+1]⁺, 1.71 min. ¹H NMR (400 MHz, DMSO-d₆) δ 11.65 (s, 1H), 8.05 (d, J=8.9 Hz, 1H), 7.68 (dd, J=8.7, 7.2 Hz, 1H), 7.31-7.18 (m, 3H), 7.14-7.01 (m, 2H), 6.82 (d, J=8.8 Hz, 1H), 4.44 (t, J=5.1 Hz, 1H), 4.05-3.95 (m, 1H), 3.95-3.86 (m, 1H), 3.86-3.75 (m, 1H), 3.47-3.34 (m, 3H), 3.22-3.10 (m, 1H), 3.04-2.90 (m, 1H), 1.76-1.59 (m, 1H), 1.57-1.34 (m, 6H), 1.29-1.09 (m, 1H).

### Example 24: Synthesis of 2³-chloro-6-(3-hydroxypropyl)-4-thia-3,6-diaza-2,5(2,6)-dipyridina-1(1,2)-benzenacycloundecaphane 4,4-dioxide (24)

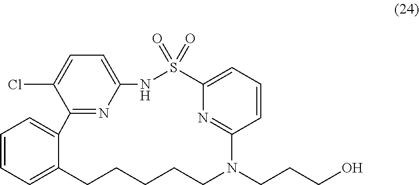 [SMILES: O=S1(=O)Nc2ccc(Cl)c(n2)-c2ccccc2CCCCCN(CCCO)c2cccc1n2]

2³-chloro-6-(3-hydroxypropyl)-4-thia-3,6-diaza-2,5(2,6)-dipyridina-1(1,2)-benzenacycloundecaphane 4,4-dioxide (24) was synthesized using the procedure described in Example 12 except 6-fluoro-N-(5-(trifluoromethyl)-6-(2-vinylphenyl)pyridin-2-yl)pyridine-2-sulfonamide (int-b2) was replaced with N-(5-chloro-6-(2-vinylphenyl)pyridin-2-yl)-6-fluoropyridine-2-sulfonamide (int-b3) and 5-(hex-5-en-1-ylamino)pentan-1-ol (int-a34) was replaced with 3-(pent-4-en-1-ylamino)propan-1-ol (int-a24). LC-MS (Condition 1): m/z 487.2 [M+1]⁺, 1.75 min. ¹H NMR (400 MHz, DMSO-d₆) δ 11.30 (s, 1H), 7.86 (d, J=8.9 Hz, 1H), 7.69 (dd, J=8.7, 7.2 Hz, 1H), 7.40-7.22 (m, 3H), 7.20-7.13 (m, 3H), 6.82 (d, J=8.7 Hz, 1H), 4.56 (t, J=4.9 Hz, 1H), 3.87-3.69 (m, 1H), 3.44-3.34 (m, 3H), 3.32-3.24 (m, 2H), 3.05-2.88 (m, 1H), 2.19-1.97 (m, 1H), 1.71-1.45 (m, 4H), 1.40-0.83 (m, 4H).

### Example 25: Synthesis of 6-(4-hydroxybutyl)-2³-(trifluoromethyl)-4-thia-3,6-diaza-2,5(2,6)-dipyridina-1(1,2)-benzenacycloundecaphane 4,4-dioxide (25)

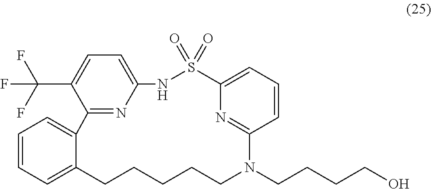 [SMILES: O=S1(=O)Nc2ccc(C(F)(F)F)c(n2)-c2ccccc2CCCCCN(CCCCO)c2cccc1n2]

6-(4-hydroxybutyl)-2³-(trifluoromethyl)-4-thia-3,6-diaza-2,5(2,6)-dipyridina-1(1,2)-benzenacycloundecaphane 4,4-dioxide (25) was synthesized using the procedure described in Example 12 except 5-(hex-5-en-1-ylamino)pentan-1-ol (int-a34) was replaced with 4-(pent-4-en-1-ylamino)butan-1-ol (int-a29). LC-MS (Condition 1): m/z 535.2 [M+1]⁺, 1.87 min. ¹H NMR (600 MHz, DMSO-d₆) δ 11.76 (s, 1H), 8.10 (d, J=8.9 Hz, 1H), 7.71 (dd, J=8.7, 7.2 Hz, 1H), 7.36 (td, J=7.5, 1.4 Hz, 1H), 7.29 (dd, J=7.7, 1.3 Hz, 1H), 7.25-7.18 (m, 3H), 7.14 (d, J=7.6 Hz, 1H), 6.82 (d, J=8.7 Hz, 1H), 4.42 (t, J=5.1 Hz, 1H), 4.01-3.89 (m, 1H), 3.40 (td, J=6.2, 5.1 Hz, 2H), 3.32-3.26 (m, 2H), 3.26-3.18 (m, 1H), 2.94-2.80 (m, 1H), 2.45-2.35 (m, 1H), 1.94-1.84 (m, 1H), 1.76-1.66 (m, 1H), 1.56-1.26 (m, 5H), 1.19-1.09 (m, 2H), 1.07-0.98 (m, 1H).

### Example 26: Synthesis of 1⁵-fluoro-6-(3-hydroxypropyl)-2³-(trifluoromethyl)-4-thia-3,6-diaza-2,5(2,6)-dipyridina-1(1,2)-benzenacycloundecaphane 4,4-dioxide (26)

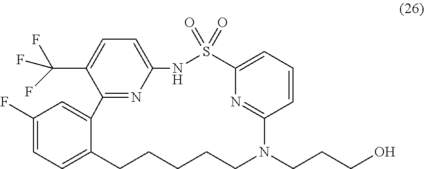 [SMILES: O=S1(=O)Nc2ccc(C(F)(F)F)c(n2)-c2cc(F)ccc2CCCCCN(CCCO)c2cccc1n2]

1⁵-fluoro-6-(3-hydroxypropyl)-2³-(trifluoromethyl)-4-thia-3,6-diaza-2,5(2,6)-dipyridina-1(1,2)-benzenacycloundecaphane 4,4-dioxide (26) was synthesized using the procedure described in Example 12 except 6-fluoro-N-(5-(trifluoromethyl)-6-(2-vinylphenyl)pyridin-2-yl)pyridine-2-sulfonamide (int-b2) was replaced with 6-fluoro-N-(6-(5-fluoro-2-vinylphenyl)-5-(trifluoromethyl)pyridin-2-yl)pyridine-2-sulfonamide (int-b1) and 5-(hex-5-en-1-ylamino)pentan-1-ol (int-a34) was replaced with 3-(pent-4-en-1-ylamino)propan-1-ol (int-a24). LCMS (Condition 1): m/z 539.2 [M+H]⁺, 1.58 min.

### Example 27: Synthesis of 6-(3-hydroxy-2,2-dimethylpropyl)-2³-(trifluoromethyl)-4-thia-3,6-diaza-2,5(2,6)-dipyridina-1(1,2)-benzenacycloundecaphane 4,4-dioxide (27)

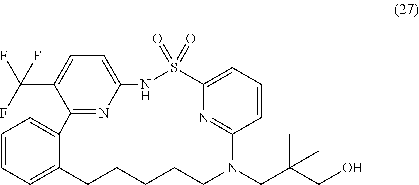 [SMILES: CC(C)(CO)CN1CCCCCc2ccccc2-c2nc(ccc2C(F)(F)F)NS(=O)(=O)c2cccc1n2]

6-(3-hydroxy-2,2-dimethylpropyl)-2³-(trifluoromethyl)-4-thia-3,6-diaza-2,5(2,6)-dipyridina-1(1,2)-benzenacycloundecaphane 4,4-dioxide (27) was synthesized using the procedure described in Example 12 except 5-(hex-5-en-1-ylamino)pentan-1-ol (int-a34) was replaced with 2,2-dimethyl-3-(pent-4-en-1-ylamino)propan-1-ol (int-a25). LC-MS (Condition 1): m/z 549.3 [M+1]⁺, 1.83 min. ¹H NMR (400 MHz, DMSO-d₆) δ 11.77 (s, 1H), 8.10 (d, J=8.8 Hz, 1H), 7.67 (dd, J=8.8, 7.2 Hz, 1H), 7.35 (td, J=7.5, 1.4 Hz, 1H), 7.30-7.15 (m, 4H), 7.13 (d, J=7.6 Hz, 1H), 7.08 (d, J=8.9 Hz, 1H), 4.77 (t, J=4.8 Hz, 1H), 3.93-3.76 (m, 1H), 3.15-3.03 (m, 3H), 3.01-2.88 (m, 1H), 2.41-2.27 (m, 1H), 1.96-1.69 (m, 2H), 1.45-1.18 (m, 3H), 1.17-0.91 (m, 2H), 0.89-0.83 (m, 1H), 0.81 (s, 3H), 0.80 (s, 3H).

### Example 28: Synthesis of 6-(3-hydroxy-2,2-dimethylpropyl)-2³-(trifluoromethyl)-4-thia-3,6-diaza-2,5(2,6)-dipyridina-1(1,2)-benzenacyclododecaphane 4,4-dioxide (28)

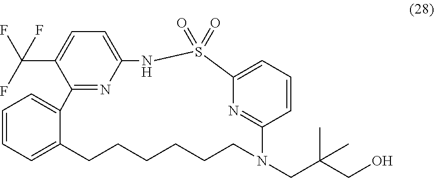 [SMILES: CC(C)(CO)CN1CCCCCCc2ccccc2-c2nc(ccc2C(F)(F)F)NS(=O)(=O)c2cccc1n2]

6-(3-hydroxy-2,2-dimethylpropyl)-2³-(trifluoromethyl)-4-thia-3,6-diaza-2,5(2,6)-dipyridina-1(1,2)-benzenacyclododecaphane 4,4-dioxide (28) was synthesized using the procedure described in Example 12 except 5-(hex-5-en-1-ylamino)pentan-1-ol (int-a34) was replaced with 3-(hex-5-en-1-ylamino)-2,2-dimethylpropan-1-ol (int-a26). LC-MS (Condition 1): m/z 563.2 [M+1]⁺, 1.96 min. ¹H NMR (400 MHz, DMSO-d₆) δ 11.78-11.31 (m, 1H), 8.19 (d, J=8.9 Hz, 1H), 7.65 (dd, J=8.8, 7.2 Hz, 1H), 7.61-7.44 (m, 1H), 7.38-7.30 (m, 1H), 7.28-7.19 (m, 2H), 7.15 (d, J=7.3 Hz, 1H), 7.11 (d, J=7.2 Hz, 1H), 7.03 (d, J=8.7 Hz, 1H), 4.74-4.53 (m, 1H), 3.75-3.52 (m, 1H), 3.50-3.34 (m, 2H), 3.13-3.05 (m, 2H), 2.45-2.20 (m, 1H), 2.16-1.92 (m, 1H), 1.43-0.98 (m, 6H), 0.92-0.82 (m, 1H), 0.81 (s, 3H), 0.79 (s, 3H), 0.75-0.60 (m, 1H).

### Example 29: Synthesis of 6-(3-hydroxypropyl)-2³-(trifluoromethyl)-4-thia-3,6-diaza-2,5(2,6)-dipyridina-1(1,2)-benzenacycloundecaphane 4,4-dioxide (29)

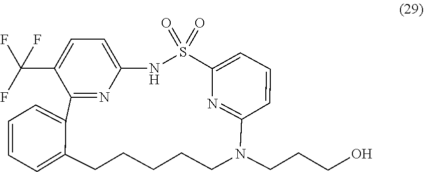 [SMILES: O=S1(=O)Nc2ccc(C(F)(F)F)c(n2)-c2ccccc2CCCCCN(CCCO)c2cccc1n2]

6-(3-hydroxypropyl)-2³-(trifluoromethyl)-4-thia-3,6-diaza-2,5(2,6)-dipyridina-1(1,2)-benzenacycloundecaphane 4,4-dioxide (29) was synthesized using the procedure described in Example 12 except 5-(hex-5-en-1-ylamino)pentan-1-ol (int-a34) was replaced with 3-(pent-4-en-1-ylamino)propan-1-ol (int-a24). LC-MS (Condition 1): m/z 521.3 [M+1]⁺, 1.72 min. ¹H NMR (400 MHz, DMSO-d₆) δ 11.78 (s, 1H), 8.10 (d, J=9.0 Hz, 1H), 7.71 (dd, J=8.7, 7.2 Hz, 1H), 7.36 (td, J=7.4, 1.4 Hz, 1H), 7.31-7.26 (m, 1H), 7.26-7.17 (m, 3H), 7.14 (d, J=7.6 Hz, 1H), 6.85 (d, J=8.8 Hz, 1H), 4.56 (t, J=4.9 Hz, 1H), 4.03-3.89 (m, 1H), 3.44-3.34 (m, 3H), 3.33-3.27 (m, 1H), 2.92-2.81 (m, 1H), 2.46-2.35 (m, 1H), 1.95-1.81 (m, 2H), 1.78-1.66 (m, 1H), 1.66-1.52 (m, 2H), 1.45-1.24 (m, 2H), 1.24-1.08 (m, 1H), 1.08-0.95 (m, 1H).

### Example 30: Synthesis of 6-(3-hydroxypropyl)-2³-(trifluoromethyl)-9-oxa-4-thia-3,6-diaza-2.5(2.6)-dipyridina-1(1.2)-benzenacyclododecaphane 4,4-dioxide (30)

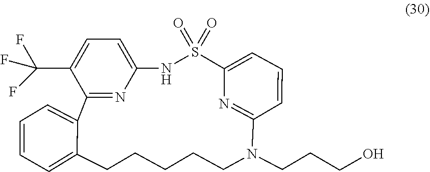 [SMILES: O=S1(=O)Nc2ccc(C(F)(F)F)c(n2)-c2ccccc2CCCCCN(CCCO)c2cccc1n2]

6-(3-hydroxypropyl)-2³-(trifluoromethyl)-9-oxa-4-thia-3,6-diaza-2,5(2,6)-dipyridina-1(1,2)-benzenacyclododecaphane 4,4-dioxide (30) was synthesized using the procedure described in Example 12 except 5-(hex-5-en-1-ylamino)pentan-1-ol (int-a34) was replaced with 3-((2-(allyloxy)ethyl)amino)propan-1-ol (int-a9). LCMS (Condition 1): m/z 537.3 [M+H]⁺, 1.62 min. ¹H NMR (400 MHz, DMSO-d₆) δ 11.50 (s, 1H), 8.15 (d, J=8.9 Hz, 1H), 7.60 (m, 1H), 7.36 (m, 2H), 7.21 (m, 2H), 7.09 (d, J=7.0 Hz, 1H), 7.03 (d, J=7.2 Hz, 1H), 6.92 (d, J=8.7 Hz, 1H), 4.43 (t, J=4.8 Hz, 1H), 3.72 (m, 1H), 3.0.5-3.50 (m, 9H), 1.99 (m, 2H), 1.57 (m, 4H). ¹⁹F NMR (376 MHz, DMSO-d₆) δ−56.60 (s).

### Example 31: Synthesis of 1⁵-fluoro-2³-(trifluoromethyl)-4-thia-3,6-diaza-2,5(2,6)-dipyridina-1(1,2)-benzenacycloundecaphane 4,4-dioxide (31)

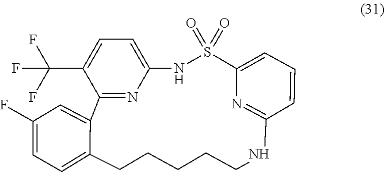 [SMILES: O=S1(=O)Nc2ccc(C(F)(F)F)c(n2)-c2cc(F)ccc2CCCCCNc2cccc1n2]

1⁵-fluoro-2³-(trifluoromethyl)-4-thia-3,6-diaza-2,5(2,6)-dipyridina-1(1,2)-benzenacycloundecaphane 4,4-dioxide (31) was synthesized using the procedure described in Example 12 except 6-fluoro-N-(5-(trifluoromethyl)-6-(2-vinylphenyl)pyridin-2-yl)pyridine-2-sulfonamide (int-b2) was replaced with 6-fluoro-N-(6-(5-fluoro-2-vinylphenyl)-5-(trifluoromethyl)pyridin-2-yl)pyridine-2-sulfonamide (int-b1) and 5-(hex-5-en-1-ylamino)pentan-1-ol (int-a34) was replaced with pent-4-en-1-amine (purchased). LCMS (Condition 1): m/z 481.1 [M+H]⁺, 1.81 min, ¹H NMR (400 MHz, Methanol-d₄) δ 7.98 (d, J=8.9 Hz, 1H), 7.53-7.41 (m, 2H), 7.27 (dd, J=8.6, 5.6 Hz, 1H), 7.22 (dd, J=7.2, 0.6 Hz, 1H), 7.12-7.04 (m, 1H), 6.87 (dd, J=9.2, 2.7 Hz, 1H), 6.56 (dd, J=8.5, 0.7 Hz, 1H), 3.80-3.62 (m, 1H), 2.92-2.81 (m, 1H), 2.43-2.31 (m, 1H), 2.07-1.97 (m, 1H), 1.76-1.60 (m, 1H), 1.52-1.40 (m, 1H), 1.40-1.27 (m, 2H), 1.29-1.13 (m, 2H).

### Example 32: Synthesis of ethyl 3-(4,4-dioxido-2³-(trifluoromethyl)-4-thia-3,6-diaza-2,5(2,6)-dipyridina-1(1,2)-benzenacyclododecaphane-6-yl)-2,2-dimethylpropanoate (32)

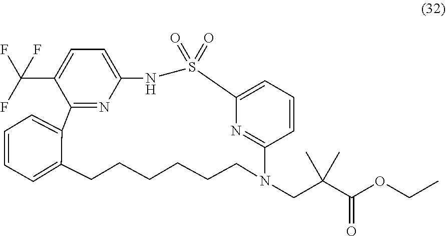 [SMILES: CCOC(=O)C(C)(C)CN1CCCCCCc2ccccc2-c2nc(ccc2C(F)(F)F)NS(=O)(=O)c2cccc1n2]

ethyl 3-(4,4-dioxido-2³-(trifluoromethyl)-4-thia-3,6-diaza-2,5(2,6)-dipyridina-1(1,2)-benzenacyclododecaphane-6-yl)-2,2-dimethylpropanoate (32) was synthesized using the procedure described in Example 12 except 5-(hex-5-en-1-ylamino)pentan-1-ol (int-a34) was replaced with ethyl 3-(hex-5-en-1-ylamino)-2,2-dimethylpropanoate (int-a20). LC-MS (Condition 1): m/z 605.3 [M+1]⁺, 1.89 min.

### Example 33: Synthesis of 8-hydroxy-2³-(trifluoromethyl)-4-thia-3,6-diaza-2,5(2,6)-dipyridina-1(1,2)-benzenacyclodecaphane 4,4-dioxide (33)

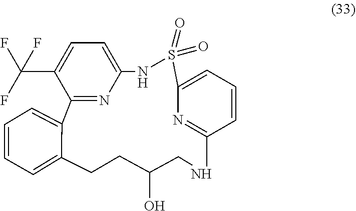 [SMILES: O=S1(=O)Nc2ccc(C(F)(F)F)c(n2)-c2ccccc2CCC(O)CNc2cccc1n2]

8-hydroxy-2³-(trifluoromethyl)-4-thia-3,6-diaza-2,5(2,6)-dipyridina-1(1,2)-benzenacyclodecaphane 4,4-dioxide (33) was synthesized using the procedure described in Example 12 except 5-(hex-5-en-1-ylamino)pentan-1-ol (int-a34) was replaced with 1-aminobut-3-en-2-ol (ChemBridge (cat. #4055871)). LCMS (Condition 1): m/z 465.2 [M+H]⁺, 1.47 min. ¹H NMR (500 MHz, DMSO-d₆) δ 11.65 (s, 1H), 11.26 (s, 0.3H), 8.19 (d, J=8.9 Hz, 0.3H), 8.16 (d, J=8.9 Hz, 1H), 7.60 (dd, J=7.3, 8.4 Hz, 1H), 7.53 (m, 0.7H), 7.35 (m, 3.7H), 7.25 (m, 3.7H), 7.14 (m, 1.3H), 7.02 (d, J=7.2 Hz, 0.3H), 6.72 (m, 1.3H), 4.67 (d, J=4.7 Hz, 0.3H), 4.56 (d, J=5.7 Hz, 1H), 3.73 (dd, J=8.6, 13.0 Hz, 1H), 3.38 (m, 0.3H), 3.03 (m, 0.6H), 2.60 (m, 3H), 2.20 (m, 0.3H), 1.84 (m, 1H), 1.70 (m, 1H), 1.54 (m, 0.3H), 1.43 (m, 1H), 1.28 (m, 0.3H). ¹⁹F NMR (376 MHz, DMSO-d₆) δ−56.68 (s, 0.3F), −57.54 (s, 1F). Diastereomers, 7:3 ratio.

### Example 34: Synthesis of 6-(((4S,5S)-5-(hydroxymethyl)-2,2-dimethyl-1,3-dioxolan-4-yl)methyl)-2³-(trifluoromethyl)-4-thia-3,6-diaza-2,5(2,6)-dipyridina-1(1,2)-benzenacycloundecaphane 4,4-dioxide (34)

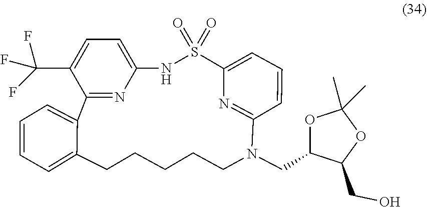 [SMILES: CC1(C)O[C@@H](CO)[C@H](CN2CCCCCc3ccccc3-c3nc(ccc3C(F)(F)F)NS(=O)(=O)c3cccc2n3)O1]

6-(((4S,5S)-5-(hydroxymethyl)-2,2-dimethyl-1,3-dioxolan-4-yl)methyl)-2³-(trifluoromethyl)-4-thia-3,6-diaza-2,5(2,6)-dipyridina-1(1,2)-benzenacycloundecaphane 4,4-dioxide (34) was synthesized using the procedure described in Example 12 except 5-(hex-5-en-1-ylamino)pentan-1-ol (int-a34) was replaced with ((4S,5S)-2,2-dimethyl-5-((pent-4-en-1-ylamino)methyl)-1,3-dioxolan-4-yl)methanol (int-a13). LCMS (Condition 1): m/z 607.3 [M+H]⁺, 1.70 min. ¹H NMR (400 MHz, DMSO-d₆) δ 11.81 (m, 1H), 8.09 (m, 1H), 7.71 (m, 1H), 7.35 (m, 1H), 7.25 (m, 4H), 7.14 (d, J=7.5 Hz, 1H), 6.96 (m, 1H), 5.03 and 4.98 (t, J=5.4 Hz, 1H), 3.98 (m, 2H), 3.71 (m, 1H), 3.55 (m, 4H), 2.98 (m, 1H), 2.39 (m, 1H), 1.88 (m, 1H), 1.73 (m, 1H), 0.99-1.45 (m, 11H). ¹⁹F NMR (376 MHz, DMSO-d₆) δ−56.80(s), −56.88 (s). Diastereomers, 1:1 ratio.

### Example 35: Synthesis of 6-methyl-2³-(trifluoromethyl)-4-thia-3,6-diaza-2,5(2,6)-dipyridina-1(1,2)-benzenacyclododecaphane 4,4-dioxide (35)

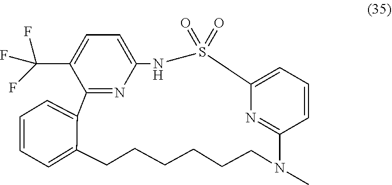 [SMILES: CN1CCCCCCc2ccccc2-c2nc(ccc2C(F)(F)F)NS(=O)(=O)c2cccc1n2]

6-methyl-2³-(trifluoromethyl)-4-thia-3,6-diaza-2,5(2,6)-dipyridina-1(1,2)-benzenacyclododecaphane 4,4-dioxide (35) was synthesized using the procedure described in Example 12 except 5-(hex-5-en-1-ylamino)pentan-1-ol (int-a34) was replaced with N-methylhex-5-en-1-amine (purchased). LC-MS (Condition 1): m/z 491.2 [M+1]⁺, 1.98 min. ¹H NMR (400 MHz, DMSO-d₆) δ 11.66 (s, 1H), 8.17 (d, J=8.9 Hz, 1H), 7.72 (dd, J=8.7, 7.2 Hz, 1H), 7.51-7.38 (m, 1H), 7.34 (td, J=7.4, 1.5 Hz, 1H), 7.28-7.20 (m, 2H), 7.20-7.10 (m, 2H), 6.85 (d, J=8.7 Hz, 1H), 3.72-3.54 (m, 1H), 3.50-3.36 (m, 1H), 3.33-3.30 (m, 1H), 2.86 (s, 3H), 2.10-1.87 (m, 1H), 1.43-1.17 (m, 3H), 1.14-0.95 (m, 3H), 0.91-0.76 (m, 1H), 0.76-0.60 (m, 1H).

### Example 36: Synthesis of 2³-(trifluoromethyl)-4-thia-3,6-diaza-2,5(2,6)-dipyridina-1(1,2)-benzenacyclododecaphane 4,4-dioxide (36)

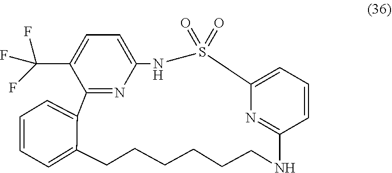 [SMILES: O=S1(=O)Nc2ccc(C(F)(F)F)c(n2)-c2ccccc2CCCCCCNc2cccc1n2]

2³-(trifluoromethyl)-4-thia-3,6-diaza-2,5(2,6)-dipyridina-1(1,2)-benzenacyclododecaphane 4,4-dioxide (36) was synthesized using the procedure described in Example 12 except 5-(hex-5-en-1-ylamino)pentan-1-ol (int-a34) was replaced with hex-5-en-1-amine (purchased). LC-MS (Condition 1): m/z 477.2 [M+1]⁺, 1.89 min. ¹H NMR (400 MHz, DMSO-d₆) δ 11.60 (s, 1H), 8.16 (d, J=9.0 Hz, 1H), 7.55 (dd, J=8.5, 7.1 Hz, 1H), 7.45 (d, J=8.8 Hz, 1H), 7.34 (td, J=7.4, 1.5 Hz, 1H), 7.29-7.24 (m, 1H), 7.23 (dd, J=7.3, 1.4 Hz, 1H), 7.19-7.12 (m, 2H), 7.10 (d, J=7.2 Hz, 1H), 6.64 (d, J=8.5 Hz, 1H), 3.25-3.13 (m, 1H), 3.12-2.97 (m, 1H), 2.71-2.58 (m, 1H), 2.15-1.98 (m, 1H), 1.36-1.14 (m, 3H), 1.14-0.97 (m, 4H), 0.97-0.80 (m, 1H).

### Example 37: Synthesis of 6-((2,2-dimethyl-1,3-dioxolan-4-yl)methyl)-2³-(trifluoromethyl)-4-thia-3,6-diaza-2,5(2,6)-dipyridina-1(1,2)-benzenacycloundecaphane 4,4-dioxide (37)

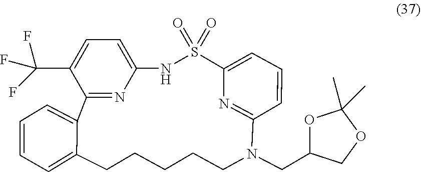 [SMILES: CC1(C)OCC(CN2CCCCCc3ccccc3-c3nc(ccc3C(F)(F)F)NS(=O)(=O)c3cccc2n3)O1]

6-((2,2-dimethyl-1,3-dioxolan-4-yl)methyl)-2³-(trifluoromethyl)-4-thia-3,6-diaza-2,5(2,6)-dipyridina-1(1,2)-benzenacycloundecaphane 4,4-dioxide (37) was synthesized using the procedure described in Example 12 except 5-(hex-5-en-1-ylamino)pentan-1-ol (int-a34) was replaced with N-((2,2-dimethyl-1,3-dioxolan-4-yl)methyl)pent-4-en-1-amine (int-a10). LCMS (Condition 1): m/z 577.3 [M+H]⁺, 1.78 min. ¹H NMR (400 MHz, DMSO-d₆) δ 11.80 (s, 1H), 8.10 (d, J=8.9 Hz, 1H), 7.71 (m, 1H), 7.35 (m, 1H), 7.28 (m, 2H), 7.22 (m, 2H), 7.14 (d, J=7.5 Hz, 1H), 6.97 (d, J=8.8 Hz, 1H), 4.23 (m, 1H), 4.01 (m, 2H), 3.28-3.57 (m, 3H), 2.97 (m, 1H), 2.38 (m, 1H), 1.89 (m, 1H), 1.72 (m, 1H), 1.24 (m, 11H). ¹⁹F NMR (376 MHz, DMSO-d₆) δ−56.82(s), −56.86 (s). Diastereomers, 52:48 ratio.

### Example 38: Synthesis of 8-hydroxy-2³-(trifluoromethyl)-4-thia-3,6-diaza-2,5(2,6)-dipyridina-1(1,2)-benzenacycloundecaphane 4,4-dioxide (38)

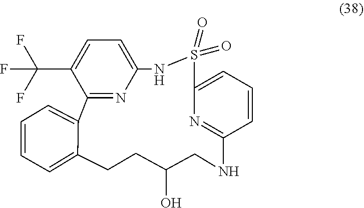 [SMILES: O=S1(=O)Nc2ccc(C(F)(F)F)c(n2)-c2ccccc2CCC(O)CNc2cccc1n2]

8-hydroxy-2³-(trifluoromethyl)-4-thia-3,6-diaza-2,5(2,6)-dipyridina-1(1,2)-benzenacycloundecaphane 4,4-dioxide (38) was synthesized using the procedure described in Example 12 except 5-(hex-5-en-1-ylamino)pentan-1-ol (int-a34) was replaced with 1-aminopent-4-en-2-ol (ChemBridge (cat. #4080175)). LCMS (Condition 1): m/z 479.2 [M+H]+. 1.52 min. ¹H NMR (400 MHz, DMSO-d₆) δ 11.60 (m, 1.7H), 8.12 (d, J=2.4 Hz, 0.8H), 8.09 (d, J=2.4 Hz, 1H), 7.54 (m, 1.8H), 7.32 (m, 5.5H), 7.23 (m, 1.8H), 7.14 (m, 3.5H), 7.07 (dd, J=4.0, 6.9 Hz, 1H), 6.95 (dd, J=3.3, 7.8 Hz, 0.8H), 6.80 (d, J=8.5 Hz, 0.8H), 6.74 (m, 1H), 4.71 (d, J=4.2 Hz, 0.8H), 4.56 (d, J=4.7 Hz, 1H), 3.73 (m, 0.8H), 3.58 (m, 1.8H), 3.23 (m, 1H), 2.90 (m, 0.8H), 2.65 (m, 1H), 2.35 (m, 1.6H), 2.07 (m, 1H), 1.89 (m, 0.8H), 1.55 (m, 1H), 1.32 (m, 5.8H). ¹⁹F NMR (376 MHz, DMSO-d₆) δ−57.05 (s, 1F), −57.17 (s, 0.75F). Diastereomers, 4:3 ratio.

### Example 39: Synthesis of 6-(3-hydroxypropyl)-2³-(trifluoromethyl)-4-thia-3,6-diaza-2,5(2,6)-dipyridina-1(1.2)-benzenacyclotridecaphane 4,4-dioxide (39)

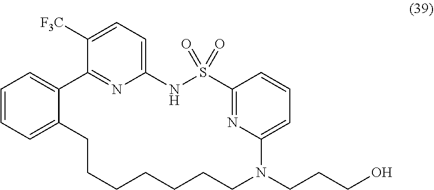 [SMILES: O=S1(=O)Nc2ccc(C(F)(F)F)c(n2)-c2ccccc2CCCCCCCN(CCCO)c2cccc1n2]

6-(3-hydroxypropyl)-2³-(trifluoromethyl)-4-thia-3,6-diaza-2,5(2,6)-dipyridina-1(1,2)-benzenacyclotridecaphane 4,4-dioxide (39) was synthesized using the procedure described in Example 12 except 5-(hex-5-en-1-ylamino)pentan-1-ol (int-a34) was replaced with 3-(hept-6-en-1-ylamino)propan-1-ol (int-a39). LCMS (Condition 1): m/z 549.2 [M+H]+. 1.88 min. ¹H NMR (400 MHz, DMSO-d₆) δ 11.73 (s, 1H), 8.19 (d, J=9.0 Hz, 1H), 7.72 (dd, J=8.7, 7.2 Hz, 1H), 7.55-7.40 (m, 1H), 7.35 (td, J=7.4, 1.5 Hz, 1H), 7.29-7.21 (m, 2H), 7.20-7.12 (m, 2H), 6.89 (d, J=8.8 Hz, 1H), 4.58 (t, J=4.9 Hz, 1H), 3.72-3.57 (m, 1H), 3.54-3.41 (m, 4H), 3.41-3.34 (m, 1H), 2.42-2.29 (m, 1H), 2.18-1.99 (m, 1H), 1.71-1.59 (m, 2H), 1.44-1.30 (m, 2H), 1.30-0.92 (m, 6H), 0.91-0.70 (m, 2H).

### Example 40: Synthesis of 6-(4-hydroxybutyl)-2³-(trifluoromethyl)-4-thia-3,6-diaza-2,5(2,6)-dipyridina-1(1,2)-benzenacyclotridecaphane 4,4-dioxide (40)

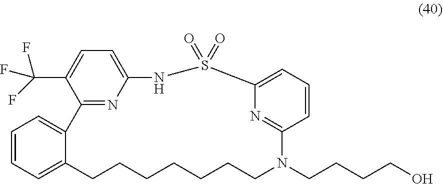 [SMILES: O=S1(=O)Nc2ccc(C(F)(F)F)c(n2)-c2ccccc2CCCCCCCN(CCCCO)c2cccc1n2]

6-(4-hydroxybutyl)-2³-(trifluoromethyl)-4-thia-3,6-diaza-2,5(2,6)-dipyridina-1(1,2)-benzenacyclotridecaphane 4,4-dioxide (40) was synthesized using the procedure described in Example 12 except 5-(hex-5-en-1-ylamino)pentan-1-ol (int-a34) was replaced with 4-(hept-6-en-1-ylamino)butan-1-ol (int-a36). LCMS (Condition 1): m/z 563.2 [M+H]+. 1.92 min. ¹H NMR (400 MHz, DMSO-d₆) δ 11.73 (s, 1H), 8.19 (d, J=8.9 Hz, 1H), 7.72 (dd, J=8.7, 7.2 Hz, 1H), 7.55-7.39 (m, 1H), 7.35 (td, J=7.4, 1.5 Hz, 1H), 7.30-7.21 (m, 2H), 7.21-7.11 (m, 2H), 6.87 (d, J=8.8 Hz, 1H), 4.45 (t, J=5.1 Hz, 1H), 3.76-3.56 (m, 1H), 3.55-3.45 (m, 1H), 3.42 (td, J=6.2, 4.9 Hz, 2H), 3.31-3.20 (m, OH), 2.45-2.27 (m, 1H), 2.19-1.99 (m, 1H), 1.63-1.29 (m, 6H), 1.29-0.92 (m, 6H), 0.91-0.67 (m, 2H).

### Example 41: Synthesis of 6-(5-hydroxypentyl)-2³-(trifluoromethyl)-4-thia-3,6-diaza-2,5(2,6)-dipyridina-1(1,2)-benzenacyclotridecaphane 4,4-dioxide (41)

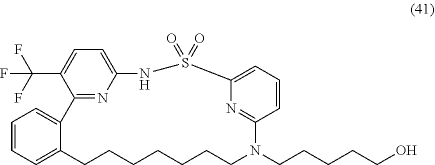 [SMILES: O=S1(=O)Nc2ccc(C(F)(F)F)c(n2)-c2ccccc2CCCCCCCN(CCCCCO)c2cccc1n2]

6-(5-hydroxypentyl)-2³-(trifluoromethyl)-4-thia-3,6-diaza-2,5(2,6)-dipyridina-1(1,2)-benzenacyclotridecaphane 4,4-dioxide (41) was synthesized using the procedure described in Example 12 except 5-(hex-5-en-1-ylamino)pentan-1-ol (int-a34) was replaced with 5-(hept-6-en-1-ylamino)pentan-1-ol (int-a12). LCMS (Condition 1): m/z 577.3 [M+H]+. 1.97 min. ¹H NMR (400 MHz, DMSO-d₆) δ 11.72 (s, 1H), 8.19 (d, J=9.0 Hz, 1H), 7.72 (m, 1H), 7.45 (m, 1H), 7.35 (m, 1H), 7.25 (m, 2H), 7.16 (m, 2H), 6.86 (d, J=8.8 Hz, 1H), 4.38 (t, J=5.1 Hz, 1H), 3.64 (m, 1H), 3.48 (m, 1H), 3.38 (m, 2H), 3.28 (m, 1H), 2.35 (m, 1H), 2.08 (m, 1H), 0.97-1.54 (m, 16H), 0.80 (m, 1H). ¹⁹F NMR (376 MHz, DMSO-d₆) δ−56.70 (s).

### Example 42: Synthesis of 6-(4-hydroxybutyl)-2³-(trifluoromethyl)-4-thia-3,6-diaza-2,5(2,6)-dipyridina-1(1,2)-benzenacyclopentadecaphane 4,4-dioxide (42)

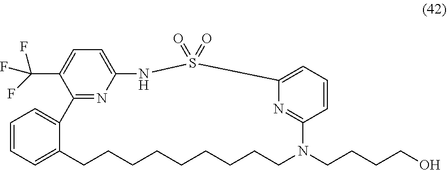 [SMILES: O=S1(=O)Nc2ccc(C(F)(F)F)c(n2)-c2ccccc2CCCCCCCCCN(CCCCO)c2cccc1n2]

6-(4-hydroxybutyl)-2³-(trifluoromethyl)-4-thia-3,6-diaza-2,5(2,6)-dipyridina-1(1,2)-benzenacyclopentadecaphane 4,4-dioxide (42) was synthesized using the procedure described in Example 12 except 5-(hex-5-en-1-ylamino)pentan-1-ol (int-a34) was replaced with 4-(non-8-en-1-ylamino)butan-1-ol (int-a38). LCMS (Condition 1): m/z 591.3 [M+H]+. 1.96 min. ¹H NMR (400 MHz, DMSO-d₆) δ 11.53 (s, 1H), 8.22 (d, J=8.9 Hz, 1H), 7.75-7.49 (m, 2H), 7.35 (t, J=7.5 Hz, 1H), 7.30-7.19 (m, 2H), 7.19-7.05 (m, 2H), 6.91 (d, J=8.7 Hz, 1H), 4.46 (t, J=5.1 Hz, 1H), 3.98-3.61 (m, 1H), 3.45-3.38 (m, 3H), 3.33-3.31 (m, 2H), 2.26-2.01 (m, 2H), 1.63-1.37 (m, 6H), 1.37-0.76 (m, 12H).

### Example 43: Synthesis of 6-(3-hydroxypropyl)-2³-(trifluoromethyl)-4-thia-3,6-diaza-2,5(2,6)-dipyridina-1(1,2)-benzenacyclotetradecaphane 4,4-dioxide (43)

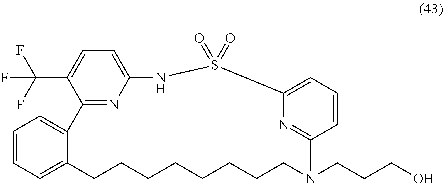 [SMILES: O=S1(=O)Nc2ccc(C(F)(F)F)c(n2)-c2ccccc2CCCCCCCCN(CCCO)c2cccc1n2]

6-(3-hydroxypropyl)-2³-(trifluoromethyl)-4-thia-3,6-diaza-2,5(2,6)-dipyridina-1(1,2)-benzenacyclotetradecaphane 4,4-dioxide (43) was synthesized using the procedure described in Example 12 except 5-(hex-5-en-1-ylamino)pentan-1-ol (int-a34) was replaced with 3-(oct-7-en-1-ylamino)propan-1-ol (int-a40). LCMS (Condition 1): m/z 563.2 [M+H]+. 1.91 min. ¹H NMR (400 MHz, DMSO-d₆) δ 11.60 (s, 1H), 8.22 (d, J=8.8 Hz, 1H), 7.68 (dd, J=8.7, 7.2 Hz, 1H), 7.62-7.43 (m, 1H), 7.39-7.30 (m, 1H), 7.29-7.19 (m, 2H), 7.16 (d, J=7.5 Hz, 1H), 7.11 (d, J=7.2 Hz, 1H), 6.90 (d, J=8.8 Hz, 1H), 4.58 (t, J=5.0 Hz, 1H), 3.93-3.68 (m, 1H), 3.49-3.42 (m, 2H), 3.42-3.36 (m, 1H), 2.46-2.25 (m, 3H), 2.13-1.91 (m, 1H), 1.75-1.58 (m, 2H), 1.42-1.21 (m, 3H), 1.21-0.70 (m, 9H).

### Example 44: Synthesis of 6-(4-hydroxybutyl)-2³-(trifluoromethyl)-4-thia-3,6-diaza-2,5(2,6)-dipyridina-1(1,2)-benzenacyclotetradecaphane 4,4-dioxide (44)

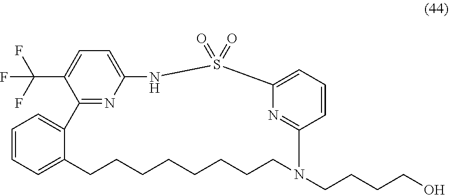 [SMILES: O=S1(=O)Nc2ccc(C(F)(F)F)c(n2)-c2ccccc2CCCCCCCCN(CCCCO)c2cccc1n2]

6-(4-hydroxybutyl)-2³-(trifluoromethyl)-4-thia-3,6-diaza-2,5(2,6)-dipyridina-1(1,2)-benzenacyclotetradecaphane 4,4-dioxide (44) was synthesized using the procedure described in Example 12 except 5-(hex-5-en-1-ylamino)pentan-1-ol (int-a34) was replaced with 4-(oct-7-en-1-ylamino)butan-1-ol (int-a37). LCMS (Condition 1): m/z 577.3 [M+H]+. 1.96 min. ¹H NMR (400 MHz, DMSO-d₆) δ 11.60 (s, 1H), 8.21 (d, J=8.9 Hz, 1H), 7.68 (dd, J=8.7, 7.2 Hz, 1H), 7.54 (s, 1H), 7.39-7.30 (m, 1H), 7.29-7.20 (m, 2H), 7.16 (d, J=7.6 Hz, 1H), 7.11 (d, J=7.2 Hz, 1H), 6.89 (d, J=8.8 Hz, 1H), 4.45 (t, J=5.1 Hz, 1H), 3.92-3.68 (m, 1H), 3.46-3.35 (m, 3H), 3.32-3.16 (m, 2H), 2.42-2.27 (m, 1H), 2.12-1.91 (m, 1H), 1.64-1.49 (m, 2H), 1.49-1.39 (m, 2H), 1.39-1.20 (m, 3H), 1.19-0.73 (m, 7H).

### Example 45: Synthesis of 6-(3-hydroxypropyl)-2³-(trifluoromethyl)-4-thia-3,6-diaza-2,5(2,6)-dipyridina-1(1,2)-benzenacyclopentadecaphane 4,4-dioxide (45)

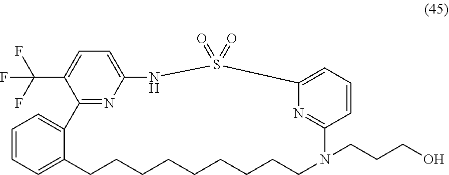 [SMILES: O=S1(=O)Nc2ccc(C(F)(F)F)c(n2)-c2ccccc2CCCCCCCCCN(CCCO)c2cccc1n2]

6-(3-hydroxypropyl)-2³-(trifluoromethyl)-4-thia-3,6-diaza-2,5(2,6)-dipyridina-1(1,2)-benzenacyclopentadecaphane 4,4-dioxide (45) was synthesized using the procedure described in Example 12 except 5-(hex-5-en-1-ylamino)pentan-1-ol (int-a34) was replaced with 3-(non-8-en-1-ylamino)propan-1-ol (int-a41). LCMS (Condition 1): m/z 577.3 [M+H]+. 1.97 min. ¹H NMR (400 MHz, DMSO-d₆) δ 11.52 (s, 1H), 8.22 (d, J=8.9 Hz, 1H), 7.82-7.49 (m, 2H), 7.35 (t, J=7.5 Hz, 1H), 7.29-7.19 (m, 2H), 7.19-7.03 (m, 2H), 6.92 (d, J=8.7 Hz, 1H), 4.57 (t, J=4.9 Hz, 1H), 4.01-3.61 (m, 1H), 3.55-3.34 (m, 3H), 2.57-2.51 (m, 2H), 2.29-1.99 (m, 2H), 1.79-1.56 (m, 2H), 1.54-1.39 (m, 2H), 1.37-0.72 (m, 12H).

### Example 46: Synthesis of 2³-chloro-6-(4-hydroxybutyl)-4-thia-3,6-diaza-2,5(2,6)-dipyridina-1(1,2)-benzenacycloundecaphane 4,4-dioxide (46)

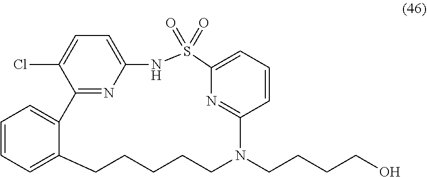 [SMILES: O=S1(=O)Nc2ccc(Cl)c(n2)-c2ccccc2CCCCCN(CCCCO)c2cccc1n2]

2³-chloro-6-(4-hydroxybutyl)-4-thia-3,6-diaza-2,5(2,6)-dipyridina-1(1,2)-benzenacycloundecaphane 4,4-dioxide (46) was synthesized using the procedure described in Example 12 except 6-fluoro-N-(5-(trifluoromethyl)-6-(2-vinylphenyl)pyridin-2-yl)pyridine-2-sulfonamide (int-b2) was replaced with N-(5-chloro-6-(2-vinylphenyl)pyridin-2-yl)-6-fluoropyridine-2-sulfonamide (int-b3) and 5-(hex-5-en-1-ylamino)pentan-1-ol (int-a34) was replaced with 4-(pent-4-en-1-ylamino)butan-1-ol (int-a29). LCMS (Condition 1): m/z 501.2 [M+H]⁺. 1.77 min.

### Example 47: Synthesis of 1⁵-fluoro-6-(4-hydroxybutyl)-2³-(trifluoromethyl)-4-thia-3,6-diaza-2,5(2,6)-dipyridina-1(1,2)-benzenacyclododecaphane 4,4-dioxide (47)

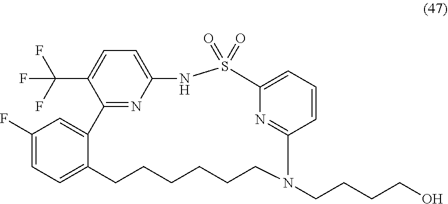 [SMILES: O=S1(=O)Nc2ccc(C(F)(F)F)c(n2)-c2cc(F)ccc2CCCCCCN(CCCCO)c2cccc1n2]

1⁵-fluoro-6-(4-hydroxybutyl)-2³-(trifluoromethyl)-4-thia-3,6-diaza-2,5(2,6)-dipyridina-1(1,2)-benzenacyclododecaphane 4,4-dioxide (47) was synthesized using the procedure described in Example 12 except 6-fluoro-N-(5-(trifluoromethyl)-6-(2-vinylphenyl)pyridin-2-yl)pyridine-2-sulfonamide (int-b2) was replaced with 6-fluoro-N-(6-(5-fluoro-2-vinylphenyl)-5-(trifluoromethyl)pyridin-2-yl)pyridine-2-sulfonamide (int-b1) and 5-(hex-5-en-1-ylamino)pentan-1-ol (int-a34) was replaced with 4-(hex-5-en-1-ylamino)butan-1-ol (int-a35). LCMS (Condition 1): m/z 566.9 [M+H]⁺. 1.79 min. ¹H NMR (400 MHz, DMSO-d₆) δ 11.73 (s, 1H), 8.19 (d, J=9.0 Hz, 1H), 7.70 (dd, J=7.4, 8.6 Hz, 1H), 7.44 (d, J=8.1 Hz, 1H), 7.31 (dd, J=5.9, 8.5 Hz, 1H), 7.23-7.18 (m, 1H), 7.17 (d, J=7.3 Hz, 1H), 7.07 (d, J=8.4 Hz, 1H), 6.86 (d, J=8.7 Hz, 1H), 4.45 (t, J=5.1 Hz, 1H), 3.69-3.50 (m, 1H), 3.41 (q, J=6.1 Hz, 2H), 3.32-3.14 (m, 2H), 2.02 (d, J=3.5 Hz, 1H), 1.59-1.47 (m, 2H), 1.44 (q, J=6.5 Hz, 2H), 1.32 (ddt, J=8.3, 11.9, 30.0 Hz, 3H), 1.15-0.95 (m, 3H), 0.84 (dd, J=6.7, 9.4 Hz, 1H), 0.80-0.66 (m, 1H). ¹⁹F NMR (376 MHz, DMSO-d₆) δ−56.02 (s, 3F), −117.97 (s, 1F)

### Example 48: Synthesis of 6-(6-hydroxyhexyl)-2³-(trifluoromethyl)-4-thia-3,6-diaza-2,5(2,6)-dipyridina-1(1,2)-benzenacycloundecaphane 4,4-dioxide (48)

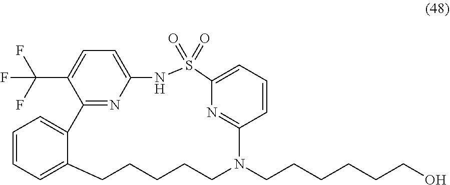 [SMILES: O=S1(=O)Nc2ccc(C(F)(F)F)c(n2)-c2ccccc2CCCCCN(CCCCCCO)c2cccc1n2]

6-(6-hydroxyhexyl)-2³-(trifluoromethyl)-4-thia-3,6-diaza-2,5(2,6)-dipyridina-1(1,2)-benzenacycloundecaphane 4,4-dioxide (48) was synthesized using the procedure described in Example 12 except 5-(hex-5-en-1-ylamino)pentan-1-ol (int-a34) was replaced with 6-(pent-4-en-1-ylamino)hexan-1-ol (int-a3). LCMS (Condition 1): m/z 563.2 [M+H]⁺. 1.89 min.

### Example 49: Synthesis of 6-(2-(2-hydroxyethoxy)ethyl)-2³-(trifluoromethyl)-4-thia-3,6-diaza-2,5(2,6)-dipyridina-1(1,2)-benzenacycloundecaphane 4,4-dioxide (49)

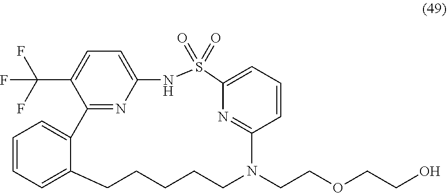 [SMILES: O=S1(=O)Nc2ccc(C(F)(F)F)c(n2)-c2ccccc2CCCCCN(CCOCCO)c2cccc1n2]

6-(2-(2-hydroxyethoxy)ethyl)-2³-(trifluoromethyl)-4-thia-3,6-diaza-2,5(2,6)-dipyridina-1(1,2)-benzenacycloundecaphane 4,4-dioxide (49) was synthesized using the procedure described in Example 12 except 5-(hex-5-en-1-ylamino)pentan-1-ol (int-a34) was replaced with 2-(2-(pent-4-en-1-ylamino)ethoxy)ethan-1-ol (int-a4). LCMS (Condition 1): m/z 551.2 [M+H]⁺. 1.82 min.

### Example 50: Synthesis of 6-(5-hydroxypentyl)-2³-(trifluoromethyl)-4-thia-3,6-diaza-2,5(2,6)-dipyridina-1(1,2)-benzenacyclodecaphane 4,4-dioxide (50)

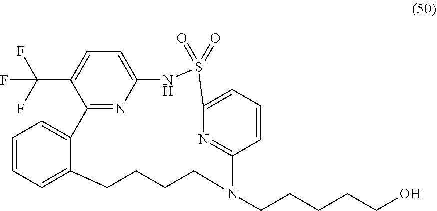 [SMILES: O=S1(=O)Nc2ccc(C(F)(F)F)c(n2)-c2ccccc2CCCCN(CCCCCO)c2cccc1n2]

6-(5-hydroxypentyl)-2³-(trifluoromethyl)-4-thia-3,6-diaza-2,5(2,6)-dipyridina-1(1,2)-benzenacyclodecaphane 4,4-dioxide (50) was synthesized using the procedure described in Example 12 except 5-(hex-5-en-1-ylamino)pentan-1-ol (int-a34) was replaced with 5-(but-3-en-1-ylamino)pentan-1-ol (int-a5). LCMS (Condition 1): m/z 535.2 [M+H]⁺. 1.82 min.

### Example 51: Synthesis of 2³-chloro-6-(6-hydroxyhexyl)-4-thia-3,6-diaza-2,5(2,6)-dipyridina-1(1,2)-benzenacycloundecaphane 4,4-dioxide (51)

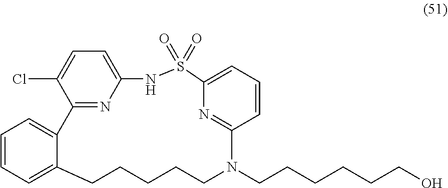 [SMILES: O=S1(=O)Nc2ccc(Cl)c(n2)-c2ccccc2CCCCCN(CCCCCCO)c2cccc1n2]

2³-chloro-6-(6-hydroxyhexyl)-4-thia-3,6-diaza-2,5(2,6)-dipyridina-1(1,2)-benzenacycloundecaphane 4,4-dioxide (51) was synthesized using the procedure described in Example 12 except 6-fluoro-N-(5-(trifluoromethyl)-6-(2-vinylphenyl)pyridin-2-yl)pyridine-2-sulfonamide (int-b2) was replaced with N-(5-chloro-6-(2-vinylphenyl)pyridin-2-yl)-6-fluoropyridine-2-sulfonamide (int-b3) and 5-(hex-5-en-1-ylamino)pentan-1-ol (int-a34) was replaced with 6-(pent-4-en-1-ylamino)hexan-1-ol (int-a3). LCMS (Condition 1): m/z 529.2 [M+H]⁺. 1.88 min.

### Example 52: Synthesis of 1⁵-fluoro-6-(3-hydroxypropyl)-2³-(trifluoromethyl)-4-thia-3,6-diaza-2.5(2.6)-dipyridina-1(1.2)-benzenacyclododecaphane 4,4-dioxide (52)

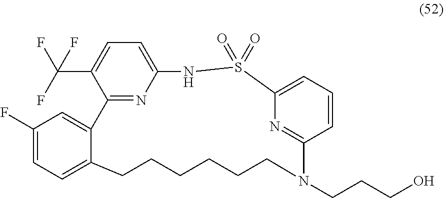 [SMILES: O=S1(=O)Nc2ccc(C(F)(F)F)c(n2)-c2cc(F)ccc2CCCCCCN(CCCO)c2cccc1n2]

1⁵-fluoro-6-(3-hydroxypropyl)-2³-(trifluoromethyl)-4-thia-3,6-diaza-2,5(2,6)-dipyridina-1(1,2)-benzenacyclododecaphane 4,4-dioxide (52) was synthesized using the procedure described in Example 12 except 6-fluoro-N-(5-(trifluoromethyl)-6-(2-vinylphenyl)pyridin-2-yl)pyridine-2-sulfonamide (int-b2) was replaced with 6-fluoro-N-(6-(5-fluoro-2-vinylphenyl)-5-(trifluoromethyl)pyridin-2-yl)pyridine-2-sulfonamide (int-b1) and 5-(hex-5-en-1-ylamino)pentan-1-ol (int-a34) was replaced with 3-(hex-5-en-1-ylamino)propan-1-ol (int-a17). LCMS (Condition 1): m/z 553.1 [M+H]⁺. 1.79 min.

### Example 53: Synthesis of 1⁵-fluoro-6-(4-hydroxybutyl)-2³-(trifluoromethyl)-4-thia-3,6-diaza-2,5(2,6)-dipyridina-1(1,2)-benzenacycloundecaphane 4,4-dioxide (53)

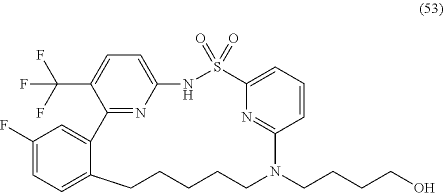 [SMILES: O=S1(=O)Nc2ccc(C(F)(F)F)c(n2)-c2cc(F)ccc2CCCCCN(CCCCO)c2cccc1n2]

1⁵-fluoro-6-(4-hydroxybutyl)-2³-(trifluoromethyl)-4-thia-3,6-diaza-2,5(2,6)-dipyridina-1(1,2)-benzenacycloundecaphane 4,4-dioxide (53) was synthesized using the procedure described in Example 12 except 6-fluoro-N-(5-(trifluoromethyl)-6-(2-vinylphenyl)pyridin-2-yl)pyridine-2-sulfonamide (int-b2) was replaced with 6-fluoro-N-(6-(5-fluoro-2-vinylphenyl)-5-(trifluoromethyl)pyridin-2-yl)pyridine-2-sulfonamide (int-b1) and 5-(hex-5-en-1-ylamino)pentan-1-ol (int-a34) was replaced with 4-(pent-4-en-1-ylamino)butan-1-ol (int-a29). LCMS (Condition 1): m/z 553.1 [M+H]⁺. 1.38 min.

### Example 54: Synthesis of 6-(4-hydroxybutyl)-2³-(trifluoromethyl)-9-oxa-4-thia-3,6-diaza-2,5(2,6)-dipyridina-1(1,2)-benzenacyclododecaphane 4,4-dioxide (54)

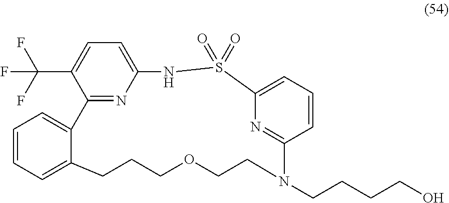 [SMILES: O=S1(=O)Nc2ccc(C(F)(F)F)c(n2)-c2ccccc2CCCOCCN(CCCCO)c2cccc1n2]

6-(4-hydroxybutyl)-2³-(trifluoromethyl)-9-oxa-4-thia-3,6-diaza-2,5(2,6)-dipyridina-1(1,2)-benzenacyclododecaphane 4,4-dioxide (54) was synthesized using the procedure described in Example 12 except 5-(hex-5-en-1-ylamino)pentan-1-ol (int-a34) was replaced with 4-((2-(allyloxy)ethyl)amino)butan-1-ol (int-a6). LCMS (Condition 1): m/z 551.2 [M+H]⁺. 1.72 min.

### Example 55: Synthesis of 6-(4-hydroxybutyl)-2³-(trifluoromethyl)-4-thia-3,6-diaza-2,5(2,6)-dipyridina-1(1.2)-benzenacyclodecaphane 4,4-dioxide (55)

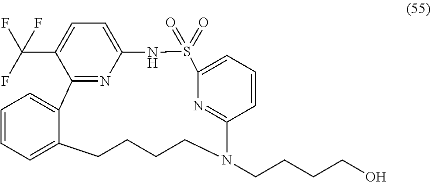 [SMILES: O=S1(=O)Nc2ccc(C(F)(F)F)c(n2)-c2ccccc2CCCCN(CCCCO)c2cccc1n2]

6-(4-hydroxybutyl)-2³-(trifluoromethyl)-4-thia-3,6-diaza-2,5(2,6)-dipyridina-1(1,2)-benzenacyclodecaphane 4,4-dioxide (55) was synthesized using the procedure described in Example 12 except 5-(hex-5-en-1-ylamino)pentan-1-ol (int-a34) was replaced with 4-(but-3-en-1-ylamino)butan-1-ol (int-a7). LCMS (Condition 1): m/z 521.2 [M+H]⁺. 1.85 min.

### Example 56: Synthesis of 2³-chloro-6-(4-hydroxybutyl)-4-thia-3,6-diaza-2,5(2,6)-dipyridina-1(1,2)-benzenacyclodecaphane 4,4-dioxide (56)

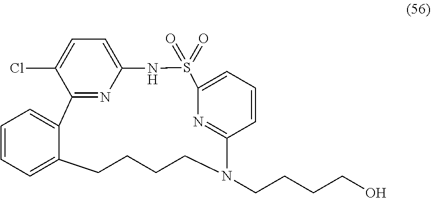 [SMILES: O=S1(=O)Nc2ccc(Cl)c(n2)-c2ccccc2CCCCN(CCCCO)c2cccc1n2]

2³-chloro-6-(4-hydroxybutyl)-4-thia-3,6-diaza-2,5(2,6)-dipyridina-1(1,2)-benzenacyclodecaphane 4,4-dioxide (56) was synthesized using the procedure described in Example 12 except 6-fluoro-N-(5-(trifluoromethyl)-6-(2-vinylphenyl)pyridin-2-yl)pyridine-2-sulfonamide (int-b2) was replaced with N-(5-chloro-6-(2-vinylphenyl)pyridin-2-yl)-6-fluoropyridine-2-sulfonamide (int-b3) and 5-(hex-5-en-1-ylamino)pentan-1-ol (int-a34) was replaced with 4-(but-3-en-1-ylamino)butan-1-ol (int-a7). LCMS (Condition 1): m/z 487.2 [M+H]⁺. 1.74 min.

### Example 57: Synthesis of 6-(3-hydroxypropyl)-4-thia-3,6-diaza-2,5(2,6)-dipyridina-1(1,2)-benzenacycloundecaphane 4,4-dioxide (57)

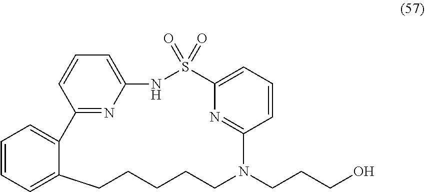 [SMILES: O=S1(=O)Nc2cccc(n2)-c2ccccc2CCCCCN(CCCO)c2cccc1n2]

6-(3-hydroxypropyl)-4-thia-3,6-diaza-2,5(2,6)-dipyridina-1(1,2)-benzenacycloundecaphane 4,4-dioxide (57) was synthesized using the procedure described in Example 12 except 6-fluoro-N-(5-(trifluoromethyl)-6-(2-vinylphenyl)pyridin-2-yl)pyridine-2-sulfonamide (int-b2) was replaced with 6-fluoro-N-(6-(2-vinylphenyl)pyridin-2-yl)pyridine-2-sulfonamide (int-b6) and 5-(hex-5-en-1-ylamino)pentan-1-ol (int-a34) was replaced with 3-(pent-4-en-1-ylamino)propan-1-ol (int-a24). LCMS (Condition 1): m/z 453.1 [M+H]⁺. 1.66 min.

### Example 58: Synthesis of 6-((1-(hydroxymethyl)cyclopropyl)methyl)-2³-(trifluoromethyl)-4-thia-3,6-diaza-2.5(2.6)-dipyridina-1(1.2)-benzenacycloundecaphane 4,4-dioxide (58)

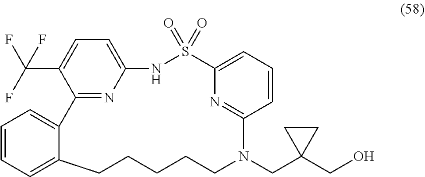 [SMILES: O=S1(=O)Nc2ccc(C(F)(F)F)c(n2)-c2ccccc2CCCCCN(CC2(CO)CC2)c2cccc1n2]

6-((1-(hydroxymethyl)cyclopropyl)methyl)-2³-(trifluoromethyl)-4-thia-3,6-diaza-2,5(2,6)-dipyridina-1(1,2)-benzenacycloundecaphane 4,4-dioxide (58) was synthesized using the procedure described in Example 12 except 5-(hex-5-en-1-ylamino)pentan-1-ol (int-a34) was replaced with (1-((pent-4-en-1-ylamino)methyl)cyclopropyl)methanol (int-a8). LCMS (Condition 1): m/z 547.3 [M+H]⁺. 1.79 min.

### Example 59: Synthesis of 6-((1-(hydroxymethyl)cyclopropyl)methyl)-2³-(trifluoromethyl)-9-oxa-4-thia-3,6-diaza-2,5(2,6)-dipyridina-1(1,2)-benzenacyclododecaphane 4,4-dioxide (59)

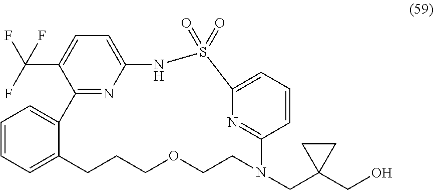 [SMILES: O=S1(=O)Nc2ccc(C(F)(F)F)c(n2)-c2ccccc2CCCOCCN(CC2(CO)CC2)c2cccc1n2]

6-((1-(hydroxymethyl)cyclopropyl)methyl)-2³-(trifluoromethyl)-9-oxa-4-thia-3,6-diaza-2,5(2,6)-dipyridina-1(1,2)-benzenacyclododecaphane 4,4-dioxide (59) was synthesized using the procedure described in Example 12 except 5-(hex-5-en-1-ylamino)pentan-1-ol (int-a34) was replaced with (1-(((2-(allyloxy)ethyl)amino)methyl)cyclopropyl)methanol (int-a11). LCMS (Condition 1): m/z 563.3 [M+H]⁺. 1.72 min.

### Example 60: Synthesis of 1⁵-fluoro-6-(3-hydroxy-2,2-dimethylpropyl)-2³-(trifluoromethyl)-4-thia-3,6-diaza-2,5(2,6)-dipyridina-1(1,2)-benzenacycloundecaphane 4,4-dioxide (60)

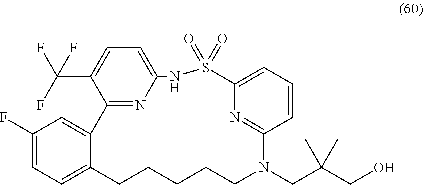 [SMILES: CC(C)(CO)CN1CCCCCc2ccc(F)cc2-c2nc(ccc2C(F)(F)F)NS(=O)(=O)c2cccc1n2]

1⁵-fluoro-6-(3-hydroxy-2,2-dimethylpropyl)-2³-(trifluoromethyl)-4-thia-3,6-diaza-2,5(2,6)-dipyridina-1(1,2)-benzenacycloundecaphane 4,4-dioxide (60) was synthesized using the procedure described in Example 12 except 6-fluoro-N-(5-(trifluoromethyl)-6-(2-vinylphenyl)pyridin-2-yl)pyridine-2-sulfonamide (int-b2) was replaced with 6-fluoro-N-(6-(5-fluoro-2-vinylphenyl)-5-(trifluoromethyl)pyridin-2-yl)pyridine-2-sulfonamide (int-b1) and 5-(hex-5-en-1-ylamino)pentan-1-ol (int-a34) was replaced with 2,2-dimethyl-3-(pent-4-en-1-ylamino)propan-1-ol (int-a25). LCMS (Condition 1): m/z 567.2 [M+H]⁺. 1.98 min.

### Example 61: Synthesis of methyl 2-(4,4-dioxido-2³-(trifluoromethyl)-4-thia-3,6-diaza-2,5(2,6)-dipyridina-1(1.2)-benzenacyclododecaphane-6-yl)acetate (61)

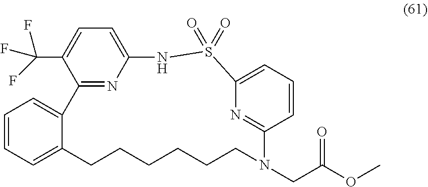 [SMILES: COC(=O)CN1CCCCCCc2ccccc2-c2nc(ccc2C(F)(F)F)NS(=O)(=O)c2cccc1n2]

methyl 2-(4,4-dioxido-2³-(trifluoromethyl)-4-thia-3,6-diaza-2,5(2,6)-dipyridina-1(1,2)-benzenacyclododecaphane-6-yl)acetate (61) was synthesized using the procedure described in Example 12 except 5-(hex-5-en-1-ylamino)pentan-1-ol (int-a34) was replaced with methyl hex-5-en-1-ylglycinate (int-a19). LC-MS (Condition 1): m/z 549.3 [M+1]⁺, 1.74 min.

### Example 62: Synthesis of 3-(4,4-dioxido-2³-(trifluoromethyl)-4-thia-3,6-diaza-2,5(2,6)-dipyridina-1(1,2)-benzenacyclotridecaphane-6-yl)propanoic acid (62)

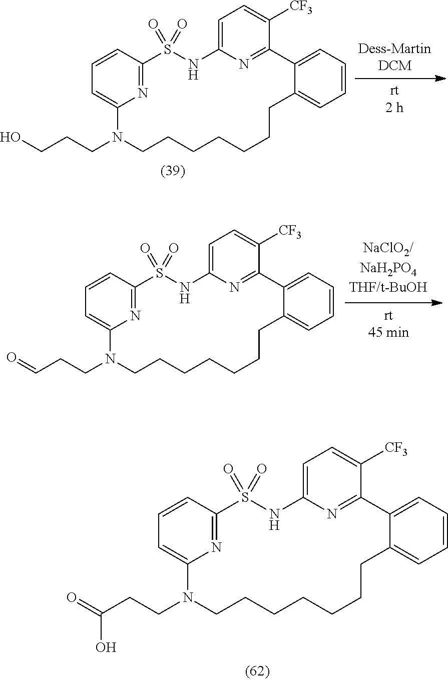 [SMILES: O=S1(=O)Nc2ccc(C(F)(F)F)c(n2)-c2ccccc2CCCCCCCN(CCCO)c2cccc1n2 ; O=CCCN1CCCCCCCc2ccccc2-c2nc(ccc2C(F)(F)F)NS(=O)(=O)c2cccc1n2 ; O=C(O)CCN1CCCCCCCc2ccccc2-c2nc(ccc2C(F)(F)F)NS(=O)(=O)c2cccc1n2]

### Step 1. Synthesis of 3-(4,4-dioxido-2³-(trifluoromethyl)-4-thia-3,6-diaza-2,5(2,6)-dipyridina-1(1,2)-benzenacyclotridecaphane-6-yl)propanal

To a solution of 6-(3-hydroxypropyl)-2³-(trifluoromethyl)-4-thia-3,6-diaza-2,5(2,6)-dipyridina-1(1,2)-benzenacyclotridecaphane 4,4-dioxide (39) (56 mg, 0.10 mmol) in DCM (6 mL) was added in one portion Dess-Martin periodinane (56 mg, 0.13 mmol) at room temperature. The mixture was stirred for 2 h. LCMS indicated that the reaction was complete. Saturated NaHCO₃ (1 mL) and saturated Na₂S2O₃ (1 mL) were added. The mixture was stirred vigorously for 15 min, the reaction mixture was poured into 100 mL of DCM in a 250-mL separation funnel and the layers were separated. The combined organic phases were washed with brine and dried over Na₂SO₄, filtered and concentrated. The residue was subject to ISCO purification (40 g column, EtOAc/heptane 0-100%) to give the title compound (55 mg, 98% yield) as a white solid. LC-MS: m/z 547.20 [M+1]⁺, 1.91 min.

### Step 2. Synthesis of 3-(4,4-dioxido-2³-(trifluoromethyl)-4-thia-3,6-diaza-2,5(2,6)-dipyridina-1(1,2)-benzenacyclotridecaphane-6-yl)propanoic acid (62)

To a solution of 3-(4,4-dioxido-2³-(trifluoromethyl)-4-thia-3,6-diaza-2,5(2,6)-dipyridina-1(1,2)-benzenacyclotridecaphane-6-yl)propanal (54 mg, 0.10 mmol) in t-BuOH (Volume: 3 mL) and THE (Volume: 3 mL) was added 2-methyl-2-butene (0.63 mL, 5.93 mmol), followed by adding NaH₂PO₄ (67 mg, 0.59 mmol) in water (0.5 mL) and NaClO₂ (71 mg, 0.59 mmol) in water (0.5 mL). The resulting mixture was stirred at room temperature for 45 min. LC-MS indicated that the reaction was complete. The reaction mixture was poured into 100 mL of EtOAc in a 250-mL separation funnel, washed with water (10 mL) and brine (10 mL). The organic phase was dried over Na₂SO₄, filtered and concentrated, the residue was then subjected to ISCO purification (40 g column, MeOH/DCM 0-10%) to get the title compound as a white solid. LC-MS (Condition 1): m/z 563.20 [M+1]⁺, 1.86 min. ¹H NMR (400 MHz, DMSO-d₆) δ 12.26 (s, 1H), 11.74 (s, 1H), 8.17 (d, J=9.0 Hz, 1H), 7.73 (dd, J=8.7, 7.3 Hz, 1H), 7.55-7.39 (m, 1H), 7.35 (td, J=7.4, 1.5 Hz, 1H), 7.30-7.21 (m, 2H), 7.21-7.11 (m, 2H), 6.93 (d, J=8.8 Hz, 1H), 3.71-3.41 (m, 4H), 2.59-2.51 (m, 2H), 2.39-2.29 (m, 1H), 2.20-1.99 (m, 1H), 1.42-1.05 (m, 6H), 1.05-0.92 (m, 2H), 0.91-0.76 (m, 2H).

### Example 63: Synthesis of 4-(1⁵-fluoro-4,4-dioxido-2³-(trifluoromethyl)-11-oxa-4-thia-3,6-diaza-2,5(2,6)-dipyridina-1(1,2)-benzenacycloundecaphane-6-yl)butanoic acid (63)

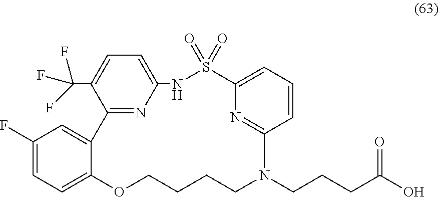 [SMILES: O=C(O)CCCN1CCCCOc2ccc(F)cc2-c2nc(ccc2C(F)(F)F)NS(=O)(=O)c2cccc1n2]

4-(1⁵-fluoro-4,4-dioxido-2³-(trifluoromethyl)-11-oxa-4-thia-3,6-diaza-2,5(2,6)-dipyridina-1(1,2)-benzenacycloundecaphane-6-yl)butanoic acid (63) was synthesized using the procedure described in Example 62 except 6-(3-hydroxypropyl)-2³-(trifluoromethyl)-4-thia-3,6-diaza-2,5(2,6)-dipyridina-1(1,2)-benzenacyclotridecaphane 4,4-dioxide (39) was replaced with 15-fluoro-6-(4-hydroxybutyl)-2³-(trifluoromethyl)-11-oxa-4-thia-3,6-diaza-2,5(2,6)-dipyridina-1(1,2)-benzenacycloundecaphane 4,4-dioxide (23). LC-MS (Condition 1): m/z 569.2 [M+1]⁺, 1.70 min. ¹H NMR (400 MHz, DMSO-d₆) δ 12.15 (s, 1H), 11.67 (s, 1H), 8.05 (d, J=8.9 Hz, 1H), 7.69 (t, J=7.9 Hz, 1H), 7.35-7.15 (m, 3H), 7.15-7.00 (m, 2H), 6.90 (d, J=8.8 Hz, 1H), 4.05-3.72 (m, 3H), 3.47-3.35 (m, 1H), 3.23-3.09 (m, 1H), 3.05-2.88 (m, 1H), 2.26 (t, J=7.1 Hz, 2H), 1.79-1.56 (m, 3H), 1.55-1.34 (m, 2H), 1.30-1.10 (m, 1H).

### Example 64: Synthesis of 3-(2-(4,4-dioxido-2³-(trifluoromethyl)-4-thia-3,6-diaza-2,5(2,6)-dipyridina-1(1,2)-benzenacycloundecaphane-6-yl)ethoxy)propanoic acid (64)

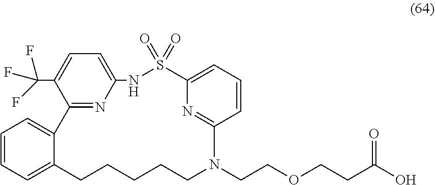 [SMILES: O=C(O)CCOCCN1CCCCCc2ccccc2-c2nc(ccc2C(F)(F)F)NS(=O)(=O)c2cccc1n2]

3-(2-(4,4-dioxido-2³-(trifluoromethyl)-4-thia-3,6-diaza-2,5(2,6)-dipyridina-1(1,2)-benzenacycloundecaphane-6-yl)ethoxy)propanoic acid (64) was synthesized using the procedure described in Example 62 except 6-(3-hydroxypropyl)-2³-(trifluoromethyl)-4-thia-3,6-diaza-2,5(2,6)-dipyridina-1(1,2)-benzenacyclotridecaphane 4,4-dioxide (39) was replaced with 6-(2-(3-hydroxypropoxy)ethyl)-2³-(trifluoromethyl)-4-thia-3,6-diaza-2,5(2,6)-dipyridina-1(1,2)-benzenacycloundecaphane 4,4-dioxide (14). LCMS (Condition 1): m/z 579.2 [M+H]⁺, 1.84 min. ¹H NMR (400 MHz, DMSO-d₆) δ 12.16 (s, 1H), 11.78 (s, 1H), 8.09 (d, J=8.9 Hz, 1H), 7.69 (m, 1H), 7.36 (m, 1H), 7.24 (m, 4H), 7.14 (d, J=7.4 Hz, 1H), 6.88 (d, J=8.7 Hz, 1H), 3.93 (m, 1H), 3.57 (t, J=6.3 Hz, 2H), 3.45 (m, 4H), 2.91 (m, 1H), 2.40 (m, 3H), 1.89 (m, 1H), 1.72 (m, 1H), 1.33 (m, 2H), 1.09 (m, 3H). ¹⁹F NMR (376 MHz, DMSO-d₆) δ−56.86 (s).

### Example 65: Synthesis of 4-(4,4-dioxido-2³-(trifluoromethyl)-4-thia-3,6-diaza-2,5(2,6)-dipyridina-1(1,2)-benzenacyclotridecaphane-6-yl)butanoic acid (65)

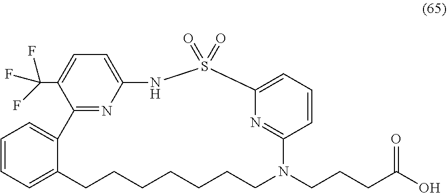 [SMILES: O=C(O)CCCN1CCCCCCCc2ccccc2-c2nc(ccc2C(F)(F)F)NS(=O)(=O)c2cccc1n2]

4-(4,4-dioxido-2³-(trifluoromethyl)-4-thia-3,6-diaza-2,5(2,6)-dipyridina-11(1,2)-benzenacyclotridecaphane-6-yl)butanoic acid (65) was synthesized using the procedure described in Example 62 except 6-(3-hydroxypropyl)-2³-(trifluoromethyl)-4-thia-3,6-diaza-2,5(2,6)-dipyridina-1(1,2)-benzenacyclotridecaphane 4,4-dioxide (39) was replaced with 6-(4-hydroxybutyl)-2³-(trifluoromethyl)-4-thia-3,6-diaza-2,5(2,6)-dipyridina-1(1,2)-benzenacyclotridecaphane 4,4-dioxide (40). LC-MS (Condition 1): m/z 577.2 [M+1]⁺, 1.89 min. ¹H NMR (400 MHz, DMSO-d₆) δ 12.14 (s, 1H), 11.75 (s, 1H), 8.19 (d, J=9.0 Hz, 1H), 7.73 (dd, J=8.7, 7.2 Hz, 1H), 7.53-7.39 (m, 1H), 7.35 (td, J=7.4, 1.5 Hz, 1H), 7.30-7.21 (m, 2H), 7.21-7.12 (m, 2H), 6.95 (d, J=8.8 Hz, 1H), 3.72-3.56 (m, 1H), 3.56-3.41 (m, 1H), 3.37-3.34 (m, 1H), 3.32-3.26 (m, 1H), 2.45-2.22 (m, 3H), 2.17-2.01 (m, 1H), 1.79-1.62 (m, 2H), 1.44-1.30 (m, 3H), 1.29-0.92 (m, 5H), 0.90-0.70 (m, 2H).

### Example 66: Synthesis of 4-(4,4-dioxido-2³-(trifluoromethyl)-4-thia-3,6-diaza-2,5(2,6)-dipyridina-1(1,2)-benzenacyclopentadecaphane-6-yl)butanoic acid (66)

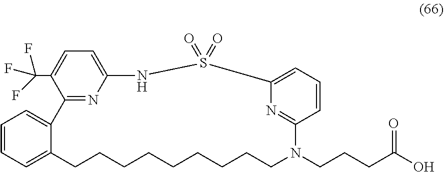 [SMILES: O=C(O)CCCN1CCCCCCCCCc2ccccc2-c2nc(ccc2C(F)(F)F)NS(=O)(=O)c2cccc1n2]

4-(4,4-dioxido-2³-(trifluoromethyl)-4-thia-3,6-diaza-2,5(2,6)-dipyridina-1(1,2)-benzenacyclopentadecaphane-6-yl)butanoic acid (66) was synthesized using the procedure described in Example 62 except 6-(3-hydroxypropyl)-2³-(trifluoromethyl)-4-thia-3,6-diaza-2,5(2,6)-dipyridina-1(1,2)-benzenacyclotridecaphane 4,4-dioxide (39) was replaced with 6-(4-hydroxybutyl)-2³-(trifluoromethyl)-4-thia-3,6-diaza-2,5(2,6)-dipyridina-1(1,2)-benzenacyclopentadecaphane 4,4-dioxide (42). LCMS (Condition 1): m/z 605.3 [M+1]⁺, 1.97 min. ¹H NMR (400 MHz, DMSO-d₆) δ 12.14 (s, 1H), 11.55 (s, 1H), 8.22 (d, J=8.9 Hz, 1H), 7.75-7.50 (m, 2H), 7.35 (td, J=7.5, 1.5 Hz, 1H), 7.29-7.19 (m, 2H), 7.18-7.09 (m, 2H), 6.96 (d, J=8.8 Hz, 1H), 3.93-3.59 (m, 1H), 3.53-3.34 (m, 3H), 2.35-2.21 (m, 2H), 2.21-2.02 (m, 2H), 1.82-1.64 (m, 2H), 1.53-1.37 (m, 2H), 1.37-0.76 (m, 12H).

### Example 67: Synthesis of 4-(4,4-dioxido-2³-(trifluoromethyl)-4-thia-3,6-diaza-2,5(2,6)-dipyridina-1(1,2)-benzenacycloundecaphane-6-yl)butanoic acid (67)

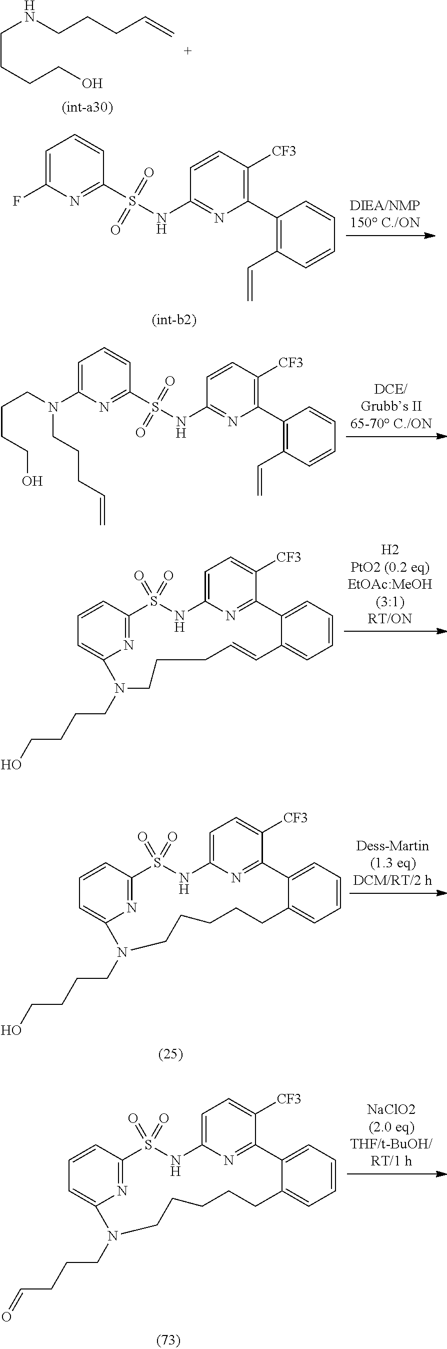 [SMILES: C=CCCCNCCCCO ; C=Cc1ccccc1-c1nc(NS(=O)(=O)c2cccc(F)n2)ccc1C(F)(F)F ; C=CCCCN(CCCCO)c1cccc(S(=O)(=O)Nc2ccc(C(F)(F)F)c(-c3ccccc3C=C)n2)n1 ; O=S1(=O)Nc2ccc(C(F)(F)F)c(n2)-c2ccccc2/C=C/CCCN(CCCCO)c2cccc1n2 ; O=S1(=O)Nc2ccc(C(F)(F)F)c(n2)-c2ccccc2CCCCCN(CCCCO)c2cccc1n2 ; O=CCCCN1CCCCCc2ccccc2-c2nc(ccc2C(F)(F)F)NS(=O)(=O)c2cccc1n2]

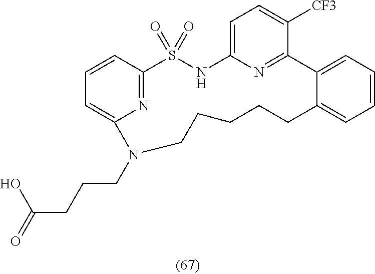 [SMILES: O=C(O)CCCN1CCCCCc2ccccc2-c2nc(ccc2C(F)(F)F)NS(=O)(=O)c2cccc1n2]

### Step 1. Synthesis of 6-((4-hydroxybutyl)(pent-4-en-1-yl)amino)-N-(5-(trifluoromethyl)-6-(2-vinylphenyl)pyridin-2-yl)pyridine-2-sulfonamide

In a 100-mL pressure tube, a mixture of 4-(pent-4-en-1-ylamino)butan-1-ol (int-a29) (4.25 g, 27.00 mmol), 6-fluoro-N-(5-(trifluoromethyl)-6-(2-vinylphenyl)pyridin-2-yl)pyridine-2-sulfonamide (int-b2) (3.81 g, 9 mmol) and DIEA (4.72 mL, 27 mmol) in NMP (25 mL) was stirred at 150° C. for overnight, LC-MS indicated the reaction was almost complete. The reaction mixture was poured into 500 mL of EtOAc in a 1-L separation funnel, washed with 10% citric acid (50 mL×1), water (50 mL×4) and brine (50 mL×1). The organic phase was dried (over Na₂SO₄), filtered and concentrated, the residue was then subjected to ISCO purification (240-g column, heptane in EtOAc 0-100%) to yield 6-((4-hydroxybutyl)(pent-4-en-1-yl)amino)-N-(5-(trifluoromethyl)-6-(2-vinylphenyl)pyridin-2-yl)pyridine-2-sulfonamide as white glassy solid. LC-MS (Condition 1): m/z 561.20, RT 1.886 min. 1H NMR (400 MHz, DMSO-d6) δ 11.53 (s, 1H), 8.16 (d, J=8.9 Hz, 1H), 7.67 (dd, J=8.0, 1.2 Hz, 1H), 7.61 (dd, J=8.7, 7.3 Hz, 1H), 7.49 (d, J=8.8 Hz, 1H), 7.42 (td, J=7.6, 1.4 Hz, 1H), 7.30 (td, J=7.5, 1.2 Hz, 1H), 7.05 (d, J=7.4 Hz, 1H), 7.02 (d, J=7.2 Hz, 1H), 6.81 (d, J=8.7 Hz, 1H), 5.97 (dd, J=17.5, 11.0 Hz, 1H), 5.75 (ddt, J=16.8, 10.2, 6.5 Hz, 1H), 5.61 (d, J=17.4 Hz, 1H), 5.04-4.88 (m, 3H), 4.42 (t, J=5.1 Hz, 1H), 3.45-3.20 (m, 6H), 1.93 (q, J=7.2 Hz, 2H), 1.50-1.30 (m, 6H).

### Step 2. Synthesis of (E)-6-(4-hydroxybutyl)-2³-(trifluoromethyl)-4-thia-3,6-diaza-2,5(2,6)-dipyridina-1(1,2)-benzenacycloundecaphan-10-ene 4,4-dioxide

In a 1 L-flask a solution of 6-((4-hydroxybutyl)(pent-4-en-1-yl)amino)-N-(5-(trifluoromethyl)-6-(2-vinylphenyl)pyridin-2-yl)pyridine-2-sulfonamide (3.24 g, 5.78 mmol) in DCE (500 mL) was purged with argon. Grubbs II (0.368 g, 0.433 mmol) catalyst was added and further purged with argon. The mixture was stirred at 65° C. for 19 hr. LC-MS indicated the reaction was complete. The mixture was concentrated and the residue was subjected to ISCO purification (330-g column, EtOAc in hexane 0-100%) to give a trans-product (E)-6-(4-hydroxybutyl)-2³-(trifluoromethyl)-4-thia-3,6-diaza-2,5(2,6)-dipyridina-1(1,2)-benzenacycloundecaphan-10-ene 4,4-dioxide as a white solid. LC-MS (Condition 1): m/z 533.20, RT 1.810 min. 1H NMR (400 MHz, DMSO-d6) δ 11.65 (s, 1H), 8.13 (d, J=9.0 Hz, 1H), 7.70 (dd, J=8.7, 7.2 Hz, 1H), 7.44-7.36 (m, 2H), 7.34-7.25 (m, 2H), 7.25-7.15 (m, 2H), 6.87 (d, J=8.8 Hz, 1H), 5.89 (d, J=16.0 Hz, 1H), 5.64 (dt, J=16.0, 6.5 Hz, 1H), 4.44 (t, J=5.1 Hz, 1H), 3.62-3.45 (m, 1H), 3.43-3.34 (m, 3H), 3.28-2.98 (m, 2H), 2.11-1.87 (m, 2H), 1.72-1.34 (m, 6H).

### Step 3. Synthesis of 6-(4-hydroxybutyl)-2³-(trifluoromethyl)-4-thia-3,6-diaza-2,5(2,6)-dipyridina-1(1,2)-benzenacycloundecaphane 4,4-dioxide (25)

A mixture of (E)-6-(4-hydroxybutyl)-2³-(trifluoromethyl)-4-thia-3,6-diaza-2,5(2,6)-dipyridina-1(1,2)-benzenacycloundecaphan-10-ene 4,4-dioxide (2.50 g, 4.69 mmol) and PtO₂ (0.213 g, 0.939 mmol) in EtOAc (72 mL) and MeOH (24.0 mL) was stirred under hydrogen at room temperature for overnight, LC-MS indicated the reaction was complete. The mixture was filtered through a layer of celite to remove platinum and then concentrated. The residue was dissolved in EtOAc, then hexane was added, precipitating a white solid which was filtered to yield 6-(4-hydroxybutyl)-2³-(trifluoromethyl)-4-thia-3,6-diaza-2,5(2,6)-dipyridina-1(1,2)-benzenacycloundecaphane 4,4-dioxide as white solid product. The filtrate was concentrated and the residue was subjected to ISCO purification (80-g column, EtOAc in heptane 0-100%) to yield additional 6-(4-hydroxybutyl)-2³-(trifluoromethyl)-4-thia-3,6-diaza-2,5(2,6)-dipyridina-1(1,2)-benzenacycloundecaphane 4,4-dioxide as a white solid. LC-MS (Condition 1): m/z 535.20, RT 1.873 min. 1H NMR (600 MHz, DMSO-d6) δ 11.76 (s, 1H), 8.10 (d, J=9.0 Hz, 1H), 7.71 (dd, J=8.7, 7.2 Hz, 1H), 7.36 (td, J=7.5, 1.4 Hz, 1H), 7.29 (dd, J=7.9, 1.3 Hz, 1H), 7.25-7.18 (m, 3H), 7.14 (d, J=7.6 Hz, 1H), 6.82 (d, J=8.7 Hz, 1H), 4.42 (t, J=5.1 Hz, 1H), 4.00-3.87 (m, 1H), 3.40 (td, J=6.3, 5.1 Hz, 2H), 3.31-3.27 (m, 2H), 3.26-3.16 (m, 1H), 2.93-2.80 (m, 1H), 2.45-2.35 (m, 1H), 1.94-1.83 (m, 1H), 1.77-1.65 (m, 1H), 1.56-1.21 (m, 5H), 1.17-1.09 (m, 2H), 1.07-0.98 (m, 1H).

### Step 4. Synthesis of 4-(4,4-dioxido-2³-(trifluoromethyl)-4-thia-3,6-diaza-2,5(2,6)-dipyridina-1(1,2)-benzenacycloundecaphane-6-yl)butanal (73)

To a solution of 6-(4-hydroxybutyl)-2³-(trifluoromethyl)-4-thia-3,6-diaza-2,5(2,6)-dipyridina-1(1,2)-benzenacycloundecaphane 4,4-dioxide (2.30 g, 4.30 mmol) in DCM (200 mL) was added in one portion of Dess-Martin periodinane (2.372 g, 5.59 mmol) at room temperature. The mixture was stirred for 2 h. LC-MS indicated that the reaction was complete. Saturated NaHCO₃ (20 mL) and saturated Na₂S2O₃ (20 mL) were added. The mixture was stirred vigorously for 15 min before the layers were separated, and the aqueous layer were extracted with DCM (20 mL×3). The combined organic phases were washed with brine and dried over Na₂SO₄, filtered and concentrated. The residue was subject to ISCO purification (80-g column, EtOAc in heptane 0-100%) to give 4-(4,4-dioxido-2³-(trifluoromethyl)-4-thia-3,6-diaza-2,5(2,6)-dipyridina-1(1,2)-benzenacycloundecaphane-6-yl)butanal as a white solid. LC-MS (Condition 1): m/z 533.20, RT 1.940 min. 1H NMR (600 MHz, DMSO-d6) δ 11.77 (s, 1H), 9.67 (t, J=1.2 Hz, 1H), 8.10 (d, J=9.0 Hz, 1H), 7.74 (dd, J=8.7, 7.2 Hz, 1H), 7.36 (td, J=7.5, 1.4 Hz, 1H), 7.29 (dd, J=7.8, 1.3 Hz, 1H), 7.27-7.19 (m, 3H), 7.14 (d, J=7.6 Hz, 1H), 6.91 (d, J=8.7 Hz, 1H), 4.00-3.86 (m, 1H), 3.31-3.25 (m, 1H), 3.25-3.17 (m, 1H), 2.93-2.82 (m, 1H), 2.44-2.35 (m, 1H), 1.94-1.84 (m, 1H), 1.76-1.61 (m, 3H), 1.41-1.20 (m, 4H), 1.17-1.10 (m, 2H), 1.08-0.98 (m, 1H).

### Step 5. Synthesis of 4-(4,4-dioxido-2³-(trifluoromethyl)-4-thia-3,6-diaza-2,5(2,6)-dipyridina-1(1,2)-benzenacycloundecaphane-6-yl)butanoic acid

To a solution of 4-(4,4-dioxido-2³-(trifluoromethyl)-4-thia-3,6-diaza-2,5(2,6)-dipyridina-1(1,2)-benzenacycloundecaphane-6-yl)butanal (2.2 g, 4.13 mmol) in t-BuOH (60 mL) and THE (60 mL) was added 2-methyl-2-butene (8.75 mL, 83 mmol), followed by the addition of NaH₂PO₄ (991 mg, 8.26 mmol) in water (10 mL) and NaClO₂ (934 mg, 8.26 mmol) in water (10 mL). The resulting mixture was stirred at room temperature for 45 min. LC-MS indicated that the reaction was complete. The reaction mixture was poured into 500 mL of EtOAc in a 1-L separation funnel, washed with water (50 mL×1) and brine (50 mL×1). The organic phase was dried over Na₂SO₄, filtered and concentrated The residue was then subjected to ISCO purification (80-g column, MeOH in DCM, 0-10%), and pure fraction was collected and concentrated. To this residue 24 mL of ethyl ether was added, precipitating a white solid, which was filtered to yield 4-(4,4-dioxido-2³-(trifluoromethyl)-4-thia-3,6-diaza-2,5(2,6)-dipyridina-1(1,2)-benzenacycloundecaphane-6-yl)butanoic acid (67) as a white solid. The filtrate was concentrated to yield additional 4-(4,4-dioxido-2³-(trifluoromethyl)-4-thia-3,6-diaza-2,5(2,6)-dipyridina-1(1,2)-benzenacycloundecaphane-6-yl)butanoic acid (67) as a white/very slightly yellow solid. LC-MS (Condition 1): m/z 549.20, RT 1.841 min. 1H NMR (600 MHz, DMSO-d6) δ 12.14 (s, 1H), 11.77 (s, 1H), 8.10 (d, J=9.0 Hz, 1H), 7.72 (dd, J=8.7, 7.2 Hz, 1H), 7.36 (td, J=7.5, 1.4 Hz, 1H), 7.29 (dd, J=7.8, 1.3 Hz, 1H), 7.26-7.19 (m, 3H), 7.14 (d, J=7.6 Hz, 1H), 6.91 (d, J=8.7 Hz, 1H), 4.04-3.88 (m, 1H), 3.31-3.26 (m, 1H), 3.26-3.18 (m, 1H), 2.94-2.80 (m, 1H), 2.45-2.34 (m, 1H), 2.26 (t, J=7.1 Hz, 2H), 1.97-1.82 (m, 1H), 1.76-1.58 (m, 3H), 1.42-1.25 (m, 2H), 1.20-1.11 (m, 2H), 1.07-0.95 (m, 1H). Compound (67) was isolated as the sodium salt by adding 1.05 equivalents of NaOH to a slurry of the free acid in 95.5% IPA and 4.5% H₂O and stirring overnight. Subsequent filtration yielded Compound (67) as a sodium salt.

### Example 68: Synthesis of 4-(1⁵-fluoro-4,4-dioxido-2³-(trifluoromethyl)-4-thia-3,6-diaza-2,5(2,6)-dipyridina-1(1,2)-benzenacyclododecaphane-6-yl)butanoic acid (68)

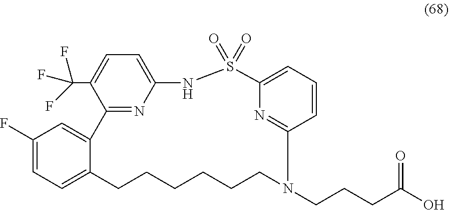 [SMILES: O=C(O)CCCN1CCCCCCc2ccc(F)cc2-c2nc(ccc2C(F)(F)F)NS(=O)(=O)c2cccc1n2]

4-(1⁵-fluoro-4,4-dioxido-2³-(trifluoromethyl)-4-thia-3,6-diaza-2,5(2,6)-dipyridina-1(1,2)-benzenacyclododecaphane-6-yl)butanoic acid (68) was synthesized using the procedure described in Example 62 except 6-(3-hydroxypropyl)-2³-(trifluoromethyl)-4-thia-3,6-diaza-2,5(2,6)-dipyridina-1(1,2)-benzenacyclotridecaphane 4,4-dioxide (39) was replaced with 15-fluoro-6-(4-hydroxybutyl)-2³-(trifluoromethyl)-4-thia-3,6-diaza-2,5(2,6)-dipyridina-1(1,2)-benzenacyclododecaphane 4,4-dioxide (47). LC-MS (Condition 1): m/z 580.9 [M+1]⁺, 1.82 min. ¹H NMR (400 MHz, DMSO-d₆) δ 12.15 (s, 1H), 11.74 (s, 1H), 8.19 (d, J=9.0 Hz, 1H), 7.72 (dd, J=7.3, 8.7 Hz, 1H), 7.44 (d, J=8.7 Hz, 1H), 7.31 (dd, J=5.9, 8.5 Hz, 1H), 7.24-7.15 (m, 2H), 7.07 (d, J=7.7 Hz, 1H), 6.94 (d, J=8.8 Hz, 1H), 3.57 (s, 1H), 3.33 (dq, J=7.4, 8.3, 23.4 Hz, 3H), 2.61-2.52 (m, 1H), 2.28 (t, J=7.1 Hz, 2H), 2.02 (s, 1H), 1.70 (p, J=6.9 Hz, 2H), 1.45-1.16 (m, 3H), 1.08 (s, 3H), 0.94-0.66 (m, 2H). ¹⁹F NMR (376 MHz, DMSO-d₆) δ−56.03 (s, 3F), −117.96 (s, 1F).

### Example 69: Synthesis of 3-(4,4-dioxido-2³-(trifluoromethyl)-4-thia-3,6-diaza-2,5(2,6)-dipyridina-1(1,2)-benzenacyclotetradecaphane-6-yl)propanoic acid (69)

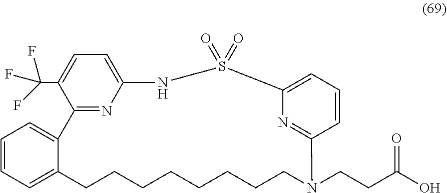 [SMILES: O=C(O)CCN1CCCCCCCCc2ccccc2-c2nc(ccc2C(F)(F)F)NS(=O)(=O)c2cccc1n2]

3-(4,4-dioxido-2³-(trifluoromethyl)-4-thia-3,6-diaza-2,5(2,6)-dipyridina-1(1,2)-benzenacyclotetradecaphane-6-yl)propanoic acid (69) was synthesized using the procedure described in Example 62 except 6-(3-hydroxypropyl)-2³-(trifluoromethyl)-4-thia-3,6-diaza-2,5(2,6)-dipyridina-1(1,2)-benzenacyclotridecaphane 4,4-dioxide (39) was replaced with 6-(3-hydroxypropyl)-2³-(trifluoromethyl)-4-thia-3,6-diaza-2,5(2,6)-dipyridina-1(1,2)-benzenacyclotetradecaphane 4,4-dioxide (43). LC-MS (Condition 1): m/z 577.2 [M+1]⁺, 1.91 min. ¹H NMR (400 MHz, DMSO-d₆) δ 12.32 (s, 1H), 11.62 (s, 1H), 8.20 (d, J=9.0 Hz, 1H), 7.70 (dd, J=8.7, 7.2 Hz, 1H), 7.65-7.45 (m, 1H), 7.35 (td, J=7.4, 1.5 Hz, 1H), 7.29-7.19 (m, 2H), 7.19-7.10 (m, 2H), 6.94 (d, J=8.8 Hz, 1H), 3.83-3.65 (m, 1H), 3.64-3.48 (m, 2H), 3.48-3.35 (m, 1H), 2.54 (dt, J=7.4, 3.5 Hz, 2H), 2.39-2.24 (m, 1H), 2.08-1.94 (m, 1H), 1.45-1.20 (m, 3H), 1.20-0.89 (m, 7H), 0.89-0.76 (m, 2H).

### Example 70: Synthesis of 6-(4,4-dioxido-2³-(trifluoromethyl)-4-thia-3,6-diaza-2,5(2,6)-dipyridina-1(1,2)-benzenacycloundecaphane-6-yl)hexanoic acid (70)

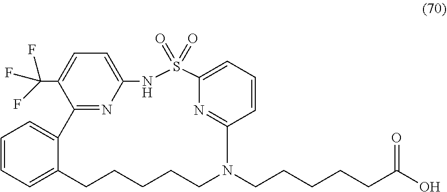 [SMILES: O=C(O)CCCCCN1CCCCCc2ccccc2-c2nc(ccc2C(F)(F)F)NS(=O)(=O)c2cccc1n2]

6-(4,4-dioxido-2³-(trifluoromethyl)-4-thia-3,6-diaza-2,5(2,6)-dipyridina-1(1,2)-benzenacycloundecaphane-6-yl)hexanoic acid (70) was synthesized using the procedure described in Example 62 except 6-(3-hydroxypropyl)-2³-(trifluoromethyl)-4-thia-3,6-diaza-2,5(2,6)-dipyridina-1(1,2)-benzenacyclotridecaphane 4,4-dioxide (3) was replaced with 6-(6-hydroxyhexyl)-2³-(trifluoromethyl)-4-thia-3,6-diaza-2,5(2,6)-dipyridina-1(1,2)-benzenacycloundecaphane 4,4-dioxide (48). LCMS (Condition 1): m/z 577.2 [M+H]⁺, 1.85 min. ¹H NMR (400 MHz, DMSO-d₆) δ 11.99 (s, 1H), 11.77 (s, 1H), 8.10 (d, J=9.0 Hz, 1H), 7.71 (dd, J=8.6, 7.3 Hz, 1H), 7.36 (td, J=7.5, 1.2 Hz, 1H), 7.28 (m, 1H), 7.22 (m, 3H), 7.14 (d, J=7.5 Hz, 1H), 6.81 (d, J=8.8 Hz, 1H), 3.94 (m, 1H), 3.26 (m, 2H), 2.87 (m, 1H), 2.40 (m, 1H), 2.19 (t, J=7.3 Hz, 2H), 1.89 (m, 1H), 1.69 (m, 1H), 0.96-1.54 (m, 11H). ¹⁹F NMR (376 MHz, DMSO-d₆) δ−56.88 (s).

### Example 71: Synthesis of 4-(4,4-dioxido-2³-(trifluoromethyl)-4-thia-3,6-diaza-2,5(2,6)-dipyridina-1(1,2)-benzenacyclotetradecaphane-6-yl)butanoic acid (71)

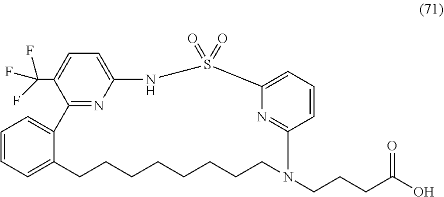 [SMILES: O=C(O)CCCN1CCCCCCCCc2ccccc2-c2nc(ccc2C(F)(F)F)NS(=O)(=O)c2cccc1n2]

4-(4,4-dioxido-2³-(trifluoromethyl)-4-thia-3,6-diaza-2,5(2,6)-dipyridina-1(1,2)-benzenacyclotetradecaphane-6-yl)butanoic acid (71) was synthesized using the procedure described in Example 62 except 6-(3-hydroxypropyl)-2³-(trifluoromethyl)-4-thia-3,6-diaza-2,5(2,6)-dipyridina-1(1,2)-benzenacyclotridecaphane 4,4-dioxide (39) was replaced with 6-(4-hydroxybutyl)-2³-(trifluoromethyl)-4-thia-3,6-diaza-2,5(2,6)-dipyridina-1(1,2)-benzenacyclotetradecaphane 4,4-dioxide (44). LC-MS (Condition 1): m/z 591.2 [M+1]⁺, 1.93 min. ¹H NMR (400 MHz, DMSO-d₆) δ 12.15 (s, 1H), 11.62 (s, 1H), 8.21 (d, J=8.9 Hz, 1H), 7.70 (dd, J=8.8, 7.2 Hz, 1H), 7.62-7.44 (m, 1H), 7.35 (td, J=7.4, 1.5 Hz, 1H), 7.29-7.19 (m, 2H), 7.19-7.08 (m, 2H), 6.96 (d, J=8.7 Hz, 1H), 3.90-3.66 (m, 1H), 3.42-3.22 (m, 3H), 2.40-2.21 (m, 3H), 2.10-1.95 (m, 1H), 1.80-1.67 (m, 2H), 1.42-1.20 (m, 4H), 1.20-0.74 (m, 8H).

### Example 72: Synthesis of 4-(2³-Methyl-4,4-dioxido-4-thia-3,6-diaza-2,5(2,6)-dipyridina-1(1,2)-benzenacycloundecaphane-6-yl)butanoic acid (72)

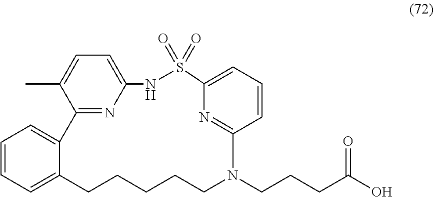 [SMILES: Cc1ccc2nc1-c1ccccc1CCCCCN(CCCC(=O)O)c1cccc(n1)S(=O)(=O)N2]

4-(2³-methyl-4,4-dioxido-4-thia-3,6-diaza-2,5(2,6)-dipyridina-1(1,2)-benzenacycloundecaphane-6-yl)butanoic acid (72) was synthesized using the procedure described in Example 62 except 6-(3-hydroxypropyl)-2³-(trifluoromethyl)-4-thia-3,6-diaza-2,5(2,6)-dipyridina-1(1,2)-benzenacyclotridecaphane 4,4-dioxide (39) was replaced with 6-(4-hydroxybutyl)-2³-methyl-4-thia-3,6-diaza-2,5(2,6)-dipyridina-1(1,2)-benzenacycloundecaphane 4,4-dioxide (19). LCMS (Condition 1): m/z 495.2 [M+H]⁺, 1.72 min. ¹H NMR (400 MHz, DCM-d₂+MeOH-d₄) δ 7.57 (dd, J=7.2, 1.5 Hz, 1H), 7.55 (d, J=8.8 Hz, 1H), 7.44-7.37 (m, 1H), 7.35-7.27 (m, 2H), 7.23 (d, J=7.0 Hz, 1H), 7.17 (dd, J=7.7, 1.4 Hz, 1H), 7.12 (d, J=8.7 Hz, 1H), 6.71 (d, J=8.6 Hz, 1H), 3.77 (s, 1H), 3.30-3.26 (m, 2H), 3.14 (s, 1H), 2.57 (s, 1H), 2.32 (t, J=6.9 Hz, 2H), 2.18 (s, 1H), 1.96 (s, 3H), 1.81 (p, J=7.1 Hz, 2H), 1.67-1.42 (m, 2H), 1.31 (s, 2H), 1.12 (m, 2H).

### Example 73: Synthesis of 4-(4,4-dioxido-2³-(trifluoromethyl)-4-thia-3,6-diaza-2,5(2,6)-dipyridina-1(1,2)-benzenacycloundecaphane-6-yl)butanal (73)

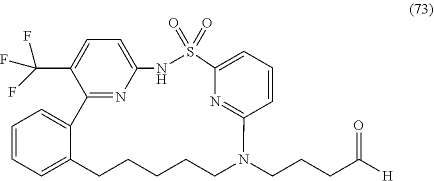 [SMILES: O=CCCCN1CCCCCc2ccccc2-c2nc(ccc2C(F)(F)F)NS(=O)(=O)c2cccc1n2]

4-(4,4-dioxido-2³-(trifluoromethyl)-4-thia-3,6-diaza-2,5(2,6)-dipyridina-1(1,2)-benzenacycloundecaphane-6-yl)butanal (73) was synthesized as described in Example 67. LC-MS (Condition 1): m/z 533.2 [M+1]⁺, 1.94 min. ¹H NMR (600 MHz, DMSO-d₆) δ 11.77 (s, 1H), 9.67 (t, J=1.2 Hz, 1H), 8.10 (d, J=9.0 Hz, 1H), 7.74 (dd, J=8.7, 7.2 Hz, 1H), 7.36 (td, J=7.5, 1.4 Hz, 1H), 7.29 (dd, J=7.8, 1.3 Hz, 1H), 7.27-7.19 (m, 3H), 7.14 (d, J=7.6 Hz, 1H), 6.91 (d, J=8.7 Hz, 1H), 4.00-3.86 (m, 1H), 3.31-3.25 (m, 1H), 3.25-3.17 (m, 1H), 2.93-2.82 (m, 1H), 2.44-2.35 (m, 1H), 1.94-1.84 (m, 1H), 1.76-1.61 (m, 3H), 1.41-1.20 (m, 4H), 1.17-1.10 (m, 2H), 1.08-0.98 (m, 1H).

### Example 74: Synthesis of 2-(2-(4,4-dioxido-2³-(trifluoromethyl)-4-thia-3,6-diaza-2,5(2,6)-dipyridina-1(1.2)-benzenacycloundecaphane-6-yl)ethoxy)acetic acid (74)

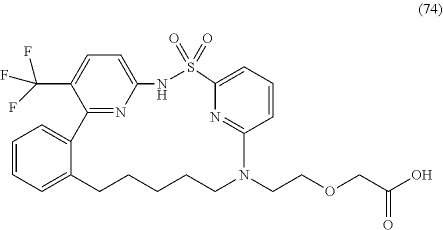 [SMILES: O=C(O)COCCN1CCCCCc2ccccc2-c2nc(ccc2C(F)(F)F)NS(=O)(=O)c2cccc1n2]

2-(2-(4,4-dioxido-2³-(trifluoromethyl)-4-thia-3,6-diaza-2,5(2,6)-dipyridina-1(1,2)-benzenacycloundecaphane-6-yl)ethoxy)acetic acid (74) was synthesized using the procedure described in Example 62 except 6-(3-hydroxypropyl)-2³-(trifluoromethyl)-4-thia-3,6-diaza-2,5(2,6)-dipyridina-1(1,2)-benzenacyclotridecaphane 4,4-dioxide (39) was replaced with 6-(2-(2-hydroxyethoxy)ethyl)-2³-(trifluoromethyl)-4-thia-3,6-diaza-2,5(2,6)-dipyridina-1(1,2)-benzenacycloundecaphane 4,4-dioxide (49). LCMS (Condition 1): m/z 565.2 [M+H]⁺, 1.77 min. ¹H NMR (400 MHz, DMSO-d₆) δ 12.61 (s, 1H), 11.79 (s, 1H), 8.09 (d, J=9.0 Hz, 1H), 7.70 (dd, J=8.7, 7.2 Hz, 1H), 7.36 (td, J=7.4, 1.4 Hz, 1H), 7.29 (m, 1H), 7.22 (m, 3H), 7.14 (d, J=7.7 Hz, 1H), 6.91 (d, J=8.8 Hz, 1H), 4.00 (s, 2H), 3.96 (m, 1H), 3.57 (m, 2H), 3.48 (m, 2H), 2.92 (m, 1H), 2.40 (m, 1H), 1.90 (m, 1H), 1.72 (m, 1H), 1.35 (m, 2H), 1.15 (m, 2H), 1.04 (m, 1H). ¹⁹F NMR (376 MHz, DMSO-d6) δ −56.85 (s).

### Example 75: Synthesis of 3-(4,4-dioxido-2³-(trifluoromethyl)-4-thia-3,6-diaza-2,5(2,6)-dipyridina-1(1,2)-benzenacyclopentadecaphane-6-yl)propanoic acid (75)

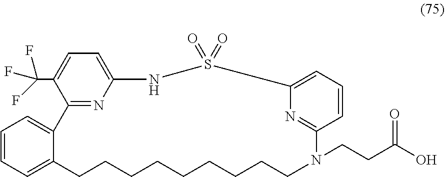 [SMILES: O=C(O)CCN1CCCCCCCCCc2ccccc2-c2nc(ccc2C(F)(F)F)NS(=O)(=O)c2cccc1n2]

3-(4,4-dioxido-2³-(trifluoromethyl)-4-thia-3,6-diaza-2,5(2,6)-dipyridina-1(1,2)-benzenacyclopentadecaphane-6-yl)propanoic acid (75) was synthesized using the procedure described in Example 62 except 6-(3-hydroxypropyl)-2³-(trifluoromethyl)-4-thia-3,6-diaza-2,5(2,6)-dipyridina-1(1,2)-benzenacyclotridecaphane 4,4-dioxide (39) was replaced with 6-(3-hydroxypropyl)-2³-(trifluoromethyl)-4-thia-3,6-diaza-2,5(2,6)-dipyridina-1(1,2)-benzenacyclopentadecaphane 4,4-dioxide (45). LC-MS (Condition 1): m/z 591.3 [M+1]⁺, 1.95 min. ¹H NMR (400 MHz, DMSO-d₆) δ 12.33 (s, 1H), 11.57 (s, 1H), 8.18 (d, J=8.8 Hz, 1H), 7.89-7.49 (m, 2H), 7.35 (td, J=7.5, 1.5 Hz, 1H), 7.30-7.19 (m, 2H), 7.19-7.07 (m, 2H), 6.96 (d, J=8.7 Hz, 1H), 3.84-3.41 (m, 4H), 2.65-2.52 (m, 2H), 2.22-2.04 (m, 2H), 1.55-1.41 (m, 2H), 1.38-1.14 (m, 2H), 1.14-0.94 (m, 8H), 0.94-0.80 (m, 2H).

### Example 76: Synthesis of 4-(4,4-dioxido-2³-(trifluoromethyl)-4-thia-3,6-diaza-2,5(2,6)-dipyridina-1(1.2)-benzenacyclododecaphane-6-yl)butanoic acid (76)

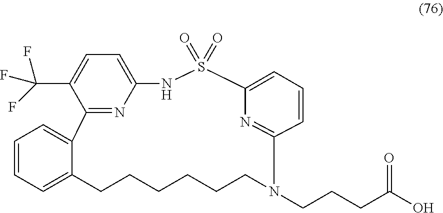 [SMILES: O=C(O)CCCN1CCCCCCc2ccccc2-c2nc(ccc2C(F)(F)F)NS(=O)(=O)c2cccc1n2]

4-(4,4-dioxido-2³-(trifluoromethyl)-4-thia-3,6-diaza-2,5(2,6)-dipyridina-1(1,2)-benzenacyclododecaphane-6-yl)butanoic acid (76) was synthesized using the procedure described in Example 62 except 6-(3-hydroxypropyl)-2³-(trifluoromethyl)-4-thia-3,6-diaza-2,5(2,6)-dipyridina-1(1,2)-benzenacyclotridecaphane 4,4-dioxide (39) was replaced with 6-(4-hydroxybutyl)-2³-(trifluoromethyl)-4-thia-3,6-diaza-2,5(2,6)-dipyridina-1(1,2)-benzenacyclododecaphane 4,4-dioxide (16). LC-MS (Condition 1): m/z 563.2 [M+1]⁺, 1.88 min. ¹H NMR (400 MHz, DMSO-d₆) δ 12.14 (s, 1H), 11.70 (s, 1H), 8.16 (d, J=9.0 Hz, 1H), 7.71 (dd, J=8.7, 7.2 Hz, 1H), 7.41 (d, J=8.7 Hz, 1H), 7.34 (td, J=7.4, 1.5 Hz, 1H), 7.29-7.20 (m, 2H), 7.20-7.10 (m, 2H), 6.93 (d, J=8.7 Hz, 1H), 3.69-3.49 (m, 1H), 3.44-3.35 (m, 1H), 3.31-3.12 (m, 2H), 2.65-2.53 (m, 1H), 2.27 (t, J=7.1 Hz, 2H), 2.11-1.92 (m, 1H), 1.79-1.60 (m, 2H), 1.42-1.00 (m, 6H), 0.94-0.67 (m, 2H).

### Example 77: Synthesis of 5-(4,4-dioxido-2³-(trifluoromethyl)-4-thia-3,6-diaza-2,5(2,6)-dipyridina-1(1,2)-benzenacycloundecaphane-6-yl)pentanoic acid (77)

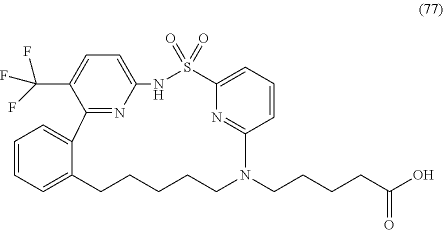 [SMILES: O=C(O)CCCCN1CCCCCc2ccccc2-c2nc(ccc2C(F)(F)F)NS(=O)(=O)c2cccc1n2]

5-(4,4-dioxido-2³-(trifluoromethyl)-4-thia-3,6-diaza-2,5(2,6)-dipyridina-1(1,2)-benzenacycloundecaphane-6-yl)pentanoic acid (77) was synthesized using the procedure described in Example 62 except 6-(3-hydroxypropyl)-2³-(trifluoromethyl)-4-thia-3,6-diaza-2,5(2,6)-dipyridina-1(1,2)-benzenacyclotridecaphane 4,4-dioxide (39) was replaced with 6-(5-hydroxypentyl)-2³-(trifluoromethyl)-4-thia-3,6-diaza-2,5(2,6)-dipyridina-1(1,2)-benzenacycloundecaphane 4,4-dioxide (15). LC-MS (Condition 1): m/z 563.2 [M+1]⁺, 1.82 min. ¹H NMR (400 MHz, DMSO-d₆) δ 12.03 (s, 1H), 11.78 (s, 1H), 8.10 (d, J=8.9 Hz, 1H), 7.71 (dd, J=8.7, 7.2 Hz, 1H), 7.36 (td, J=7.4, 1.5 Hz, 1H), 7.31-7.26 (m, 1H), 7.26-7.17 (m, 3H), 7.14 (d, J=7.5 Hz, 1H), 6.83 (d, J=8.8 Hz, 1H), 4.04-3.85 (m, 1H), 3.31-3.13 (m, 1H), 2.93-2.78 (m, 1H), 2.46-2.34 (m, 1H), 2.27-2.18 (m, 2H), 1.96-1.80 (m, 1H), 1.78-1.62 (m, 1H), 1.56-1.22 (m, 6H), 1.20-0.94 (m, 4H).

### Example 78: Synthesis of 5-(4,4-dioxido-2³-(trifluoromethyl)-4-thia-3,6-diaza-2,5(2,6)-dipyridina-1(1.2)-benzenacyclododecaphane-6-yl)pentanoic acid (78)

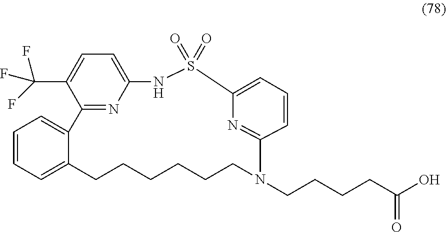 [SMILES: O=C(O)CCCCN1CCCCCCc2ccccc2-c2nc(ccc2C(F)(F)F)NS(=O)(=O)c2cccc1n2]

5-(4,4-dioxido-2³-(trifluoromethyl)-4-thia-3,6-diaza-2,5(2,6)-dipyridina-1(1,2)-benzenacyclododecaphane-6-yl)pentanoic acid (78) was synthesized using the procedure described in Example 62 except 6-(3-hydroxypropyl)-2³-(trifluoromethyl)-4-thia-3,6-diaza-2,5(2,6)-dipyridina-1(1,2)-benzenacyclotridecaphane 4,4-dioxide (39) was replaced with 6-(5-hydroxypentyl)-2³-(trifluoromethyl)-4-thia-3,6-diaza-2,5(2,6)-dipyridina-1(1,2)-benzenacyclododecaphane 4,4-dioxide (12). LC-MS (Condition 1): m/z 577.2 [M+1]⁺, 1.85 min. ¹H NMR (400 MHz, DMSO-d₆) δ 12.03 (s, 1H), 11.70 (s, 1H), 8.16 (d, J=9.0 Hz, 1H), 7.69 (dd, J=8.7, 7.2 Hz, 1H), 7.49-7.38 (m, 1H), 7.34 (td, J=7.4, 1.5 Hz, 1H), 7.29-7.20 (m, 2H), 7.20-7.10 (m, 2H), 6.86 (d, J=8.8 Hz, 1H), 3.69-3.49 (m, 1H), 3.47-3.35 (m, 1H), 3.32-3.10 (m, 2H), 2.65-2.54 (m, 1H), 2.29-2.19 (m, 2H), 2.10-1.94 (m, 1H), 1.58-1.43 (m, 4H), 1.42-0.95 (m, 6H), 0.94-0.82 (m, 1H), 0.82-0.67 (m, 1H).

### Example 79: Synthesis of 4-(2³-chloro-4,4-dioxido-4-thia-3,6-diaza-2,5(2,6)-dipyridina-1(1,2)-benzenacycloundecaphane-6-yl)butanoic acid (79)

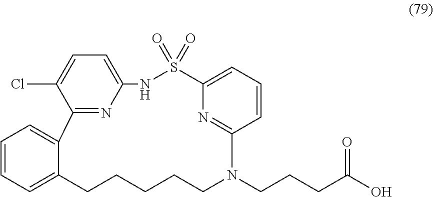 [SMILES: O=C(O)CCCN1CCCCCc2ccccc2-c2nc(ccc2Cl)NS(=O)(=O)c2cccc1n2]

4-(2³-chloro-4,4-dioxido-4-thia-3,6-diaza-2,5(2,6)-dipyridina-1(1,2)-benzenacycloundecaphane-6-yl)butanoic acid (79) was synthesized using the procedure described in Example 62 except 6-(3-hydroxypropyl)-2³-(trifluoromethyl)-4-thia-3,6-diaza-2,5(2,6)-dipyridina-1(1,2)-benzenacyclotridecaphane 4,4-dioxide (39) was replaced with 23-chloro-6-(4-hydroxybutyl)-4-thia-3,6-diaza-2,5(2,6)-dipyridina-1(1,2)-benzenacycloundecaphane 4,4-dioxide (46). LCMS (Condition 1): m/z 515.2 [M+H]⁺, 1.75 min. ¹H NMR (400 MHz, DMSO-d₆) δ 12.11 (s, 1H), 11.31 (s, 1H), 7.86 (d, J=8.9 Hz, 1H), 7.70 (dd, J=8.7, 7.3 Hz, 1H), 7.35 (td, J=7.4, 1.5 Hz, 1H), 7.27 (m, 2H), 7.17 (m, 3H), 6.88 (d, J=8.7 Hz, 1H), 3.77 (m, 1H), 3.25 (t, J=7.8 Hz, 2H), 2.97 (m, 1H), 2.52 (m, 1H), 2.26 (t, J=7.1 Hz, 2H), 2.07 (m, 1H), 1.66 (s, 2H), 1.57 (m, 1H), 0.87-1.35 (m, 5H).

### Example 80: Synthesis of 5-(4,4-dioxido-2³-(trifluoromethyl)-4-thia-3,6-diaza-2,5(2,6)-dipyridina-1(1.2)-benzenacyclodecaphane-6-yl)pentanoic acid (80)

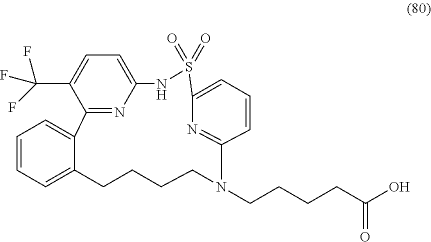 [SMILES: O=C(O)CCCCN1CCCCc2ccccc2-c2nc(ccc2C(F)(F)F)NS(=O)(=O)c2cccc1n2]

5-(4,4-dioxido-2³-(trifluoromethyl)-4-thia-3,6-diaza-2,5(2,6)-dipyridina-1(1,2)-benzenacyclodecaphane-6-yl)pentanoic acid (80) was synthesized using the procedure described in Example 62 except 6-(3-hydroxypropyl)-2³-(trifluoromethyl)-4-thia-3,6-diaza-2,5(2,6)-dipyridina-1(1,2)-benzenacyclotridecaphane 4,4-dioxide (39) was replaced with 6-(5-hydroxypentyl)-2³-(trifluoromethyl)-4-thia-3,6-diaza-2,5(2,6)-dipyridina-1(1,2)-benzenacyclodecaphane 4,4-dioxide (50). LCMS (Condition 1): m/z 549.2 [M+H]⁺, 1.77 min. ¹H NMR (400 MHz, DMSO-d₆) δ 12.01 (s, 1H), 11.67 (s, 1H), 8.15 (d, J=8.9 Hz, 1H), 7.70 (dd, J=8.6, 7.3 Hz, 1H), 7.34 (m, 2H), 7.26 (m, 3H), 7.16 (d, J=7.2 Hz, 1H), 6.81 (d, J=8.7 Hz, 1H), 3.91 (t, J=11.5 Hz, 1H), 3.30 (m, 1H), 3.22 (m, 1H), 2.85 (m, 1H), 2.54 (m, 1H), 2.22 (m, 2H), 1.74 (m, 1H), 1.62 (m, 1H), 1.48 (m, 4H), 1.30 (m, 2H), 0.67 (m, 1H). ¹⁹F NMR (376 MHz, DMSO-d₆) δ−57.00 (s).

### Example 81: Synthesis of 3-(1⁵-fluoro-4,4-dioxido-2³-(trifluoromethyl)-11-oxa-4-thia-3,6-diaza-2,5(2,6)-dipyridina-1(1,2)-benzenacycloundecaphane-6-yl)propanoic acid (81)

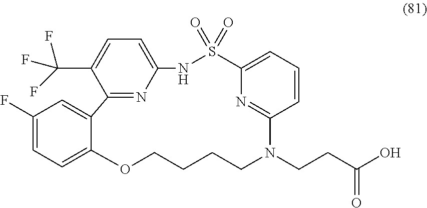 [SMILES: O=C(O)CCN1CCCCOc2ccc(F)cc2-c2nc(ccc2C(F)(F)F)NS(=O)(=O)c2cccc1n2]

3-(1⁵-fluoro-4,4-dioxido-2³-(trifluoromethyl)-11-oxa-4-thia-3,6-diaza-2,5(2,6)-dipyridina-1(1,2)-benzenacycloundecaphane-6-yl)propanoic acid (81) was synthesized using the procedure described in Example 62 except 6-(3-hydroxypropyl)-2³-(trifluoromethyl)-4-thia-3,6-diaza-2,5(2,6)-dipyridina-1(1,2)-benzenacyclotridecaphane 4,4-dioxide (39) was replaced with 15-fluoro-6-(3-hydroxypropyl)-2³-(trifluoromethyl)-11-oxa-4-thia-3,6-diaza-2,5(2,6)-dipyridina-1(1,2)-benzenacycloundecaphane 4,4-dioxide (22). LC-MS (Condition 1): m/z 555.1 [M+1]⁺, 1.70 min. ¹H NMR (400 MHz, DMSO-d₆) δ 12.33 (s, 1H), 11.65 (s, 1H), 8.06 (d, J=9.0 Hz, 1H), 7.71 (dd, J=8.7, 7.2 Hz, 1H), 7.35-7.18 (m, 3H), 7.15-7.00 (m, 2H), 6.89 (d, J=8.8 Hz, 1H), 4.05-3.93 (m, 1H), 3.93-3.72 (m, 2H), 3.68-3.51 (m, 1H), 3.48-3.38 (m, 1H), 3.11-2.94 (m, 1H), 2.49-2.40 (m, 2H), 1.67 (d, J=6.2 Hz, 1H), 1.53-1.37 (m, 2H), 1.32-1.16 (m, 1H).

### Example 82: Synthesis of 6-(2³-chloro-4,4-dioxido-4-thia-3,6-diaza-2,5(2,6)-dipyridina-1(1,2)-benzenacycloundecaphane-6-yl)hexanoic acid (82)

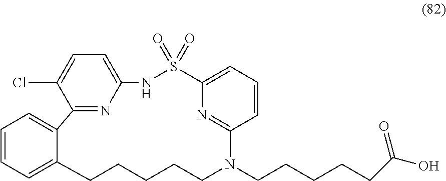 [SMILES: O=C(O)CCCCCN1CCCCCc2ccccc2-c2nc(ccc2Cl)NS(=O)(=O)c2cccc1n2]

6-(2³-chloro-4,4-dioxido-4-thia-3,6-diaza-2,5(2,6)-dipyridina-1(1,2)-benzenacycloundecaphane-6-yl)hexanoic acid (82) was synthesized using the procedure described in Example 62 except 6-(3-hydroxypropyl)-2³-(trifluoromethyl)-4-thia-3,6-diaza-2,5(2,6)-dipyridina-1(1,2)-benzenacyclotridecaphane 4,4-dioxide (39) was replaced with 23-chloro-6-(6-hydroxyhexyl)-4-thia-3,6-diaza-2,5(2,6)-dipyridina-1(1,2)-benzenacycloundecaphane 4,4-dioxide (51). LCMS (Condition 1): m/z 543.2 [M+H]⁺, 1.92 min. ¹H NMR (400 MHz, DMSO-d₆) δ 12.00 (s, 1H), 11.29 (s, 1H), 7.86 (d, J=8.9 Hz, 1H), 7.68 (dd, J=8.7, 7.2 Hz, 1H), 7.35 (td, J=7.4, 1.5 Hz, 1H), 7.27 (m, 2H), 7.17 (m, 3H), 6.78 (d, J=8.8 Hz, 1H), 3.76 (s, 1H), 3.23 (t, J=7.4 Hz, 2H), 2.96 (s, 1H), 2.52 (m, 1H), 2.19 (t, J=7.3 Hz, 2H), 2.08 (m, 1H), 0.85-1.65 (m, 12H).

### Example 83: Synthesis of 4-(2³-methoxy-4,4-dioxido-4-thia-3,6-diaza-2,5(2,6)-dipyridina-1(1,2)-benzenacycloundecaphane-6-yl)butanoic acid (83)

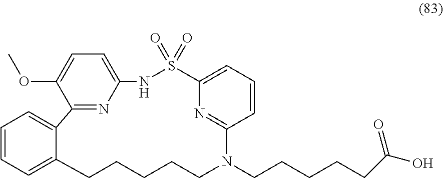 [SMILES: COc1ccc2nc1-c1ccccc1CCCCCN(CCCCCC(=O)O)c1cccc(n1)S(=O)(=O)N2]

4-(2³-methoxy-4,4-dioxido-4-thia-3,6-diaza-2,5(2,6)-dipyridina-1(1,2)-benzenacycloundecaphane-6-yl)butanoic acid (83) was synthesized using the procedure described in Example 62 except 6-(3-hydroxypropyl)-2³-(trifluoromethyl)-4-thia-3,6-diaza-2,5(2,6)-dipyridina-1(1,2)-benzenacyclotridecaphane 4,4-dioxide (39) was replaced with 6-(4-hydroxybutyl)-2³-methoxy-4-thia-3,6-diaza-2,5(2,6)-dipyridina-1(1,2)-benzenacycloundecaphane 4,4-dioxide (17). LC-MS (Condition 1): m/z 511.2 [M+1]⁺, 1.62 min. ¹H NMR (400 MHz, Methanol-d₄) δ 7.99 (s, 1H), 7.7.70-7.58 (m, 1H), 7.39 (d, J=9.0 Hz, 1H), 7.29 (ddd, J=7.7, 7.0, 1.5 Hz, 1H), 7.26-7.21 (m, 2H), 7.19 (dd, J=7.1, 1.5 Hz, 1H), 7.13 (dd, J=7.5, 1.5 Hz, 1H), 6.87-6.79 (m, 1H), 3.69 (s, 3H), 3.51-3.38 (m, 2H), 2.41 (t, J=7.3 Hz, 2H), 2.33 (t, J=6.9 Hz, 2H), 1.77-1.73 (m, 2H), 1.47-1.42 (m, 3H), 1.32-1.18 (m, 2H), 1.12-1.00 (m, 2H), 0.94-0.83 (m, 1H).

### Example 84: Synthesis of 3-(4,4-dioxido-2³-(trifluoromethyl)-4-thia-3,6-diaza-2,5(2,6)-dipyridina-1(1.2)-benzenacyclododecaphane-6-yl)propanoic acid (84)

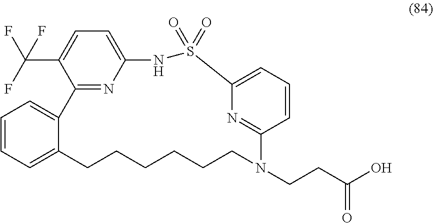 [SMILES: O=C(O)CCN1CCCCCCc2ccccc2-c2nc(ccc2C(F)(F)F)NS(=O)(=O)c2cccc1n2]

3-(4,4-dioxido-2³-(trifluoromethyl)-4-thia-3,6-diaza-2,5(2,6)-dipyridina-1(1,2)-benzenacyclododecaphane-6-yl)propanoic acid (84) was synthesized using the procedure described in Example 62 except 6-(3-hydroxypropyl)-2³-(trifluoromethyl)-4-thia-3,6-diaza-2,5(2,6)-dipyridina-1(1,2)-benzenacyclotridecaphane 4,4-dioxide (39) was replaced with 6-(3-hydroxypropyl)-2³-(trifluoromethyl)-4-thia-3,6-diaza-2,5(2,6)-dipyridina-1(1,2)-benzenacyclododecaphane 4,4-dioxide (21). LCMS (Condition 1): m/z 549.2 [M+H]⁺, 1.55 min. ¹H NMR (400 MHz, DMSO-d₆) δ 12.31 (s, 1H), 11.68 (s, 1H), 8.16 (d, J=9.0 Hz, 1H), 7.71 (dd, J=8.7, 7.2 Hz, 1H), 7.44 (m, 1H), 7.34 (td, J=7.5, 1.5 Hz, 1H), 7.21 (m, 4H), 6.92 (d, J=8.8 Hz, 1H), 3.53 (m, 3H), 3.38 (m, 1H), 3.33 (m, 1H), 2.47 (m, 2H), 2.01 (m, 1H), 1.35 (m, 3H), 1.09 (m, 3H), 0.80 (m, 2H). ¹⁹F NMR (376 MHz, DMSO-d₆) δ−55.94 (s).

### Example 85: Synthesis of 3-(1⁵-fluoro-4,4-dioxido-2³-(trifluoromethyl)-4-thia-3,6-diaza-2,5(2,6)-dipyridina-1(1,2)-benzenacyclododecaphane-6-yl)propanoic acid (85)

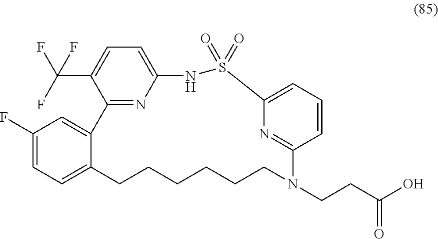 [SMILES: O=C(O)CCN1CCCCCCc2ccc(F)cc2-c2nc(ccc2C(F)(F)F)NS(=O)(=O)c2cccc1n2]

3-(1⁵-fluoro-4,4-dioxido-2³-(trifluoromethyl)-4-thia-3,6-diaza-2,5(2,6)-dipyridina-1(1,2)-benzenacyclododecaphane-6-yl)propanoic acid (85) was synthesized using the procedure described in Example 62 except 6-(3-hydroxypropyl)-2³-(trifluoromethyl)-4-thia-3,6-diaza-2,5(2,6)-dipyridina-1(1,2)-benzenacyclotridecaphane 4,4-dioxide (39) was replaced with 15-fluoro-6-(3-hydroxypropyl)-²³-(trifluoromethyl)-4-thia-3,6-diaza-2,5(2,6)-dipyridina-1(1,2)-benzenacyclododecaphane 4,4-dioxide (52). LCMS (Condition 1): m/z 567.1 [M+H]⁺, 1.40 min. ¹H NMR (400 MHz, Methanol-d₄) δ 8.05 (d, J=8.9 Hz, 1H), 7.67 (dd, J=8.7, 7.3 Hz, 1H), 7.58 (d, J=8.9 Hz, 1H), 7.32-7.21 (m, 2H), 7.12-7.01 (m, 1H), 6.97-6.79 (m, 2H), 3.89-3.74 (m, 1H), 3.73-3.51 (m, 2H), 3.46-3.33 (m, 1H), 2.61-2.55 (m, 2H), 2.50 (s, 1H), 2.16-2.02 (m, 1H), 1.52-1.35 (m, 3H), 1.25-1.10 (m, 3H), 1.02-0.92 (m, 2H).

### Example 86: Synthesis of 4-(1⁵-fluoro-4,4-dioxido-2³-(trifluoromethyl)-4-thia-3,6-diaza-2.5(2.6)-dipyridina-1(1.2)-benzenacycloundecaphane-6-yl)butanoic acid (86)

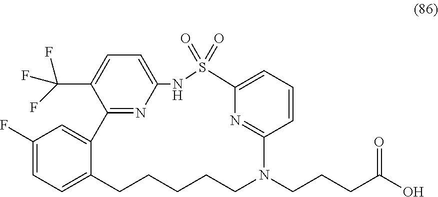 [SMILES: O=C(O)CCCN1CCCCCc2ccc(F)cc2-c2nc(ccc2C(F)(F)F)NS(=O)(=O)c2cccc1n2]

4-(1⁵-fluoro-4,4-dioxido-2³-(trifluoromethyl)-4-thia-3,6-diaza-2,5(2,6)-dipyridina-1(1,2)-benzenacycloundecaphane-6-yl)butanoic acid (86) was synthesized using the procedure described in Example 62 except 6-(3-hydroxypropyl)-2³-(trifluoromethyl)-4-thia-3,6-diaza-2,5(2,6)-dipyridina-1(1,2)-benzenacyclotridecaphane 4,4-dioxide (39 was replaced with 15-fluoro-6-(4-hydroxybutyl)-2³-(trifluoromethyl)-4-thia-3,6-diaza-2,5(2,6)-dipyridina-1(1,2)-benzenacycloundecaphane 4,4-dioxide (53). LCMS (Condition 1): m/z 567.2 [M+H]⁺, 1.79 min. ¹H NMR (400 MHz, DMSO-d₆) δ 12.14 (s, 1H), 11.84 (s, 1H), 8.12 (d, J=9.0 Hz, 1H), 7.73 (dd, J=8.7, 7.2 Hz, 1H), 7.33 (dd, J=8.6, 5.8 Hz, 1H), 7.27-7.16 (m, 3H), 7.06 (dd, J=9.4, 2.7 Hz, 1H), 6.91 (d, J=8.8 Hz, 1H), 4.01-3.90 (m, 1H), 3.29-3.16 (m, 2H), 2.87 (t, J=9.2 Hz, 1H), 2.42-2.34 (m, 1H), 2.26 (t, J=7.0 Hz, 2H), 1.92-1.76 (m, 1H), 1.75-1.55 (m, 3H), 1.42-1.25 (m, 2H), 1.19-1.06 (m, 2H), 1.01 (d, J=6.1 Hz, 1H).

### Example 87: Synthesis of 3-(4,4-dioxido-2³-(trifluoromethyl)-4-thia-3,6-diaza-2,5(2,6)-dipyridina-1(1,2)-benzenacyclodecaphane-6-yl)propanoic acid (87)

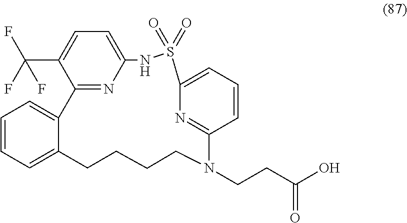 [SMILES: O=C(O)CCN1CCCCc2ccccc2-c2nc(ccc2C(F)(F)F)NS(=O)(=O)c2cccc1n2]

3-(4,4-dioxido-2³-(trifluoromethyl)-4-thia-3,6-diaza-2,5(2,6)-dipyridina-1(1,2)-benzenacyclodecaphane-6-yl)propanoic acid (87) was synthesized using the procedure described in Example 62 except 6-(3-hydroxypropyl)-2³-(trifluoromethyl)-4-thia-3,6-diaza-2,5(2,6)-dipyridina-1(1,2)-benzenacyclotridecaphane 4,4-dioxide (39) was replaced with 6-(3-hydroxypropyl)-²³-(trifluoromethyl)-4-thia-3,6-diaza-2,5(2,6)-dipyridina-1(1,2)-benzenacyclodecaphane 4,4-dioxide (20). LC-MS (Condition 1): m/z 521.2 [M+1]⁺, 1.74 min. ¹H NMR (400 MHz, DMSO-d₆) δ 12.31 (s, 1H), 11.68 (s, 1H), 8.14 (d, J=8.8 Hz, 1H), 7.73 (dd, J=8.7, 7.3 Hz, 1H), 7.39-7.30 (m, 2H), 7.29-7.15 (m, 4H), 6.86 (d, J=8.7 Hz, 1H), 4.00-3.78 (m, 1H), 3.62-3.41 (m, 2H), 3.02-2.85 (m, 1H), 2.49-2.41 (m, 2H), 1.81-1.65 (m, 1H), 1.68-1.52 (m, 1H), 1.41-1.11 (m, 3H), 0.79-0.59 (m, 1H).

### Example 88: Synthesis of 4-(4,4-dioxido-2³-(trifluoromethyl)-9-oxa-4-thia-3,6-diaza-2,5(2,6)-dipyridina-1(1.2)-benzenacyclododecaphane-6-yl)butanoic acid (88)

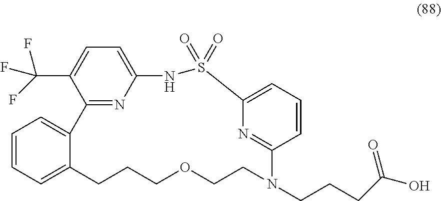 [SMILES: O=C(O)CCCN1CCOCCCc2ccccc2-c2nc(ccc2C(F)(F)F)NS(=O)(=O)c2cccc1n2]

4-(4,4-dioxido-2³-(trifluoromethyl)-9-oxa-4-thia-3,6-diaza-2,5(2,6)-dipyridina-1(1,2)-benzenacyclododecaphane-6-yl)butanoic acid (88) was synthesized using the procedure described in Example 62 except 6-(3-hydroxypropyl)-2³-(trifluoromethyl)-4-thia-3,6-diaza-2,5(2,6)-dipyridina-1(1,2)-benzenacyclotridecaphane 4,4-dioxide (39) was replaced with 6-(4-hydroxybutyl)-2³-(trifluoromethyl)-9-oxa-4-thia-3,6-diaza-2,5(2,6)-dipyridina-1(1,2)-benzenacyclododecaphane 4,4-dioxide (54). LCMS (Condition 1): m/z 565.2 [M+H]⁺, 1.93 min. ¹H NMR (400 MHz, DMSO-d₆) δ 11.98 (s, 1H), 11.51 (s, 1H), 8.13 (d, J=8.9 Hz, 1H), 7.60 (m, 1H), 7.35 (m, 2H), 7.20 (m, 2H), 7.08 (d, J=7.6 Hz, 1H), 7.03 (d, J=7.2 Hz, 1H), 6.95 (d, J=8.8 Hz, 1H), 3.75 (m, 1H), 3.02-3.53 (m, 7H), 2.18 (m, 2H), 1.95 (m, 2H), 1.61 (m, 3H), 1.34 (m, 1H). ¹⁹F NMR (376 MHz, DMSO-d₆) δ−56.68(s).

### Example 89: Synthesis of 3-(2³-chloro-4,4-dioxido-4-thia-3,6-diaza-2,5(2,6)-dipyridina-1(1,2)-benzenacycloundecaphane-6-yl)propanoic acid (89)

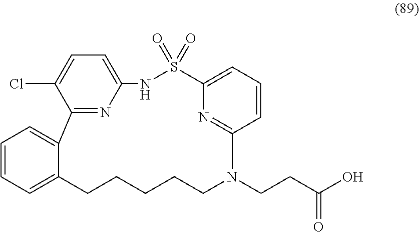 [SMILES: O=C(O)CCN1CCCCCc2ccccc2-c2nc(ccc2Cl)NS(=O)(=O)c2cccc1n2]

3-(2³-chloro-4,4-dioxido-4-thia-3,6-diaza-2,5(2,6)-dipyridina-1 (1,2)-benzenacycloundecaphane-6-yl)propanoic acid (89) was synthesized using the procedure described in Example 62 except 6-(3-hydroxypropyl)-2³-(trifluoromethyl)-4-thia-3,6-diaza-2,5(2,6)-dipyridina-1(1,2)-benzenacyclotridecaphane 4,4-dioxide (39) was replaced with 23-chloro-6-(3-hydroxypropyl)-4-thia-3,6-diaza-2,5(2,6)-dipyridina-1(1,2)-benzenacycloundecaphane 4,4-dioxide (24). LC-MS (Condition 1): m/z 501.1 [M+1]⁺, 1.72 min. ¹H NMR (400 MHz, DMSO-d₆) δ 12.33 (s, 1H), 11.31 (s, 1H), 7.85 (d, J=8.9 Hz, 1H), 7.71 (dd, J=8.7, 7.2 Hz, 1H), 7.38-7.31 (m, 1H), 7.31-7.22 (m, 2H), 7.22-7.13 (m, 3H), 6.85 (d, J=8.7 Hz, 1H), 3.87-3.63 (m, 1H), 3.50 (t, J=7.1 Hz, 2H), 3.12-2.91 (m, 1H), 2.61-2.51 (m, 1H), 2.44 (dt, J=7.1, 3.8 Hz, 2H), 2.16-2.01 (m, 1H), 1.69-1.41 (m, 1H), 1.37-0.88 (m, 5H).

### Example 90: Synthesis of 4-(4,4-dioxido-2³-(trifluoromethyl)-4-thia-3,6-diaza-2,5(2,6)-dipyridina-1(1.2)-benzenacyclodecaphane-6-yl)butanoic acid (90)

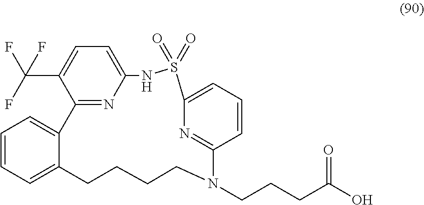 [SMILES: O=C(O)CCCN1CCCCc2ccccc2-c2nc(ccc2C(F)(F)F)NS(=O)(=O)c2cccc1n2]

4-(4,4-dioxido-2³-(trifluoromethyl)-4-thia-3,6-diaza-2,5(2,6)-dipyridina-1(1,2)-benzenacyclodecaphane-6-yl)butanoic acid (90) was synthesized using the procedure described in Example 62 except 6-(3-hydroxypropyl)-2³-(trifluoromethyl)-4-thia-3,6-diaza-2,5(2,6)-dipyridina-1(1,2)-benzenacyclotridecaphane 4,4-dioxide (39) was replaced with 6-(4-hydroxybutyl)-2³-(trifluoromethyl)-4-thia-3,6-diaza-2,5(2,6)-dipyridina-1(1,2)-benzenacyclodecaphane 4,4-dioxide (55). LCMS (Condition 1): m/z 535.1 [M+H]⁺, 1.78 min. ¹H NMR (400 MHz, DMSO-d₆) δ 12.10 (s, 1H), 11.68 (s, 1H), 8.15 (d, J=9.0 Hz, 1H), 7.72 (dd, J=8.6, 7.3 Hz, 1H), 7.35 (m, 2H), 7.26 (m, 3H), 7.17 (d, J=7.2 Hz, 1H), 6.89 (d, J=8.7 Hz, 1H), 3.90 (m, 1H), 3.34 (m, 2H), 2.87 (m, 1H), 2.54 (m, 1H), 2.25 (t, J=7.2 Hz, 2H), 1.70 (m, 3H), 1.27 (m, 3H), 0.68 (m, 1H). ¹⁹F NMR (376 MHz, DMSO-d₆) δ−57.42 (s).

### Example 91: Synthesis of 4-(2³-chloro-4,4-dioxido-4-thia-3,6-diaza-2,5(2,6)-dipyridina-1(1,2)-benzenacyclodecaphane-6-yl)butanoic acid (91)

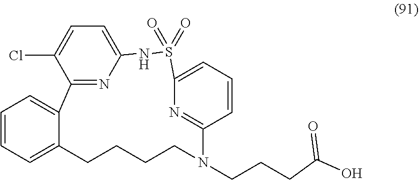 [SMILES: O=C(O)CCCN1CCCCc2ccccc2-c2nc(ccc2Cl)NS(=O)(=O)c2cccc1n2]

4-(2³-chloro-4,4-dioxido-4-thia-3,6-diaza-2,5(2,6)-dipyridina-1(1,2)-benzenacyclodecaphane-6-yl)butanoic acid (91) was synthesized using the procedure described in Example 62 except 6-(3-hydroxypropyl)-2³-(trifluoromethyl)-4-thia-3,6-diaza-2,5(2,6)-dipyridina-1(1,2)-benzenacyclotridecaphane 4,4-dioxide (39) was replaced with 2³-chloro-6-(4-hydroxybutyl)-4-thia-3,6-diaza-2,5(2,6)-dipyridina-1(1,2)-benzenacyclodecaphane 4,4-dioxide (56). LCMS (Condition 1): m/z 501.1 [M+H]⁺, 1.71 min. ¹H NMR (400 MHz, DMSO-d₆) δ 12.10 (s, 1H), 11.17 (s, 1H), 7.91 (d, J=8.8 Hz, 1H), 7.70 (dd, J=8.7, 7.3 Hz, 1H), 7.28 (m, 5H), 7.13 (d, J=7.2 Hz, 1H), 6.86 (d, J=8.7 Hz, 1H), 3.58 (m, 1H), 3.24 (m, 2H), 3.02 (m, 1H), 2.61 (m, 1H), 2.25 (t, J=7.1 Hz, 2H), 2.05 (m, 1H), 1.66 (p, J=7.3 Hz, 2H), 1.28 (m, 4H).

### Example 92: Synthesis of 3-(4,4-dioxido-2³-(trifluoromethyl)-4-thia-3,6-diaza-2,5(2,6)-dipyridina-1(1.2)-benzenacycloundecaphane-6-yl)Propanoic acid (92)

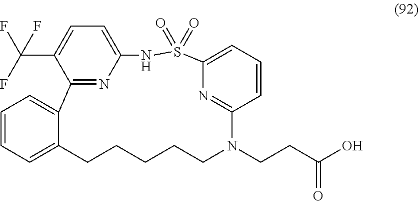 [SMILES: O=C(O)CCN1CCCCCc2ccccc2-c2nc(ccc2C(F)(F)F)NS(=O)(=O)c2cccc1n2]

3-(4,4-dioxido-2³-(trifluoromethyl)-4-thia-3,6-diaza-2,5(2,6)-dipyridina-1(1,2)-benzenacycloundecaphane-6-yl)propanoic acid (92) was synthesized using the procedure described in Example 62 except 6-(3-hydroxypropyl)-2³-(trifluoromethyl)-4-thia-3,6-diaza-2,5(2,6)-dipyridina-1(1,2)-benzenacyclotridecaphane 4,4-dioxide (39) was replaced with 6-(3-hydroxypropyl)-2³-(trifluoromethyl)-4-thia-3,6-diaza-2,5(2,6)-dipyridina-1(1,2)-benzenacycloundecaphane 4,4-dioxide (29). LC-MS (Condition 1): m/z 535.2 [M+1]⁺, 1.69 min. ¹H NMR (400 MHz, DMSO-d₆) δ 12.33 (s, 1H), 11.78 (s, 1H), 8.10 (d, J=9.0 Hz, 1H), 7.73 (dd, J=8.7, 7.2 Hz, 1H), 7.36 (td, J=7.5, 1.5 Hz, 1H), 7.31-7.17 (m, 4H), 7.14 (d, J=7.5 Hz, 1H), 6.88 (d, J=8.8 Hz, 1H), 4.05-3.82 (m, 1H), 3.62-3.42 (m, 2H), 3.00-2.85 (m, 1H), 2.48-2.28 (m, 2H), 1.96-1.82 (m, 1H), 1.78-1.60 (m, 1H), 1.44-1.21 (m, 3H), 1.21-1.08 (m, 2H), 1.08-0.95 (m, 1H).

### Example 93: Synthesis of 3-(4,4-dioxido-4-thia-3,6-diaza-2,5(2,6)-dipyridina-1(1,2)-benzenacycloundecaphane-6-yl)propanoic acid (93)

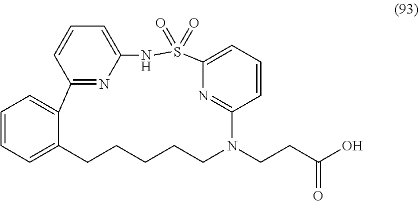 [SMILES: O=C(O)CCN1CCCCCc2ccccc2-c2cccc(n2)NS(=O)(=O)c2cccc1n2]

3-(4,4-dioxido-4-thia-3,6-diaza-2,5(2,6)-dipyridina-1(1,2)-benzenacycloundecaphane-6-yl)propanoic acid (93) was synthesized using the procedure described in Example 62 except 6-(3-hydroxypropyl)-2³-(trifluoromethyl)-4-thia-3,6-diaza-2,5(2,6)-dipyridina-1(1,2)-benzenacyclotridecaphane 4,4-dioxide (39) was replaced with 6-(3-hydroxypropyl)-4-thia-3,6-diaza-2,5(2,6)-dipyridina-1(1,2)-benzenacycloundecaphane 4,4-dioxide (57). LCMS (Condition 1): m/z 467.1 [M+H]⁺, 1.42 min, ¹H NMR (400 MHz, Methanol-d₄) δ 7.73-7.61 (m, 2H), 7.38-7.31 (m, 1H), 7.31-7.23 (m, 4H), 7.19 (dd, J=8.5, 0.8 Hz, 1H), 6.92 (d, J=7.4 Hz, 1H), 6.81 (d, J=8.7 Hz, 1H), 3.62-3.56 (m, 2H), 3.44 (t, J=7.2 Hz, 2H), 2.67 (dd, J=8.6, 6.7 Hz, 2H), 2.48-2.42 (m, 2H), 1.48 (dd, J=8.6, 6.5 Hz, 2H), 1.33 (d, J=7.2 Hz, 2H), 1.18-1.14 (m, 2H).

### Example 94: Synthesis of 1-((4,4-dioxido-2³-(trifluoromethyl)-4-thia-3,6-diaza-2,5(2,6)-dipyridina-1(1,2)-benzenacycloundecaphane-6-yl)methyl)cyclopropane-1-carboxylic acid (94)

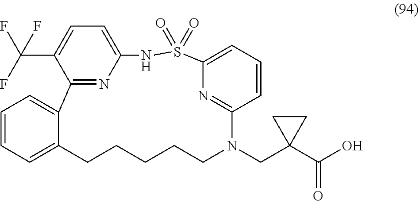 [SMILES: O=C(O)C1(CN2CCCCCc3ccccc3-c3nc(ccc3C(F)(F)F)NS(=O)(=O)c3cccc2n3)CC1]

1-((4,4-dioxido-2³-(trifluoromethyl)-4-thia-3,6-diaza-2,5(2,6)-dipyridina-1(1,2)-benzenacycloundecaphane-6-yl)methyl)cyclopropane-1-carboxylic acid (94) was synthesized using the procedure described in Example 62 except 6-(3-hydroxypropyl)-2³-(trifluoromethyl)-4-thia-3,6-diaza-2,5(2,6)-dipyridina-1(1,2)-benzenacyclotridecaphane 4,4-dioxide (39) was replaced with 6-((1-(hydroxymethyl)cyclopropyl)methyl)-2³-(trifluoromethyl)-4-thia-3,6-diaza-2,5(2,6)-dipyridina-1(1,2)-benzenacycloundecaphane 4,4-dioxide (58). LCMS (Condition 1): m/z 561.3 [M+H]⁺, 1.73 min. ¹H NMR (400 MHz, DMSO-d₆) δ 12.36 (s, 1H), 11.78 (s, 1H), 8.10 (d, J=8.9 Hz, 1H), 7.71 (dd, J=8.7, 7.2 Hz, 1H), 7.36 (td, J=7.4, 1.5 Hz, 1H), 7.27 (m, 2H), 7.22 (td, J=7.4, 1.4 Hz, 1H), 7.18 (d, J=8.9 Hz, 1H), 7.13 (d, J=7.6 Hz, 1H), 6.96 (d, J=8.8 Hz, 1H), 3.83 (m, 1H), 3.74 (d, J=15.4 Hz, 1H), 3.62 (d, J=15.4 Hz, 1H), 2.96 (m, 1H), 2.37 (m, 1H), 1.88 (m, 1H), 1.73 (m, 1H), 1.39 (m, 1H), 1.25 (m, 1H), 1.07 (m, 5H), 0.77 (m, 2H). ¹⁹F NMR (376 MHz, DMSO-d₆) δ−56.75 (s).

### Example 95: Synthesis of 1-((4,4-dioxido-2³-(trifluoromethyl)-9-oxa-4-thia-3,6-diaza-2,5(2,6)-dipyridina-1(1,2)-benzenacyclododecaphane-6-yl)methyl)cyclopropane-1-carboxylic acid (95)

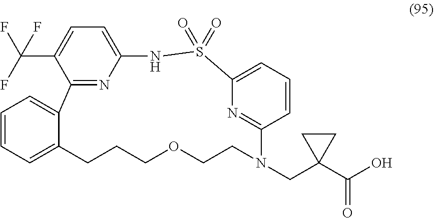 [SMILES: O=C(O)C1(CN2CCOCCCc3ccccc3-c3nc(ccc3C(F)(F)F)NS(=O)(=O)c3cccc2n3)CC1]

1-((4,4-dioxido-2³-(trifluoromethyl)-9-oxa-4-thia-3,6-diaza-2,5(2,6)-dipyridina-1(1,2)-benzenacyclododecaphane-6-yl)methyl)cyclopropane-1-carboxylic acid (95) was synthesized using the procedure described in Example 62 except 6-(3-hydroxypropyl)-2³-(trifluoromethyl)-4-thia-3,6-diaza-2,5(2,6)-dipyridina-1(1,2)-benzenacyclotridecaphane 4,4-dioxide (39) was replaced with 6-((1-(hydroxymethyl)cyclopropyl)methyl)-2³-(trifluoromethyl)-9-oxa-4-thia-3,6-diaza-2,5(2,6)-dipyridina-1(1,2)-benzenacyclododecaphane 4,4-dioxide (59). LCMS (Condition 1): m/z 577.3 [M+H]⁺, 1.67 min. ¹H NMR (400 MHz, DMSO-d₆) δ 12.27 (s, 1H), 11.51 (s, 1H), 8.16 (d, J=8.8 Hz, 1H), 7.55 (m, 1H), 7.32 (m, 2H), 7.19 (m, 2H), 7.03 (m, 3H), 3.90 (m, 1H), 3.74 (m, 2H), 3.53 (m, 2H), 3.34 (m, 2H), 3.02 (m, 1H), 1.82 (m, 3H), 1.32 (m, 1H), 1.15 (m, 1H), 1.03 (m, 1H), 0.90 (m, 1H), 0.67 (m, 1H). ¹⁹F NMR (376 MHz, DMSO-d₆) δ−56.77 (s).

### Example 96: Synthesis of 3-(1⁵-fluoro-4,4-dioxido-2³-(trifluoromethyl)-4-thia-3,6-diaza-2,5(2,6)-dipyridina-1(1,2)-benzenacycloundecaphane-6-yl)-2,2-dimethylpropanoic acid (96)

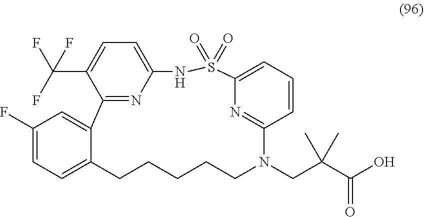 [SMILES: CC(C)(CN1CCCCCc2ccc(F)cc2-c2nc(ccc2C(F)(F)F)NS(=O)(=O)c2cccc1n2)C(=O)O]

3-(1⁵-fluoro-4,4-dioxido-2³-(trifluoromethyl)-4-thia-3,6-diaza-2,5(2,6)-dipyridina-1(1,2)-benzenacycloundecaphane-6-yl)-2,2-dimethylpropanoic acid (96) was synthesized using the procedure described in Example 62 except 6-(3-hydroxypropyl)-2³-(trifluoromethyl)-4-thia-3,6-diaza-2,5(2,6)-dipyridina-1(1,2)-benzenacyclotridecaphane 4,4-dioxide (39) was replaced with 1⁵-fluoro-6-(3-hydroxy-2,2-dimethylpropyl)-2³-(trifluoromethyl)-4-thia-3,6-diaza-2,5(2,6)-dipyridina-1(1,2)-benzenacycloundecaphane 4,4-dioxide (60). LCMS (Condition 1): m/z 581.2 [M+H]⁺, 1.78 min, ¹H NMR (400 MHz, Methanol-d₄) δ 7.96 (d, J=8.9 Hz, 1H), 7.64 (dd, J=8.8, 7.2 Hz, 1H), 7.37-7.22 (m, 3H), 7.11-7.04 (m, 1H), 7.00 (d, J=8.8 Hz, 1H), 6.87 (dd, J=9.4, 2.8 Hz, 1H), 4.06-3.94 (m, 1H), 3.68-3.48 (m, 2H), 3.02-2.90 (m, 1H), 2.44-2.30 (m, 1H), 2.05-1.90 (m, 1H), 1.86-1.72 (m, 1H), 1.55-1.42 (m, 2H), 1.35-1.25 (m, 2H), 1.20-1.10 (m, 7H).

### Example 97: Synthesis of 3-(1⁵-fluoro-4,4-dioxido-2³-(trifluoromethyl)-4-thia-3,6-diaza-2,5(2,6)-dipyridina-1(1,2)-benzenacycloundecaphane-6-yl)propanoic acid (97)

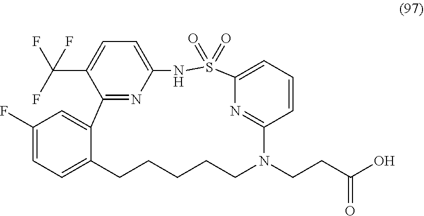 [SMILES: O=C(O)CCN1CCCCCc2ccc(F)cc2-c2nc(ccc2C(F)(F)F)NS(=O)(=O)c2cccc1n2]

3-(1⁵-fluoro-4,4-dioxido-2³-(trifluoromethyl)-4-thia-3,6-diaza-2,5(2,6)-dipyridina-1(1,2)-benzenacycloundecaphane-6-yl)propanoic acid (97) was synthesized using the procedure described in Example 62 except 6-(3-hydroxypropyl)-2³-(trifluoromethyl)-4-thia-3,6-diaza-2,5(2,6)-dipyridina-1(1,2)-benzenacyclotridecaphane 4,4-dioxide (39) was replaced with 15-fluoro-6-(3-hydroxypropyl)-²³-(trifluoromethyl)-4-thia-3,6-diaza-2,5(2,6)-dipyridina-1(1,2)-benzenacycloundecaphane 4,4-dioxide (26). LCMS (Condition 1): m/z 553.1 [M+H]⁺, 1.79 min, ¹H NMR (400 MHz, Methanol-d₄) δ 7.97 (d, J=8.9 Hz, 1H), 7.69 (dd, J=8.7, 7.3 Hz, 1H), 7.38-7.31 (m, 2H), 7.27 (dd, J=8.6, 5.6 Hz, 1H), 7.13-7.05 (m, 1H), 6.91-6.79 (m, 2H), 4.22-4.08 (m, 1H), 3.63 (t, J=7.2 Hz, 2H), 3.02-2.91 (m, 1H), 2.60-2.47 (m, 2H), 2.49-2.34 (m, 1H), 2.03-1.89 (m, 1H), 1.82-1.66 (m, 1H), 1.53-1.39 (m, 2H), 1.26-1.15 (m, 2H), 1.17-1.02 (m, 1H).

### Example 98: Synthesis of 3-(4,4-dioxido-2³-(trifluoromethyl)-9-oxa-4-thia-3,6-diaza-2,5(2,6)-dipyridina-1(1,2)-benzenacyclododecaphane-6-yl)propanoic acid (98)

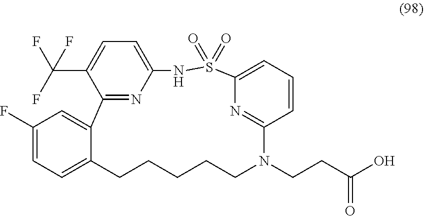 [SMILES: O=C(O)CCN1CCCCCc2ccc(F)cc2-c2nc(ccc2C(F)(F)F)NS(=O)(=O)c2cccc1n2]

3-(4,4-dioxido-2³-(trifluoromethyl)-9-oxa-4-thia-3,6-diaza-2,5(2,6)-dipyridina-1(1,2)-benzenacyclododecaphane-6-yl)propanoic acid (98) was synthesized using the procedure described in Example 62 except 6-(3-hydroxypropyl)-2³-(trifluoromethyl)-4-thia-3,6-diaza-2,5(2,6)-dipyridina-1(1,2)-benzenacyclotridecaphane 4,4-dioxide (39) was replaced with 6-(3-hydroxypropyl)-2³-(trifluoromethyl)-9-oxa-4-thia-3,6-diaza-2,5(2,6)-dipyridina-1(1,2)-benzenacyclododecaphane 4,4-dioxide (30). LCMS (Condition 1): m/z 551.2 [M+H]⁺, 1.59 min. ¹H NMR (400 MHz, DMSO-d₆) δ 12.17 (s, 1H), 11.54 (s, 1H), 8.13 (d, J=8.9 Hz, 1H), 7.61 (m, 1H), 7.41 (m, 1H), 7.33 (m, 1H), 7.21 (m, 2H), 7.06 (m, 2H), 6.97 (d, J=8.7 Hz, 1H), 3.82 (m, 1H), 3.00-3.60 (m, 7H), 2.44 (m, 1H), 2.31 (m, 1H), 1.91 (m, 2H), 1.66 (m, 1H), 1.36 (m, 1H). ¹⁹F NMR (376 MHz, DMSO-d₆) δ−56.69 (s).

### Example 99: Synthesis of 3-(4,4-dioxido-2³-(trifluoromethyl)-4-thia-3,6-diaza-2,5(2,6)-dipyridina-1(1,2)-benzenacyclododecaphane-6-yl)-2,2-dimethylpropanoic acid (99)

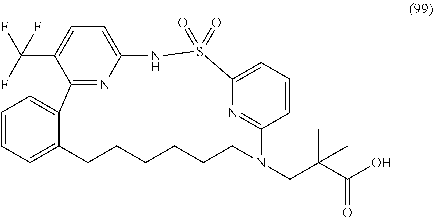 [SMILES: CC(C)(CN1CCCCCCc2ccccc2-c2nc(ccc2C(F)(F)F)NS(=O)(=O)c2cccc1n2)C(=O)O]

3-(4,4-dioxido-2³-(trifluoromethyl)-4-thia-3,6-diaza-2,5(2,6)-dipyridina-1(1,2)-benzenacyclododecaphane-6-yl)-2,2-dimethylpropanoic acid (99) was synthesized using the procedure described in Example 62 except 6-(3-hydroxypropyl)-2³-(trifluoromethyl)-4-thia-3,6-diaza-2,5(2,6)-dipyridina-1(1,2)-benzenacyclotridecaphane 4,4-dioxide (39) was replaced with 6-(3-hydroxy-2,2-dimethylpropyl)-2³-(trifluoromethyl)-4-thia-3,6-diaza-2,5(2,6)-dipyridina-1(1,2)-benzenacyclododecaphane 4,4-dioxide (28). LC-MS (Condition 1): m/z 577.25 [M+1]⁺, 1.81 min. ¹H NMR (400 MHz, DMSO-d₆) δ 12.44 (s, 1H), 11.59 (s, 1H), 8.18 (d, J=8.9 Hz, 1H), 7.67 (dd, J=8.7, 7.2 Hz, 1H), 7.63-7.44 (m, 1H), 7.34 (td, J=7.5, 1.5 Hz, 1H), 7.28-7.19 (m, 2H), 7.18-7.10 (m, 2H), 7.05 (d, J=8.8 Hz, 1H), 3.79-3.63 (m, 1H), 3.62-3.48 (m, 2H), 3.45-3.34 (m, 2H), 2.40-2.23 (m, 1H), 2.10-1.93 (m, 1H), 1.41-1.27 (m, 2H), 1.27-1.19 (m, 1H), 1.19-1.11 (m, 2H), 1.10 (s, 3H), 1.07 (s, 3H), 0.89-0.76 (m, 1H), 0.74-0.60 (m, 1H).

### Example 100: Synthesis of 3-(4,4-dioxido-2³-(trifluoromethyl)-4-thia-3,6-diaza-2,5(2,6)-dipyridina-1(1,2)-benzenacycloundecaphane-6-yl)-2,2-dimethylpropanoic acid (100)

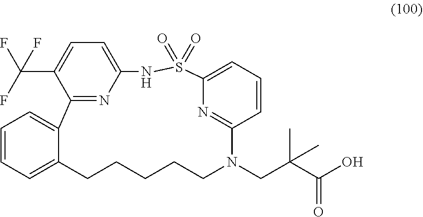 [SMILES: CC(C)(CN1CCCCCc2ccccc2-c2nc(ccc2C(F)(F)F)NS(=O)(=O)c2cccc1n2)C(=O)O]

3-(4,4-dioxido-2³-(trifluoromethyl)-4-thia-3,6-diaza-2,5(2,6)-dipyridina-1(1,2)-benzenacycloundecaphane-6-yl)-2,2-dimethylpropanoic acid (100) was synthesized using the procedure described in Example 62 except 6-(3-hydroxypropyl)-2³-(trifluoromethyl)-4-thia-3,6-diaza-2,5(2,6)-dipyridina-1(1,2)-benzenacyclotridecaphane 4,4-dioxide (39) was replaced with 6-(3-hydroxy-2,2-dimethylpropyl)-2³-(trifluoromethyl)-4-thia-3,6-diaza-2,5(2,6)-dipyridina-1(1,2)-benzenacycloundecaphane 4,4-dioxide (27). LC-MS (Condition 1): m/z 563.3 [M+1]⁺, 1.78 min. ¹H NMR (400 MHz, DMSO-d₆) δ 12.48 (s, 1H), 11.78 (s, 1H), 8.09 (d, J=8.9 Hz, 1H), 7.69 (dd, J=8.8, 7.2 Hz, 1H), 7.35 (td, J=7.5, 1.5 Hz, 1H), 7.30-7.25 (m, 2H), 7.22 (td, J=7.4, 1.3 Hz, 1H), 7.18 (d, J=8.9 Hz, 1H), 7.13 (d, J=7.6 Hz, 1H), 7.08 (d, J=8.9 Hz, 1H), 3.94-3.73 (m, 1H), 3.58 (d, J=15.3 Hz, 1H), 3.45 (d, J=15.2 Hz, 1H), 3.38-3.34 (m, 1H), 2.93-2.80 (m, 1H), 2.39-2.28 (m, 1H), 1.93-1.82 (m, 1H), 1.81-1.69 (m, 1H), 1.42-1.20 (m, 2H), 1.19-1.11 (m, 1H), 1.10 (s, 3H), 1.07 (s, 3H), 1.05-0.90 (m, 1H).

### Example 101: Synthesis of 5-(4,4-dioxido-2³-(trifluoromethyl)-4-thia-3,6-diaza-2,5(2,6)-dipyridina-1(1,2)-benzenacyclotridecaphane-6-yl)pentanoic acid (101)

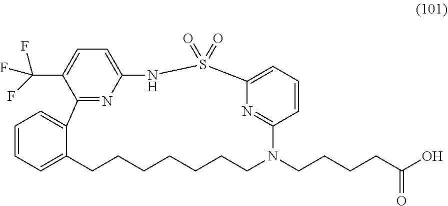 [SMILES: O=C(O)CCCCN1CCCCCCCc2ccccc2-c2nc(ccc2C(F)(F)F)NS(=O)(=O)c2cccc1n2]

5-(4,4-dioxido-2³-(trifluoromethyl)-4-thia-3,6-diaza-2,5(2,6)-dipyridina-1(1,2)-benzenacyclotridecaphane-6-yl)pentanoic acid (101) was synthesized using the procedure described in Example 62 except 6-(3-hydroxypropyl)-2³-(trifluoromethyl)-4-thia-3,6-diaza-2,5(2,6)-dipyridina-1(1,2)-benzenacyclotridecaphane 4,4-dioxide (39) was replaced with 6-(5-hydroxypentyl)-2³-(trifluoromethyl)-4-thia-3,6-diaza-2,5(2,6)-dipyridina-1(1,2)-benzenacyclotridecaphane 4,4-dioxide (41). LC-MS (Condition 1): m/z 591.2 [M+1]⁺, 1.93 min. ¹H NMR (400 MHz, DMSO-d₆) δ 12.04 (s, 1H), 11.73 (s, 1H), 8.19 (d, J=9.0 Hz, 1H), 7.71 (dd, J=8.6, 7.4 Hz, 1H), 7.46 (d, J=6.6 Hz, 1H), 7.35 (m, 1H), 7.25 (m, 2H), 7.17 (m, 2H), 6.87 (d, J=8.8 Hz, 1H), 3.61 (m, 1H), 3.47 (m, 1H), 3.30 (m, 1H), 2.37 (m, 1H), 2.25 (m, 2H), 2.08 (m, 1H), 1.52 (m, 4H), 1.36 (m, 2H), 1.15 (m, 7H), 0.80 (m, 2H). ¹⁹F NMR (376 MHz, DMSO-d₆) δ−56.70 (s).

### Example 102: Synthesis of 4-(4,4-dioxido-2³-(trifluoromethyl)-10-oxa-4-thia-3,6-diaza-2,5(2,6)-dipyridina-1(1,2)-benzenacycloundecaphane-6-yl)butanoic acid (102)

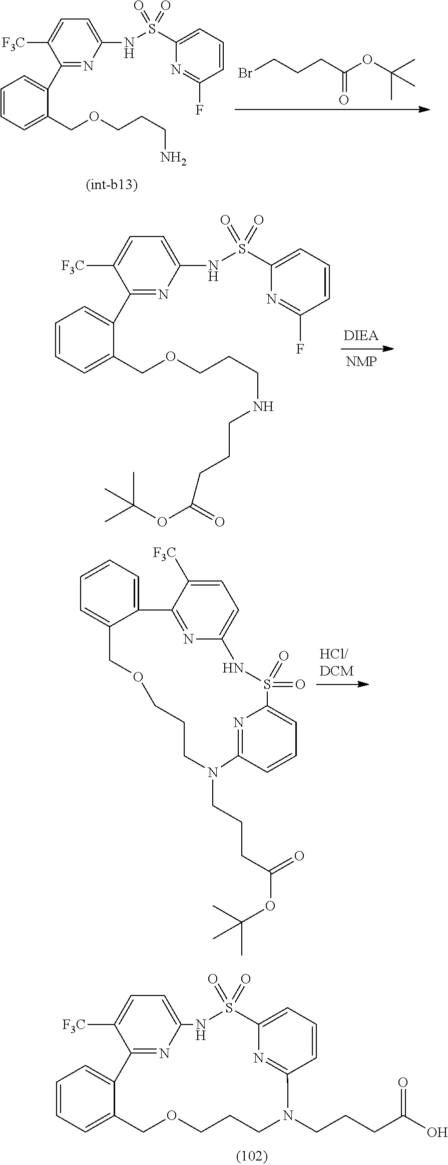

### Step 1. Synthesis of tert-butyl 4-((3-((2-(6-((6-fluoropyridine)-2-sulfonamido)-3-(trifluoromethyl)pyridin-2-yl)benzyl)oxy)propyl)amino)butanoate

N-(6-(2-((3-aminopropoxy)methyl)phenyl)-5-(trifluoromethyl)pyridin-2-yl)-6-fluoropyridine-2-sulfonamide (int-b13) (70 mg, 0.14 mmol) was dissolved in acetonitrile (2 mL), tert-butyl 4-bromobutanoate (36 mg, 0.16 mmol) and DIEA (50 μL, 0.29 mmol) was added and the reaction mixture was stirred at 55° C. for 16 h. It was then concentrated and purified by silica gel column chromatography (ISCO, EtOAc/heptane 0-100%) to give the title compound as white solid. (51 mg, 0.08 mmol, 56% yield). LC-MS (Condition 1): m/z 627.2 [M+H]⁺, 1.80 min.

### Step 2. Synthesis of tert-butyl 4-(4,4-dioxido-2³-(trifluoromethyl)-10-oxa-4-thia-3,6-diaza-2,5(2,6)-dipyridina-1(1,2)-benzenacycloundecaphane-6-yl)butanoate

tert-Butyl 4-((3-((2-(6-((6-fluoropyridine)-2-sulfonamido)-3-(trifluoromethyl)pyridin-2-yl)benzyl)oxy)propyl)amino)butanoate (50 mg, 0.08 mmol) dissolved in NMP (2 mL) in a vial and DIEA (0.05 mL, 0.29 mmol) was added. The vial was closed after flushing with N₂ and stirred at 135° C. for 16 h. The reaction was cooled to room temperature and was poured into water and extracted with EtOAc. The combined organic layers were dried over Na₂SO₄, filtered and concentrated. The residue was purified by flash column chromatography (ISCO, 12 g column, MeOH/DCM 0-10%, dry load) to give the title compound (36 mg, 0.06 mmol, 75% yield) as a white solid. LC-MS (Condition 1): m/z 607.1 [M+H]⁺, 1.72 min.

### Step 3. Synthesis of 4-(4,4-dioxido-2³-(trifluoromethyl)-10-oxa-4-thia-3,6-diaza-2,5(2,6)-dipyridina-1(1,2)-benzenacycloundecaphane-6-yl)butanoic acid (102)

tert-Butyl 4-(4,4-dioxido-2³-(trifluoromethyl)-10-oxa-4-thia-3,6-diaza-2,5(2,6)-dipyridina-1(1,2)-benzenacycloundecaphane-6-yl)butanoate (12 mg, 0.02 mmol) was suspended in DCM (1 mL) and HCl in dioxane (6M, 100 μL, 0.6 mmol) was added. Stirred at 65° C. for 16 h in a closed vial. The reaction was then concentrated and passed through a small silica gel column using DCM/MeOH as eluent to give the title compound as a white solid. LC-MS (Condition 1): m/z 551.2 [M+H]⁺, 1.78 min. ¹H NMR (400 MHz, Methanol-d₄) δ 8.06-7.86 (m, 2H), 7.68 (dd, J=8.7, 7.3 Hz, 1H), 7.56-7.33 (m, 2H), 7.29-7.15 (m, 2H), 6.90 (d, J=8.7 Hz, 1H), 6.59 (d, J=8.5 Hz, 1H), 4.45-4.42 (m, 4H), 4.18-3.76 (m, 3H), 3.02-3.00 (m, 1H), 2.45-2.19 (m, 2H), 1.82 (p, J=7.4 Hz, 1H), 1.63 (d, J=7.0 Hz, 1H), 1.50 (s, 2H).

### Example 103: Synthesis of 2³-(trifluoromethyl)-6,12-dioxa-4-thia-3-aza-1(3,2),2,5(2,6)-tripyridinacyclododecaphane 4,4-dioxide (103)

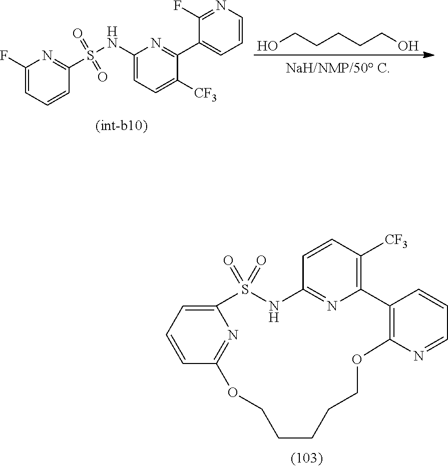

In a vial, 6-fluoro-N-(2′-fluoro-3-(trifluoromethyl)-[2,3′-bipyridin]-6-yl)pyridine-2-sulfonamide (int-b10) (100 mg, 0.24 mmol) and 1,5-pentanediol (25.0 mg, 0.24 mmol) were taken up in NMP (3 mL) and then sodium hydride (28.8 mg, 0.72 mol, 60%) was added and the reaction was heated to 50° C. overnight. The reaction was quenched with 1 M HCl and extracted into EtOAc (3×). The organics were then washed with water and brine, dried over MgSO₄, and concentrated in vacuo. The crude material was purified by flash column chromatography (ISCO, 4 g RediSep Rf Gold column, EtOAc/heptane 0-60%) to give the product 23-(trifluoromethyl)-6,12-dioxa-4-thia-3-aza-1(3,2),2,5(2,6)-tripyridinacyclododecaphane 4,4-dioxide (103) as a white solid. LCMS (Condition 1): m/z 481.1 [M+H]⁺, 1.76 min. ¹H NMR (400 MHz, DMSO-d₆) δ 11.69 (s, 1H), 8.22 (dd, J=5.3, 2.0 Hz, 2H), 8.02-7.89 (m, 1H), 7.76 (d, J=7.7 Hz, 1H), 7.69 (d, J=6.4 Hz, 1H), 7.63-7.52 (m, 1H), 7.06 (dd, J=7.3, 5.0 Hz, 2H), 4.30-4.25 (m, 1H), 4.11 (s, 2H), 3.95 (s, 1H), 1.51 (s, 2H), 1.42-1.21 (m, 4H).

### Example 104: Synthesis of (4¹s,4⁵s)-1³-(trifluoromethyl)-3,5-dioxa-7-thia-8-aza-1,6(2,6),2(3,2)-tripyridina-4(1,5)-cyclooctanacyclooctaphane 7,7-dioxide (104)

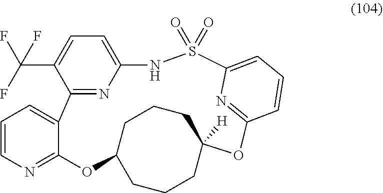 [SMILES: O=S1(=O)Nc2ccc(C(F)(F)F)c(n2)-c2cccnc2O[C@H]2CCC[C@H](CCC2)Oc2cccc1n2]

(4¹s,4⁵s)-1³-(trifluoromethyl)-3,5-dioxa-7-thia-8-aza-1,6(2,6),2(3,2)-tripyridina-4(1,5)-cyclooctanacyclooctaphane 7,7-dioxide (104) was synthesized using the procedure described in Example 103 except 1,5-pentanediol was replaced with cyclooctane-1,5-diol. LCMS (Condition 1): m/z 521.1 [M+H]⁺, 1.80 min. ¹H NMR (400 MHz, DMSO-d₆) δ 11.74 (s, 1H), 8.19 (s, 2H), 7.83 (s, 1H), 7.53-7.48 (m, 3H), 7.02-6.91 (m, 2H), 5.12-4.98 (m, 2H), 1.85-1.03 (m, 12H).

### Example 105: Synthesis of 2³-chloro-4-thia-3,6,12-triaza-1(3,2),2,5(2,6)-tripyridinacyclododecaphane 4,4-dioxide (105)

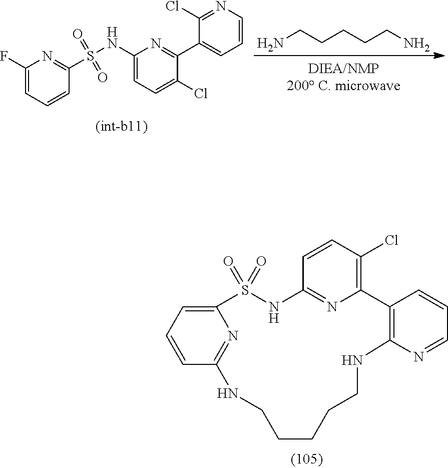

In a microwave vial, N-(2′,3-dichloro-[2,3′-bipyridin]-6-yl)-6-fluoropyridine-2-sulfonamide (int-b11) (100 mg, 0.25 mmol) and pentane-1,5-diamine (25.6 mg, 0.25 mmol) were taken up in NMP (3 mL) and then DIEA (0.13 mL, 0.75 mmol) was added and the reaction was heated to 200° C. for 1 h. The reaction was filtered and purified by mass-triggered preparatory HPLC (20-40% MeCN/H₂O+TFA, 100 mL/min). Fractions were concentrated by Genevac, residue taken up in MeOH, 1 M HCl in iPrOH was added, and concentrated in vacuo to give the product 2³-chloro-4-thia-3,6,12-triaza-1(3,2),2,5(2,6)-tripyridinacyclododecaphane 4,4-dioxide (105) as a tan solid. LCMS (Condition 1): m/z 445.1 [M+H]⁺, 1.25 min. ¹H NMR (400 MHz, DMSO-d₆) δ 11.39 (s, 1H), 8.10-7.82 (m, 3H), 7.52-7.44 (m, 1H), 7.39 (s, 1H), 7.19-6.95 (m, 3H), 6.88 (s, 1H), 6.58 (d, J=8.5 Hz, 1H), 3.39 (s, 4H), 1.61 (s, 2H), 1.41 (s, 2H), 1.24-1.22 (m, 2H).

### Example 106: Synthesis of 2³-chloro-1⁴-methyl-4-thia-3,6,12-triaza-1(3,2),2,5(2,6)-tripyridinacyclododecaphane 4,4-dioxide (106)

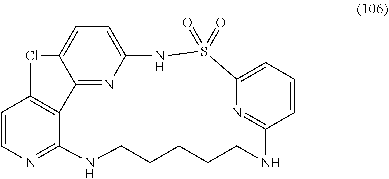

2³-chloro-1⁴-methyl-4-thia-3,6,12-triaza-1(3,2),2,5(2,6)-tripyridinacyclododecaphane 4,4-dioxide (106) was synthesized using the procedure described in Example 106 except N-(2′,3-dichloro-[2,3′-bipyridin]-6-yl)-6-fluoropyridine-2-sulfonamide (int-b11) was replaced with N-(2′,3-dichloro-4′-methyl-[2,3′-bipyridin]-6-yl)-6-fluoropyridine-2-sulfonamide (int-b12). LCMS (Condition 1): m/z 459.1 [M+H]⁺, 1.44 min. ¹H NMR (400 MHz, Methanol-d₄) δ 7.86 (d, J=5.4 Hz, 1H), 7.81 (d, J=8.9 Hz, 1H), 7.53 (d, J=8.9 Hz, 1H), 7.47 (dd, J=8.5, 7.1 Hz, 1H), 7.17 (dd, J=7.2, 0.7 Hz, 1H), 6.57 (dd, J=8.5, 0.8 Hz, 1H), 6.53 (d, J=5.4 Hz, 1H), 3.84 (s, 1H), 3.58 (dd, J=11.7, 7.3 Hz, 1H), 3.12-3.02 (m, 1H), 2.99-2.93 (m, 1H), 1.95 (s, 3H), 1.65-1.63 (m, 2H), 1.47-1.37 (m, 2H), 1.29-1.18 (m, 2H).

### Example 107: Synthesis of 2³-chloro-12-oxa-4-thia-3,6-diaza-1(3,2),2,5(2,6)-tripyridinacyclododecaphane 4,4-dioxide (107)

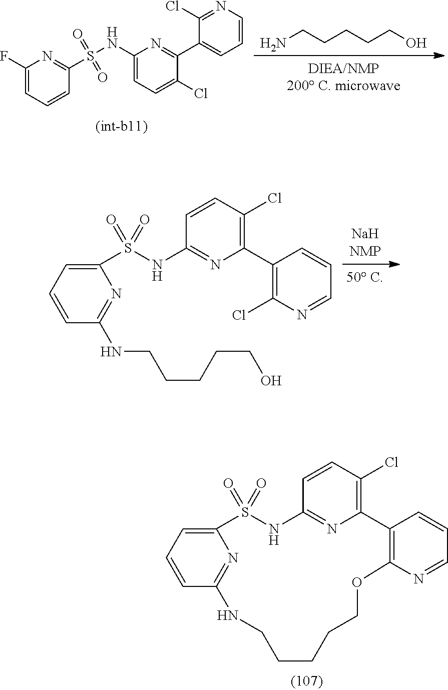

### Step 1. Synthesis of N-(2′,3-dichloro-[2,3′-bipyridin]-6-yl)-6-((5-hydroxypentyl)amino)pyridine-2-sulfonamide

In a microwave vial, N-(2′,3-dichloro-[2,3′-bipyridin]-6-yl)-6-fluoropyridine-2-sulfonamide (int-b11) (100 mg, 0.25 mmol) and 5-amino-1-pentanol (51.7 mg, 0.25 mmol, 50%) were taken up in NMP (3 mL) and then DIEA (0.13 mL, 0.75 mmol) was added and the reaction was heated to 200° C. for 1 h in microwave. The reaction was quenched with 1 M HCl and extracted into EtOAc (3×). The organics were then washed with water and brine, dried over MgSO₄, and concentrated in vacuo. The crude material was purified by flash column chromatography (ISCO, 4 g RediSep Rf Gold column, 0-80% EtOAc/heptanes) to give the title compound (86 mg, 0.18 mmol, 72% yield) as an off-white solid. LCMS (Condition 1): m/z 482.0 [M+H]⁺, 1.47 min. ¹H NMR (400 MHz, Methanol-d₄) δ 8.43 (dd, J=4.9, 1.9 Hz, 1H), 7.84 (d, J=8.9 Hz, 1H), 7.71 (dd, J=7.6, 1.9 Hz, 1H), 7.56 (d, J=8.9 Hz, 1H), 7.50-7.41 (m, 2H), 7.09 (dd, J=7.2, 0.6 Hz, 1H), 6.59 (dd, J=8.5, 0.7 Hz, 1H), 3.52 (t, J=6.6 Hz, 2H), 3.14 (t, J=7.0 Hz, 2H), 1.57-1.41 (m, 4H), 1.41-1.29 (m, 2H).

### Step 2. 2³-chloro-12-oxa-4-thia-3,6-diaza-1(3,2),2,5(2,6)-tripyridinacyclododecaphane 4,4-dioxide

In a vial, N-(2′,3-dichloro-[2,3′-bipyridin]-6-yl)-6-((5-hydroxypentyl)amino)pyridine-2-sulfonamide (86 mg, 0.18 mmol) was taken up in NMP (3 mL) and then sodium hydride (22 mg, 0.54 mmol, 60%) was added and the reaction was heated to 50° C. overnight. The reaction was quenched with 1 M HCl and extracted into EtOAc (3×). The organics were then washed with water and brine, dried over MgSO₄, and concentrated in vacuo. The crude material was purified by flash column chromatography (ISCO, 4 g RediSep Rf Gold column, EtOAc/heptane 0-80%) to give the title compound (107) as a white solid. LCMS (Condition 1): m/z 446.1 [M+H]⁺, 1.77 min. ¹H NMR (400 MHz, DMSO-d₆) δ 10.94 (s, 1H), 8.21 (dd, J=5.0, 1.9 Hz, 1H), 7.91 (d, J=8.9 Hz, 1H), 7.67 (dd, J=7.2, 1.7 Hz, 1H), 7.58 (d, J=8.9 Hz, 1H), 7.51 (dd, J=8.5, 7.2 Hz, 1H), 7.06 (dd, J=7.2, 5.0 Hz, 2H), 7.00 (d, J=7.1 Hz, 1H), 6.60 (d, J=8.5 Hz, 1H), 4.24-4.22 (m, 2H), 3.06 (s, 2H), 1.55 (s, 2H), 1.24-1.19 (m, 2H), 1.10 (s, 2H).

### Example 108: Synthesis of 2³-(trifluoromethyl)-1²-oxa-4-thia-3,6-diaza-1(3,2),2,5(2,6)-tripyridinacyclododecaphane 4,4-dioxide (108)

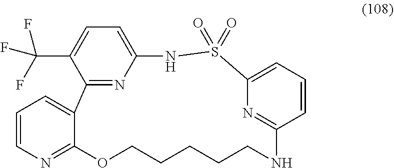 [SMILES: O=S1(=O)Nc2ccc(C(F)(F)F)c(n2)-c2cccnc2OCCCCCNc2cccc1n2]

2³-(trifluoromethyl)-1²-oxa-4-thia-3,6-diaza-1(3,2),2,5(2,6)-tripyridinacyclododecaphane 4,4-dioxide (108) was synthesized using the procedure described in Example 107 except N-(2′,3-dichloro-[2,3′-bipyridin]-6-yl)-6-fluoropyridine-2-sulfonamide (int-b11) was replaced with 6-fluoro-N-(2′-fluoro-3-(trifluoromethyl)-[2,3′-bipyridin]-6-yl)pyridine-2-sulfonamide (int-b10). LCMS (Condition 1): m/z 480.1 [M+H]⁺, 1.82 min. ¹H NMR (400 MHz, DMSO-d₆) δ 11.39 (s, 1H), 8.28-8.10 (m, 2H), 7.70 (dd, J=15.7, 8.1 Hz, 2H), 7.53 (dd, J=8.5, 7.2 Hz, 1H), 7.12 (s, 1H), 7.08-6.98 (m, 2H), 6.63 (d, J=8.5 Hz, 1H), 4.28-4.10 (m, 2H), 3.08-3.03 (m, 2H), 1.53-1.45 (m, 2H), 1.19-1.14 (m, 4H).

### Example 109: Synthesis of 7-(3-hydroxypropyl)-2³-(trifluoromethyl)-6-oxa-4-thia-3-aza-2,5(2,6)-dipyridina-1(1,2)-benzenacycloundecaphane 4,4-dioxide (109)

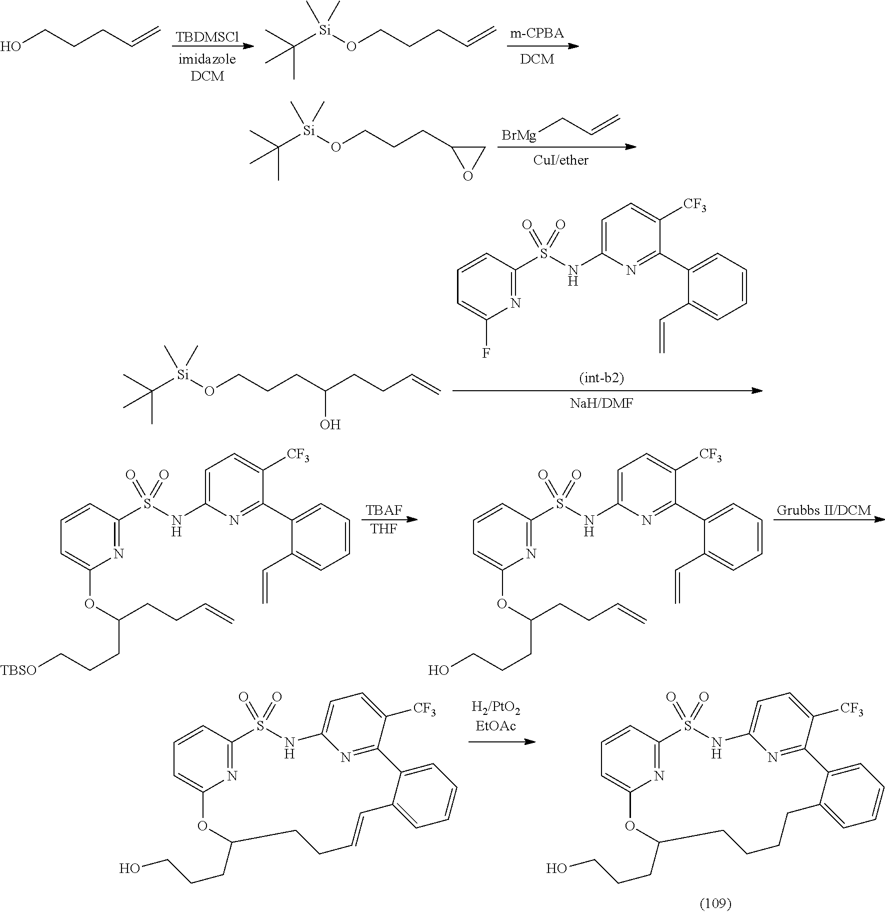

### Step 1. Synthesis of tert-butyldimethyl(pent-4-en-1-yloxy)silane

To a solution of pent-4-en-1-ol (3.00 g, 34.8 mmol) in DCM (200 mL) at 0° C. were added tert-butyldimethylsilyl chloride (11.6 g, 76.7 mmol) and imidazole (5.70 g, 83.7 mmol). The reaction mixture was stirred at 0° C. for 20 min, then warmed to room temperature and stirred for 1.5 h. Then the reaction was quenched with water. The layers were separated and the aqueous layer was extracted with dichloromethane (2×) and washed with brine. The combined organic extracts were dried over Na₂SO₄ and concentrated to give the title compound (7.10 g, 34.7 mmol, 100% yield) as an oil. The crude product was taken to the next step without further purification. ¹H NMR (400 MHz, Chloroform-d) δ 5.82 (ddt, J=6.6, 10.2, 16.9 Hz, 1H), 5.02 (dq, J=1.7, 17.1 Hz, 1H), 4.95 (ddt, J=1.2, 2.2, 10.2 Hz, 1H), 3.62 (t, J=6.5 Hz, 2H), 2.09 (m, 2H), 1.61 (dq, J=6.5, 8.3 Hz, 2H), 0.89 (s, 9H), 0.05 (s, 6H).

### Step 2. Synthesis of tert-butyldimethyl(3-(oxiran-2-yl)propoxy)silane

m-CPBA (5.87 g, 26.2 mmol) (77% w/w in H₂O) was added to a solution of tert-butyldimethyl(pent-4-en-1-yloxy)silane (3.50 g, 17.5 mmol) in DCM (100 mL) at 0° C. The reaction mixture was stirred at 0° C. for 20 min, then warmed to room temperature and stirred overnight followed by adding saturated aqueous Na₂CO₃ and NaHSO₃ (1:1) solution. The layers were separated and aqueous layer was further extracted with DCM. The combined organic layers were dried, washed with saturated NaHCO₃ solution, dried over Na₂SO₄, and concentrated. Purification by ISCO silica gel column (EtOAc/heptane 5%) gave the title compound (1.84 g, 49% yield) as a colorless oil. ¹H NMR (400 MHz, Chloroform-d) δ 3.64 (m, 2H), 2.95 (m, 1H), 2.75 (dd, J=4.1, 4.9 Hz, 1H), 2.48 (dd, J=2.7, 5.0 Hz, 1H), 1.64 (m, 4H), 0.89 (s, 9H), 0.05 (s, 6H).

### Step 3. Synthesis of 1-((tert-butyldimethylsilyl)oxy)oct-7-en-4-ol

A flask was charged with copper(I) iodide (0.317 g, 1.66 mmol), gently heated under vacuum, and then slowly cooled under a flow of nitrogen. Dry ether (50 mL) was added, and the resulting suspension was cooled to −78° C., stirred, and allylmagnesium bromide (1 M in diethyl ether, 15 ml, 15 mmol) was added. A solution of tert-butyldimethyl(3-(oxiran-2-yl)propoxy)silane (1.69 g, 6.54 mmol) in dry ether (10 mL) was added to the above mixture and stirred at −78° C. for 4 h. The reaction was quenched with saturated aqueous NH₄Cl, extracted with EA (×3), washed with brine, and dried over Na₂SO₄. Purification on an ISCO silica gel column (EtOAc/heptane 0-15%) gave the title compound (1.69 g, 79% yield) as an oil. ¹H NMR (400 MHz, DMSO-d₆) δ 5.81 (ddt, J=6.6, 10.2, 16.9 Hz, 1H), 4.99 (m, 1H), 4.92 (ddt, J=1.2, 2.3, 10.2 Hz, 1H), 4.35 (d, J=5.5 Hz, 1H), 3.56 (t, J=6.4 Hz, 2H), 3.38 (m, 1H), 2.06 (m, 2H), 1.39 (m, 6H), 0.86 (s, 9H), 0.02 (s, 6H).

### Step 4. Synthesis of 6-((1-((tert-butyldimethylsilyl)oxy)oct-7-en-4-yl)oxy)-N-(5-(trifluoromethyl)-6-(2-vinylphenyl)pyridin-2-yl)pyridine-2-sulfonamide

To a solution of 6-fluoro-N-(5-(trifluoromethyl)-6-(2-vinylphenyl)pyridin-2-yl)pyridine-2-sulfonamide (int-b2) (200 mg, 0.47 mmol) and 1-((tert-butyldimethylsilyl)oxy)oct-7-en-4-ol (610 mg, 2.36 mmol) in DMF (5 mL) was added NaH (189 mg, 4.7 mmol) under nitrogen atmosphere. The mixture was stirred at room temperature for 45 min, quenched with water, acidified with 10% solution of citric acid, extracted with EtOAc. The combined layers were washed with water, NaHCO₃, brine (×2) and dried over Na₂SO₄. Purification on an ISCO silica gel column (EtOAc/heptane 0-100%) gave the title compound (185 mg, 0.165 mmol, 59% yield) as an oil. Conditions 1, LCMS: m/z 662.3 [M+H]⁺, 2.06 min.

### Step 5. Synthesis of 6-((1-hydroxyoct-7-en-4-yl)oxy)-N-(5-(trifluoromethyl)-6-(2-vinylphenyl)pyridin-2-yl)pyridine-2-sulfonamide

To a solution of 6-((1-((tert-butyldimethylsilyl)oxy)oct-7-en-4-yl)oxy)-N-(5-(trifluoromethyl)-6-(2-vinylphenyl)pyridin-2-yl)pyridine-2-sulfonamide (185 mg, 0.28 mmol) in THE (2 mL) was added TBAF (1 M in THF, 2 mL, 2 mmol) under nitrogen atmosphere. The mixture stirred at room temperature for 45 min. Water was added, extracted with EtOAc. The combined extracts were washed with brine and dried over Na₂SO₄. Purification on an ISCO silica gel column (EtOAc/heptane 0-100%) gave the title compound (101 mg, 66% yield) as an oil: LCMS (Condition 1): m/z 548.3 [M+H]⁺, 1.69 min. ¹⁹F NMR (471 MHz, DMSO-d₆) δ −56.97 (s).

### Step 6. Synthesis of (E)-7-(3-hydroxypropyl)-2³-(trifluoromethyl)-6-oxa-4-thia-3-aza-2,5(2,6)-dipyridina-1(1,2)-benzenacycloundecaphan-10-ene 4,4-dioxide

A 100 mL flask with a solution of 6-((1-hydroxyoct-7-en-4-yl)oxy)-N-(5-(trifluoromethyl)-6-(2-vinylphenyl)pyridin-2-yl)pyridine-2-sulfonamide (101 mg, 0.184 mmol) in DCM (50 mL) was purged with argon. Grubbs II catalyst (16 mg, 0.018 mmol) was added and purged with argon again. The flask was capped and the reaction mixture was stirred at 45° C. for 40 h. The reaction mixture was directly loaded onto silica and purified on an ISCO silica gel column (EtOAc/heptane 0-50%) to give the title compound (93 mg, 95% yield) as white crystals. LCMS (Condition 1): m/z 520.2 [M+H]⁺, 1.65 min. ¹⁹F NMR (376 MHz, DMSO-d₆) δ−57.16 (s), 57.51 (s). Diastereomers, 55:45 ratio.

### Step 7. Synthesis of 7-(3-hydroxypropyl)-2³-(trifluoromethyl)-6-oxa-4-thia-3-aza-2,5(2,6)-dipyridina-1(1,2)-benzenacycloundecaphane 4,4-dioxide (109)

A solution of (E)-7-(3-hydroxypropyl)-2³-(trifluoromethyl)-6-oxa-4-thia-3-aza-2,5(2,6)-dipyridina-1(1,2)-benzenacycloundecaphan-10-ene 4,4-dioxide (95 mg, 0.183 mmol) in EtOAc (10 mL) was hydrogenated overnight over PtO₂ hydrate (10 mg, 0.044 mmol) with a hydrogen balloon at room temperature. The slurry was filtered to remove catalyst and the filtrate was concentrated. Purification on ISCO silica gel column (EtOAc/heptane 0-50%) gave the title compound as white crystals. LCMS (Condition 1): m/z 522.2 [M+H]⁺, 1.68 min. ¹⁹F NMR (376 MHz, DMSO-d₆) δ−56.83(s), −57.56 (s). Diastereomers, 42:58 ratio.

### Example 110: Synthesis of 3-(4,4-dioxido-2³-(trifluoromethyl)-6-oxa-4-thia-3-aza-2,5(2,6)-dipyridina-1(1,2)-benzenacycloundecaphane-7-yl)propanoic acid (110)

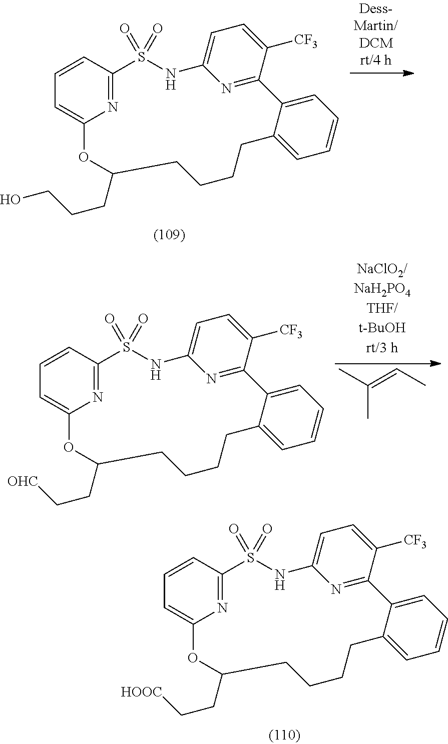

### Step 1. Synthesis of 3-(4,4-dioxido-2³-(trifluoromethyl)-6-oxa-4-thia-3-aza-2,5(2,6)-dipyridina-1(1,2)-benzenacycloundecaphane-7-yl)propanal

To a solution of 7-(3-hydroxypropyl)-2³-(trifluoromethyl)-6-oxa-4-thia-3-aza-2,5(2,6)-dipyridina-1(1,2)-benzenacycloundecaphane 4,4-dioxide (109) (66 mg, 0.127 mmol) in DCM (2 mL) was added in one portion Dess-Martin periodinane (70 mg, 0.17 mmol) at 0° C. The mixture was allowed to slowly warm to room temperature and stirred for 4 h. Saturated aqueous NaHCO₃ (1 mL) and saturated aqueous Na₂S2O₃ (1 mL) were added. The mixture was stirred vigorously for 15 min before the layers were separated. The aqueous layer was extracted with DCM (3×), the combined organic layers were washed with brine and dried over Na₂SO₄, filtered and concentrated. The residue was purified by ISCO silica gel column (EtOAc/heptane 0-60%) to give the title compound (59 mg, 0.113 mmol, 89% yield) as a white solid. Conditions 1, LCMS: m/z 520.2 [M+H]⁺, 1.73 min.

### Step 2. Synthesis of 3-(4,4-dioxido-2³-(trifluoromethyl)-6-oxa-4-thia-3-aza-2,5(2,6)-dipyridina-1(1,2)-benzenacycloundecaphane-7-yl)propanoic acid (110)

To a solution of 3-(4,4-dioxido-2³-(trifluoromethyl)-6-oxa-4-thia-3-aza-2,5(2,6)-dipyridina-1(1,2)-benzenacycloundecaphane-7-yl)propanal (52 mg, 0.10 mmol) in THE (2 mL) and t-BuOH (2 mL) and was added 2-methyl-2-butene (0.5 mL, 4.7 mmol) followed by NaH₂PO₄ (72 mg, 0.6 mmol) in water (0.25 mL) and NaClO₂ (68 mg, 0.6 mmol) in water (0.25 mL). The solution was stirred at room temperature for 3 h. Water was added and extracted with DCM (3×), the combined organic layers were washed with brine and dried over Na₂SO₄, filtered and concentrated. The residue was purified by ISCO silica gel column (EtOAc/heptane 0-100%) to give the title compound as a white solid. LCMS (Condition 1): m/z 536.2 [M+H]⁺, 1.65 min, ¹H NMR (400 MHz, DMSO-d₆) δ 12.08 (s, 1.6H), 11.53 (s, 0.4H), 8.28 (d, J=9.0 Hz, 0.4H), 8.08 (d, J=8.9 Hz, 0.6H), 7.97 (m, 1H), 7.83 (m, 0.4H), 7.75 (d, J=7.0 Hz, 0.6H), 7.55 (d, J=7.2 Hz, 0.4H), 7.29 (m, 3.6H), 7.15 (m, 1H), 7.03 (m, 1H), 5.42 (m, 0.6H), 4.34 (m, 0.4H), 2.24 (m, 3.4H), 1.86 (m, 0.6H), 0.9-1.8 (m, 8H). ¹⁹F NMR (376 MHz, DMSO-d₆) δ−56.74(s, 0.4F), −57.52 (s, 0.6F). Diastereomers, 2:3 ratio.

### Example 111: Synthesis of 2³-chloro-6-oxa-4-thia-3-aza-2,5(2,6)-dipyridina-1(1,2)-benzenacyclododecaphane 4,4-dioxide (111)

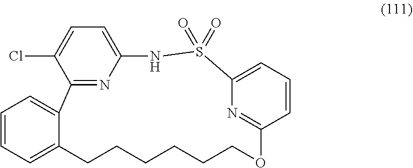 [SMILES: O=S1(=O)Nc2ccc(Cl)c(n2)-c2ccccc2CCCCCCOc2cccc1n2]

2³-chloro-6-oxa-4-thia-3-aza-2,5(2,6)-dipyridina-1(1,2)-benzenacyclododecaphane 4,4-dioxide (111) was synthesized using the procedure described in Example 109 except 6-fluoro-N-(5-(trifluoromethyl)-6-(2-vinylphenyl)pyridin-2-yl)pyridine-2-sulfonamide (int-b2) was replaced with N-(5-chloro-6-(2-vinylphenyl)pyridin-2-yl)-6-fluoropyridine-2-sulfonamide (int-b3) and 1-((tert-butyldimethylsilyl)oxy)oct-7-en-4-ol was replaced with hex-5-en-1-ol. LCMS (Condition 1): m/z 444.1 [M+H]⁺, 1.79 min. ¹H NMR (400 MHz, DMSO-d₆) δ 11.51 (s, 1H), 7.95 (m, 2H), 7.61 (d, J=7.3 Hz, 1H), 7.38 (d, J=8.5 Hz, 1H), 7.32 (m, 1H), 7.26 (m, 2H), 7.19 (m, 1H), 7.05 (d, J=8.3 Hz, 1H), 4.24 (s, 2H), 2.55 (m, 1H), 2.17 (m, 1H), 1.38 (s, 2H), 1.09 (m, 6H).

### Example 112: Synthesis of 2³-chloro-6-oxa-4-thia-3-aza-2,5(2,6)-dipyridina-1(1,2)-benzenacyclodecaphane 4,4-dioxide (112)

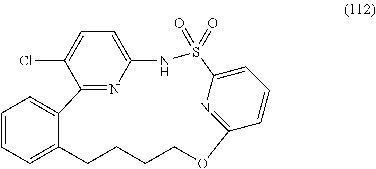 [SMILES: O=S1(=O)Nc2ccc(Cl)c(n2)-c2ccccc2CCCCOc2cccc1n2]

2³-chloro-6-oxa-4-thia-3-aza-2,5(2,6)-dipyridina-1(1,2)-benzenacyclodecaphane 4,4-dioxide (112) was synthesized using the procedure described in Example 109 except 6-fluoro-N-(5-(trifluoromethyl)-6-(2-vinylphenyl)pyridin-2-yl)pyridine-2-sulfonamide (int-b2) was replaced with N-(5-chloro-6-(2-vinylphenyl)pyridin-2-yl)-6-fluoropyridine-2-sulfonamide (int-b3) and 1-((tert-butyldimethylsilyl)oxy)oct-7-en-4-ol was replaced with but-3-en-1-ol. LCMS (Condition 1): m/z 416.1 [M+H]⁺, 1.69 min. ¹H NMR (400 MHz, DMSO-d₆) δ 11.25 (s, 1H), 7.95 (m, 2H), 7.61 (d, J=7.3 Hz, 1H), 7.35 (m, 3H), 7.28 (td, J=1.8, 7.2 Hz, 1H), 7.21 (dd, J=1.1, 7.5 Hz, 1H), 7.02 (d, J=8.3 Hz, 1H), 4.10 (s, 2H), 2.65 (m, 1H), 2.04 (m, 1H), 1.48 (m, 4H).

### Example 113: Synthesis of 2³-chloro-6-oxa-4-thia-3-aza-2,5(2,6)-dipyridina-1(1,2)-benzenacycloundecaphane 4,4-dioxide (113)

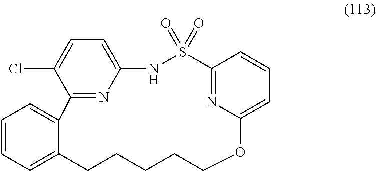 [SMILES: O=S1(=O)Nc2ccc(Cl)c(n2)-c2ccccc2CCCCCOc2cccc1n2]

2³-chloro-6-oxa-4-thia-3-aza-2,5(2,6)-dipyridina-1(1,2)-benzenacycloundecaphane 4,4-dioxide (113) was synthesized using the procedure described in Example 109 except 6-fluoro-N-(5-(trifluoromethyl)-6-(2-vinylphenyl)pyridin-2-yl)pyridine-2-sulfonamide (int-b2) was replaced with N-(5-chloro-6-(2-vinylphenyl)pyridin-2-yl)-6-fluoropyridine-2-sulfonamide (int-b3) and 1-((tert-butyldimethylsilyl)oxy)oct-7-en-4-ol was replaced with pent-4-en-1-ol. LCMS (Condition 1): m/z 430.2 [M+H]⁺, 1.75 min. ¹H NMR (400 MHz, DMSO-d₆) δ 11.32 (s, 1H), 7.95 (m, 2H), 7.62 (d, J=7.3 Hz, 1H), 7.44 (d, J=8.9 Hz, 1H), 7.34 (td, J=1.4, 7.4 Hz, 1H), 7.27 (m, 2H), 7.13 (m, 1H), 7.03 (d, J=8.4 Hz, 1H), 4.11 (m, 1H), 3.83 (m, 1H), 2.37 (m, 1H), 2.24 (m, 1H), 1.33 (m, 6H).

### Example 114: Synthesis of 2³-chloro-6-oxa-4-thia-3-aza-2,5(2,6)-dipyridina-1(1,2)-benzenacyclononaphane 4,4-dioxide (114)

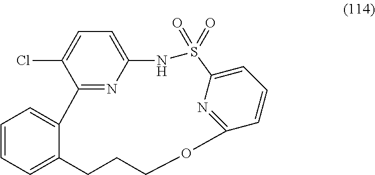 [SMILES: O=S1(=O)Nc2ccc(Cl)c(n2)-c2ccccc2CCCOc2cccc1n2]

2³-chloro-6-oxa-4-thia-3-aza-2,5(2,6)-dipyridina-1(1,2)-benzenacyclononaphane 4,4-dioxide (114) was synthesized using the procedure described in Example 109 except 6-fluoro-N-(5-(trifluoromethyl)-6-(2-vinylphenyl)pyridin-2-yl)pyridine-2-sulfonamide (int-b2) was replaced with N-(5-chloro-6-(2-vinylphenyl)pyridin-2-yl)-6-fluoropyridine-2-sulfonamide (int-b3) and 1-((tert-butyldimethylsilyl)oxy)oct-7-en-4-ol was replaced with prop-2-en-1-ol. LCMS (Condition 1): m/z 402.1 [M+H]⁺, 1.62 min, ¹H NMR (400 MHz, DMSO-d₆) δ 10.97 (s, 1H), 8.06 (d, J=8.7 Hz, 1H), 7.93 (dd, J=7.4, 8.3 Hz, 1H), 7.60 (m, 1H), 7.46 (d, J=8.7 Hz, 1H), 7.36 (m, 2H), 7.30 (m, 1H), 7.23 (m, 1H), 7.01 (dd, J=0.5, 8.4 Hz, 1H), 3.96 (m, 2H), 1.3-2.5 (m, 4H).

### Example 115: Synthesis of 2³-(trifluoromethyl)-4-thia-3,6-diaza-2,5(2,6)-dipyridina-1(1,2)-benzenacyclodecaphane 4,4-dioxide (115)

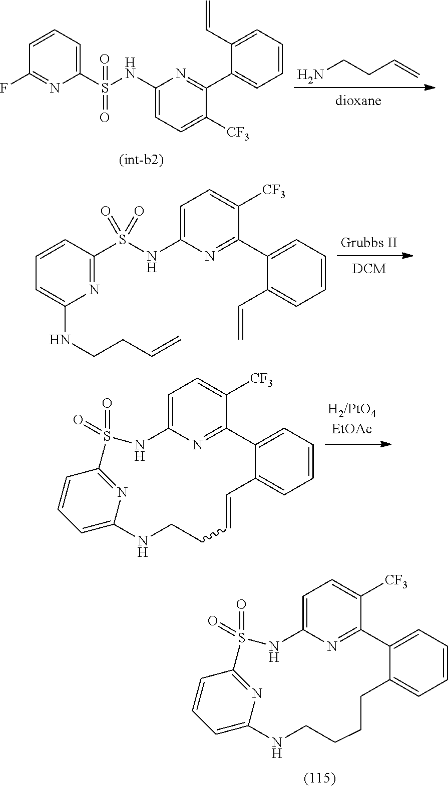

### Step 1. Synthesis of 6-(but-3-en-1-yl(2-hydroxyethyl)amino)-N-(5-(trifluoromethyl)-6-(2-vinylphenyl)pyridin-2-yl)pyridine-2-sulfonamide

A mixture of 6-fluoro-N-(5-(trifluoromethyl)-6-(2-vinylphenyl)pyridin-2-yl)pyridine-2-sulfonamide (int-b2) (60 mg, 0.14 mmol) and but-3-en-1-amine (50 mg, 0.71 mmol) in dioxane (4 mL) was heated overnight at 120° C. The mixture was partitioned between EtOAc and water and acidified with 1 N aqueous HCl (2 mL). The organic layer was collected, dried over MgSO₄, filtered and concentrated. The residue was purified by ISCO (EtOAc/heptane 0-100%) to afford the title compound (50 mg, 0.105 mmol, 74% yield) as a white solid. LCMS (Condition 1): m/z 475.2 [M+H]⁺, 1.69 min.

### Step 2. Synthesis of 2³-(trifluoromethyl)-4-thia-3,6-diaza-2,5(2,6)-dipyridina-1(1,2)-benzenacyclodecaphan-9-ene 4,4-dioxide

A solution of 6-(but-3-en-1-yl(2-hydroxyethyl)amino)-N-(5-(trifluoromethyl)-6-(2-vinylphenyl)pyridin-2-yl)pyridine-2-sulfonamide) (50 mg, 0.105 mmol) in DCM (50 mL) was purged with nitrogen. Grubbs II catalyst (9 mg, 11 μmol) was added and the mixture was purged with nitrogen again. The flask was sealed and heated overnight at 45° C. LCMS indicated that the reaction was not complete. Additional 15 mg of Grubbs II catalyst was added and the mixture was allowed to continue heating at 45° C. for 3 days. The reaction mixture was concentrated in vacuo and the residue was purified on an ISCO column (EtOAc/heptane 0-25%) to afford the title compound (45 mg, 0.096 mmol, 91% yield) as a mixture of E/Z isomers in 3:1 ratio. LCMS (Condition 1): m/z 447.2 [M+H]⁺, 1.6 min.

### Step 3. Synthesis of 2³-(trifluoromethyl)-4-thia-3,6-diaza-2,5(2,6)-dipyridina-1(1,2)-benzenacyclodecaphane 4,4-dioxide (115)

To a solution of 2³-(trifluoromethyl)-4-thia-3,6-diaza-2,5(2,6)-dipyridina-1(1,2)-benzenacyclodecaphan-9-ene 4,4-dioxide (45 mg, 0.096 mmol) in EtOAc (10 mL) was hydrogenated overnight over PtO₂ hydrate (50 mg, 0.22 mmol) at room temperature. The mixture was filtered through Celite and the filtrate was concentrated. The residue was purified by ISCO (EtOAc/heptane 0-25%) to afford the title compound as a white solid. LCMS (Condition 1): m/z 449.2 [M+H]⁺, 1.65 min. ¹H NMR (400 MHz, DMSO-d₆) δ 11.50 (s, 1H), 8.16 (d, J=8.9 Hz, 1H), 7.56 (dd, J=8.4, 7.2 Hz, 1H), 7.43-7.32 (m, 2H), 7.32-7.19 (m, 3H), 7.14 (t, J=5.9 Hz, 1H), 7.10 (d, J=7.1 Hz, 1H), 6.61 (d, J=8.4 Hz, 1H), 2.89-2.75 (m, 1H), 2.63-2.55 (m, 1H), 1.83 (m, 1H), 1.58 (m, 1H), 1.42-1.31 (m, 1H), 1.31-1.20 (m, 2H), 0.98-0.89 (m, 1H).

### Example 116: Synthesis of 6-(2-hydroxyethyl)-2³-(trifluoromethyl)-4-thia-3,6-diaza-2,5(2,6)-dipyridina-1(1,2)-benzenacyclodecaphane 4,4-dioxide (116)

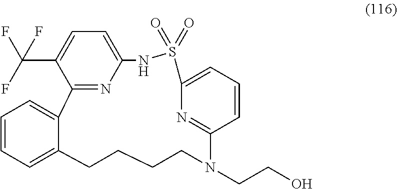 [SMILES: O=S1(=O)Nc2ccc(C(F)(F)F)c(n2)-c2ccccc2CCCCN(CCO)c2cccc1n2]

6-(2-hydroxyethyl)-2³-(trifluoromethyl)-4-thia-3,6-diaza-2,5(2,6)-dipyridina-1(1,2)-benzenacyclodecaphane 4,4-dioxide (116) was synthesized using the procedure described in Example 115 except but-3-en-1-amine was replaced with 2-(but-3-en-1-ylamino)ethan-1-ol (purchased). LCMS (Condition 1): m/z 493.2 [M+H]⁺, 1.61 min; ¹H NMR (400 MHz, DMSO-d₆) δ 11.69 (s, 1H), 8.16 (d, J=8.9 Hz, 1H), 7.70 (dd, J=8.7, 7.3 Hz, 1H), 7.39-7.31 (m, 2H), 7.30-7.22 (m, 3H), 7.17 (d, J=7.2 Hz, 1H), 6.88 (d, J=8.7 Hz, 1H), 4.76 (t, J=5.4 Hz, 1H), 3.94 (t, J=10.3 Hz, 1H), 3.50 (q, J=5.8 Hz, 2H), 3.45-3.34 (m, 1H), 2.94-2.87 (m, 1H), 2.60-2.54 (m, 1H), 1.75 (t, J=10.9 Hz, 1H), 1.67-1.60 (m, 1H), 1.40-1.21 (m, 2H), 0.89-0.83 (m, 1H), 0.68 (s, 1H).

### Example 117: Synthesis of 6-(2-hydroxyethyl)-2³-(trifluoromethyl)-4-thia-3,6-diaza-2,5(2,6)-dipyridina-1(1,2)-benzenacycloundecaphane 4,4-dioxide (117)

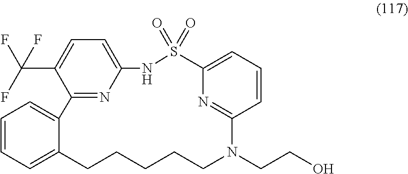 [SMILES: O=S1(=O)Nc2ccc(C(F)(F)F)c(n2)-c2ccccc2CCCCCN(CCO)c2cccc1n2]

6-(2-hydroxyethyl)-2³-(trifluoromethyl)-4-thia-3,6-diaza-2,5(2,6)-dipyridina-1(1,2)-benzenacycloundecaphane 4,4-dioxide (117) was synthesized using the procedure described in Example 115 except but-3-en-1-amine was replaced with 2-(pent-4-en-1-ylamino)ethan-1-ol (int-a42). LCMS (Condition 1): m/z 507.2 [M+H]⁺, 1.64 min. ¹H NMR (400 MHz, DMSO-d₆) δ 11.79 (s, 1H), 8.10 (d, J=9.0 Hz, 1H), 7.70 (dd, J=8.7, 7.2 Hz, 1H), 7.39-7.35 (m, 1H), 7.29 (d, J=6.9 Hz, 1H), 7.26-7.12 (m, 4H), 6.90 (d, J=8.8 Hz, 1H), 4.77 (t, J=5.4 Hz, 1H), 4.02-3.91 (m, 1H), 3.51 (q, J=6.1 Hz, 2H), 3.46-3.35 (m, 2H), 2.97-2.87 (m, 1H), 2.45-2.36 (m, 1H), 1.97-1.84 (m, 1H), 1.82-1.67 (m, 1H), 1.45-1.26 (m, 2H), 1.21-0.99 (m, 3H).

### Example 118: Synthesis of 2³-(trifluoromethyl)-4-thia-3,6-diaza-2,5(2,6)-dipyridina-1(1,2)-benzenacycloundecaphane 4,4-dioxide (118)

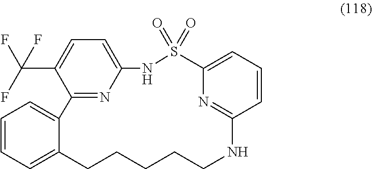 [SMILES: O=S1(=O)Nc2ccc(C(F)(F)F)c(n2)-c2ccccc2CCCCCNc2cccc1n2]

2³-(trifluoromethyl)-4-thia-3,6-diaza-2,5(2,6)-dipyridina-1(1,2)-benzenacycloundecaphane 4,4-dioxide (118) was synthesized using the procedure described in Example 115 except but-3-en-1-amine was replaced with pent-4-en-1-amine. LCMS (Condition 1): m/z 463.2 [M+H]⁺, 1.69 min. ¹H NMR (400 MHz, DMSO-d₆) δ 11.60 (s, 1H), 8.13 (d, J=9.0 Hz, 1H), 7.55 (dd, J=8.5, 7.2 Hz, 1H), 7.39-7.26 (m, 3H), 7.25-7.21 (m, 1H), 7.14 (d, J=7.2 Hz, 2H), 7.10-7.03 (m, 1H), 6.62 (d, J=8.4 Hz, 1H), 3.41 (m, 1H), 2.81 (m, 1H), 2.41-2.31 (m, 1H), 2.05-1.93 (m, 1H), 1.59 (m, 1H), 1.36 (m, 1H), 1.30-1.13 (m, 4H).

### Example 119: Synthesis of 2³-(trifluoromethyl)-9-oxa-4-thia-3,6-diaza-2,5(2,6)-dipyridina-1(1,2)-benzenacyclododecaphane 4,4-dioxide (119)

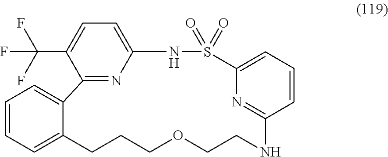 [SMILES: O=S1(=O)Nc2ccc(C(F)(F)F)c(n2)-c2ccccc2CCCOCCNc2cccc1n2]

2³-(trifluoromethyl)-9-oxa-4-thia-3,6-diaza-2,5(2,6)-dipyridina-1(1,2)-benzenacyclododecaphane 4,4-dioxide (119) was synthesized using the procedure described in Example 115 except but-3-en-1-amine was replaced with 2-(allyloxy)ethan-1-amine. LCMS (Condition 1): m/z 479.2 [M+H]⁺, 1.57 min. ¹H NMR (400 MHz, DMSO-d₆) δ 11.62 (s, 1H), 8.15 (d, J=8.9 Hz, 1H), 7.55 (dd, J=8.4, 7.3 Hz, 1H), 7.40-7.31 (m, 2H), 7.28-7.20 (m, 2H), 7.15 (d, J=6.9 Hz, 1H), 7.09-7.01 (m, 2H), 6.68 (d, J=8.5 Hz, 1H), 3.33-3.24 (m, 4H), 3.21-3.14 (m, 2H), 2.40-2.29 (m, 1H), 2.11-1.98 (m, 1H), 1.58-1.47 (m, 1H), 1.40-1.28 (m, 1H).

### Example 120: Synthesis of 2-(4,4-dioxido-2³-(trifluoromethyl)-4-thia-3,6-diaza-2,5(2,6)-dipyridina-1(1,2)-benzenacyclododecaphane-6-yl)acetic acid (120)

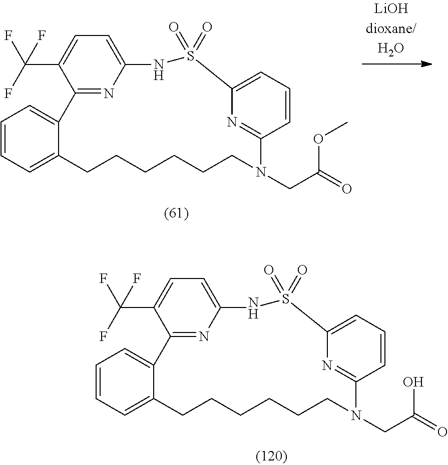 [SMILES: COC(=O)CN1CCCCCCc2ccccc2-c2nc(ccc2C(F)(F)F)NS(=O)(=O)c2cccc1n2 ; O=C(O)CN1CCCCCCc2ccccc2-c2nc(ccc2C(F)(F)F)NS(=O)(=O)c2cccc1n2]

The mixture of methyl 2-(4,4-dioxido-2³-(trifluoromethyl)-4-thia-3,6-diaza-2,5(2,6)-dipyridina-1(1,2)-benzenacyclododecaphane-6-yl)acetate (61) (80 mg, 0.15 mmol), LiOH (150 mg, 6.26 mmol), Dioxane (3 mL) and Water (3.00 mL) was stirred overnight at room temperature. LCMS indicated that the reaction was complete. The reaction was acidified with 10% citric acid, aqueous work-up followed by ISCO purification (MeOH/DCM 0-10%) to give the product 2-(4,4-dioxido-2³-(trifluoromethyl)-4-thia-3,6-diaza-2,5(2,6)-dipyridina-1(1,2)-benzenacyclododecaphane-6-yl)acetic acid (120) as a white solid. LCMS (Condition 1): m/z 535.2 [M+1]⁺, 1.80 min. ¹H NMR (400 MHz, DMSO-d₆) δ 12.79 (s, 1H), 11.77 (s, 1H), 8.29-7.97 (m, 1H), 7.79-7.62 (m, 1H), 7.50-7.30 (m, 3H), 7.30-7.19 (m, 2H), 7.19-7.08 (m, 1H), 6.89-6.51 (m, 1H), 4.09 (s, 2H), 3.80-3.54 (m, 1H), 3.52-3.33 (m, 2H), 2.12-1.94 (m, 1H), 1.45-1.17 (m, 3H), 1.17-0.99 (m, 3H), 0.99-0.60 (m, 2H).

### Example 121: Synthesis of 6-(2-(piperazin-1-yl)ethyl)-2³-(trifluoromethyl)-4-thia-3,6-diaza-2,5(2,6)-dipyridina-1(1,2)-benzenacycloundecaphane 4,4-dioxide (121)

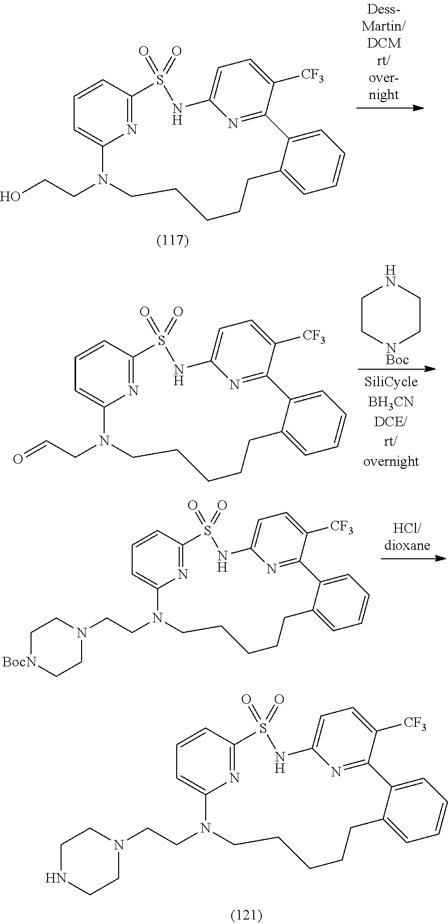

### Step 1. Synthesis of 2-(4,4-dioxido-2³-(trifluoromethyl)-4-thia-3,6-diaza-2,5(2,6)-dipyridina-1(1,2)-benzenacycloundecaphane-6-yl)acetaldehyde

To a solution of 6-(2-hydroxyethyl)-2³-(trifluoromethyl)-4-thia-3,6-diaza-2,5(2,6)-dipyridina-1(1,2)-benzenacycloundecaphane 4,4-dioxide (117) (320 mg, 0.632 mmol) in CH₂Cl₂ (10 mL) was added in one portion Dess-Martin Periodinane (348 mg, 0.821 mmol) at 0° C. The mixture was allowed to slowly warm to room temperature overnight. LCMS indicated that the reaction was complete. Then saturated aqueous NaHCO₃ (2.5 mL) and saturated aqueous Na₂S2O₃ (2.5 mL) were added. The mixture was stirred vigorously for 15 min before the layers were separated. The aqueous layer was extracted with DCM. The combined organic layers were dried over MgSO₄, filtered and concentrated. The residue was purified using an ISCO column (EtOAc/heptane 0-100%) to give the title compound (220 mg, 0.436 mmol, 69% yield) as a light brown solid. LCMS (Condition 1): m/z 505.2 [M+H]⁺, 1.63 min.

### Step 2. Synthesis of tert-butyl 4-(2-(4,4-dioxido-2³-(trifluoromethyl)-4-thia-3,6-diaza-2,5(2,6)-dipyridina-1(1,2)-benzenacycloundecaphane-6-yl)ethyl)piperazine-1-carboxylate

To a solution of 2-(4,4-dioxido-2³-(trifluoromethyl)-4-thia-3,6-diaza-2,5(2,6)-dipyridina-1(1,2)-benzenacycloundecaphane-6-yl)acetaldehyde (20 mg, 0.04 mmol) in DCE (3 mL) was added tert-butyl piperazine-1-carboxylate (11 mg, 0.06 mmol) and the resulting mixture was stirred at room temperature for 15 min. Then, SiliCycle BH₃CN (40 mg, 0.04 mmol) was added in one portion and the mixture stirred overnight at room temperature. The reaction mixture was filtered through a syringe filter and rinsed with DCM. The filtrate was concentrated in vacuo. The residue was purified via an ISCO column (EtOAc/heptane 0-100%) to give the title compound (20 mg, 0.03 mmol, 75% yield). LCMS (Condition 1): m/z 675.4 [M+H]⁺, 1.62 min.

### Step 3. Synthesis of 6-(2-(piperazin-1-yl)ethyl)-2³-(trifluoromethyl)-4-thia-3,6-diaza-2,5(2,6)-dipyridina-1(1,2)-benzenacycloundecaphane 4,4-dioxide (121)

To tert-butyl 4-(2-(4,4-dioxido-2³-(trifluoromethyl)-4-thia-3,6-diaza-2,5(2,6)-dipyridina-1(1,2)-benzenacycloundecaphane-6-yl)ethyl)piperazine-1-carboxylate (20 mg, 0.03 mmol). was added 4 N HCl in dioxane (2 mL) and the mixture was stirred for 2 h. The mixture was then diluted with ether and allowed to sit overnight. The resulting precipitate was collected and the solids were washed with ether and dried under high vacuum to afford the title compound as a HCl salt. LCMS (Condition 1): m/z 575.3 [M+H]⁻, 1.46 min. ¹H NMR (400 MHz, DMSO-d₆) δ 11.81 (s, 1H), 9.32 (s, 2H), 8.12 (d, J=9.0 Hz, 1H), 7.81-7.72 (m, 1H), 7.39-7.35 (m, 1H), 7.32-7.29 (m, 2H), 7.26-7.22 (m, 2H), 7.15 (d, J=7.6 Hz, 1H), 7.08 (s, 1H), 3.91-3.85 (m, 1H), 3.84-3.61 (m, 3H), 3.32-3.12 (m, 9H), 3.06-2.95 (m, 1H), 2.43-2.35 (m, 1H), 1.92-1.83 (m, 1H), 1.72-1.63 (m, 1H), 1.42-1.31 (m, 2H), 1.26-1.03 (m, 3H).

### Example 122: Synthesis of 6-(2-(pyrrolidin-1-yl)ethyl)-2³-(trifluoromethyl)-4-thia-3,6-diaza-2,5(2,6)-dipyridina-1(1,2)-benzenacycloundecaphane 4,4-dioxide (122)

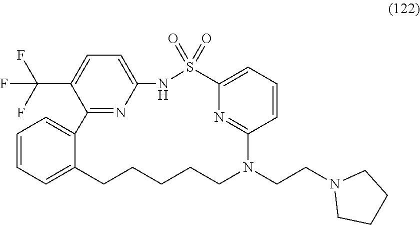 [SMILES: O=S1(=O)Nc2ccc(C(F)(F)F)c(n2)-c2ccccc2CCCCCN(CCN2CCCC2)c2cccc1n2]

6-(2-(pyrrolidin-1-yl)ethyl)-2³-(trifluoromethyl)-4-thia-3,6-diaza-2,5(2,6)-dipyridina-1(1,2)-benzenacycloundecaphane 4,4-dioxide (122) was synthesized using the procedure described in Example 121 except tert-butyl piperazine-1-carboxylate was replaced with tert-butyl pyrrolidine-1-carboxylate. LCMS (Condition 1): m/z 560.3 [M+H]⁺, 1.51 min. ¹H NMR (400 MHz, DMSO-d₆) δ 7.94 (d, J=8.9 Hz, 1H), 7.68-7.55 (m, 1H), 7.31-7.25 (m, 1H), 7.20 (d, J=7.6 Hz, 1H), 7.18-7.12 (m, 2H), 7.07 (t, J=8.3 Hz, 2H), 6.75 (d, J=8.7 Hz, 1H), 3.86-3.75 (m, 1H), 3.41-3.35 (m, 3H), 2.93-2.83 (m, 1H), 2.70-2.51 (m, 5H), 2.37-2.27 (m, 1H), 1.90-1.77 (m, 1H), 1.71-1.53 (m, 5H), 1.33-1.21 (m, 2H), 1.15-0.88 (m, 3H).

### Example 123: Synthesis of 6-(2-((3S,4S)-3,4-dihydroxypyrrolidin-1-yl)ethyl)-2³-(trifluoromethyl)-4-thia-3,6-diaza-2,5(2,6)-dipyridina-1(1,2)-benzenacycloundecaphane 4,4-dioxide (123)

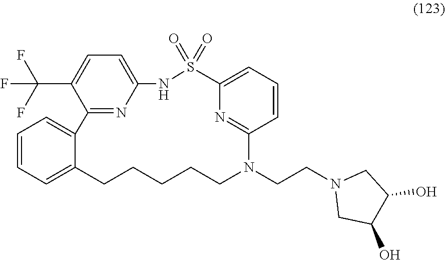 [SMILES: O=S1(=O)Nc2ccc(C(F)(F)F)c(n2)-c2ccccc2CCCCCN(CCN2C[C@H](O)[C@@H](O)C2)c2cccc1n2]

6-(2-((3S,4S)-3,4-dihydroxypyrrolidin-1-yl)ethyl)-2³-(trifluoromethyl)-4-thia-3,6-diaza-2,5(2,6)-dipyridina-1(1,2)-benzenacycloundecaphane 4,4-dioxide (123) was synthesized using the procedure described in Example 121 except tert-butyl piperazine-1-carboxylate was replaced with tert-butyl (3S,4S)-3,4-dihydroxypyrrolidine-1-carboxylate. LCMS (Condition 1): m/z 592.3 [M+H]⁺, 1.40 min. ¹H NMR (400 MHz, DMSO-d₆) 11.65 (s, 1H), 8.05 (d, J=8.0 Hz, 1H), 7.71 (t, J=7.8 Hz, 1H), 7.35 (m, 1H), 7.28 (d, J=7.4 Hz, 1H), 7.19 (m, 4H), 6.81 (d, J=8.5 Hz, 1H), 4.96 (m, 2H), 3.88 (m, 3H), 3.40 (m, 1H), 2.92 (m, 3H), 2.30-2.70 (m, 5H), 2.40, 1.89 (m, 1H), 1.69 (m, 1H), 0.96-1.42 (m, 6H). ¹⁹F NMR (376 MHz, DMSO-d₆) δ−56.77 (s).

### Example 124: Synthesis of 6-(2-(3-hydroxyazetidin-1-yl)ethyl)-2³-(trifluoromethyl)-4-thia-3,6-diaza-2,5(2,6)-dipyridina-1(1,2)-benzenacycloundecaphane 4,4-dioxide (124)

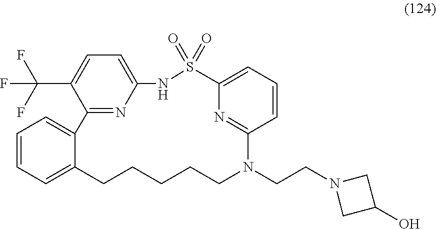 [SMILES: O=S1(=O)Nc2ccc(C(F)(F)F)c(n2)-c2ccccc2CCCCCN(CCN2CC(O)C2)c2cccc1n2]

6-(2-(3-hydroxyazetidin-1-yl)ethyl)-2³-(trifluoromethyl)-4-thia-3,6-diaza-2,5(2,6)-dipyridina-1(1,2)-benzenacycloundecaphane 4,4-dioxide (124) was synthesized using the procedure described in Example 121 except tert-butyl piperazine-1-carboxylate was replaced with tert-butyl 3-hydroxyazetidine-1-carboxylate. LCMS (Condition 1): m/z 562.3 [M+H]⁺, 1.43 min. ¹H NMR (400 MHz, DMSO-d₆) 11.67 (s, 1H), 8.00 (m, 1H), 7.68 (m, 1H), 7.34 (td, J=7.5, 1.5 Hz, 1H), 7.27 (m, 1H), 7.19 (m, 4H), 6.78 (d, J=8.7 Hz, 1H), 5.36 (s, 1H), 4.17 (m, 1H), 3.83 (m, 1H), 3.60 (m, 2H), 3.20 (m, 2H), 2.88 (m, 3H), 2.58 (m, 2H), 2.39 (m, 1H), 1.90 (m, 1H), 1.65 (m, 1H), 1.32 (m, 2H), 1.13 (m, 2H), 1.01 (m, 1H).

### Example 125: Synthesis of 6-(2-(4-methylpiperazin-1-yl)ethyl)-2³-(trifluoromethyl)-4-thia-3,6-diaza-2,5(2,6)-dipyridina-1(1,2)-benzenacycloundecaphane 4,4-dioxide 125)

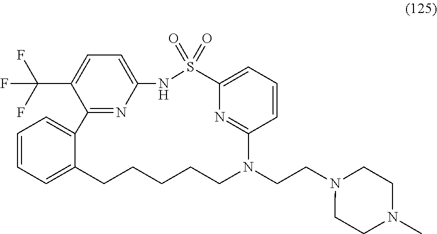 [SMILES: CN1CCN(CCN2CCCCCc3ccccc3-c3nc(ccc3C(F)(F)F)NS(=O)(=O)c3cccc2n3)CC1]

6-(2-(4-methylpiperazin-1-yl)ethyl)-2³-(trifluoromethyl)-4-thia-3,6-diaza-2,5(2,6)-dipyridina-1(1,2)-benzenacycloundecaphane 4,4-dioxide (125) was synthesized using the procedure described in Example 121 except tert-butyl piperazine-1-carboxylate was replaced with 1-methylpiperazine. LCMS (Condition 1): m/z 589.3 [M+H]⁺, 1.49 min. ¹H NMR (400 MHz, DMSO-d₆) δ 11.63 (brs, 1 h), 8.05-7.96 (m, 1H), 7.70 (dd, J=8.6, 7.3 Hz, 1H), 7.39-7.32 (m, 1H), 7.30-7.26 (m, 1H), 7.26-7.19 (m, 2H), 7.16-7.12 (m, 2H), 6.79 (d, J=8.7 Hz, 1H), 3.95-3.84 (m, 1H), 2.94-2.83 (m, 1H), 2.45-2.20 (m, 9H), 2.19 (s, 1H), 2.17 (s, 3H), 1.97-1.88 (m, 1H), 1.77-1.65 (m, 1H), 1.42-1.29 (m, 2H), 1.22-0.97 (m, 3H).

### Example 126: Synthesis of methyl (2-(4,4-dioxido-2³-(trifluoromethyl)-4-thia-3,6-diaza-2,5(2,6)-dipyridina-1(1,2)-benzenacycloundecaphane-6-yl)ethyl)glycinate (126)1

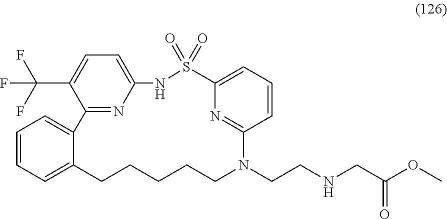 [SMILES: COC(=O)CNCCN1CCCCCc2ccccc2-c2nc(ccc2C(F)(F)F)NS(=O)(=O)c2cccc1n2]

methyl (2-(4,4-dioxido-2³-(trifluoromethyl)-4-thia-3,6-diaza-2,5(2,6)-dipyridina-1(1,2)-benzenacycloundecaphane-6-yl)ethyl)glycinate (126) was synthesized using the procedure described in Example 121 except tert-butylpiperazine-1-carboxylate was replaced with methyl 2-aminoacetate (purchased). LCMS (Condition 1): m/z 578.3 [M+H]⁺, 1.49 min.

### Example 127: Synthesis of 2-(4,4-dioxido-2³-(trifluoromethyl)-4-thia-3,6-diaza-2,5(2,6)-dipyridina-1(1,2)-benzenacycloundecaphane-6-yl)ethyl)glycine (127)

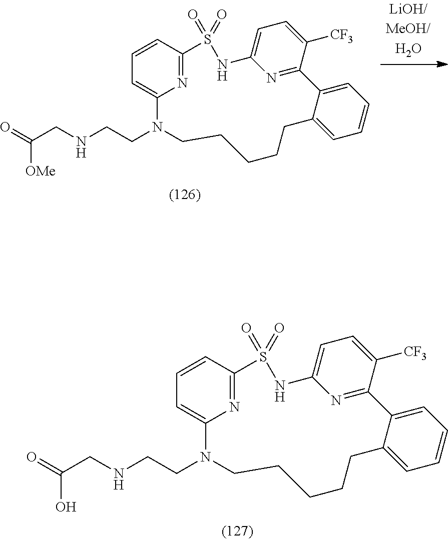 [SMILES: COC(=O)CNCCN1CCCCCc2ccccc2-c2nc(ccc2C(F)(F)F)NS(=O)(=O)c2cccc1n2 ; O=C(O)CNCCN1CCCCCc2ccccc2-c2nc(ccc2C(F)(F)F)NS(=O)(=O)c2cccc1n2]

A mixture of methyl (2-(4,4-dioxido-2³-(trifluoromethyl)-4-thia-3,6-diaza-2,5(2,6)-dipyridina-1(1,2)-benzenacycloundecaphane-6-yl)ethyl)glycinate (126) (15 mg, 0.026 mmol) and LiOH (3.2 mg, 0.13 mmol) in MeOH (3 mL) and water (1 mL). The mixture was stirred at room temperature for 90 min. The reaction volume was reduced in vacuo to remove MeOH. The mixture was then acidified with 1 N HCl and then extracted with DCM (mixed with 5-10% MeOH). The organic layer was collected, dried over MgSO₄, filtered, concentrated and dried under high vacuum heating at 70° C. for 1 h to afford (2-(4,4-dioxido-2³-(trifluoromethyl)-4-thia-3,6-diaza-2,5(2,6)-dipyridina-1(1,2)-benzenacycloundecaphane-6-yl)ethyl)glycine (127) as a white powder. LCMS (Condition 1): m/z 564.2 [M+H]⁺, 1.44 min. ¹H NMR (400 MHz, DMSO-d) δ 8.11 (d, J=9.0 Hz, 1H), 7.79 (dd, J=8.7, 7.3 Hz, 1H), 7.40-7.35 (m, 1H), 7.33-7.28 (m, 2H), 7.26-7.19 (m, 2H), 7.15 (d, J=7.6 Hz, 1H), 6.98 (d, J=8.7 Hz, 1H), 4.03-3.92 (m, 1H), 3.61-3.46 (m, 2H), 3.00-2.85 (m, 3H), 2.46-2.34 (m, 1H), 1.95-1.83 (m, 1H), 1.79-1.64 (m, 1H), 1.44-1.27 (m, 2H), 1.21-0.96 (m, 3H).

### Example 128: Synthesis of 6-(2,3-dihydroxypropyl)-2³-(trifluoromethyl)-4-thia-3,6-diaza-2,5(2,6)-dipyridina-1(1,2)-benzenacycloundecaphane 4,4-dioxide (128)

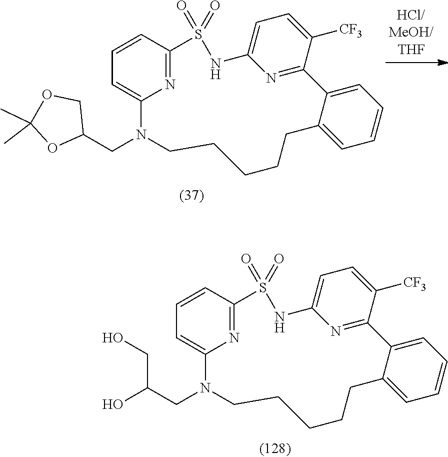 [SMILES: CC1(C)OCC(CN2CCCCCc3ccccc3-c3nc(ccc3C(F)(F)F)NS(=O)(=O)c3cccc2n3)O1 ; O=S1(=O)Nc2ccc(C(F)(F)F)c(n2)-c2ccccc2CCCCCN(CC(O)CO)c2cccc1n2]

To a solution of 6-((2,2-dimethyl-1,3-dioxolan-4-yl)methyl)-2³-(trifluoromethyl)-4-thia-3,6-diaza-2,5(2,6)-dipyridina-1(1,2)-benzenacycloundecaphane 4,4-dioxide (37) (34 mg, 0.059 mmol) in MeOH (2 mL) and THE (1 mL) 1 N HCl (1 mL, 1 mmol) was added and then the reaction was stirred at room temperature for 3 h. The solution was concentrated, and saturated NaHCO₃ solution was added. The resulting mixture was extracted with EtOAc (3×) and the combined extracts were dried over Na₂SO₄, filtered and concentrated. The residue was then purified on an ISCO silica gel column (EtOAc/heptane 0-100%) to give 6-(2,3-dihydroxypropyl)-2³-(trifluoromethyl)-4-thia-3,6-diaza-2,5(2,6)-dipyridina-1(1,2)-benzenacycloundecaphane 4,4-dioxide (128) as white crystals. LCMS (Condition 1): m/z 537.3 [M+H]⁺, 1.52 min. ¹H NMR (400 MHz, Methanol-d₄) δ 7.95 (d, J=8.9 Hz, 1H), 7.65 (dd, J=7.5, 8.5 Hz, 1H), 7.31 (m, 4H), 7.20 (td, J=1.1, 7.5 Hz, 1H), 7.11 (d, J=7.5 Hz, 1H), 6.93 (m, 1H), 4.14 (m, 1H), 3.81 (m, 1H), 3.23-3.55 (m, 4H), 3.03 (m, 1H), 2.43 (ddd, J=5.4, 9.9, 13.5 Hz, 1H), 2.03 (m, 1H), 1.81 (m, 1H), 1.51 (m, 2H), 1.24 (m, 3H). ¹⁹F NMR (376 MHz, Methanol-d₄) δ −59.57 (s), −59.62 (s). Diastereomers, 1:1 ratio.

### Example 129: Synthesis of 2³-(trifluoromethyl)-6-((2S,3S)-2,3,4-trihydroxybutyl)-4-thia-3,6-diaza-2.5(2.6)-dipyridina-1(1.2)-benzenacycloundecaphane 4,4-dioxide (129)

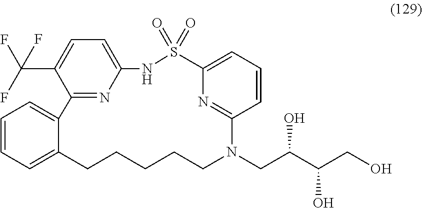 [SMILES: O=S1(=O)Nc2ccc(C(F)(F)F)c(n2)-c2ccccc2CCCCCN(C[C@H](O)[C@@H](O)CO)c2cccc1n2]

2³-(trifluoromethyl)-6-((2S,3S)-2,3,4-trihydroxybutyl)-4-thia-3,6-diaza-2,5(2,6)-dipyridina-1(1,2)-benzenacycloundecaphane 4,4-dioxide (129) was synthesized using the procedure described in Example 128 except 6-((2,2-dimethyl-1,3-dioxolan-4-yl)methyl)-2³-(trifluoromethyl)-4-thia-3,6-diaza-2,5(2,6)-dipyridina-1(1,2)-benzenacycloundecaphane 4,4-dioxide (37) was replaced with 6-(((4S,5S)-5-(hydroxymethyl)-2,2-dimethyl-1,3-dioxolan-4-yl)methyl)-2³-(trifluoromethyl)-4-thia-3,6-diaza-2,5(2,6)-dipyridina-1(1,2)-benzenacycloundecaphane 4,4-dioxide (34). LCMS (Condition 1): m/z 567.3 [M+H]⁺, 1.49 min. ¹H NMR (400 MHz, DMSO-d₆) δ 11.80 (s, 1H), 8.10 (d, J=8.9 Hz, 1H), 7.70 (m, 1H), 7.36 (m, 1H), 7.29 (d, J=7.5 Hz, 1H), 7.23 (m, 2H), 7.19 (d, J=8.9 Hz, 1H), 7.14 (d, J=7.4 Hz, 1H), 6.91 (m, 1H), 4.65 (m, 1H), 4.58 (m, 1H), 4.47 (m, 1H), 3.98 (m, 1H), 3.69 (m, 1H), 3.17-3.48 (m, 5H), 2.95 (m, 1H), 2.39 (m, 1H), 1.91 (m, 1H), 1.76 (m, 1H), 1.35 (m, 2H), 1.10 (m, 3H). ¹⁹F NMR (376 MHz, DMSO-d₆) δ−56.78 (s), −56.81 (s). Diastereomers, 1:1 ratio.

### Example 130: Synthesis of 6-(2-aminoethyl)-2³-(trifluoromethyl)-4-thia-3,6-diaza-2,5(2,6)-dipyridina-1(1,2)-benzenacyclodecaphane 4,4-dioxide (130) and 23-(trifluoromethyl)-4-thia-3,6,9-triaza-2,5(2,6)-dipyridina-1(1,2)-benzenacyclotridecaphane 4,4-dioxide (131)

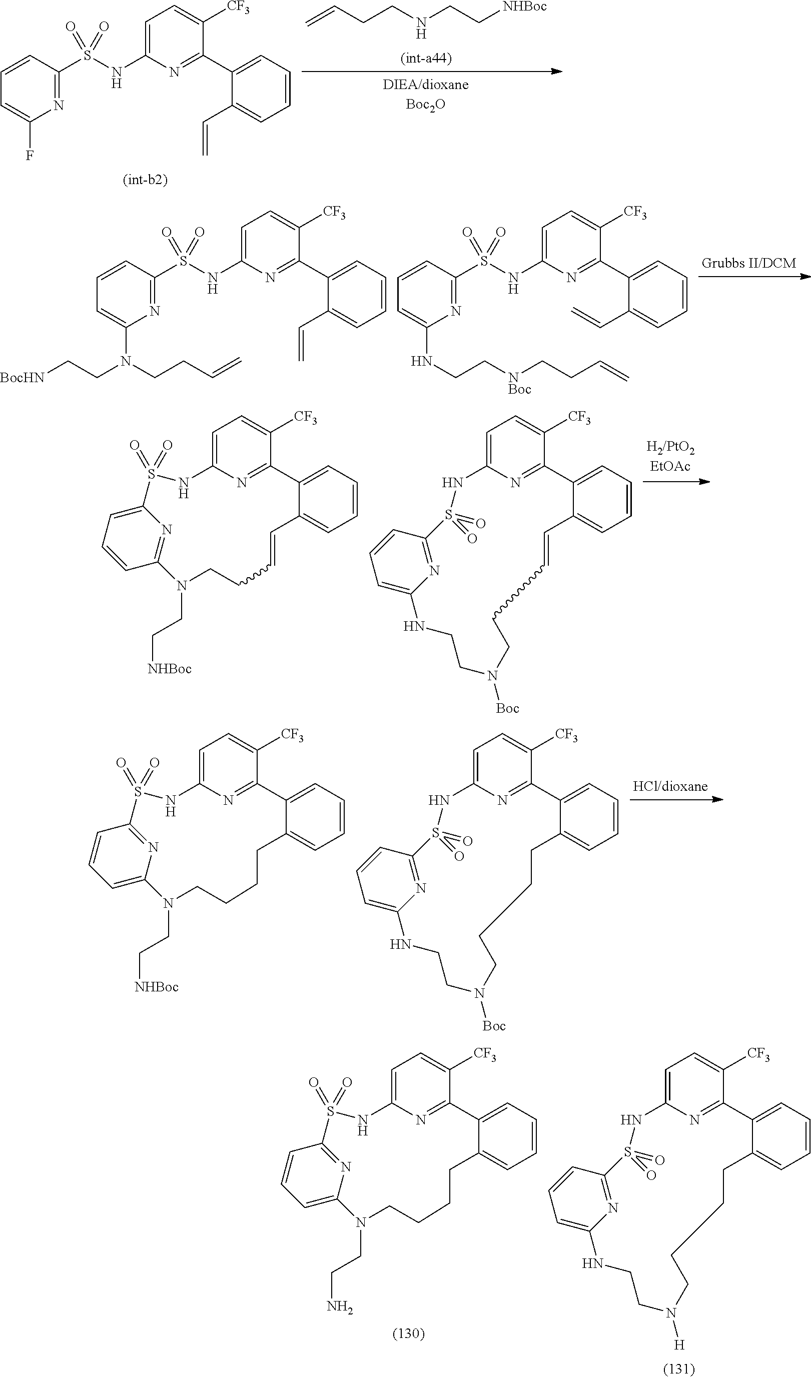

### Step 1

A solution of 6-fluoro-N-(5-(trifluoromethyl)-6-(2-vinylphenyl)pyridin-2-yl)pyridine-2-sulfonamide (int-b2) (100 mg, 0.236 mmol) and tert-butyl (2-(but-3-en-1-ylamino)ethyl)carbamate (int-a43) (93 mg, 0.283 mmol) (TFA salt) and DIEA (206 μL, 1.2 mmol) in dioxane (3 mL) was heated overnight at 110° C. Afterwards additional 200 mg more amine and 800 μL DIEA was added. The mixture was continued stirring for additional 2 days. Then to the crude reaction mixture was added Boc₂O (100 mg) and stirred for 30 min. The mixture was extracted with EtOAc and the combined extracts were washed with water and 1N HCl. The organic layer was collected, dried over MgSO₄, filtered and concentrated. The residue was purified using an ISCO column (EtOAc/heptane 0-100%) to afford a mixture of two products. LCMS (Condition 1): m/z 618.3 [M+H]⁺, 1.83 min.

### Step 2

A mixture of the starting material (130 mg, 0.21 mmol) in DCM (100 mL) was added Grubbs II catalyst (50 mg, 0.059 mmol) under nitrogen atmosphere and then heated overnight in an oil bath at 55° C. The mixture was concentrated in vacuo and the residue was purified by an ISCO column (EtOAc/heptane 0-100%) to afford a mixture of cyclized products as brown solids. LCMS (Condition 1): m/z 590.3 [M+H]⁺, 1.77 min.

### Step 3

A mixture of the starting material (100 mg, 0.17 mmol) in EtOAc (15 mL) was hydrogenated over PtO₂ hydrate (50 mg, 0.220 mmol) at room temperature for 2 days. The mixture was filtered through Celite, rinsed with EtOAc, and then the filtrate was concentrated. The residue was purified on an ISCO column (EtOAc/hexane 0-100%) to afford tert-butyl (2-(4,4-dioxido-2³-(trifluoromethyl)-4-thia-3,6-diaza-2,5(2,6)-dipyridina-1(1,2)-benzenacyclodecaphane-6-yl)ethyl)carbamate (35 mg, 0.059 mmol, 35% yield) (Peak 1) as a white solid and tert-butyl 2³-(trifluoromethyl)-4-thia-3,6,9-triaza-2,5(2,6)dipyridina-1(1,2)-benzenacyclotridecaphane-9-carboxylate 4,4-dioxide (35 mg, 0.059 mmol, 35% yield) (Peak 2) as a white solid. Structures are tentatively assigned. Peak 1: LCMS (Condition 1): m/z 592.3 [M+H]⁺, 1.80 min; Peak 2: LCMS (Condition 1): m/z 592.3 [M+H]⁺, 1.76 min.

### Step 4. Synthesis of 6-(2-aminoethyl)-2³-(trifluoromethyl)-4-thia-3,6-diaza-2,5(2,6)-dipyridina-1(1,2)-benzenacyclodecaphane 4,4-dioxide (130)

To Peak 1 (35 mg, 0.059 mmol) was added 4M HCl in dioxane (2 mL, 8.00 mmol) and the mixture was stirred at room temperature for 1 h. The mixture was precipitated by addition of ether and stirred overnight. The solids were filtered and dried under high vacuum to afford 6-(2-aminoethyl)-2³-(trifluoromethyl)-4-thia-3,6-diaza-2,5(2,6)-dipyridina-1(1,2)-benzenacyclodecaphane 4,4-dioxide (130) as a white solid (HCl salt). LCMS (Condition 1): m/z 492.2 [M+H]⁺, 1.41 min. ¹H NMR (400 MHz, DMSO-d₆) δ 11.71 (s, 1H), 8.17 (d, J=8.9 Hz, 1H), 7.94-7.87 (m, 3H), 7.79 (dd, J=8.6, 7.4 Hz, 1H), 7.39-7.33 (m, 2H), 7.30-7.20 (m, 4H), 7.00 (d, J=8.7 Hz, 1H), 3.92 (brs, 1H), 3.45-3.25 (m, 1H), 3.06-2.86 (m, 3H), 2.55-2.45 (m, 2H), 1.75 (t, J=10.8 Hz, 1H), 1.61-1.56 (m, 1H), 1.42-1.20 (m, 2H), 0.72 (s, 1H).

### Step 4. Synthesis 2³-(trifluoromethyl)-4-thia-3,6,9-triaza-2,5(2,6)-dipyridina-1(1,2)-benzenacyclotridecaphane 4,4-dioxide (131)

To Peak 2 (35.0 mg, 0.059 mmol) was added 4M HCl in dioxane (2 mL, 8.00 mmol) and the mixture was stirred at room temperature for 1 h. The mixture was precipitated by addition of ether and stirred overnight. The solids were filtered and dried under high vacuum to afford 2³-(trifluoromethyl)-4-thia-3,6,9-triaza-2,5(2,6)-dipyridina-1(1,2)-benzenacyclotridecaphane 4,4-dioxide (131) as a white solid (HCl salt). LCMS (Condition 1): m/z 492.2 [M+H]⁺, 1.35 min. ¹H NMR (400 MHz, DMSO-d₆) δ 11.65 (s, 1H), 8.57-8.47 (m, 1H), 8.38-8.28 (m, 1H), 8.18 (d, J=9.0 Hz, 1H), 7.70 (dd, J=8.5, 7.2 Hz, 1H), 7.46-7.35 (m, 2H), 7.36-7.22 (m, 4H), 7.17 (d, J=7.5 Hz, 1H), 6.79 (d, J=8.5 Hz, 1H), 3.95-3.80 (m, 1H), 3.37-3.26 (m, 1H), 3.12-3.02 (m, 1H), 2.98-2.86 (m, 1H), 2.71-2.60 (m, 2H), 2.45-2.39 (m, 1H), 1.96-1.87 (m, 1H), 1.63-1.52 (m, 1H), 1.45-1.28 (m, 3H).

### Example 131: Synthesis of 6-(2-aminoethyl)-2³-(trifluoromethyl)-4-thia-3,6-diaza-2,5(2,6)-dipyridina-1(1,2)-benzenacycloundecaphane 4,4-dioxide (132)

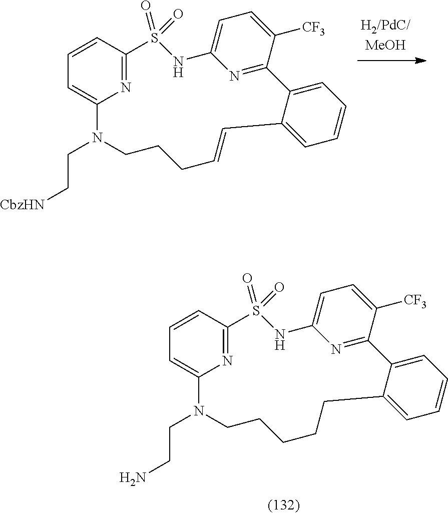 [SMILES: O=C(NCCN1CCC/C=C/c2ccccc2-c2nc(ccc2C(F)(F)F)NS(=O)(=O)c2cccc1n2)OCc1ccccc1 ; NCCN1CCCCCc2ccccc2-c2nc(ccc2C(F)(F)F)NS(=O)(=O)c2cccc1n2]

A solution of benzyl (E)-(2-(4,4-dioxido-2³-(trifluoromethyl)-4-thia-3,6-diaza-2,5(2,6)-dipyridina-1(1,2)-benzenacycloundecaphan-10-en-6-yl)ethyl)carbamate (80 mg, 0.125 mmol) in MeOH (15 mL) was hydrogenated over Pd/C (10 wt. %) (100 mg, 0.09 mmol) at room temperature for 5 h. The mixture was filtered through celite, rinsed with MeOH, and the filtrate was concentrated. The residue was purified using an ISCO reverse phase column (C18 column, H₂O/ACN gradient) to afford 6-(2-aminoethyl)-2³-(trifluoromethyl)-4-thia-3,6-diaza-2,5(2,6)-dipyridina-1(1,2)-benzenacycloundecaphane 4,4-dioxide (132) as a white solid. LCMS (Condition 1): m/z 506.3 [M+H]⁺, 1.38 min. ¹H NMR (400 MHz, DMSO-d₆) δ 8.91 (brs, 1H), 7.60-7.53 (m, 2H), 7.32 (s, 1H), 7.30-7.21 (m, 2H), 7.13-7.08 (m, 1H), 7.04-7.00 (m, 1H), 6.77 (d, J=7.5 Hz, 1H), 6.72 (d, J=8.7 Hz, 1H), 6.64 (d, J=8.3 Hz, 1H), 3.45-3.25 (m, 2H), 2.95-2.88 (m, 1H), 2.85-2.63 (m, 3H), 2.55-2.45 (m, 1H), 2.15-2.04 (m, 1H), 1.71-1.60 (m, 1H), 1.46-1.35 (m, 1H), 1.30-1.19 (m, 3H), 1.16-1.03 (m, 2H).

### Example 132: Synthesis of 2-(3-(4,4-dioxido-2³-(trifluoromethyl)-4-thia-3,6-diaza-2,5(2,6)-dipyridina-1(1.2)-benzenacycloundecaphane-6-yl)propyl)isoindoline-1,3-dione (133) and 2-(3-(4,4-dioxido-2³-(trifluoromethyl)-4-thia-3,6-diaza-2,5(2,6)-dipyridina-1(1,2)-benzenacycloundecaphane-6-yl)propyl)hexahydro-1H-isoindole-1,3(2H)-dione (134)

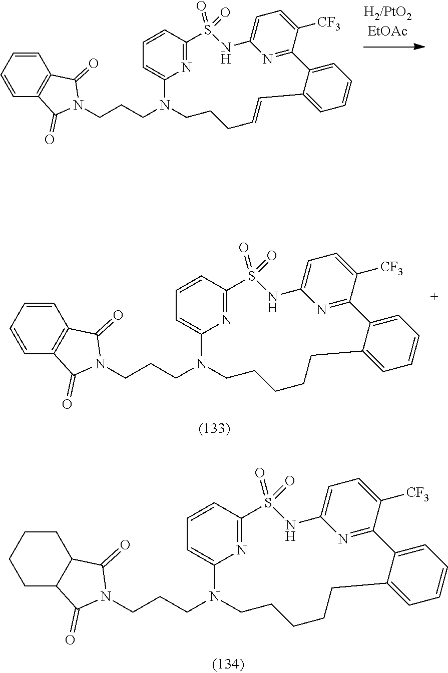 [SMILES: O=C1c2ccccc2C(=O)N1CCCN1CCC/C=C/c2ccccc2-c2nc(ccc2C(F)(F)F)NS(=O)(=O)c2cccc1n2 ; O=C1c2ccccc2C(=O)N1CCCN1CCCCCc2ccccc2-c2nc(ccc2C(F)(F)F)NS(=O)(=O)c2cccc1n2 ; O=C1C2CCCCC2C(=O)N1CCCN1CCCCCc2ccccc2-c2nc(ccc2C(F)(F)F)NS(=O)(=O)c2cccc1n2]

A solution of (E)-2-(3-(4,4-dioxido-2³-(trifluoromethyl)-4-thia-3,6-diaza-2,5(2,6)-dipyridina-1(1,2)-benzenacycloundecaphan-10-en-6-yl)propyl)isoindoline-1,3-dione (102 mg, 0.157 mmol) in EtOAc (10 mL) was hydrogenated overnight over PtO₂ hydrate (10 mg, 0.04 mmol) with a hydrogen balloon at room temperature. The catalyst was filtered off and the filtrate was concentrated. Purification of residue on an ISCO silica gel column (EtOAc/heptane 0-45%) gave two products: 2-(3-(4,4-dioxido-2³-(trifluoromethyl)-4-thia-3,6-diaza-2,5(2,6)-dipyridina-1(1,2)-benzenacycloundecaphane-6-yl)propyl)isoindoline-1,3-dione (133) as white crystals. Conditions 1, LCMS: m/z 650.2 [M+H]⁺, 1.82 min, ¹H NMR (400 MHz, DMSO-d₆) δ 11.78 (s, 1H), 8.10 (d, J=8.9 Hz, 1H), 7.85 (m, 4H), 7.72 (dd, J=8.7, 7.3 Hz, 1H), 7.36 (td, J=7.5, 1.5 Hz, 1H), 7.29 (m, 1H), 7.22 (m, 3H), 7.14 (d, J=7.6 Hz, 1H), 6.88 (d, J=8.8 Hz, 1H), 3.96 (m, 1H), 3.62 (t, J=7.1 Hz, 2H), 3.36 (m, 2H), 2.90 (m, 1H), 2.39 (m, 1H), 1.80 (m, 4H), 1.31 (m, 2H), 1.14 (d, J=10.1 Hz, 2H), 1.01 (m, 1H).

2-(3-(4,4-dioxido-2³-(trifluoromethyl)-4-thia-3,6-diaza-2,5(2,6)-dipyridina-1(1,2)-benzenacycloundecaphane-6-yl)propyl)hexahydro-1H-isoindole-1,3(2H)-dione (134) as a white solid. Condition 1, LCMS: m/z 656.2 [M+H]⁺, 1.95 min, ¹H NMR (400 MHz, DMSO-d₆) δ 11.79 (s, 1H), 8.09 (d, J=8.9 Hz, 1H), 7.73 (dd, J=8.7, 7.2 Hz, 1H), 7.36 (td, J=7.5, 1.5 Hz, 1H), 7.29 (m, 1H), 7.22 (m, 3H), 7.14 (d, J=7.6 Hz, 1H), 6.82 (d, J=8.8 Hz, 1H), 3.95 (m, 1H), 3.40 (t, J=7.1 Hz, 2H), 3.24 (m, 2H), 2.91 (m, 3H), 2.39 (m, 1H), 1.88 (m, 1H), 1.70 (m, 5H), 1.55 (m, 2H), 1.38 (m, 4H), 1.25 (m, 2H), 1.12 (m, 2H), 1.01 (m, 1H). ¹⁹F NMR (376 MHz, DMSO-d₆) δ−56.90 (s).

### Example 133: Synthesis of (R)-3-(4,4-dioxido-2³-(trifluoromethyl)-4-thia-3,6-diaza-2,5(2,6)-dipyridina-1(1,2)-benzenacycloundecaphane-7-yl)propanoic acid (135)

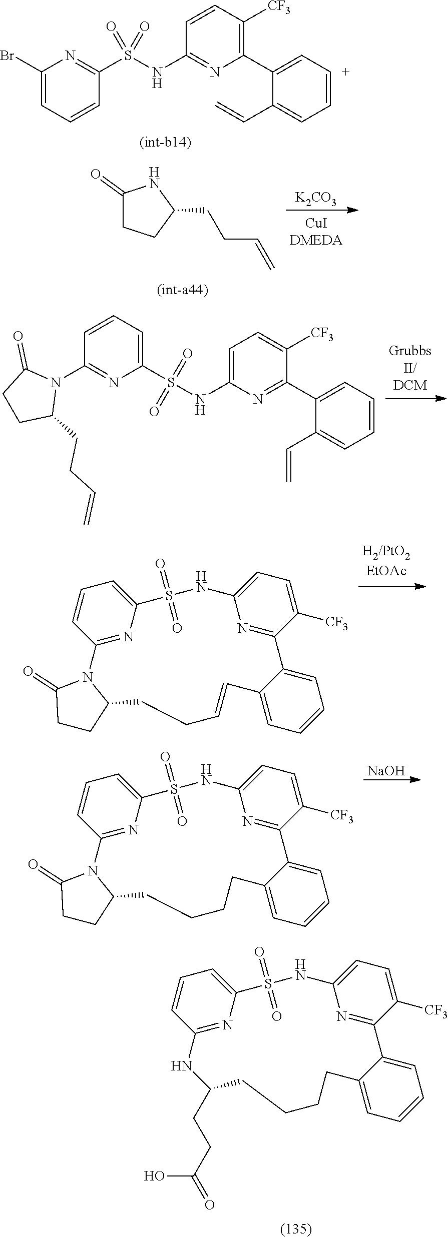

Step 1. A mixture of 6-bromo-N-(5-(trifluoromethyl)-6-(2-vinylphenyl)pyridin-2-yl)pyridine-2-sulfonamide (int-b14) (102 mg, 0.211 mmol), (R)-5-(but-3-en-1-yl)pyrrolidin-2-one (int-a44) (44.0 mg, 0.316 mmol), N1,N2-dimethylethane-1,2-diamine (DMEDA) (5.57 mg, 0.063 mmol), copper(I) iodide (1.805 mg, 9.48 μmol) and potassium carbonate (58.2 mg, 0.421 mmol) in toluene (2 mL) was degassed and then stirred at 120° C. for 38 hours. The reaction mixture was then diluted with EtOAc (50 mL), washed water (2×5 mL), and then brine (5 mL) and then extracted. The aqueous was back extracted with EtOAc (20 mL) and the EtOAc phases were combined, dried over Na₂SO₄ and evaporated. The residue was purified by flash chromatography (EtOAc:hex/0-50%) to give (R)-6-(2-(but-3-en-1-yl)-5-oxopyrrolidin-1-yl)-N-(5-(trifluoromethyl)-6-(2-vinylphenyl)pyridin-2-yl)pyridine-2-sulfonamide: MS 543.1 [M+H]⁺, rt=1.49 min.

Step 2. A solution of (S)-6-(2-(but-3-en-1-yl)-5-oxopyrrolidin-1-yl)-N-(5-(trifluoromethyl)-6-(2-vinylphenyl)pyridin-2-yl)pyridine-2-sulfonamide (89 mg, 0.164 mmol) and Grubbs II (13.93 mg, 0.016 mmol) in dichloroethane (15 mL) was purged with argon. The mixture was then stirred for 18 h at 80° C. and the solvent was then evaporated and the residue purified by flash chromatography (EtOAC:hex/0-30%) to give the (R,E)-5⁵-(trifluoromethyl)-3-thia-4-aza-2,5(2,6)-dipyridina-1(1,2)-pyrrolidina-6(1,2)-benzenacyclodecaphan-7-en-15-one 3,3-dioxide as a white solid: MS 515.1 [M+H]⁺, rt=1.39 min

Step 3. A suspension of (R,E)-5⁵-(trifluoromethyl)-3-thia-4-aza-2,5(2,6)-dipyridina-1(1,2)-pyrrolidina-6(1,2)-benzenacyclodecaphan-7-en-15-one 3,3-dioxide (66 mg, 0.128 mmol), platinum(IV) oxide (5.83 mg, 0.026 mmol) in EtOAc (2 mL) was stirred under hydrogen (1 atm) overnight at room temperature. The reaction mixture was then filtered through a syringe filter and the solvent from the filtrate was evaporated. The resulting residue was purified by flash chromatography (12 g silica gel column, EtOAc:hex/20-50%) to give (R)-5⁵-(trifluoromethyl)-3-thia-4-aza-2,5(2,6)-dipyridina-1(1,2)-pyrrolidina-6(1,2)-benzenacyclodecaphan-15-one 3,3-dioxide as a white solid. LCMS: m/z 517.1 [M+H]⁺, 1.43 min; 1H NMR (400 MHz, Methylene Chloride-d₂) δ 8.57 (dd, J=8.5, 0.8 Hz, 1H), 8.01 (d, J=8.8 Hz, 1H), 7.89 (dd, J=8.5, 7.5 Hz, 1H), 7.75 (dd, J=7.5, 0.9 Hz, 1H), 7.66 (dd, J=7.9, 1.2 Hz, 1H), 7.59 (s, 1H), 7.49-7.41 (m, 1H), 7.31 (td, J=7.5, 1.3 Hz, 1H), 7.11 (d, J=7.7 Hz, 1H), 6.12 (dd, J=17.4, 11.0 Hz, 1H), 5.77 (ddt, J=16.9, 10.2, 6.6 Hz, 1H), 5.61 (dd, J=17.4, 1.0 Hz, 1H), 5.06 (dd, J=11.0, 1.0 Hz, 1H), 5.02-4.89 (m, 2H), 4.57 (s, 1H), 2.71 (ddd, J=17.7, 10.2, 9.4 Hz, 1H), 2.49 (ddd, J=17.7, 9.7, 3.2 Hz, 1H), 2.22-2.06 (m, 1H), 2.06-1.94 (m, 3H), 1.87 (dtd, J=16.1, 8.3, 7.6, 2.8 Hz, 2H), 1.50 (dq, J=14.2, 7.6 Hz, 1H). LCMS: m/z 517.1 [M+H]⁺, 1.43 min; 1H NMR (400 MHz, Methanol-d4) δ 8.43 (dd, J=7.1, 2.3 Hz) and 8.32 (d, J=8.4 Hz, 1H), 8.15-7.98 (m, 2H), 7.93 (d, J=8.9 Hz) and 7.81 (d, J=7.6 Hz, 1H), 7.35 (td, J=7.5, 1.5 Hz, 1H), 7.27 (dd, J=7.7, 1.3 Hz, 1H), 7.25-7.13 (m, 2H), 7.11 (d, J=7.6 Hz, 1H), 4.67-4.49 (m, 1H), 2.74-2.58 (m, 1H), 2.58-2.43 (m, 1H), 2.40-2.01 (m, 3H), 1.99-1.83 (m, 2H), 1.84-1.70 (m, 1H), 1.70-1.53 (m, 1H), 1.48-1.27 (m, 2H), 1.19 (br s, 1H); 19F NMR (376 MHz, Methanol-d4) δ −58.61 (minor atropisomer), −59.54 (major atropisomer).

Step 4. A solution of (R)-5⁵-(trifluoromethyl)-3-thia-4-aza-2,5(2,6)-dipyridina-1(1,2)-pyrrolidina-6(1,2)-benzenacyclodecaphan-15-one 3,3-dioxide (30 mg, 0.058 mmol) in NaOH solution (1 N, 1.2 mL) was stirred at 80° C. for 12 h. and then acidified with 1 N HCl to pH-5 and extracted with EtOAc (4×50 mL). The EtOAc phase was dried over Na₂SO₄ and then evaporated. The resulting residue was purified by flash chromatography (EtOAc:heptane/30-50%) to give (R)-3-(4,4-dioxido-23-(trifluoromethyl)-4-thia-3,6-diaza-2,5(2,6)-dipyridina-1(1,2)-benzenacycloundecaphane-7-yl)propanoic acid (135) as a white solid. MS: 535.2 [M+H]⁺, 1.41 min; 1H NMR (400 MHz, Methylene Chloride-d₂+CD₃OD) (major atropisomer) δ 7.87 (d, J=8.9 Hz, 1H), 7.46 (dd, J=8.5, 7.2 Hz, 1H), 7.42-7.34 (m, 2H), 7.31-7.26 (m, 2H), 7.23 (td, J=7.4, 1.3 Hz, 1H), 7.11 (d, J=7.6 Hz, 1H), 6.51 (dd, J=8.5, 0.8 Hz, 1H), 4.34 (tt, J=9.4, 4.5 Hz, 1H), 2.44-2.24 (m, 3H), 1.95-1.75 (m, 2H), 1.69 (m, 1H), 1.58 (m, 1H), 1.43 (dq, J=9.4, 4.5 Hz, 2H), 1.37-1.21 (m, 2H), 1.15 (m, 1H). 19F NMR (376 MHz, Methylene Chloride-d₂+CD₃OD) δ −58.69, −59.41

### Administration and Pharmaceutical Compositions

For the therapeutic uses of compounds of the present invention, such compounds are administered either alone or as part of a pharmaceutical composition. Accordingly, in another aspect of the present invention provides a pharmaceutical composition, which comprises a compound of the present invention, or pharmaceutically acceptable salt thereof, and one or more pharmaceutically acceptable carriers. In a further embodiment, the composition comprises at least two pharmaceutically acceptable carriers, such as those described herein. The pharmaceutical composition can be formulated for particular routes of administration such as oral administration, parenteral administration (e.g. by injection, infusion, transdermal or topical administration), and rectal administration. Topical administration may also pertain to inhalation or intranasal application. In certain embodiments, the pharmaceutical composition comprising a compound of the present invention can be formulated for intramuscularly, intravenously, subcutaneously, orally, pulmonary, intrathecally, topically or intranasally administration.

The pharmaceutical compositions of the present invention can be made up in a solid form (including without limitation capsules, tablets, pills, granules, powders or suppositories), or in a liquid form (including without limitation solutions, suspensions or emulsions). Tablets may be either film coated or enteric coated according to methods known in the art.

Typically, the pharmaceutical compositions are tablets or gelatin capsules comprising the active ingredient together with
a) diluents, e.g., lactose, dextrose, sucrose, mannitol, sorbitol, cellulose and/or glycine;
b) lubricants, e.g., silica, talcum, stearic acid, its magnesium or calcium salt and/or polyethyleneglycol; for tablets also
c) binders, e.g., magnesium aluminum silicate, starch paste, gelatin, tragacanth, methylcellulose, sodium carboxymethylcellulose and/or polyvinylpyrrolidone; if desired
d) disintegrants, e.g., starches, agar, alginic acid or its sodium salt, or effervescent mixtures; and/or
e) absorbents, colorants, flavors and sweeteners.

Suitable compositions for oral administration include a compound of the present invention in the form of tablets, lozenges, aqueous or oily suspensions, dispersible powders or granules, emulsion, hard or soft capsules, or syrups or elixirs. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets may contain the active ingredient in admixture with nontoxic pharmaceutically acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

The parenteral compositions (e.g, intravenous (IV) formulation) are aqueous isotonic solutions or suspensions. The parenteral compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. The compositions are generally prepared according to conventional mixing, granulating or coating methods, respectively, and contain about 0.1-75%, or contain about 1-50%, of the active ingredient.

The compound of the present invention or pharmaceutical composition thereof for use in a subject (e.g., human) is typically administered orally or parenterally at a therapeutic dose of less than or equal to about 100 mg/kg, 75 mg/kg, 50 mg/kg, 25 mg/kg, 10 mg/kg, 7.5 mg/kg, 5.0 mg/kg, 3.0 mg/kg, 1.0 mg/kg, 0.5 mg/kg, 0.05 mg/kg or 0.01 mg/kg, but preferably not less than about 0.0001 mg/kg. When administered intravenously via infusion, the dosage may depend upon the infusion rate at which an iv formulation is administered. In general, the therapeutically effective dosage of a compound, the pharmaceutical composition, or the combinations thereof, is dependent on the species of the subject, the body weight, age and individual condition, the disorder or disease or the severity thereof being treated. A physician, pharmacist, clinician or veterinarian of ordinary skill can readily determine the effective amount of each of the active ingredients necessary to prevent, treat or inhibit the progress of the disorder or disease.

The above-cited dosage properties are demonstrable in vitro and in vivo tests using advantageously mammals, e.g., mice, rats, dogs, monkeys or isolated organs, tissues and preparations thereof. The compounds of the present invention can be applied in vitro in the form of solutions, e.g., aqueous solutions, and in vivo either enterally, parenterally, advantageously intravenously, e.g., as a suspension or in aqueous solution. The dosage in vitro may range between about 10-3 molar and 10-9 molar concentrations. A therapeutically effective amount in vivo may range depending on the route of administration, between about 0.1-500 mg/kg, or between about 1-100 mg/kg.

Certain aspects and examples of the pharmaceutical compositions of the present invention are provided in the following listing of enumerated embodiments. It will be recognized that features specified in each embodiment may be combined with other specified features to provide further embodiments of the present invention.

Embodiment 120. A pharmaceutical composition comprising a compound of any one of Embodiments 1 to 119, or a pharmaceutically acceptable salt thereof, and one or more pharmaceutically acceptable carriers.

Embodiment 121. A pharmaceutical composition comprising a compound of any one of Embodiments 118 to 119, or a pharmaceutically acceptable salt thereof, and one or more pharmaceutically acceptable carriers.

Embodiment 122. The pharmaceutical composition of Embodiment 120 or 121 comprising one or more additional pharmaceutical agents.

Embodiment 123. The pharmaceutical composition of Embodiment 122, wherein the additional pharmaceutical agent(s) is selected from a mucolytic agent, nebulized hypertonic saline, bronchodilator, an antibiotic, an anti-infective agent, a CFTR modulator, and an anti-inflammatory agent.

Embodiment 124. The pharmaceutical composition of Embodiment 122, wherein the one or more additional pharmaceutical agents is a CFTR modulator.

Embodiment 125. The pharmaceutical composition of Embodiment 122, wherein the one or more additional pharmaceutical agents a CFTR corrector.

Embodiment 126. The pharmaceutical composition of Embodiment 122, wherein the one or more additional pharmaceutical agents is a CFTR potentiator.

Embodiment 127. The pharmaceutical composition of Embodiment 122, wherein the one or more additional pharmaceutical agent is a CFTR modulator and a CFTR potentiator.

Embodiment 128. The pharmaceutical composition of Embodiment 122, wherein the one or more additional pharmaceutical agent is a CFTR corrector and a CFTR potentiator.

### Pharmacology and Utility

Compounds of the present invention have been found to modulate CFTR activity and may be beneficial for the treatment of cystic fibrosis and additional diseases not directly caused by mutations in CFTR, such as secretory diseases and other protein folding diseases mediated by CFTR. These include, but are not limited to, chronic obstructive pulmonary disease (COPD), dry eye disease, and Sjogren's Syndrome.

COPD is characterized by airflow limitation that is progressive and not fully reversible. The airflow limitation is due to mucus hypersecretion, emphysema, and bronchiolitis. Activators of mutant or wild-type CFTR offer a potential treatment of mucus hypersecretion and impaired mucociliary clearance that is common in COPD. Specifically, increasing anion secretion across CFTR may facilitate fluid transport into the airway surface liquid to hydrate the mucus and optimized periciliary fluid viscosity. This would lead to enhanced mucociliary clearance and a reduction in the symptoms associated with COPD.

Dry eye disease is characterized by a decrease in tear aqueous production and abnormal tear film lipid, protein and mucin profiles. There are many causes of dry eye, some of which include age, Lasik eye surgery, arthritis, medications, chemical/thermal burns, allergies, and diseases, such as cystic fibrosis and Sjogrens's syndrome. Increasing anion secretion via CFTR would enhance fluid transport from the corneal endothelial cells and secretory glands surrounding the eye to increase corneal hydration. This would help to alleviate the symptoms associated with dry eye disease.

Sjogrens's syndrome is an autoimmune disease in which the immune system attacks moisture-producing glands throughout the body, including the eye, mouth, skin, respiratory tissue, liver, vagina, and gut. Symptoms, include, dry eye, mouth, and vagina, as well as lung disease. The disease is also associated with rheumatoid arthritis, systemic lupus, systemic sclerosis, and polymypositis/dermatomyositis. Defective protein trafficking is believed to cause the disease, for which treatment options are limited. Augmenters or inducers of CFTR activity may hydrate the various organs afflicted by the disease and help to elevate the associated symptoms.

The compounds of the present invention, in free form or in pharmaceutically acceptable salt form, exhibit valuable pharmacological properties, e.g. CFTR modulating properties, as indicated by the in vitro tests provided herein, and are therefore indicated for therapy or for use as research chemicals, e.g. as tool compounds.

Compounds of the present invention may be useful in the treatment of cystic fibrosis, chronic bronchitis, recurrent bronchitis, acute bronchitis, male infertility caused by congenital bilateral absence of the vas deferens (CBAVD), female infertility caused by congenital absence of the uterus and vagina (CAUV), chronic rhinosinusitis, primary sclerosing cholangitis, allergic bronchopulmonary aspergillosis, diabetes, dry eye, constipation, allergic bronchopulmonary aspergillosis (ABPA), bone diseases (e.g., osteoporosis), asthma, chronic obstructive pulmonary disease (COPD), chronic bronchitis or dyspnea associated therewith, recurrent bronchitis, acute bronchitis, rhinosinusitis, constipation, pancreatitis including idiopathic chronic pancreatitis (ICP), idiopathic recurrent pancreatitis, idiopathic acute pancreatitis, chronic pancreatitis, recurrent pancreatitis, acute pancreatitis and pancreatic insufficiency, male infertility caused by congenital bilateral absence of the vas deferens (CBAVD), mild pulmonary disease, idiopathic pancreatitis, liver disease, emphysema, hereditary emphysema, gallstones, gastro-esophageal reflux disease, gastrointestinal malignancies, inflammatory bowel disease, constipation, diabetes, arthritis, osteoporosis, osteopenia, dry eye disease, and Sjogren's Syndrome.

Compounds of the present invention may be useful in the treatment of pancreatitis, including idiopathic chronic pancreatitis (ICP), idiopathic recurrent pancreatitis, idiopathic acute pancreatitis, chronic pancreatitis, recurrent pancreatitis, acute pancreatitis and pancreatic insufficiency.

Thus, as a further aspect, the present invention provides the use of a compound of the present invention, or pharmaceutically acceptable salt thereof, in therapy. In a further embodiment, the therapy is selected from a disease which may be treated by modulation of CFTR activity. In another embodiment, the disease is selected from the afore-mentioned list, suitably cystic fibrosis, asthma, COPD, chronic bronchitis and emphysema, more suitably cystic fibrosis, asthma, COPD and chronic bronchitis, more suitably cystic fibrosis, COPD and emphysema, even more suitably cystic fibrosis.

Thus, as a further aspect, the present invention provides a compound of the present invention or pharmaceutically acceptable salt thereof, for use in therapy. In a further embodiment, the therapy is selected from a disease which may be treated by modulation of CFTR activity. In another embodiment, the disease is selected from the afore-mentioned list, suitably cystic fibrosis, asthma, COPD, chronic bronchitis and emphysema, more suitably cystic fibrosis, asthma, COPD and chronic bronchitis, more suitably cystic fibrosis, COPD and emphysema, even more suitably cystic fibrosis.

Thus, as a further aspect, the present invention provides the use of a compound of the present invention, or pharmaceutically acceptable salt thereof, in therapy for the treatment of pancreatitis, including idiopathic chronic pancreatitis (ICP), idiopathic recurrent pancreatitis, idiopathic acute pancreatitis, chronic pancreatitis, recurrent pancreatitis, acute pancreatitis and pancreatic insufficiency.

In another aspect, the invention provides a method of treating a disease which is treated by modulation of CFTR comprising administration of a therapeutically acceptable amount of a compound of the present invention, or pharmaceutically acceptable salt thereof. In further embodiment, the disease is selected from the afore-mentioned list, suitably cystic fibrosis, asthma, COPD, chronic bronchitis and emphysema, more suitably cystic fibrosis, asthma, COPD and chronic bronchitis, more suitably cystic fibrosis, COPD and emphysema, even more suitably cystic fibrosis.

In another aspect, the invention provides a method of treating pancreatitis, including idiopathic chronic pancreatitis (ICP), idiopathic recurrent pancreatitis, idiopathic acute pancreatitis, chronic pancreatitis, recurrent pancreatitis, acute pancreatitis and pancreatic insufficiency, wherein the method comprises administration of a therapeutically acceptable amount of a compound of the present invention, or pharmaceutically acceptable salt thereof.

Thus, as a further aspect, the present invention provides the use of a compound of the present invention, or pharmaceutically acceptable salt thereof, for the manufacture of a medicament. In a further embodiment, the medicament is for treatment of a disease which may be treated by modulation of CFTR. In another embodiment, the disease is selected from the afore-mentioned list, suitably cystic fibrosis, asthma, COPD, chronic bronchitis and emphysema, more suitably cystic fibrosis, asthma, COPD and chronic bronchitis, more suitably cystic fibrosis, COPD and emphysema, even more suitably cystic fibrosis.

Thus, as a further aspect, the present invention provides the use of a compound of the present invention, or pharmaceutically acceptable salt thereof, for the manufacture of a medicament for the treatment of pancreatitis, including idiopathic chronic pancreatitis (ICP), idiopathic recurrent pancreatitis, idiopathic acute pancreatitis, chronic pancreatitis, recurrent pancreatitis, acute pancreatitis and pancreatic insufficiency.

In one embodiment of the present invention, there is provided 4-(4,4-dioxido-2³-(trifluoromethyl)-4-thia-3,6-diaza-2,5(2,6)-dipyridina-1(1,2)-benzenacyclodecaphane-6-yl)butanoic acid for use in the treatment of cystic fibrosis, asthma, COPD, chronic bronchitis, pancreatitis and emphysema.

In one embodiment of the present invention, there is provided 4-(4,4-dioxido-2³-(trifluoromethyl)-4-thia-3,6-diaza-2,5(2,6)-dipyridina-1(1,2)-benzenacycloundecaphane-6-yl)butanoic acid for use in the treatment of cystic fibrosis, asthma, COPD, chronic bronchitis, pancreatitis and emphysema.

In one embodiment of the present invention, there is provided 4-(4,4-dioxido-2³-(trifluoromethyl)-4-thia-3,6-diaza-2,5(2,6)-dipyridina-1(1,2)-benzenacyclododecaphane-6-yl)butanoic acid for use in the treatment of cystic fibrosis, asthma, COPD, chronic bronchitis, pancreatitis and emphysema.

In one embodiment of the present invention, there is provided 4-(4,4-dioxido-2³-(trifluoromethyl)-4-thia-3,6-diaza-2,5(2,6)-dipyridina-1(1,2)-benzenacyclotridecaphane-6-yl)butanoic acid for use in the treatment of cystic fibrosis, asthma, COPD, chronic bronchitis, pancreatitis and emphysema.

In one embodiment of the present invention, there is provided 4-(4,4-dioxido-2³-(trifluoromethyl)-4-thia-3,6-diaza-2,5(2,6)-dipyridina-1(1,2)-benzenacyclotetradecaphane-6-yl)butanoic acid for use in the treatment of cystic fibrosis, asthma, COPD, chronic bronchitis, pancreatitis and emphysema.

In one embodiment of the present invention, there is provided 4-(4,4-dioxido-2³-(trifluoromethyl)-4-thia-3,6-diaza-2,5(2,6)-dipyridina-1(1,2)-benzenacyclopentadecaphane-6-yl)butanoic acid for use in the treatment of cystic fibrosis, asthma, COPD, chronic bronchitis, pancreatitis and emphysema.

In one embodiment of the present invention, there is provided 4-(4,4-dioxido-2³-(trifluoromethyl)-4-thia-3,6-diaza-2,5(2,6)-dipyridina-1(1,2)-benzenacyclodecaphane-6-yl)butanoic acid for use in the treatment of pancreatitis, including idiopathic chronic pancreatitis (ICP), idiopathic recurrent pancreatitis, idiopathic acute pancreatitis, chronic pancreatitis, recurrent pancreatitis, acute pancreatitis and pancreatic insufficiency.

In one embodiment of the present invention, there is provided 4-(4,4-dioxido-2³-(trifluoromethyl)-4-thia-3,6-diaza-2,5(2,6)-dipyridina-1(1,2)-benzenacycloundecaphane-6-yl)butanoic acid for use in the treatment of pancreatitis, including idiopathic chronic pancreatitis (ICP), idiopathic recurrent pancreatitis, idiopathic acute pancreatitis, chronic pancreatitis, recurrent pancreatitis, acute pancreatitis and pancreatic insufficiency.

In one embodiment of the present invention, there is provided 4-(4,4-dioxido-2³-(trifluoromethyl)-4-thia-3,6-diaza-2,5(2,6)-dipyridina-1(1,2)-benzenacyclododecaphane-6-yl)butanoic acid for use in the treatment of pancreatitis, including idiopathic chronic pancreatitis (ICP), idiopathic recurrent pancreatitis, idiopathic acute pancreatitis, chronic pancreatitis, recurrent pancreatitis, acute pancreatitis and pancreatic insufficiency.

In one embodiment of the present invention, there is provided 4-(4,4-dioxido-2³-(trifluoromethyl)-4-thia-3,6-diaza-2,5(2,6)-dipyridina-1(1,2)-benzenacyclotridecaphane-6-yl)butanoic acid for use in the treatment of pancreatitis, including idiopathic chronic pancreatitis (ICP), idiopathic recurrent pancreatitis, idiopathic acute pancreatitis, chronic pancreatitis, recurrent pancreatitis, acute pancreatitis and pancreatic insufficiency.

In one embodiment of the present invention, there is provided 4-(4,4-dioxido-2³-(trifluoromethyl)-4-thia-3,6-diaza-2,5(2,6)-dipyridina-1(1,2)-benzenacyclotetradecaphane-6-yl)butanoic acid for use in the treatment of pancreatitis, including idiopathic chronic pancreatitis (ICP), idiopathic recurrent pancreatitis, idiopathic acute pancreatitis, chronic pancreatitis, recurrent pancreatitis, acute pancreatitis and pancreatic insufficiency.

In one embodiment of the present invention, there is provided 4-(4,4-dioxido-2³-(trifluoromethyl)-4-thia-3,6-diaza-2,5(2,6)-dipyridina-1(1,2)-benzenacyclopentadecaphane-6-yl)butanoic acid for use in the treatment of pancreatitis, including idiopathic chronic pancreatitis (ICP), idiopathic recurrent pancreatitis, idiopathic acute pancreatitis, chronic pancreatitis, recurrent pancreatitis, acute pancreatitis and pancreatic insufficiency.

Another aspect of the present invention provides a method for treating or lessening the severity of a disease, disorder, or condition associated with the modulation of CFTR in a subject, which comprises administering to the subject a compound of the present invention, or a pharmaceutically acceptable salt thereof.

In certain embodiments, the present invention provides a method of treating a condition, disease, or disorder implicated by a deficiency of the CFTR activity, the method comprising administering a compound of the present invention to a subject in need of treatment thereof. In certain embodiments, the present invention provides a method of treating a condition, disease, or disorder implicated by a deficiency of the CFTR activity, the method comprising administering a composition comprising a compound of the present invention to a subject in need of treatment thereof.

In certain embodiments, the present invention provides a method of treating diseases associated with reduced CFTR function due to mutations in the gene encoding CFTR or environmental factors (e.g., smoke), the method comprising administering a compound of the present invention to a subject in need of treatment thereof. In certain embodiments, the present invention provides a method of treating diseases associated with reduced CFTR function due to mutations in the gene encoding CFTR or environmental factors (e.g., smoke), the method comprising administering a composition comprising a compound of the present invention to a subject in need of treatment thereof. The diseases associated with reduced CFTR function due to mutations in the gene encoding CFTR or environmental factors (e.g., smoke) include, cystic fibrosis, chronic bronchitis, recurrent bronchitis, acute bronchitis, male infertility caused by congenital bilateral absence of the vas deferens (CBAVD), female infertility caused by congenital absence of the uterus and vagina (CAUV), idiopathic chronic pancreatitis (ICP), idiopathic recurrent pancreatitis, idiopathic acute pancreatitis, chronic rhinosinusitis, primary sclerosing cholangitis, allergic bronchopulmonary aspergillosis, diabetes, dry eye, constipation, allergic bronchopulmonary aspergillosis (ABPA), bone diseases (e.g., osteoporosis), and asthma.

In certain embodiments, the present invention provides a method for treating diseases associated with normal CFTR function, the method comprising administering a compound of the present invention to a subject in need of treatment thereof. In certain embodiments, the present invention provides a method for treating diseases associated with normal CFTR function,), the method comprising administering a composition comprising a compound of the present invention to a subject in need of treatment thereof. The diseases associated with normal CFTR function include, chronic obstructive pulmonary disease (COPD), chronic bronchitis or dyspnea associated therewith, recurrent bronchitis, acute bronchitis, rhinosinusitis, constipation, pancreatitis including chronic pancreatitis, recurrent pancreatitis, acute pancreatitis and pancreatic insufficiency, male infertility caused by congenital bilateral absence of the vas deferens (CBAVD), mild pulmonary disease, idiopathic pancreatitis, liver disease, emphysema, hereditary emphysema, gallstones, gastro-esophageal reflux disease, gastrointestinal malignancies, inflammatory bowel disease, constipation, diabetes, arthritis, osteoporosis, and osteopenia.

According to an alternative preferred embodiment, the present invention provides a method of treating cystic fibrosis comprising administering to a subject in need of such treatment a compound of the present invention, or a pharmaceutically acceptable salt thereof. According to an alternative preferred embodiment, the present invention provides a method of treating cystic fibrosis comprising administering to a subject in need of such treatment a composition comprising a compound of the present invention, or a pharmaceutically acceptable salt thereof.

According to an alternative preferred embodiment, the present invention provides a method of treating chronic obstructive pulmonary disease (COPD) comprising administering to a subject in need of such treatment a compound of the present invention, or a pharmaceutically acceptable salt thereof. According to an alternative preferred embodiment, the present invention provides a method of treating chronic obstructive pulmonary disease (COPD) comprising administering to a subject in need of such treatment a composition comprising a compound of the present invention, or a pharmaceutically acceptable salt thereof.

According to an alternative preferred embodiment, the present invention provides a method of treating pancreatitis, including idiopathic chronic pancreatitis (ICP), idiopathic recurrent pancreatitis, idiopathic acute pancreatitis, chronic pancreatitis, recurrent pancreatitis, acute pancreatitis and pancreatic insufficiency, comprising administering to a subject in need of such treatment a compound of the present invention, or a pharmaceutically acceptable salt thereof. According to an alternative preferred embodiment, the present invention provides a method of treating pancreatitis, including idiopathic chronic pancreatitis (ICP), idiopathic recurrent pancreatitis, idiopathic acute pancreatitis, chronic pancreatitis, recurrent pancreatitis, acute pancreatitis and pancreatic insufficiency, comprising administering to a subject in need of such treatment a composition comprising a compound of the present invention, or a pharmaceutically acceptable salt thereof.

According to the invention an “effective dose” or an “effective amount” of the compound or pharmaceutical composition is that amount effective for treating or lessening the severity of one or more of the diseases, disorders or conditions as recited above.

The compounds and compositions, according to the methods of the present invention, may be administered using any amount and any route of administration effective for treating or lessening the severity of one or more of the diseases, disorders or conditions recited above.

In certain embodiments, the present invention relates to the aforementioned methods, wherein said compound is administered parenterally.

In certain embodiments, the present invention relates to the aforementioned methods, wherein said compound is administered intramuscularly, intravenously, subcutaneously, orally, pulmonary, intrathecally, topically or intranasally.

In certain embodiments, the present invention relates to the aforementioned methods, wherein said compound is administered systemically.

In certain embodiments, the present invention relates to the aforementioned methods, wherein said subject is a mammal.

In certain embodiments, the present invention relates to the aforementioned methods, wherein said subject is a primate.

In certain embodiments, the present invention relates to the aforementioned methods, wherein said subject is a human.

Certain aspects and examples of the use of compounds of the present invention and pharmaceutical compositions of the present invention are provided in the following listing of enumerated embodiments. It will be recognized that features specified in each embodiment may be combined with other specified features to provide further embodiments of the present invention.

Embodiment 129. A compound of any one of Embodiments 1-119, or a pharmaceutically acceptable salt thereof, for use in the treatment of a CFTR mediated disease which is selected cystic fibrosis, asthma, COPD, chronic bronchitis and emphysema.

Embodiment 130. A compound of any one of Embodiments 1-119, or a pharmaceutically acceptable salt thereof, for use in the treatment of a CFTR mediated disease which is selected from cystic fibrosis, asthma, COPD and chronic bronchitis.

Embodiment 131. A compound of any one of Embodiments 1-119, or a pharmaceutically acceptable salt thereof, for use in the treatment of a CFTR mediated disease which is selected from cystic fibrosis, COPD and emphysema.

Embodiment 132. A compound of any one of Embodiments 1-119, or a pharmaceutically acceptable salt thereof, for use in the treatment of a CFTR mediated disease which is cystic fibrosis.

Embodiment 133. A compound of any one of Embodiments 1-119, or a pharmaceutically acceptable salt thereof, for use in the treatment of pancreatitis.

Embodiment 134. A method of treating a CFTR mediated disease in a subject comprising administering to the subject a compound of any one of Embodiments 1-119, or a pharmaceutically acceptable salt thereof, or administering a pharmaceutical composition of any one of Embodiments 120 to 128.

Embodiment 135. A method of treating a CFTR mediated disease in a subject comprising administering to the subject a therapeutically effective amount of a compound of any one of Embodiments 1-119, or a pharmaceutically acceptable salt thereof, or administering a pharmaceutical composition of any one of Embodiments 120 to 128.

Embodiment 136. The method of Embodiments 134 or 135, wherein the CFTR mediated disease is selected from cystic fibrosis, asthma, COPD and chronic bronchitis.

Embodiment 137. The method of Embodiments 134 or 135, wherein the CFTR mediated disease is selected from cystic fibrosis, COPD and emphysema.

Embodiment 138. The method of Embodiments 134 or 135, wherein the CFTR mediated disease is cystic fibrosis.

Embodiment 139. A method of treating a pancreatitis in a subject comprising administering to the subject a compound of any one of Embodiments 1-119, or a pharmaceutically acceptable salt thereof, or administering a pharmaceutical composition of any one of Embodiments 120 to 128.

Embodiment 140. The method of any one of Embodiments 134 to 139, further comprising administering to the subject one or more additional pharmaceutical agent(s) prior to, concurrent with, or subsequent to the administration of a compound of any one of Embodiments 1 to 119 or the pharmaceutical composition of any one of Embodiments 120 to 128.

Embodiment 141. The method of Embodiment 140, wherein additional pharmaceutical agent(s) is selected from a mucolytic agent, nebulized hypertonic saline, bronchodilator, an antibiotic, an anti-infective agent, a CFTR modulator, and an anti-inflammatory agent.

Embodiment 142. The method of Embodiment 140, wherein the additional pharmaceutical agent(s) is selected from a CFTR modulator.

Embodiment 143. The method of Embodiment 140, wherein the additional pharmaceutical agent(s) is selected from a CFTR potentiator.

Embodiment 144. The method of Embodiment 140, wherein the additional pharmaceutical agent(s) is selected from a CFTR modulator and a CFTR potentiator.

Embodiment 145. The method of Embodiment 140, wherein the additional pharmaceutical agent(s) is selected from a CFTR corrector.

Embodiment 146. The method of Embodiment 140, wherein the additional pharmaceutical agent(s) is selected from a CFTR corrector and a CFTR potentiator.

Embodiment 147. The use of a compound of any one of Embodiments 1 to 119, or pharmaceutically acceptable salt thereof, in the manufacture of a medicament for treating a disease in which CFTR modulation contributes to the pathology and/or symptomology of a disease.

Embodiment 148. The use of a compound of any one of Embodiments 1 to 119, or pharmaceutically acceptable salt thereof, in the manufacture of a medicament for treating a pancreatitis.

### Combination Therapy

In certain instances, it may be advantageous to administer a compound of the present invention in combination with, before, or after, one or more additional therapeutic agent(s). The compound of the present invention may be administered separately, by the same or different route of administration, or together in the same pharmaceutical composition as the other therapeutic agents. A therapeutic agent is, for example, a chemical compound, peptide, antibody, antibody fragment or nucleic acid, which is therapeutically active or enhances the therapeutic activity when administered to a patient in combination with a compound of the present invention.

In one embodiment, the invention provides a product comprising a compound of the present invention and at least one other therapeutic agent as a combined preparation for simultaneous, separate or sequential use in therapy. In one embodiment, the therapy is the treatment of a disease or condition mediated by CFTR. Products provided as a combined preparation include a composition comprising the compound of the present invention and the other therapeutic agent(s) together in the same pharmaceutical composition, or the compound of the present invention and the other therapeutic agent(s) in separate form, e.g. in the form of a kit.

In one embodiment, the invention provides a pharmaceutical composition comprising a compound of the present invention and another therapeutic agent(s). Optionally, the pharmaceutical composition may comprise a pharmaceutically acceptable carrier, as described above.

In one embodiment, the invention provides a kit comprising two or more separate pharmaceutical compositions, at least one of which contains a compound of the present invention. In one embodiment, the kit comprises means for separately retaining said compositions, such as a container, divided bottle, or divided foil packet. An example of such a kit is a blister pack, as typically used for the packaging of tablets, capsules and the like.

The kit of the invention may be used for administering different dosage forms, for example, oral and parenteral, for administering the separate compositions at different dosage intervals, or for titrating the separate compositions against one another. To assist compliance, the kit of the invention typically comprises directions for administration.

In the combination therapies of the invention, the compound of the present invention and the other therapeutic agent may be manufactured and/or formulated by the same or different manufacturers. Moreover, the compound of the present invention and the other therapeutic may be brought together into a combination therapy: (i) prior to release of the combination product to physicians (e.g. in the case of a kit comprising the compound of the present invention and the other therapeutic agent); (ii) by the physician themselves (or under the guidance of the physician) shortly before administration; (iii) in the patient themselves, e.g. during sequential administration of the compound of the present invention and the other therapeutic agent.

Accordingly, the invention provides the use of a compound of the present invention for treating a disease or condition mediated by CFTR, wherein the medicament is prepared for administration with another therapeutic agent. The invention also provides the use of another therapeutic agent for treating a disease or condition mediated by CFTR, wherein the medicament is administered with a compound of the present invention.

The invention also provides a compound of the present invention for use in a method of treating a disease or condition mediated by CFTR, wherein the compound of the present invention is prepared for administration with another therapeutic agent. The invention also provides another therapeutic agent for use in a method of treating a disease or condition mediated by CFTR, wherein the other therapeutic agent is prepared for administration with a compound of the present invention. The invention also provides a compound of the present invention for use in a method of treating a disease or condition mediated by CFTR, wherein the compound of the present invention is administered with another therapeutic agent. The invention also provides another therapeutic agent for use in a method of treating a disease or condition mediated by CFTR, wherein the other therapeutic agent is administered with a compound of the present invention.

The invention also provides the use of a compound of the present invention for treating a disease or condition mediated by CFTR, wherein the patient has previously (e.g. within 24 hours) been treated with another therapeutic agent. The invention also provides the use of another therapeutic agent for treating a disease or condition mediated by CFTR, wherein the patient has previously (e.g. within 24 hours) been treated with a compound of the present invention.

In one embodiment, the other therapeutic agent is selected from osmotic agents, ion channel modulating agents, mucolytic agents, bronchodilators, antihistamines, antibiotics, anti-inflammatory agents and CFTR modulators.

In another embodiment the other therapeutic agent is an osmotic agent, for example, nebulized hypertonic saline, dextran, mannitol or Xylitol.

In another embodiment the other therapeutic agent is a mucolytic agent, for example, Pulmozyme™.

In another embodiment, the other therapeutic agent is a bronchodilator, for example, albuterol, metaprotenerol sulfate, pirbuterol acetate, salmeterol, indacaterol or tetrabuline sulfate; suitable bronchodilatory agents also include anticholinergic and antimuscarinic agents, in particular, ipratropium bromide, oxitropium bromide, glycopyrronium salts or tiotropium salts.

In another embodiment, the other therapeutic agent is an antihistamine, for example, cetirizine hydrochloride, clemastine fumarate, promethazine, loratidine, desloratidine, diphenhydramine fexofenadine hydrochloride, activastine, astemizole, azelastine, ebastine, epinastine, mizolastine or tefenadine

In another embodiment the other therapeutic agent is an antibiotic, for example tobramycin, including tobramycin inhaled powder, azithromycin, cayston, aztreonam, including the aerosolized for of aztreonam, amikacin, including liposomal formulations thereof, ciprofloxacin, including formulations thereof suitable of administration by inhalation, levofloxacin, including aerosolized formulations thereof and combinations of two antibiotics, for example, fosfomycin and tobramycin.

In another embodiment the other therapeutic agent is an anti-inflammatory agent, for example ibuprofen, docosahexanoic acid, sildenafil, inhaled glutathione, pioglitazone, hydroxychloroquine or simavastatin; a steroid, for example, glucocorticosteroids, such as budesonide, beclamethasone dipropionate, fluticasone propionate, ciclesonide or mometasone furoate; an LTD4 antagonist, such as montelukast or zafirlukast; a PDE4 inhibitor, such as Enprofylline, Theophylline, Roflumilaste, Ariflo (Cilomilaste), Tofimilaste, Pumafentrine, Lirimilaste, Apremilaste, Arofylline, Atizorame, Oglemilasturn, or Tetomilaste.

In another embodiment the other therapeutic agent is a CFTR modulator. In another embodiment the other therapeutic agent is a CFTR potentiator. In another embodiment the other therapeutic agent is a CFTR corrector. Exemplary CFTR modulators include N-(2-(5-chloro-2-methoxy-phenylamino)-4′-methyl-[4, 5′]bithiazolyl-2′-yl)-benzamide (Corr-4a), N-(2,4-di-tert-butyl-5-hydroxyphenyl)-1,4-dihydro-4-oxoquinoline-3-carboxamide (Ivacaftor), N-[2-(1,1-Dimethylethyl)-4-[1,1-di(methyl-da)ethyl-2,2,2-d₃]-5-hydroxyphenyl]-1,4-dihydro-4-oxo-3-quinolinecarboxamide (CTP-656), (((3-((3-carbamoyl-5,5,7,7-tetramethyl-4,7-dihydro-5H-thieno[2,3-c]pyran-2-yl)carbamoyl)-1H-pyrazol-1-yl)methoxy)methyl)phosphonic acid (GLPG1833), 3-[6-({[1-(2,2-difluoro-1,3-benzodioxol-5-yl)cyclopropyl]carbonyl}amino)-3-methylpyridin-2-yl]benzoic acid (Lumacaftor), N-(3-carbamoyl-5,5,7,7-tetramethyl-4,7-dihydro-5H-thieno[2,3-c]pyran-2-yl)-1H-pyrazole-3-carboxamide, 1-(2,2-difluoro-1,3-benzodioxol-5-yl)-N-[1-[(2R)-2,3-dihydroxypropyl]-6-fluoro-2-(2-hydroxy-1,1-dimethylethyl)-1H-indol-5-yl]-cyclopropanecarboxamide (VX-661, Tezacaftor), 4-((2R,4R)-4-(1-(2,2-difluorobenzo[d][1,3]dioxol-5-yl)cyclopropane-1-carboxamido)-7-(difluoromethoxy)chroman-2-yl)benzoic acid (GLPG2222), 4-(3-(1-(2,2-difluorobenzo[d][1,3]dioxol-5-yl)cyclopropane-1-carboxamido)isoquinolin-1-yl)benzoic acid, N-(4-(7-azabicyclo[2.2.1]heptan-7-yl)-2-(trifluoromethyl)phenyl)-4-oxo-5-(trifluromethyl)-1,4-dihydroquinoline-3-carboxamide, 3-[5-(2-fluorophenyl)-1,2,4-oxadiazol-3-yl]benzoic acid (Ataluren), 5,7-Dihydroxy-3-(4-hydroxyphenyl)chromen-4-one (Genistein), N-(2-(tert-butyl)-5-hydroxy-4-(2-(methyl-d₃)propan-2-yl-1,1,1,3,3,3-d₆)phenyl)-4-oxo-1,4-dihydroquinoline-3-carboxamide (CTP-656), N-(3-carbamoyl-5,5,7,7-tetramethyl-4,7-dihydro-5H-thieno[2,3-c]pyran-2-yl)-1H-pyrazole-5-carboxamide (GLPG1837), 3-Chloro-4-(6-hydroxyquinolin-2-yl)benzoic acid (N-91115) and (S)-3-amino-6-methoxy-N-(3,3,3-trifluoro-2-hydroxy-2-methylpropyl)-5-(trifluoromethyl)picolinamide.

In one embodiment of the invention, there is provided a product comprising a compound of the present invention or a pharmaceutically acceptable salt thereof and a CFTR modulator as a combined preparation for simultaneous, separate or sequential use in therapy. In another embodiment, there is provided a product comprising a compound of the present invention and a CFTR potentiator as a combined preparation for simultaneous, separate or sequential use in therapy. In another embodiment there is provided a product comprising a compound of the present invention, a CFTR potentiator and a CFTR corrector as a combined preparation for simultaneous, separate or sequential use in therapy.

Certain aspects and examples of the combinations and combination therapy of the present invention are provided in the following listing of additional, enumerated embodiments. It will be recognized that features specified in each embodiment may be combined with other specified features to provide further embodiments of the present invention.

Embodiment 149. A product comprising a compound of any one of Embodiments 1 to 119, or pharmaceutically acceptable salt thereof, and (S)-3-amino-6-methoxy-N-(3,3,3-trifluoro-2-hydroxy-2-methylpropyl)-5-(trifluoromethyl)picolinamide or a pharmaceutically acceptable salt thereof, as a combined preparation for simultaneous, separate or sequential use in therapy.

Embodiment 150. A product comprising 4-(4,4-dioxido-2³-(trifluoromethyl)-4-thia-3,6-diaza-2,5(2,6)-dipyridina-1(1,2)-benzenacyclodecaphane-6-yl)butanoic acid, or a pharmaceutically acceptable salt thereof, and (S)-3-amino-6-methoxy-N-(3,3,3-trifluoro-2-hydroxy-2-methylpropyl)-5-(trifluoromethyl)picolinamide or a pharmaceutically acceptable salt thereof, as a combined preparation for simultaneous, separate or sequential use in therapy.

Embodiment 151. A product comprising 4-(4,4-dioxido-2³-(trifluoromethyl)-4-thia-3,6-diaza-2,5(2,6)-dipyridina-1(1,2)-benzenacycloundecaphane-6-yl)butanoic acid, or a pharmaceutically acceptable salt thereof, and (S)-3-amino-6-methoxy-N-(3,3,3-trifluoro-2-hydroxy-2-methylpropyl)-5-(trifluoromethyl)picolinamide or a pharmaceutically acceptable salt thereof, as a combined preparation for simultaneous, separate or sequential use in therapy.

Embodiment 152. A product comprising 4-(4,4-dioxido-2³-(trifluoromethyl)-4-thia-3,6-diaza-2,5(2,6)-dipyridina-1(1,2)-benzenacyclododecaphane-6-yl)butanoic acid, or a pharmaceutically acceptable salt thereof, and (S)-3-amino-6-methoxy-N-(3,3,3-trifluoro-2-hydroxy-2-methylpropyl)-5-(trifluoromethyl)picolinamide or a pharmaceutically acceptable salt thereof, as a combined preparation for simultaneous, separate or sequential use in therapy.

Embodiment 153. A product comprising 4-(4,4-dioxido-2³-(trifluoromethyl)-4-thia-3,6-diaza-2,5(2,6)-dipyridina-1(1,2)-benzenacyclotridecaphane-6-yl)butanoic acid, or a pharmaceutically acceptable salt thereof, and (S)-3-amino-6-methoxy-N-(3,3,3-trifluoro-2-hydroxy-2-methylpropyl)-5-(trifluoromethyl)picolinamide or a pharmaceutically acceptable salt thereof, as a combined preparation for simultaneous, separate or sequential use in therapy.

Embodiment 154. A product comprising 4-(4,4-dioxido-2³-(trifluoromethyl)-4-thia-3,6-diaza-2,5(2,6)-dipyridina-1(1,2)-benzenacyclotetradecaphane-6-yl)butanoic acid, or a pharmaceutically acceptable salt thereof, and (S)-3-amino-6-methoxy-N-(3,3,3-trifluoro-2-hydroxy-2-methylpropyl)-5-(trifluoromethyl)picolinamide or a pharmaceutically acceptable salt thereof, as a combined preparation for simultaneous, separate or sequential use in therapy.

Embodiment 155. A product comprising 4-(4,4-dioxido-2³-(trifluoromethyl)-4-thia-3,6-diaza-2,5(2,6)-dipyridina-1(1,2)-benzenacyclopentadecaphane-6-yl)butanoic acid, or a pharmaceutically acceptable salt thereof, and (S)-3-amino-6-methoxy-N-(3,3,3-trifluoro-2-hydroxy-2-methylpropyl)-5-(trifluoromethyl)picolinamide or a pharmaceutically acceptable salt thereof, as a combined preparation for simultaneous, separate or sequential use in therapy.

Embodiment 156. A product comprising a compound of any one of Embodiments 1 to 119, or pharmaceutically acceptable salt thereof, and N-(2,4-di-tert-butyl-5-hydroxyphenyl)-1,4-dihydro-4-oxoquinoline-3-carboxamide, or a pharmaceutically acceptable salt thereof, as a combined preparation for simultaneous, separate or sequential use in therapy.

Embodiment 157. A product comprising 4-(4,4-dioxido-2³-(trifluoromethyl)-4-thia-3,6-diaza-2,5(2,6)-dipyridina-1(1,2)-benzenacyclodecaphane-6-yl)butanoic acid, or pharmaceutically acceptable salt thereof, and N-(2,4-di-tert-butyl-5-hydroxyphenyl)-1,4-dihydro-4-oxoquinoline-3-carboxamide, or a pharmaceutically acceptable salt thereof, as a combined preparation for simultaneous, separate or sequential use in therapy.

Embodiment 158. A product comprising 4-(4,4-dioxido-2³-(trifluoromethyl)-4-thia-3,6-diaza-2,5(2,6)-dipyridina-1(1,2)-benzenacycloundecaphane-6-yl)butanoic acid, or pharmaceutically acceptable salt thereof, and N-(2,4-di-tert-butyl-5-hydroxyphenyl)-1,4-dihydro-4-oxoquinoline-3-carboxamide, or a pharmaceutically acceptable salt thereof, as a combined preparation for simultaneous, separate or sequential use in therapy.

Embodiment 159. A product comprising 4-(4,4-dioxido-2³-(trifluoromethyl)-4-thia-3,6-diaza-2,5(2,6)-dipyridina-1(1,2)-benzenacyclododecaphane-6-yl)butanoic acid, or pharmaceutically acceptable salt thereof, and N-(2,4-di-tert-butyl-5-hydroxyphenyl)-1,4-dihydro-4-oxoquinoline-3-carboxamide, or a pharmaceutically acceptable salt thereof, as a combined preparation for simultaneous, separate or sequential use in therapy.

Embodiment 160. A product comprising 4-(4,4-dioxido-2³-(trifluoromethyl)-4-thia-3,6-diaza-2,5(2,6)-dipyridina-1(1,2)-benzenacyclotridecaphane-6-yl)butanoic acid, or pharmaceutically acceptable salt thereof, and N-(2,4-di-tert-butyl-5-hydroxyphenyl)-1,4-dihydro-4-oxoquinoline-3-carboxamide, or a pharmaceutically acceptable salt thereof, as a combined preparation for simultaneous, separate or sequential use in therapy.

Embodiment 161. A product comprising 4-(4,4-dioxido-2³-(trifluoromethyl)-4-thia-3,6-diaza-2,5(2,6)-dipyridina-1(1,2)-benzenacyclotetradecaphane-6-yl)butanoic acid, or pharmaceutically acceptable salt thereof, and N-(2,4-di-tert-butyl-5-hydroxyphenyl)-1,4-dihydro-4-oxoquinoline-3-carboxamide, or a pharmaceutically acceptable salt thereof, as a combined preparation for simultaneous, separate or sequential use in therapy.

Embodiment 162. A product comprising 4-(4,4-dioxido-2³-(trifluoromethyl)-4-thia-3,6-diaza-2,5(2,6)-dipyridina-1(1,2)-benzenacyclopentadecaphane-6-yl)butanoic acid, or pharmaceutically acceptable salt thereof, and N-(2,4-di-tert-butyl-5-hydroxyphenyl)-1,4-dihydro-4-oxoquinoline-3-carboxamide, or a pharmaceutically acceptable salt thereof, as a combined preparation for simultaneous, separate or sequential use in therapy.

Embodiment 163. A product comprising a compound of any one of Embodiments 1 to 119, or pharmaceutically acceptable salt thereof, N-(2,4-di-tert-butyl-5-hydroxyphenyl)-1,4-dihydro-4-oxoquinoline-3-carboxamide, or a pharmaceutically acceptable salt thereof, and 3-[6-({[1-(2,2-difluoro-1,3-benzodioxol-5-yl)cyclopropyl]carbonyl}amino)-3-methylpyridin-2-yl]benzoic acid, or a pharmaceutically acceptable salt thereof, as a combined preparation for simultaneous, separate or sequential use in therapy.

Embodiment 164. A product comprising 4-(4,4-dioxido-2³-(trifluoromethyl)-4-thia-3,6-diaza-2,5(2,6)-dipyridina-1(1,2)-benzenacyclodecaphane-6-yl)butanoic acid, or pharmaceutically acceptable salt thereof, N-(2,4-di-tert-butyl-5-hydroxyphenyl)-1,4-dihydro-4-oxoquinoline-3-carboxamide, or a pharmaceutically acceptable salt thereof, and 3-[6-({[1-(2,2-difluoro-1,3-benzodioxol-5-yl)cyclopropyl]carbonyl}amino)-3-methylpyridin-2-yl]benzoic acid, or a pharmaceutically acceptable salt thereof, as a combined preparation for simultaneous, separate or sequential use in therapy.

Embodiment 165. A product comprising 4-(4,4-dioxido-2³-(trifluoromethyl)-4-thia-3,6-diaza-2,5(2,6)-dipyridina-1(1,2)-benzenacycloundecaphane-6-yl)butanoic acid, or pharmaceutically acceptable salt thereof, N-(2,4-di-tert-butyl-5-hydroxyphenyl)-1,4-dihydro-4-oxoquinoline-3-carboxamide, or a pharmaceutically acceptable salt thereof, and 3-[6-({[1-(2,2-difluoro-1,3-benzodioxol-5-yl)cyclopropyl]carbonyl}amino)-3-methylpyridin-2-yl]benzoic acid, or a pharmaceutically acceptable salt thereof, as a combined preparation for simultaneous, separate or sequential use in therapy.

Embodiment 166. A product comprising 4-(4,4-dioxido-2³-(trifluoromethyl)-4-thia-3,6-diaza-2,5(2,6)-dipyridina-1(1,2)-benzenacyclododecaphane-6-yl)butanoic acid, or pharmaceutically acceptable salt thereof, N-(2,4-di-tert-butyl-5-hydroxyphenyl)-1,4-dihydro-4-oxoquinoline-3-carboxamide, or a pharmaceutically acceptable salt thereof, and 3-[6-({[1-(2,2-difluoro-1,3-benzodioxol-5-yl)cyclopropyl]carbonyl}amino)-3-methylpyridin-2-yl]benzoic acid, or a pharmaceutically acceptable salt thereof, as a combined preparation for simultaneous, separate or sequential use in therapy.

Embodiment 167. A product comprising 4-(4,4-dioxido-2³-(trifluoromethyl)-4-thia-3,6-diaza-2,5(2,6)-dipyridina-1(1,2)-benzenacyclotridecaphane-6-yl)butanoic acid, or pharmaceutically acceptable salt thereof, N-(2,4-di-tert-butyl-5-hydroxyphenyl)-1,4-dihydro-4-oxoquinoline-3-carboxamide, or a pharmaceutically acceptable salt thereof, and 3-[6-({[1-(2,2-difluoro-1,3-benzodioxol-5-yl)cyclopropyl]carbonyl}amino)-3-methylpyridin-2-yl]benzoic acid, or a pharmaceutically acceptable salt thereof, as a combined preparation for simultaneous, separate or sequential use in therapy.

Embodiment 168. A product comprising 4-(4,4-dioxido-2³-(trifluoromethyl)-4-thia-3,6-diaza-2,5(2,6)-dipyridina-1(1,2)-benzenacyclotetradecaphane-6-yl)butanoic acid, or pharmaceutically acceptable salt thereof, N-(2,4-di-tert-butyl-5-hydroxyphenyl)-1,4-dihydro-4-oxoquinoline-3-carboxamide, or a pharmaceutically acceptable salt thereof, and 3-[6-({[1-(2,2-difluoro-1,3-benzodioxol-5-yl)cyclopropyl]carbonyl}amino)-3-methylpyridin-2-yl]benzoic acid, or a pharmaceutically acceptable salt thereof, as a combined preparation for simultaneous, separate or sequential use in therapy.

Embodiment 169. A product comprising 4-(4,4-dioxido-2³-(trifluoromethyl)-4-thia-3,6-diaza-2,5(2,6)-dipyridina-1(1,2)-benzenacyclopentadecaphane-6-yl)butanoic acid, or pharmaceutically acceptable salt thereof, N-(2,4-di-tert-butyl-5-hydroxyphenyl)-1,4-dihydro-4-oxoquinoline-3-carboxamide, or a pharmaceutically acceptable salt thereof, and 3-[6-({[1-(2,2-difluoro-1,3-benzodioxol-5-yl)cyclopropyl]carbonyl}amino)-3-methylpyridin-2-yl]benzoic acid, or a pharmaceutically acceptable salt thereof, as a combined preparation for simultaneous, separate or sequential use in therapy.

Embodiment 170. A product comprising a compound of any one of Embodiments 1 to 119, or pharmaceutically acceptable salt thereof, N-(2,4-di-tert-butyl-5-hydroxyphenyl)-1,4-dihydro-4-oxoquinoline-3-carboxamide, or a pharmaceutically acceptable salt thereof, and 1-(2,2-difluoro-1,3-benzodioxol-5-yl)-N-[1-[(2R)-2,3-dihydroxypropyl]-6-fluoro-2-(2-hydroxy-1,1-dimethylethyl)-1H-indol-5-yl]-cyclopropanecarboxamide, or a pharmaceutically acceptable salt thereof, as a combined preparation for simultaneous, separate or sequential use in therapy.

Embodiment 171. A product comprising 4-(4,4-dioxido-2³-(trifluoromethyl)-4-thia-3,6-diaza-2,5(2,6)-dipyridina-1(1,2)-benzenacyclodecaphane-6-yl)butanoic acid, or pharmaceutically acceptable salt thereof, N-(2,4-di-tert-butyl-5-hydroxyphenyl)-1,4-dihydro-4-oxoquinoline-3-carboxamide, or a pharmaceutically acceptable salt thereof, and 1-(2,2-difluoro-1,3-benzodioxol-5-yl)-N-[1-[(2R)-2,3-dihydroxypropyl]-6-fluoro-2-(2-hydroxy-1,1-dimethylethyl)-1H-indol-5-yl]-cyclopropanecarboxamide, or a pharmaceutically acceptable salt thereof, as a combined preparation for simultaneous, separate or sequential use in therapy.

Embodiment 172. A product comprising 4-(4,4-dioxido-2³-(trifluoromethyl)-4-thia-3,6-diaza-2,5(2,6)-dipyridina-1(1,2)-benzenacycloundecaphane-6-yl)butanoic acid, or pharmaceutically acceptable salt thereof, N-(2,4-di-tert-butyl-5-hydroxyphenyl)-1,4-dihydro-4-oxoquinoline-3-carboxamide, or a pharmaceutically acceptable salt thereof, and 1-(2,2-difluoro-1,3-benzodioxol-5-yl)-N-[1-[(2R)-2,3-dihydroxypropyl]-6-fluoro-2-(2-hydroxy-1,1-dimethylethyl)-1H-indol-5-yl]-cyclopropanecarboxamide, or a pharmaceutically acceptable salt thereof, as a combined preparation for simultaneous, separate or sequential use in therapy.

Embodiment 173. A product comprising 4-(4,4-dioxido-2³-(trifluoromethyl)-4-thia-3,6-diaza-2,5(2,6)-dipyridina-1(1,2)-benzenacyclododecaphane-6-yl)butanoic acid, or pharmaceutically acceptable salt thereof, N-(2,4-di-tert-butyl-5-hydroxyphenyl)-1,4-dihydro-4-oxoquinoline-3-carboxamide, or a pharmaceutically acceptable salt thereof, and 1-(2,2-difluoro-1,3-benzodioxol-5-yl)-N-[1-[(2R)-2,3-dihydroxypropyl]-6-fluoro-2-(2-hydroxy-1,1-dimethylethyl)-1H-indol-5-yl]-cyclopropanecarboxamide, or a pharmaceutically acceptable salt thereof, as a combined preparation for simultaneous, separate or sequential use in therapy.

Embodiment 174. A product comprising 4-(4,4-dioxido-2³-(trifluoromethyl)-4-thia-3,6-diaza-2,5(2,6)-dipyridina-1(1,2)-benzenacyclotridecaphane-6-yl)butanoic acid, or pharmaceutically acceptable salt thereof, N-(2,4-di-tert-butyl-5-hydroxyphenyl)-1,4-dihydro-4-oxoquinoline-3-carboxamide, or a pharmaceutically acceptable salt thereof, and 1-(2,2-difluoro-1,3-benzodioxol-5-yl)-N-[1-[(2R)-2,3-dihydroxypropyl]-6-fluoro-2-(2-hydroxy-1,1-dimethylethyl)-1H-indol-5-yl]-cyclopropanecarboxamide, or a pharmaceutically acceptable salt thereof, as a combined preparation for simultaneous, separate or sequential use in therapy.

Embodiment 175. A product comprising 4-(4,4-dioxido-2³-(trifluoromethyl)-4-thia-3,6-diaza-2,5(2,6)-dipyridina-1(1,2)-benzenacyclotetradecaphane-6-yl)butanoic acid, or pharmaceutically acceptable salt thereof, N-(2,4-di-tert-butyl-5-hydroxyphenyl)-1,4-dihydro-4-oxoquinoline-3-carboxamide, or a pharmaceutically acceptable salt thereof, and 1-(2,2-difluoro-1,3-benzodioxol-5-yl)-N-[1-[(2R)-2,3-dihydroxypropyl]-6-fluoro-2-(2-hydroxy-1,1-dimethylethyl)-1H-indol-5-yl]-cyclopropanecarboxamide, or a pharmaceutically acceptable salt thereof, as a combined preparation for simultaneous, separate or sequential use in therapy.

Embodiment 176. A product comprising 4-(4,4-dioxido-2³-(trifluoromethyl)-4-thia-3,6-diaza-2,5(2,6)-dipyridina-1(1,2)-benzenacyclopentadecaphane-6-yl)butanoic acid, or pharmaceutically acceptable salt thereof, N-(2,4-di-tert-butyl-5-hydroxyphenyl)-1,4-dihydro-4-oxoquinoline-3-carboxamide, or a pharmaceutically acceptable salt thereof, and 1-(2,2-difluoro-1,3-benzodioxol-5-yl)-N-[1-[(2R)-2,3-dihydroxypropyl]-6-fluoro-2-(2-hydroxy-1,1-dimethylethyl)-1H-indol-5-yl]-cyclopropanecarboxamide, or a pharmaceutically acceptable salt thereof, as a combined preparation for simultaneous, separate or sequential use in therapy.

Embodiment 177. A pharmaceutical composition comprising a compound of any one of Embodiments 1 to 119, or a pharmaceutically acceptable salt thereof, a CFTR modulator and a pharmaceutically acceptable carrier.

Embodiment 178. A pharmaceutical composition comprising a compound of any one of Embodiments 1 to 119, or a pharmaceutically acceptable salt thereof, a CFTR potentiator and a pharmaceutically acceptable carrier.

Embodiment 179. A pharmaceutical composition comprising a compound of any one of Embodiments 1 to 119, or a pharmaceutically acceptable salt thereof, a CFTR corrector and a pharmaceutically acceptable carrier.

Embodiment 180. A pharmaceutical composition comprising a compound of any one of Embodiments 1 to 119, or pharmaceutically acceptable salt thereof, and (S)-3-amino-6-methoxy-N-(3,3,3-trifluoro-2-hydroxy-2-methylpropyl)-5-(trifluoromethyl)picolinamide or a pharmaceutically acceptable salt thereof, and a pharmaceutically acceptable carrier.

Embodiment 181. A pharmaceutical composition comprising 4-(4,4-dioxido-2³-(trifluoromethyl)-4-thia-3,6-diaza-2,5(2,6)-dipyridina-1(1,2)-benzenacyclodecaphane-6-yl)butanoic acid, or a pharmaceutically acceptable salt thereof, and (S)-3-amino-6-methoxy-N-(3,3,3-trifluoro-2-hydroxy-2-methylpropyl)-5-(trifluoromethyl)picolinamide or a pharmaceutically acceptable salt thereof, and a pharmaceutically acceptable carrier.

Embodiment 182. A pharmaceutical composition comprising 4-(4,4-dioxido-2³-(trifluoromethyl)-4-thia-3,6-diaza-2,5(2,6)-dipyridina-1(1,2)-benzenacycloundecaphane-6-yl)butanoic acid, or a pharmaceutically acceptable salt thereof, and (S)-3-amino-6-methoxy-N-(3,3,3-trifluoro-2-hydroxy-2-methylpropyl)-5-(trifluoromethyl)picolinamide or a pharmaceutically acceptable salt thereof, and a pharmaceutically acceptable carrier.

Embodiment 183. A pharmaceutical composition comprising 4-(4,4-dioxido-2³-(trifluoromethyl)-4-thia-3,6-diaza-2,5(2,6)-dipyridina-1(1,2)-benzenacyclododecaphane-6-yl)butanoic acid, or a pharmaceutically acceptable salt thereof, and (S)-3-amino-6-methoxy-N-(3,3,3-trifluoro-2-hydroxy-2-methylpropyl)-5-(trifluoromethyl)picolinamide or a pharmaceutically acceptable salt thereof, and a pharmaceutically acceptable carrier.

Embodiment 184. A pharmaceutical composition comprising 4-(4,4-dioxido-2³-(trifluoromethyl)-4-thia-3,6-diaza-2,5(2,6)-dipyridina-1 (1,2)-benzenacyclotridecaphane-6-yl)butanoic acid, or a pharmaceutically acceptable salt thereof, and (S)-3-amino-6-methoxy-N-(3,3,3-trifluoro-2-hydroxy-2-methylpropyl)-5-(trifluoromethyl)picolinamide or a pharmaceutically acceptable salt thereof, and a pharmaceutically acceptable carrier.

Embodiment 185. A pharmaceutical composition comprising 4-(4,4-dioxido-2³-(trifluoromethyl)-4-thia-3,6-diaza-2,5(2,6)-dipyridina-1(1,2)-benzenacyclotetradecaphane-6-yl)butanoic acid, or a pharmaceutically acceptable salt thereof, and (S)-3-amino-6-methoxy-N-(3,3,3-trifluoro-2-hydroxy-2-methylpropyl)-5-(trifluoromethyl)picolinamide or a pharmaceutically acceptable salt thereof, and a pharmaceutically acceptable carrier.

Embodiment 186. A pharmaceutical composition comprising 4-(4,4-dioxido-2³-(trifluoromethyl)-4-thia-3,6-diaza-2,5(2,6)-dipyridina-1(1,2)-benzenacyclopentadecaphane-6-yl)butanoic acid, or a pharmaceutically acceptable salt thereof, and (S)-3-amino-6-methoxy-N-(3,3,3-trifluoro-2-hydroxy-2-methylpropyl)-5-(trifluoromethyl)picolinamide or a pharmaceutically acceptable salt thereof, and a pharmaceutically acceptable carrier.

Embodiment 187. A pharmaceutical composition comprising a compound of any one of Embodiments 1 to 119, or pharmaceutically acceptable salt thereof, and N-(2,4-di-tert-butyl-5-hydroxyphenyl)-1,4-dihydro-4-oxoquinoline-3-carboxamide, or a pharmaceutically acceptable salt thereof, and a pharmaceutically acceptable carrier.

Embodiment 188. A pharmaceutical composition comprising 4-(4,4-dioxido-2³-(trifluoromethyl)-4-thia-3,6-diaza-2,5(2,6)-dipyridina-1(1,2)-benzenacyclodecaphane-6-yl)butanoic acid, or pharmaceutically acceptable salt thereof, and N-(2,4-di-tert-butyl-5-hydroxyphenyl)-1,4-dihydro-4-oxoquinoline-3-carboxamide, or a pharmaceutically acceptable salt thereof, and a pharmaceutically acceptable carrier.

Embodiment 189. A pharmaceutical composition comprising 4-(4,4-dioxido-2³-(trifluoromethyl)-4-thia-3,6-diaza-2,5(2,6)-dipyridina-1(1,2)-benzenacycloundecaphane-6-yl)butanoic acid, or pharmaceutically acceptable salt thereof, and N-(2,4-di-tert-butyl-5-hydroxyphenyl)-1,4-dihydro-4-oxoquinoline-3-carboxamide, or a pharmaceutically acceptable salt thereof, and a pharmaceutically acceptable carrier.

Embodiment 190. A pharmaceutical composition comprising 4-(4,4-dioxido-2³-(trifluoromethyl)-4-thia-3,6-diaza-2,5(2,6)-dipyridina-1(1,2)-benzenacyclododecaphane-6-yl)butanoic acid, or pharmaceutically acceptable salt thereof, and N-(2,4-di-tert-butyl-5-hydroxyphenyl)-1,4-dihydro-4-oxoquinoline-3-carboxamide, or a pharmaceutically acceptable salt thereof, and a pharmaceutically acceptable carrier.

Embodiment 191. A pharmaceutical composition comprising 4-(4,4-dioxido-2³-(trifluoromethyl)-4-thia-3,6-diaza-2,5(2,6)-dipyridina-1(1,2)-benzenacyclotridecaphane-6-yl)butanoic acid, or pharmaceutically acceptable salt thereof, and N-(2,4-di-tert-butyl-5-hydroxyphenyl)-1,4-dihydro-4-oxoquinoline-3-carboxamide, or a pharmaceutically acceptable salt thereof, and a pharmaceutically acceptable carrier.

Embodiment 192. A pharmaceutical composition comprising 4-(4,4-dioxido-2³-(trifluoromethyl)-4-thia-3,6-diaza-2,5(2,6)-dipyridina-1(1,2)-benzenacyclotetradecaphane-6-yl)butanoic acid, or pharmaceutically acceptable salt thereof, and N-(2,4-di-tert-butyl-5-hydroxyphenyl)-1,4-dihydro-4-oxoquinoline-3-carboxamide, or a pharmaceutically acceptable salt thereof, and a pharmaceutically acceptable carrier.

Embodiment 193. A pharmaceutical composition comprising 4-(4,4-dioxido-2³-(trifluoromethyl)-4-thia-3,6-diaza-2,5(2,6)-dipyridina-1(1,2)-benzenacyclopentadecaphane-6-yl)butanoic acid, or pharmaceutically acceptable salt thereof, and N-(2,4-di-tert-butyl-5-hydroxyphenyl)-1,4-dihydro-4-oxoquinoline-3-carboxamide, or a pharmaceutically acceptable salt thereof, and a pharmaceutically acceptable carrier.

Embodiment 194. A pharmaceutical composition comprising a compound of any one of Embodiments 1 to 119, or pharmaceutically acceptable salt thereof, N-(2,4-di-tert-butyl-5-hydroxyphenyl)-1,4-dihydro-4-oxoquinoline-3-carboxamide, or a pharmaceutically acceptable salt thereof, and 3-[6-({[1-(2,2-difluoro-1,3-benzodioxol-5-yl)cyclopropyl]carbonyl}amino)-3-methylpyridin-2-yl]benzoic acid, or a pharmaceutically acceptable salt thereof, and a pharmaceutically acceptable carrier.

Embodiment 195. A pharmaceutical composition comprising 4-(4,4-dioxido-2³-(trifluoromethyl)-4-thia-3,6-diaza-2,5(2,6)-dipyridina-1(1,2)-benzenacyclodecaphane-6-yl)butanoic acid, or pharmaceutically acceptable salt thereof, N-(2,4-di-tert-butyl-5-hydroxyphenyl)-1,4-dihydro-4-oxoquinoline-3-carboxamide, or a pharmaceutically acceptable salt thereof, and 3-[6-({[1-(2,2-difluoro-1,3-benzodioxol-5-yl)cyclopropyl]carbonyl}amino)-3-methylpyridin-2-yl]benzoic acid, or a pharmaceutically acceptable salt thereof, and a pharmaceutically acceptable carrier.

Embodiment 196. A pharmaceutical composition comprising 4-(4,4-dioxido-2³-(trifluoromethyl)-4-thia-3,6-diaza-2,5(2,6)-dipyridina-1(1,2)-benzenacycloundecaphane-6-yl)butanoic acid, or pharmaceutically acceptable salt thereof, N-(2,4-di-tert-butyl-5-hydroxyphenyl)-1,4-dihydro-4-oxoquinoline-3-carboxamide, or a pharmaceutically acceptable salt thereof, and 3-[6-({[1-(2,2-difluoro-1,3-benzodioxol-5-yl)cyclopropyl]carbonyl}amino)-3-methylpyridin-2-yl]benzoic acid, or a pharmaceutically acceptable salt thereof, and a pharmaceutically acceptable carrier.

Embodiment 197. A pharmaceutical composition comprising 4-(4,4-dioxido-2³-(trifluoromethyl)-4-thia-3,6-diaza-2,5(2,6)-dipyridina-1(1,2)-benzenacyclododecaphane-6-yl)butanoic acid, or pharmaceutically acceptable salt thereof, N-(2,4-di-tert-butyl-5-hydroxyphenyl)-1,4-dihydro-4-oxoquinoline-3-carboxamide, or a pharmaceutically acceptable salt thereof, and 3-[6-({[1-(2,2-difluoro-1,3-benzodioxol-5-yl)cyclopropyl]carbonyl}amino)-3-methylpyridin-2-yl]benzoic acid, or a pharmaceutically acceptable salt thereof, and a pharmaceutically acceptable carrier.

Embodiment 198. A pharmaceutical composition comprising 4-(4,4-dioxido-2³-(trifluoromethyl)-4-thia-3,6-diaza-2,5(2,6)-dipyridina-1(1,2)-benzenacyclotridecaphane-6-yl)butanoic acid, or pharmaceutically acceptable salt thereof, N-(2,4-di-tert-butyl-5-hydroxyphenyl)-1,4-dihydro-4-oxoquinoline-3-carboxamide, or a pharmaceutically acceptable salt thereof, and 3-[6-({[1-(2,2-difluoro-1,3-benzodioxol-5-yl)cyclopropyl]carbonyl}amino)-3-methylpyridin-2-yl]benzoic acid, or a pharmaceutically acceptable salt thereof, and a pharmaceutically acceptable carrier.

Embodiment 199. A pharmaceutical composition comprising 4-(4,4-dioxido-2³-(trifluoromethyl)-4-thia-3,6-diaza-2,5(2,6)-dipyridina-1(1,2)-benzenacyclotetradecaphane-6-yl)butanoic acid, or pharmaceutically acceptable salt thereof, N-(2,4-di-tert-butyl-5-hydroxyphenyl)-1,4-dihydro-4-oxoquinoline-3-carboxamide, or a pharmaceutically acceptable salt thereof, and 3-[6-({[1-(2,2-difluoro-1,3-benzodioxol-5-yl)cyclopropyl]carbonyl}amino)-3-methylpyridin-2-yl]benzoic acid, or a pharmaceutically acceptable salt thereof, and a pharmaceutically acceptable carrier.

Embodiment 200. A pharmaceutical composition comprising 4-(4,4-dioxido-2³-(trifluoromethyl)-4-thia-3,6-diaza-2,5(2,6)-dipyridina-1(1,2)-benzenacyclopentadecaphane-6-yl)butanoic acid, or pharmaceutically acceptable salt thereof, N-(2,4-di-tert-butyl-5-hydroxyphenyl)-1,4-dihydro-4-oxoquinoline-3-carboxamide, or a pharmaceutically acceptable salt thereof, and 3-[6-({[1-(2,2-difluoro-1,3-benzodioxol-5-yl)cyclopropyl]carbonyl}amino)-3-methylpyridin-2-yl]benzoic acid, or a pharmaceutically acceptable salt thereof, and a pharmaceutically acceptable carrier.

Embodiment 201. A pharmaceutical composition comprising a compound of any one of Embodiments 1 to 119, or pharmaceutically acceptable salt thereof, N-(2,4-di-tert-butyl-5-hydroxyphenyl)-1,4-dihydro-4-oxoquinoline-3-carboxamide, or a pharmaceutically acceptable salt thereof, and 1-(2,2-difluoro-1,3-benzodioxol-5-yl)-N-[1-[(2R)-2,3-dihydroxypropyl]-6-fluoro-2-(2-hydroxy-1,1-dimethylethyl)-1H-indol-5-yl]-cyclopropanecarboxamide, or a pharmaceutically acceptable salt thereof, and a pharmaceutically acceptable carrier.

Embodiment 202. A pharmaceutical composition comprising 4-(4,4-dioxido-2³-(trifluoromethyl)-4-thia-3,6-diaza-2,5(2,6)-dipyridina-1(1,2)-benzenacyclodecaphane-6-yl)butanoic acid, or pharmaceutically acceptable salt thereof, N-(2,4-di-tert-butyl-5-hydroxyphenyl)-1,4-dihydro-4-oxoquinoline-3-carboxamide, or a pharmaceutically acceptable salt thereof, and 1-(2,2-difluoro-1,3-benzodioxol-5-yl)-N-[1-[(2R)-2,3-dihydroxypropyl]-6-fluoro-2-(2-hydroxy-1,1-dimethylethyl)-1H-indol-5-yl]-cyclopropanecarboxamide, or a pharmaceutically acceptable salt thereof, and a pharmaceutically acceptable carrier.

Embodiment 203. A pharmaceutical composition comprising 4-(4,4-dioxido-2³-(trifluoromethyl)-4-thia-3,6-diaza-2,5(2,6)-dipyridina-1(1,2)-benzenacycloundecaphane-6-yl)butanoic acid, or pharmaceutically acceptable salt thereof, N-(2,4-di-tert-butyl-5-hydroxyphenyl)-1,4-dihydro-4-oxoquinoline-3-carboxamide, or a pharmaceutically acceptable salt thereof, and 1-(2,2-difluoro-1,3-benzodioxol-5-yl)-N-[1-[(2R)-2,3-dihydroxypropyl]-6-fluoro-2-(2-hydroxy-1,1-dimethylethyl)-1H-indol-5-yl]-cyclopropanecarboxamide, or a pharmaceutically acceptable salt thereof, and a pharmaceutically acceptable carrier.

Embodiment 204. A pharmaceutical composition comprising 4-(4,4-dioxido-2³-(trifluoromethyl)-4-thia-3,6-diaza-2,5(2,6)-dipyridina-1(1,2)-benzenacyclododecaphane-6-yl)butanoic acid, or pharmaceutically acceptable salt thereof, N-(2,4-di-tert-butyl-5-hydroxyphenyl)-1,4-dihydro-4-oxoquinoline-3-carboxamide, or a pharmaceutically acceptable salt thereof, and 1-(2,2-difluoro-1,3-benzodioxol-5-yl)-N-[1-[(2R)-2,3-dihydroxypropyl]-6-fluoro-2-(2-hydroxy-1,1-dimethylethyl)-1H-indol-5-yl]-cyclopropanecarboxamide, or a pharmaceutically acceptable salt thereof, and a pharmaceutically acceptable carrier.

Embodiment 205. A pharmaceutical composition comprising 4-(4,4-dioxido-2³-(trifluoromethyl)-4-thia-3,6-diaza-2,5(2,6)-dipyridina-1(1,2)-benzenacyclotridecaphane-6-yl)butanoic acid, or pharmaceutically acceptable salt thereof, N-(2,4-di-tert-butyl-5-hydroxyphenyl)-1,4-dihydro-4-oxoquinoline-3-carboxamide, or a pharmaceutically acceptable salt thereof, and 1-(2,2-difluoro-1,3-benzodioxol-5-yl)-N-[1-[(2R)-2,3-dihydroxypropyl]-6-fluoro-2-(2-hydroxy-1,1-dimethylethyl)-1H-indol-5-yl]-cyclopropanecarboxamide, or a pharmaceutically acceptable salt thereof, and a pharmaceutically acceptable carrier.

Embodiment 206. A pharmaceutical composition comprising 4-(4,4-dioxido-2³-(trifluoromethyl)-4-thia-3,6-diaza-2,5(2,6)-dipyridina-1(1,2)-benzenacyclotetradecaphane-6-yl)butanoic acid, or pharmaceutically acceptable salt thereof, N-(2,4-di-tert-butyl-5-hydroxyphenyl)-1,4-dihydro-4-oxoquinoline-3-carboxamide, or a pharmaceutically acceptable salt thereof, and 1-(2,2-difluoro-1,3-benzodioxol-5-yl)-N-[1-[(2R)-2,3-dihydroxypropyl]-6-fluoro-2-(2-hydroxy-1,1-dimethylethyl)-1H-indol-5-yl]-cyclopropanecarboxamide, or a pharmaceutically acceptable salt thereof, and a pharmaceutically acceptable carrier.

Embodiment 207. A pharmaceutical composition comprising 4-(4,4-dioxido-2³-(trifluoromethyl)-4-thia-3,6-diaza-2,5(2,6)-dipyridina-1(1,2)-benzenacyclopentadecaphane-6-yl)butanoic acid, or pharmaceutically acceptable salt thereof, N-(2,4-di-tert-butyl-5-hydroxyphenyl)-1,4-dihydro-4-oxoquinoline-3-carboxamide, or a pharmaceutically acceptable salt thereof, and 1-(2,2-difluoro-1,3-benzodioxol-5-yl)-N-[1-[(2R)-2,3-dihydroxypropyl]-6-fluoro-2-(2-hydroxy-1,1-dimethylethyl)-1H-indol-5-yl]-cyclopropanecarboxamide, or a pharmaceutically acceptable salt thereof, and a pharmaceutically acceptable carrier.

Embodiment 208. A combination comprising a compound of any one of Embodiments 1 to 119, or pharmaceutically acceptable salt thereof, and (S)-3-amino-6-methoxy-N-(3,3,3-trifluoro-2-hydroxy-2-methylpropyl)-5-(trifluoromethyl)picolinamide or a pharmaceutically acceptable salt thereof, wherein the combination is a fixed combination or a non-fixed combination.

Embodiment 209. A combination comprising 4-(4,4-dioxido-2³-(trifluoromethyl)-4-thia-3,6-diaza-2,5(2,6)-dipyridina-1(1,2)-benzenacyclodecaphane-6-yl)butanoic acid, or a pharmaceutically acceptable salt thereof, and (S)-3-amino-6-methoxy-N-(3,3,3-trifluoro-2-hydroxy-2-methylpropyl)-5-(trifluoromethyl)picolinamide or a pharmaceutically acceptable salt thereof, wherein the combination is a fixed combination or a non-fixed combination.

Embodiment 210. A combination comprising 4-(4,4-dioxido-2³-(trifluoromethyl)-4-thia-3,6-diaza-2,5(2,6)-dipyridina-1(1,2)-benzenacycloundecaphane-6-yl)butanoic acid, or a pharmaceutically acceptable salt thereof, and (S)-3-amino-6-methoxy-N-(3,3,3-trifluoro-2-hydroxy-2-methylpropyl)-5-(trifluoromethyl)picolinamide or a pharmaceutically acceptable salt thereof, wherein the combination is a fixed combination or a non-fixed combination.

Embodiment 211. A combination comprising 4-(4,4-dioxido-2³-(trifluoromethyl)-4-thia-3,6-diaza-2,5(2,6)-dipyridina-1(1,2)-benzenacyclododecaphane-6-yl)butanoic acid, or a pharmaceutically acceptable salt thereof, and (S)-3-amino-6-methoxy-N-(3,3,3-trifluoro-2-hydroxy-2-methylpropyl)-5-(trifluoromethyl)picolinamide or a pharmaceutically acceptable salt thereof, wherein the combination is a fixed combination or a non-fixed combination.

Embodiment 212. A combination comprising 4-(4,4-dioxido-2³-(trifluoromethyl)-4-thia-3,6-diaza-2,5(2,6)-dipyridina-1(1,2)-benzenacyclotridecaphane-6-yl)butanoic acid, or a pharmaceutically acceptable salt thereof, and (S)-3-amino-6-methoxy-N-(3,3,3-trifluoro-2-hydroxy-2-methylpropyl)-5-(trifluoromethyl)picolinamide or a pharmaceutically acceptable salt thereof, wherein the combination is a fixed combination or a non-fixed combination.

Embodiment 213. A combination comprising 4-(4,4-dioxido-2³-(trifluoromethyl)-4-thia-3,6-diaza-2,5(2,6)-dipyridina-1(1,2)-benzenacyclotetradecaphane-6-yl)butanoic acid, or a pharmaceutically acceptable salt thereof, and (S)-3-amino-6-methoxy-N-(3,3,3-trifluoro-2-hydroxy-2-methylpropyl)-5-(trifluoromethyl)picolinamide or a pharmaceutically acceptable salt thereof, wherein the combination is a fixed combination or a non-fixed combination.

Embodiment 214. A combination comprising 4-(4,4-dioxido-2³-(trifluoromethyl)-4-thia-3,6-diaza-2,5(2,6)-dipyridina-1(1,2)-benzenacyclopentadecaphane-6-yl)butanoic acid, or a pharmaceutically acceptable salt thereof, and (S)-3-amino-6-methoxy-N-(3,3,3-trifluoro-2-hydroxy-2-methylpropyl)-5-(trifluoromethyl)picolinamide or a pharmaceutically acceptable salt thereof, wherein the combination is a fixed combination or a non-fixed combination.

Embodiment 215. A combination comprising a compound of any one of Embodiments 1 to 119, or pharmaceutically acceptable salt thereof, and N-(2,4-di-tert-butyl-5-hydroxyphenyl)-1,4-dihydro-4-oxoquinoline-3-carboxamide, or a pharmaceutically acceptable salt thereof, wherein the combination is a fixed combination or a non-fixed combination.

Embodiment 216. A combination comprising 4-(4,4-dioxido-2³-(trifluoromethyl)-4-thia-3,6-diaza-2,5(2,6)-dipyridina-1(1,2)-benzenacyclodecaphane-6-yl)butanoic acid, or pharmaceutically acceptable salt thereof, and N-(2,4-di-tert-butyl-5-hydroxyphenyl)-1,4-dihydro-4-oxoquinoline-3-carboxamide, or a pharmaceutically acceptable salt thereof, wherein the combination is a fixed combination or a non-fixed combination.

Embodiment 217. A combination comprising 4-(4,4-dioxido-2³-(trifluoromethyl)-4-thia-3,6-diaza-2,5(2,6)-dipyridina-1(1,2)-benzenacycloundecaphane-6-yl)butanoic acid, or pharmaceutically acceptable salt thereof, and N-(2,4-di-tert-butyl-5-hydroxyphenyl)-1,4-dihydro-4-oxoquinoline-3-carboxamide, or a pharmaceutically acceptable salt thereof, wherein the combination is a fixed combination or a non-fixed combination.

Embodiment 218. A combination comprising 4-(4,4-dioxido-2³-(trifluoromethyl)-4-thia-3,6-diaza-2,5(2,6)-dipyridina-1(1,2)-benzenacyclododecaphane-6-yl)butanoic acid, or pharmaceutically acceptable salt thereof, and N-(2,4-di-tert-butyl-5-hydroxyphenyl)-1,4-dihydro-4-oxoquinoline-3-carboxamide, or a pharmaceutically acceptable salt thereof, wherein the combination is a fixed combination or a non-fixed combination.

Embodiment 219. A combination comprising 4-(4,4-dioxido-2³-(trifluoromethyl)-4-thia-3,6-diaza-2,5(2,6)-dipyridina-1(1,2)-benzenacyclotridecaphane-6-yl)butanoic acid, or pharmaceutically acceptable salt thereof, and N-(2,4-di-tert-butyl-5-hydroxyphenyl)-1,4-dihydro-4-oxoquinoline-3-carboxamide, or a pharmaceutically acceptable salt thereof, wherein the combination is a fixed combination or a non-fixed combination.

Embodiment 220. A combination comprising 4-(4,4-dioxido-2³-(trifluoromethyl)-4-thia-3,6-diaza-2,5(2,6)-dipyridina-1(1,2)-benzenacyclotetradecaphane-6-yl)butanoic acid, or pharmaceutically acceptable salt thereof, and N-(2,4-di-tert-butyl-5-hydroxyphenyl)-1,4-dihydro-4-oxoquinoline-3-carboxamide, or a pharmaceutically acceptable salt thereof, wherein the combination is a fixed combination or a non-fixed combination.

Embodiment 221. A combination comprising 4-(4,4-dioxido-2³-(trifluoromethyl)-4-thia-3,6-diaza-2,5(2,6)-dipyridina-1(1,2)-benzenacyclopentadecaphane-6-yl)butanoic acid, or pharmaceutically acceptable salt thereof, and N-(2,4-di-tert-butyl-5-hydroxyphenyl)-1,4-dihydro-4-oxoquinoline-3-carboxamide, or a pharmaceutically acceptable salt thereof, wherein the combination is a fixed combination or a non-fixed combination.

Embodiment 222. A combination comprising a compound of any one of Embodiments 1 to 119, or pharmaceutically acceptable salt thereof, N-(2,4-di-tert-butyl-5-hydroxyphenyl)-1,4-dihydro-4-oxoquinoline-3-carboxamide, or a pharmaceutically acceptable salt thereof, and 3-[6-({[1-(2,2-difluoro-1,3-benzodioxol-5-yl)cyclopropyl]carbonyl}amino)-3-methylpyridin-2-yl]benzoic acid, or a pharmaceutically acceptable salt thereof, wherein the combination is a fixed combination or a non-fixed combination.

Embodiment 223. A combination comprising 4-(4,4-dioxido-2³-(trifluoromethyl)-4-thia-3,6-diaza-2,5(2,6)-dipyridina-1(1,2)-benzenacyclodecaphane-6-yl)butanoic acid, or pharmaceutically acceptable salt thereof, N-(2,4-di-tert-butyl-5-hydroxyphenyl)-1,4-dihydro-4-oxoquinoline-3-carboxamide, or a pharmaceutically acceptable salt thereof, and 3-[6-({[1-(2,2-difluoro-1,3-benzodioxol-5-yl)cyclopropyl]carbonyl}amino)-3-methylpyridin-2-yl]benzoic acid, or a pharmaceutically acceptable salt thereof, wherein the combination is a fixed combination or a non-fixed combination.

Embodiment 224. A combination comprising 4-(4,4-dioxido-2³-(trifluoromethyl)-4-thia-3,6-diaza-2,5(2,6)-dipyridina-1(1,2)-benzenacycloundecaphane-6-yl)butanoic acid, or pharmaceutically acceptable salt thereof, N-(2,4-di-tert-butyl-5-hydroxyphenyl)-1,4-dihydro-4-oxoquinoline-3-carboxamide, or a pharmaceutically acceptable salt thereof, and 3-[6-({[1-(2,2-difluoro-1,3-benzodioxol-5-yl)cyclopropyl]carbonyl}amino)-3-methylpyridin-2-yl]benzoic acid, or a pharmaceutically acceptable salt thereof, wherein the combination is a fixed combination or a non-fixed combination.

Embodiment 225. A combination comprising 4-(4,4-dioxido-2³-(trifluoromethyl)-4-thia-3,6-diaza-2,5(2,6)-dipyridina-1(1,2)-benzenacyclododecaphane-6-yl)butanoic acid, or pharmaceutically acceptable salt thereof, N-(2,4-di-tert-butyl-5-hydroxyphenyl)-1,4-dihydro-4-oxoquinoline-3-carboxamide, or a pharmaceutically acceptable salt thereof, and 3-[6-({[1-(2,2-difluoro-1,3-benzodioxol-5-yl)cyclopropyl]carbonyl}amino)-3-methylpyridin-2-yl]benzoic acid, or a pharmaceutically acceptable salt thereof, wherein the combination is a fixed combination or a non-fixed combination.

Embodiment 226. A combination comprising 4-(4,4-dioxido-2³-(trifluoromethyl)-4-thia-3,6-diaza-2,5(2,6)-dipyridina-1(1,2)-benzenacyclotridecaphane-6-yl)butanoic acid, or pharmaceutically acceptable salt thereof, N-(2,4-di-tert-butyl-5-hydroxyphenyl)-1,4-dihydro-4-oxoquinoline-3-carboxamide, or a pharmaceutically acceptable salt thereof, and 3-[6-({[1-(2,2-difluoro-1,3-benzodioxol-5-yl)cyclopropyl]carbonyl}amino)-3-methylpyridin-2-yl]benzoic acid, or a pharmaceutically acceptable salt thereof, wherein the combination is a fixed combination or a non-fixed combination.

Embodiment 227. A combination comprising 4-(4,4-dioxido-2³-(trifluoromethyl)-4-thia-3,6-diaza-2,5(2,6)-dipyridina-1 (1,2)-benzenacyclotetradecaphane-6-yl)butanoic acid, or pharmaceutically acceptable salt thereof, N-(2,4-di-tert-butyl-5-hydroxyphenyl)-1,4-dihydro-4-oxoquinoline-3-carboxamide, or a pharmaceutically acceptable salt thereof, and 3-[6-({[1-(2,2-difluoro-1,3-benzodioxol-5-yl)cyclopropyl]carbonyl}amino)-3-methylpyridin-2-yl]benzoic acid, or a pharmaceutically acceptable salt thereof, wherein the combination is a fixed combination or a non-fixed combination.

Embodiment 228. A combination comprising 4-(4,4-dioxido-2³-(trifluoromethyl)-4-thia-3,6-diaza-2,5(2,6)-dipyridina-1(1,2)-benzenacyclopentadecaphane-6-yl)butanoic acid, or pharmaceutically acceptable salt thereof, N-(2,4-di-tert-butyl-5-hydroxyphenyl)-1,4-dihydro-4-oxoquinoline-3-carboxamide, or a pharmaceutically acceptable salt thereof, and 3-[6-({[1-(2,2-difluoro-1,3-benzodioxol-5-yl)cyclopropyl]carbonyl}amino)-3-methylpyridin-2-yl]benzoic acid, or a pharmaceutically acceptable salt thereof, wherein the combination is a fixed combination or a non-fixed combination.

Embodiment 229. A combination comprising a compound of any one of Embodiments 1 to 119, or pharmaceutically acceptable salt thereof, N-(2,4-di-tert-butyl-5-hydroxyphenyl)-1,4-dihydro-4-oxoquinoline-3-carboxamide, or a pharmaceutically acceptable salt thereof, and 1-(2,2-difluoro-1,3-benzodioxol-5-yl)-N-[1-[(2R)-2,3-dihydroxypropyl]-6-fluoro-2-(2-hydroxy-1,1-dimethylethyl)-1H-indol-5-yl]-cyclopropanecarboxamide, or a pharmaceutically acceptable salt thereof, wherein the combination is a fixed combination or a non-fixed combination.

Embodiment 230. A combination comprising 4-(4,4-dioxido-2³-(trifluoromethyl)-4-thia-3,6-diaza-2,5(2,6)-dipyridina-1(1,2)-benzenacyclodecaphane-6-yl)butanoic acid, or pharmaceutically acceptable salt thereof, N-(2,4-di-tert-butyl-5-hydroxyphenyl)-1,4-dihydro-4-oxoquinoline-3-carboxamide, or a pharmaceutically acceptable salt thereof, and 1-(2,2-difluoro-1,3-benzodioxol-5-yl)-N-[1-[(2R)-2,3-dihydroxypropyl]-6-fluoro-2-(2-hydroxy-1,1-dimethylethyl)-1H-indol-5-yl]-cyclopropanecarboxamide, or a pharmaceutically acceptable salt thereof, wherein the combination is a fixed combination or a non-fixed combination.

Embodiment 231. A combination comprising 4-(4,4-dioxido-2³-(trifluoromethyl)-4-thia-3,6-diaza-2,5(2,6)-dipyridina-1(1,2)-benzenacycloundecaphane-6-yl)butanoic acid, or pharmaceutically acceptable salt thereof, N-(2,4-di-tert-butyl-5-hydroxyphenyl)-1,4-dihydro-4-oxoquinoline-3-carboxamide, or a pharmaceutically acceptable salt thereof, and 1-(2,2-difluoro-1,3-benzodioxol-5-yl)-N-[1-[(2R)-2,3-dihydroxypropyl]-6-fluoro-2-(2-hydroxy-1,1-dimethylethyl)-1H-indol-5-yl]-cyclopropanecarboxamide, or a pharmaceutically acceptable salt thereof, wherein the combination is a fixed combination or a non-fixed combination.

Embodiment 232. A combination comprising 4-(4,4-dioxido-2³-(trifluoromethyl)-4-thia-3,6-diaza-2,5(2,6)-dipyridina-1(1,2)-benzenacyclododecaphane-6-yl)butanoic acid, or pharmaceutically acceptable salt thereof, N-(2,4-di-tert-butyl-5-hydroxyphenyl)-1,4-dihydro-4-oxoquinoline-3-carboxamide, or a pharmaceutically acceptable salt thereof, and 1-(2,2-difluoro-1,3-benzodioxol-5-yl)-N-[1-[(2R)-2,3-dihydroxypropyl]-6-fluoro-2-(2-hydroxy-1,1-dimethylethyl)-1H-indol-5-yl]-cyclopropanecarboxamide, or a pharmaceutically acceptable salt thereof, wherein the combination is a fixed combination or a non-fixed combination.

Embodiment 233. A combination comprising 4-(4,4-dioxido-2³-(trifluoromethyl)-4-thia-3,6-diaza-2,5(2,6)-dipyridina-1(1,2)-benzenacyclotridecaphane-6-yl)butanoic acid, or pharmaceutically acceptable salt thereof, N-(2,4-di-tert-butyl-5-hydroxyphenyl)-1,4-dihydro-4-oxoquinoline-3-carboxamide, or a pharmaceutically acceptable salt thereof, and 1-(2,2-difluoro-1,3-benzodioxol-5-yl)-N-[1-[(2R)-2,3-dihydroxypropyl]-6-fluoro-2-(2-hydroxy-1,1-dimethylethyl)-1H-indol-5-yl]-cyclopropanecarboxamide, or a pharmaceutically acceptable salt thereof, wherein the combination is a fixed combination or a non-fixed combination.

Embodiment 234. A combination comprising 4-(4,4-dioxido-2³-(trifluoromethyl)-4-thia-3,6-diaza-2,5(2,6)-dipyridina-1(1,2)-benzenacyclotetradecaphane-6-yl)butanoic acid, or pharmaceutically acceptable salt thereof, N-(2,4-di-tert-butyl-5-hydroxyphenyl)-1,4-dihydro-4-oxoquinoline-3-carboxamide, or a pharmaceutically acceptable salt thereof, and 1-(2,2-difluoro-1,3-benzodioxol-5-yl)-N-[1-[(2R)-2,3-dihydroxypropyl]-6-fluoro-2-(2-hydroxy-1,1-dimethylethyl)-1H-indol-5-yl]-cyclopropanecarboxamide, or a pharmaceutically acceptable salt thereof, wherein the combination is a fixed combination or a non-fixed combination.

Embodiment 235. A combination comprising 4-(4,4-dioxido-2³-(trifluoromethyl)-4-thia-3,6-diaza-2,5(2,6)-dipyridina-1(1,2)-benzenacyclopentadecaphane-6-yl)butanoic acid, or pharmaceutically acceptable salt thereof, N-(2,4-di-tert-butyl-5-hydroxyphenyl)-1,4-dihydro-4-oxoquinoline-3-carboxamide, or a pharmaceutically acceptable salt thereof, and 1-(2,2-difluoro-1,3-benzodioxol-5-yl)-N-[1-[(2R)-2,3-dihydroxypropyl]-6-fluoro-2-(2-hydroxy-1,1-dimethylethyl)-1H-indol-5-yl]-cyclopropanecarboxamide, or a pharmaceutically acceptable salt thereof, wherein the combination is a fixed combination or a non-fixed combination.

Embodiment 236. A product comprising a compound of any one of Embodiments 1 to 119, or pharmaceutically acceptable salt thereof, 6-(piperazin-1-yl)-N-(6-(o-tolyl)-5-(trifluoromethyl)pyridin-2-yl)pyridine-2-sulfonamide or a pharmaceutically acceptable salt thereof and (S)-3-amino-6-methoxy-N-(3,3,3-trifluoro-2-hydroxy-2-methylpropyl)-5-(trifluoromethyl)picolinamide or a pharmaceutically acceptable salt thereof, as a combined preparation for simultaneous, separate or sequential use in therapy.

Embodiment 237. A product comprising 4-(4,4-dioxido-2³-(trifluoromethyl)-4-thia-3,6-diaza-2,5(2,6)-dipyridina-1(1,2)-benzenacyclodecaphane-6-yl)butanoic acid, or a pharmaceutically acceptable salt thereof, 6-(piperazin-1-yl)-N-(6-(o-tolyl)-5-(trifluoromethyl)pyridin-2-yl)pyridine-2-sulfonamide or a pharmaceutically acceptable salt thereof and (S)-3-amino-6-methoxy-N-(3,3,3-trifluoro-2-hydroxy-2-methylpropyl)-5-(trifluoromethyl)picolinamide or a pharmaceutically acceptable salt thereof, as a combined preparation for simultaneous, separate or sequential use in therapy.

Embodiment 238. A product comprising 4-(4,4-dioxido-2³-(trifluoromethyl)-4-thia-3,6-diaza-2,5(2,6)-dipyridina-1(1,2)-benzenacycloundecaphane-6-yl)butanoic acid, or a pharmaceutically acceptable salt thereof, 6-(piperazin-1-yl)-N-(6-(o-tolyl)-5-(trifluoromethyl)pyridin-2-yl)pyridine-2-sulfonamide or a pharmaceutically acceptable salt thereof and (S)-3-amino-6-methoxy-N-(3,3,3-trifluoro-2-hydroxy-2-methylpropyl)-5-(trifluoromethyl)picolinamide or a pharmaceutically acceptable salt thereof, as a combined preparation for simultaneous, separate or sequential use in therapy.

Embodiment 239. A product comprising 4-(4,4-dioxido-2³-(trifluoromethyl)-4-thia-3,6-diaza-2,5(2,6)-dipyridina-1(1,2)-benzenacyclododecaphane-6-yl)butanoic acid, or a pharmaceutically acceptable salt thereof, 6-(piperazin-1-yl)-N-(6-(o-tolyl)-5-(trifluoromethyl)pyridin-2-yl)pyridine-2-sulfonamide or a pharmaceutically acceptable salt thereof and (S)-3-amino-6-methoxy-N-(3,3,3-trifluoro-2-hydroxy-2-methylpropyl)-5-(trifluoromethyl)picolinamide or a pharmaceutically acceptable salt thereof, as a combined preparation for simultaneous, separate or sequential use in therapy.

Embodiment 240. A product comprising 4-(4,4-dioxido-2³-(trifluoromethyl)-4-thia-3,6-diaza-2,5(2,6)-dipyridina-1(1,2)-benzenacyclotridecaphane-6-yl)butanoic acid, or a pharmaceutically acceptable salt thereof, 6-(piperazin-1-yl)-N-(6-(o-tolyl)-5-(trifluoromethyl)pyridin-2-yl)pyridine-2-sulfonamide or a pharmaceutically acceptable salt thereof and (S)-3-amino-6-methoxy-N-(3,3,3-trifluoro-2-hydroxy-2-methylpropyl)-5-(trifluoromethyl)picolinamide or a pharmaceutically acceptable salt thereof, as a combined preparation for simultaneous, separate or sequential use in therapy.

Embodiment 241. A product comprising 4-(4,4-dioxido-2³-(trifluoromethyl)-4-thia-3,6-diaza-2,5(2,6)-dipyridina-1(1,2)-benzenacyclotetradecaphane-6-yl)butanoic acid, or a pharmaceutically acceptable salt thereof, 6-(piperazin-1-yl)-N-(6-(o-tolyl)-5-(trifluoromethyl)pyridin-2-yl)pyridine-2-sulfonamide or a pharmaceutically acceptable salt thereof and (S)-3-amino-6-methoxy-N-(3,3,3-trifluoro-2-hydroxy-2-methylpropyl)-5-(trifluoromethyl)picolinamide or a pharmaceutically acceptable salt thereof, as a combined preparation for simultaneous, separate or sequential use in therapy.

Embodiment 242. A product comprising 4-(4,4-dioxido-2³-(trifluoromethyl)-4-thia-3,6-diaza-2,5(2,6)-dipyridina-1(1,2)-benzenacyclopentadecaphane-6-yl)butanoic acid, or a pharmaceutically acceptable salt thereof, 6-(piperazin-1-yl)-N-(6-(o-tolyl)-5-(trifluoromethyl)pyridin-2-yl)pyridine-2-sulfonamide or a pharmaceutically acceptable salt thereof and (S)-3-amino-6-methoxy-N-(3,3,3-trifluoro-2-hydroxy-2-methylpropyl)-5-(trifluoromethyl)picolinamide or a pharmaceutically acceptable salt thereof, as a combined preparation for simultaneous, separate or sequential use in therapy.

Embodiment 243. A product comprising a compound of any one of Embodiments 1 to 119, or pharmaceutically acceptable salt thereof, 6-(piperazin-1-yl)-N-(6-(o-tolyl)-5-(trifluoromethyl)pyridin-2-yl)pyridine-2-sulfonamide or a pharmaceutically acceptable salt thereof and N-(2,4-di-tert-butyl-5-hydroxyphenyl)-1,4-dihydro-4-oxoquinoline-3-carboxamide, or a pharmaceutically acceptable salt thereof, as a combined preparation for simultaneous, separate or sequential use in therapy.

Embodiment 244. A product comprising 4-(4,4-dioxido-2³-(trifluoromethyl)-4-thia-3,6-diaza-2,5(2,6)-dipyridina-1(1,2)-benzenacyclodecaphane-6-yl)butanoic acid, or pharmaceutically acceptable salt thereof, 6-(piperazin-1-yl)-N-(6-(o-tolyl)-5-(trifluoromethyl)pyridin-2-yl)pyridine-2-sulfonamide or a pharmaceutically acceptable salt thereof and N-(2,4-di-tert-butyl-5-hydroxyphenyl)-1,4-dihydro-4-oxoquinoline-3-carboxamide, or a pharmaceutically acceptable salt thereof, as a combined preparation for simultaneous, separate or sequential use in therapy.

Embodiment 245. A product comprising 4-(4,4-dioxido-2³-(trifluoromethyl)-4-thia-3,6-diaza-2,5(2,6)-dipyridina-1(1,2)-benzenacycloundecaphane-6-yl)butanoic acid, or pharmaceutically acceptable salt thereof, 6-(piperazin-1-yl)-N-(6-(o-tolyl)-5-(trifluoromethyl)pyridin-2-yl)pyridine-2-sulfonamide or a pharmaceutically acceptable salt thereof and N-(2,4-di-tert-butyl-5-hydroxyphenyl)-1,4-dihydro-4-oxoquinoline-3-carboxamide, or a pharmaceutically acceptable salt thereof, as a combined preparation for simultaneous, separate or sequential use in therapy.

Embodiment 246. A product comprising 4-(4,4-dioxido-2³-(trifluoromethyl)-4-thia-3,6-diaza-2,5(2,6)-dipyridina-1(1,2)-benzenacyclododecaphane-6-yl)butanoic acid, or pharmaceutically acceptable salt thereof, 6-(piperazin-1-yl)-N-(6-(o-tolyl)-5-(trifluoromethyl)pyridin-2-yl)pyridine-2-sulfonamide or a pharmaceutically acceptable salt thereof and N-(2,4-di-tert-butyl-5-hydroxyphenyl)-1,4-dihydro-4-oxoquinoline-3-carboxamide, or a pharmaceutically acceptable salt thereof, as a combined preparation for simultaneous, separate or sequential use in therapy.

Embodiment 247. A product comprising 4-(4,4-dioxido-2³-(trifluoromethyl)-4-thia-3,6-diaza-2,5(2,6)-dipyridina-1(1,2)-benzenacyclotridecaphane-6-yl)butanoic acid, or pharmaceutically acceptable salt thereof, 6-(piperazin-1-yl)-N-(6-(o-tolyl)-5-(trifluoromethyl)pyridin-2-yl)pyridine-2-sulfonamide or a pharmaceutically acceptable salt thereof and N-(2,4-di-tert-butyl-5-hydroxyphenyl)-1,4-dihydro-4-oxoquinoline-3-carboxamide, or a pharmaceutically acceptable salt thereof, as a combined preparation for simultaneous, separate or sequential use in therapy.

Embodiment 248. A product comprising 4-(4,4-dioxido-2³-(trifluoromethyl)-4-thia-3,6-diaza-2,5(2,6)-dipyridina-1(1,2)-benzenacyclotetradecaphane-6-yl)butanoic acid, or pharmaceutically acceptable salt thereof, 6-(piperazin-1-yl)-N-(6-(o-tolyl)-5-(trifluoromethyl)pyridin-2-yl)pyridine-2-sulfonamide or a pharmaceutically acceptable salt thereof and N-(2,4-di-tert-butyl-5-hydroxyphenyl)-1,4-dihydro-4-oxoquinoline-3-carboxamide, or a pharmaceutically acceptable salt thereof, as a combined preparation for simultaneous, separate or sequential use in therapy.

Embodiment 249. A product comprising 4-(4,4-dioxido-2³-(trifluoromethyl)-4-thia-3,6-diaza-2,5(2,6)-dipyridina-1 (1,2)-benzenacyclopentadecaphane-6-yl)butanoic acid, or pharmaceutically acceptable salt thereof, 6-(piperazin-1-yl)-N-(6-(o-tolyl)-5-(trifluoromethyl)pyridin-2-yl)pyridine-2-sulfonamide or a pharmaceutically acceptable salt thereof and N-(2,4-di-tert-butyl-5-hydroxyphenyl)-1,4-dihydro-4-oxoquinoline-3-carboxamide, or a pharmaceutically acceptable salt thereof, as a combined preparation for simultaneous, separate or sequential use in therapy.

Embodiment 250. A pharmaceutical composition comprising a compound of any one of Embodiments 1 to 119, or a pharmaceutically acceptable salt thereof, a CFTR modulator and a pharmaceutically acceptable carrier.

Embodiment 251. A pharmaceutical composition comprising a compound of any one of Embodiments 1 to 119, or a pharmaceutically acceptable salt thereof, a CFTR potentiator and a pharmaceutically acceptable carrier.

Embodiment 252. A pharmaceutical composition comprising a compound of any one of Embodiments 1 to 119, or a pharmaceutically acceptable salt thereof, a CFTR corrector and a pharmaceutically acceptable carrier.

Embodiment 253. A pharmaceutical composition comprising a compound of any one of Embodiments 1 to 119, or pharmaceutically acceptable salt thereof, 6-(piperazin-1-yl)-N-(6-(o-tolyl)-5-(trifluoromethyl)pyridin-2-yl)pyridine-2-sulfonamide or a pharmaceutically acceptable salt thereof and (S)-3-amino-6-methoxy-N-(3,3,3-trifluoro-2-hydroxy-2-methylpropyl)-5-(trifluoromethyl)picolinamide or a pharmaceutically acceptable salt thereof, and a pharmaceutically acceptable carrier.

Embodiment 254. A pharmaceutical composition comprising 4-(4,4-dioxido-2³-(trifluoromethyl)-4-thia-3,6-diaza-2,5(2,6)-dipyridina-1(1,2)-benzenacyclodecaphane-6-yl)butanoic acid, or a pharmaceutically acceptable salt thereof, 6-(piperazin-1-yl)-N-(6-(o-tolyl)-5-(trifluoromethyl)pyridin-2-yl)pyridine-2-sulfonamide or a pharmaceutically acceptable salt thereof and (S)-3-amino-6-methoxy-N-(3,3,3-trifluoro-2-hydroxy-2-methylpropyl)-5-(trifluoromethyl)picolinamide or a pharmaceutically acceptable salt thereof, and a pharmaceutically acceptable carrier.

Embodiment 255. A pharmaceutical composition comprising 4-(4,4-dioxido-2³-(trifluoromethyl)-4-thia-3,6-diaza-2,5(2,6)-dipyridina-1(1,2)-benzenacycloundecaphane-6-yl)butanoic acid, or a pharmaceutically acceptable salt thereof, 6-(piperazin-1-yl)-N-(6-(o-tolyl)-5-(trifluoromethyl)pyridin-2-yl)pyridine-2-sulfonamide or a pharmaceutically acceptable salt thereof and (S)-3-amino-6-methoxy-N-(3,3,3-trifluoro-2-hydroxy-2-methylpropyl)-5-(trifluoromethyl)picolinamide or a pharmaceutically acceptable salt thereof, and a pharmaceutically acceptable carrier.

Embodiment 256. A pharmaceutical composition comprising 4-(4,4-dioxido-2³-(trifluoromethyl)-4-thia-3,6-diaza-2,5(2,6)-dipyridina-1(1,2)-benzenacyclododecaphane-6-yl)butanoic acid, or a pharmaceutically acceptable salt thereof, 6-(piperazin-1-yl)-N-(6-(o-tolyl)-5-(trifluoromethyl)pyridin-2-yl)pyridine-2-sulfonamide or a pharmaceutically acceptable salt thereof and (S)-3-amino-6-methoxy-N-(3,3,3-trifluoro-2-hydroxy-2-methylpropyl)-5-(trifluoromethyl)picolinamide or a pharmaceutically acceptable salt thereof, and a pharmaceutically acceptable carrier.

Embodiment 257. A pharmaceutical composition comprising 4-(4,4-dioxido-2³-(trifluoromethyl)-4-thia-3,6-diaza-2,5(2,6)-dipyridina-1 (1,2)-benzenacyclotridecaphane-6-yl)butanoic acid, or a pharmaceutically acceptable salt thereof, 6-(piperazin-1-yl)-N-(6-(o-tolyl)-5-(trifluoromethyl)pyridin-2-yl)pyridine-2-sulfonamide or a pharmaceutically acceptable salt thereof and (S)-3-amino-6-methoxy-N-(3,3,3-trifluoro-2-hydroxy-2-methylpropyl)-5-(trifluoromethyl)picolinamide or a pharmaceutically acceptable salt thereof, and a pharmaceutically acceptable carrier.

Embodiment 258. A pharmaceutical composition comprising 4-(4,4-dioxido-2³-(trifluoromethyl)-4-thia-3,6-diaza-2,5(2,6)-dipyridina-1(1,2)-benzenacyclotetradecaphane-6-yl)butanoic acid, or a pharmaceutically acceptable salt thereof, 6-(piperazin-1-yl)-N-(6-(o-tolyl)-5-(trifluoromethyl)pyridin-2-yl)pyridine-2-sulfonamide or a pharmaceutically acceptable salt thereof and (S)-3-amino-6-methoxy-N-(3,3,3-trifluoro-2-hydroxy-2-methylpropyl)-5-(trifluoromethyl)picolinamide or a pharmaceutically acceptable salt thereof, and a pharmaceutically acceptable carrier.

Embodiment 259. A pharmaceutical composition comprising 4-(4,4-dioxido-2³-(trifluoromethyl)-4-thia-3,6-diaza-2,5(2,6)-dipyridina-1(1,2)-benzenacyclopentadecaphane-6-yl)butanoic acid, or a pharmaceutically acceptable salt thereof, 6-(piperazin-1-yl)-N-(6-(o-tolyl)-5-(trifluoromethyl)pyridin-2-yl)pyridine-2-sulfonamide or a pharmaceutically acceptable salt thereof and (S)-3-amino-6-methoxy-N-(3,3,3-trifluoro-2-hydroxy-2-methylpropyl)-5-(trifluoromethyl)picolinamide or a pharmaceutically acceptable salt thereof, and a pharmaceutically acceptable carrier.

Embodiment 260. A pharmaceutical composition comprising a compound of any one of Embodiments 1 to 119, or pharmaceutically acceptable salt thereof, 6-(piperazin-1-yl)-N-(6-(o-tolyl)-5-(trifluoromethyl)pyridin-2-yl)pyridine-2-sulfonamide or a pharmaceutically acceptable salt thereof and N-(2,4-di-tert-butyl-5-hydroxyphenyl)-1,4-dihydro-4-oxoquinoline-3-carboxamide, or a pharmaceutically acceptable salt thereof, and a pharmaceutically acceptable carrier.

Embodiment 261. A pharmaceutical composition comprising 4-(4,4-dioxido-2³-(trifluoromethyl)-4-thia-3,6-diaza-2,5(2,6)-dipyridina-1(1,2)-benzenacyclodecaphane-6-yl)butanoic acid, or pharmaceutically acceptable salt thereof, 6-(piperazin-1-yl)-N-(6-(o-tolyl)-5-(trifluoromethyl)pyridin-2-yl)pyridine-2-sulfonamide or a pharmaceutically acceptable salt thereof and N-(2,4-di-tert-butyl-5-hydroxyphenyl)-1,4-dihydro-4-oxoquinoline-3-carboxamide, or a pharmaceutically acceptable salt thereof, and a pharmaceutically acceptable carrier.

Embodiment 262. A pharmaceutical composition comprising 4-(4,4-dioxido-2³-(trifluoromethyl)-4-thia-3,6-diaza-2,5(2,6)-dipyridina-1(1,2)-benzenacycloundecaphane-6-yl)butanoic acid, or pharmaceutically acceptable salt thereof, 6-(piperazin-1-yl)-N-(6-(o-tolyl)-5-(trifluoromethyl)pyridin-2-yl)pyridine-2-sulfonamide or a pharmaceutically acceptable salt thereof and N-(2,4-di-tert-butyl-5-hydroxyphenyl)-1,4-dihydro-4-oxoquinoline-3-carboxamide, or a pharmaceutically acceptable salt thereof, and a pharmaceutically acceptable carrier.

Embodiment 263. A pharmaceutical composition comprising 4-(4,4-dioxido-2³-(trifluoromethyl)-4-thia-3,6-diaza-2,5(2,6)-dipyridina-1(1,2)-benzenacyclododecaphane-6-yl)butanoic acid, or pharmaceutically acceptable salt thereof, 6-(piperazin-1-yl)-N-(6-(o-tolyl)-5-(trifluoromethyl)pyridin-2-yl)pyridine-2-sulfonamide or a pharmaceutically acceptable salt thereof and N-(2,4-di-tert-butyl-5-hydroxyphenyl)-1,4-dihydro-4-oxoquinoline-3-carboxamide, or a pharmaceutically acceptable salt thereof, and a pharmaceutically acceptable carrier.

Embodiment 264. A pharmaceutical composition comprising 4-(4,4-dioxido-2³-(trifluoromethyl)-4-thia-3,6-diaza-2,5(2,6)-dipyridina-1(1,2)-benzenacyclotridecaphane-6-yl)butanoic acid, or pharmaceutically acceptable salt thereof, 6-(piperazin-1-yl)-N-(6-(o-tolyl)-5-(trifluoromethyl)pyridin-2-yl)pyridine-2-sulfonamide or a pharmaceutically acceptable salt thereof and N-(2,4-di-tert-butyl-5-hydroxyphenyl)-1,4-dihydro-4-oxoquinoline-3-carboxamide, or a pharmaceutically acceptable salt thereof, and a pharmaceutically acceptable carrier.

Embodiment 265. A pharmaceutical composition comprising 4-(4,4-dioxido-2³-(trifluoromethyl)-4-thia-3,6-diaza-2,5(2,6)-dipyridina-1(1,2)-benzenacyclotetradecaphane-6-yl)butanoic acid, or pharmaceutically acceptable salt thereof, 6-(piperazin-1-yl)-N-(6-(o-tolyl)-5-(trifluoromethyl)pyridin-2-yl)pyridine-2-sulfonamide or a pharmaceutically acceptable salt thereof and N-(2,4-di-tert-butyl-5-hydroxyphenyl)-1,4-dihydro-4-oxoquinoline-3-carboxamide, or a pharmaceutically acceptable salt thereof, and a pharmaceutically acceptable carrier.

Embodiment 266. A pharmaceutical composition comprising 4-(4,4-dioxido-2³-(trifluoromethyl)-4-thia-3,6-diaza-2,5(2,6)-dipyridina-1(1,2)-benzenacyclopentadecaphane-6-yl)butanoic acid, or pharmaceutically acceptable salt thereof, 6-(piperazin-1-yl)-N-(6-(o-tolyl)-5-(trifluoromethyl)pyridin-2-yl)pyridine-2-sulfonamide or a pharmaceutically acceptable salt thereof and N-(2,4-di-tert-butyl-5-hydroxyphenyl)-1,4-dihydro-4-oxoquinoline-3-carboxamide, or a pharmaceutically acceptable salt thereof, and a pharmaceutically acceptable carrier.

Embodiment 267. A combination comprising a compound of any one of Embodiments 1 to 119, or pharmaceutically acceptable salt thereof, 6-(piperazin-1-yl)-N-(6-(o-tolyl)-5-(trifluoromethyl)pyridin-2-yl)pyridine-2-sulfonamide or a pharmaceutically acceptable salt thereof and (S)-3-amino-6-methoxy-N-(3,3,3-trifluoro-2-hydroxy-2-methylpropyl)-5-(trifluoromethyl)picolinamide or a pharmaceutically acceptable salt thereof, wherein the combination is a fixed combination or a non-fixed combination.

Embodiment 268. A combination comprising 4-(4,4-dioxido-2³-(trifluoromethyl)-4-thia-3,6-diaza-2,5(2,6)-dipyridina-1(1,2)-benzenacyclodecaphane-6-yl)butanoic acid, or a pharmaceutically acceptable salt thereof, 6-(piperazin-1-yl)-N-(6-(o-tolyl)-5-(trifluoromethyl)pyridin-2-yl)pyridine-2-sulfonamide or a pharmaceutically acceptable salt thereof and (S)-3-amino-6-methoxy-N-(3,3,3-trifluoro-2-hydroxy-2-methylpropyl)-5-(trifluoromethyl)picolinamide or a pharmaceutically acceptable salt thereof, wherein the combination is a fixed combination or a non-fixed combination.

Embodiment 269. A combination comprising 4-(4,4-dioxido-2³-(trifluoromethyl)-4-thia-3,6-diaza-2,5(2,6)-dipyridina-1(1,2)-benzenacycloundecaphane-6-yl)butanoic acid, or a pharmaceutically acceptable salt thereof, 6-(piperazin-1-yl)-N-(6-(o-tolyl)-5-(trifluoromethyl)pyridin-2-yl)pyridine-2-sulfonamide or a pharmaceutically acceptable salt thereof and (S)-3-amino-6-methoxy-N-(3,3,3-trifluoro-2-hydroxy-2-methylpropyl)-5-(trifluoromethyl)picolinamide or a pharmaceutically acceptable salt thereof, wherein the combination is a fixed combination or a non-fixed combination.

Embodiment 270. A combination comprising 4-(4,4-dioxido-2³-(trifluoromethyl)-4-thia-3,6-diaza-2,5(2,6)-dipyridina-1(1,2)-benzenacyclododecaphane-6-yl)butanoic acid, or a pharmaceutically acceptable salt thereof, 6-(piperazin-1-yl)-N-(6-(o-tolyl)-5-(trifluoromethyl)pyridin-2-yl)pyridine-2-sulfonamide or a pharmaceutically acceptable salt thereof and (S)-3-amino-6-methoxy-N-(3,3,3-trifluoro-2-hydroxy-2-methylpropyl)-5-(trifluoromethyl)picolinamide or a pharmaceutically acceptable salt thereof, wherein the combination is a fixed combination or a non-fixed combination.

Embodiment 271. A combination comprising 4-(4,4-dioxido-2³-(trifluoromethyl)-4-thia-3,6-diaza-2,5(2,6)-dipyridina-1(1,2)-benzenacyclotridecaphane-6-yl)butanoic acid, or a pharmaceutically acceptable salt thereof, 6-(piperazin-1-yl)-N-(6-(o-tolyl)-5-(trifluoromethyl)pyridin-2-yl)pyridine-2-sulfonamide or a pharmaceutically acceptable salt thereof and (S)-3-amino-6-methoxy-N-(3,3,3-trifluoro-2-hydroxy-2-methylpropyl)-5-(trifluoromethyl)picolinamide or a pharmaceutically acceptable salt thereof, wherein the combination is a fixed combination or a non-fixed combination.

Embodiment 272. A combination comprising 4-(4,4-dioxido-2³-(trifluoromethyl)-4-thia-3,6-diaza-2,5(2,6)-dipyridina-1(1,2)-benzenacyclotetradecaphane-6-yl)butanoic acid, or a pharmaceutically acceptable salt thereof, 6-(piperazin-1-yl)-N-(6-(o-tolyl)-5-(trifluoromethyl)pyridin-2-yl)pyridine-2-sulfonamide or a pharmaceutically acceptable salt thereof and (S)-3-amino-6-methoxy-N-(3,3,3-trifluoro-2-hydroxy-2-methylpropyl)-5-(trifluoromethyl)picolinamide or a pharmaceutically acceptable salt thereof, wherein the combination is a fixed combination or a non-fixed combination.

Embodiment 273. A combination comprising 4-(4,4-dioxido-2³-(trifluoromethyl)-4-thia-3,6-diaza-2,5(2,6)-dipyridina-1(1,2)-benzenacyclopentadecaphane-6-yl)butanoic acid, or a pharmaceutically acceptable salt thereof, 6-(piperazin-1-yl)-N-(6-(o-tolyl)-5-(trifluoromethyl)pyridin-2-yl)pyridine-2-sulfonamide or a pharmaceutically acceptable salt thereof and (S)-3-amino-6-methoxy-N-(3,3,3-trifluoro-2-hydroxy-2-methylpropyl)-5-(trifluoromethyl)picolinamide or a pharmaceutically acceptable salt thereof, wherein the combination is a fixed combination or a non-fixed combination.

Embodiment 274. A combination comprising a compound of any one of Embodiments 1 to 119, or pharmaceutically acceptable salt thereof, 6-(piperazin-1-yl)-N-(6-(o-tolyl)-5-(trifluoromethyl)pyridin-2-yl)pyridine-2-sulfonamide or a pharmaceutically acceptable salt thereof and N-(2,4-di-tert-butyl-5-hydroxyphenyl)-1,4-dihydro-4-oxoquinoline-3-carboxamide, or a pharmaceutically acceptable salt thereof, wherein the combination is a fixed combination or a non-fixed combination.

Embodiment 275. A combination comprising 4-(4,4-dioxido-2³-(trifluoromethyl)-4-thia-3,6-diaza-2,5(2,6)-dipyridina-1(1,2)-benzenacyclodecaphane-6-yl)butanoic acid, or pharmaceutically acceptable salt thereof, 6-(piperazin-1-yl)-N-(6-(o-tolyl)-5-(trifluoromethyl)pyridin-2-yl)pyridine-2-sulfonamide or a pharmaceutically acceptable salt thereof and N-(2,4-di-tert-butyl-5-hydroxyphenyl)-1,4-dihydro-4-oxoquinoline-3-carboxamide, or a pharmaceutically acceptable salt thereof, wherein the combination is a fixed combination or a non-fixed combination.

Embodiment 276. A combination comprising 4-(4,4-dioxido-2³-(trifluoromethyl)-4-thia-3,6-diaza-2,5(2,6)-dipyridina-1(1,2)-benzenacycloundecaphane-6-yl)butanoic acid, or pharmaceutically acceptable salt thereof, 6-(piperazin-1-yl)-N-(6-(o-tolyl)-5-(trifluoromethyl)pyridin-2-yl)pyridine-2-sulfonamide or a pharmaceutically acceptable salt thereof and N-(2,4-di-tert-butyl-5-hydroxyphenyl)-1,4-dihydro-4-oxoquinoline-3-carboxamide, or a pharmaceutically acceptable salt thereof, wherein the combination is a fixed combination or a non-fixed combination.

Embodiment 277. A combination comprising 4-(4,4-dioxido-2³-(trifluoromethyl)-4-thia-3,6-diaza-2,5(2,6)-dipyridina-1(1,2)-benzenacyclododecaphane-6-yl)butanoic acid, or pharmaceutically acceptable salt thereof, 6-(piperazin-1-yl)-N-(6-(o-tolyl)-5-(trifluoromethyl)pyridin-2-yl)pyridine-2-sulfonamide or a pharmaceutically acceptable salt thereof and N-(2,4-di-tert-butyl-5-hydroxyphenyl)-1,4-dihydro-4-oxoquinoline-3-carboxamide, or a pharmaceutically acceptable salt thereof, wherein the combination is a fixed combination or a non-fixed combination.

Embodiment 278. A combination comprising 4-(4,4-dioxido-2³-(trifluoromethyl)-4-thia-3,6-diaza-2,5(2,6)-dipyridina-1(1,2)-benzenacyclotridecaphane-6-yl)butanoic acid, or pharmaceutically acceptable salt thereof, 6-(piperazin-1-yl)-N-(6-(o-tolyl)-5-(trifluoromethyl)pyridin-2-yl)pyridine-2-sulfonamide or a pharmaceutically acceptable salt thereof and N-(2,4-di-tert-butyl-5-hydroxyphenyl)-1,4-dihydro-4-oxoquinoline-3-carboxamide, or a pharmaceutically acceptable salt thereof, wherein the combination is a fixed combination or a non-fixed combination.

Embodiment 279. A combination comprising 4-(4,4-dioxido-2³-(trifluoromethyl)-4-thia-3,6-diaza-2,5(2,6)-dipyridina-1(1,2)-benzenacyclotetradecaphane-6-yl)butanoic acid, or pharmaceutically acceptable salt thereof, 6-(piperazin-1-yl)-N-(6-(o-tolyl)-5-(trifluoromethyl)pyridin-2-yl)pyridine-2-sulfonamide or a pharmaceutically acceptable salt thereof and N-(2,4-di-tert-butyl-5-hydroxyphenyl)-1,4-dihydro-4-oxoquinoline-3-carboxamide, or a pharmaceutically acceptable salt thereof, wherein the combination is a fixed combination or a non-fixed combination.

Embodiment 280. A combination comprising 4-(4,4-dioxido-2³-(trifluoromethyl)-4-thia-3,6-diaza-2,5(2,6)-dipyridina-1(1,2)-benzenacyclopentadecaphane-6-yl)butanoic acid, or pharmaceutically acceptable salt thereof, 6-(piperazin-1-yl)-N-(6-(o-tolyl)-5-(trifluoromethyl)pyridin-2-yl)pyridine-2-sulfonamide or a pharmaceutically acceptable salt thereof and N-(2,4-di-tert-butyl-5-hydroxyphenyl)-1,4-dihydro-4-oxoquinoline-3-carboxamide, or a pharmaceutically acceptable salt thereof, wherein the combination is a fixed combination or a non-fixed combination.

Embodiment 236. The product of any one of Embodiments 149-176 or Embodiments 236-249 for use in treating a disease, disorder, or condition associated with the modulation of CFTR.

Embodiment 237. The product of any one of Embodiments 149-176 or Embodiments 236-249 for use in the treatment of cystic fibrosis, asthma, COPD, chronic bronchitis, pancreatitis or emphysema.

Embodiment 238. The product of any one of Embodiments 149-176 or Embodiments 236-249 for use in the treatment of pancreatitis.

Embodiment 236. The pharmaceutical composition of any one of Embodiments 177-207 or Embodiments 250-266 for use in treating a disease, disorder, or condition associated with the modulation of CFTR.

Embodiment 237. The pharmaceutical composition of any one of Embodiments 177-207 or Embodiments 250-266 for use in the treatment of cystic fibrosis, asthma, COPD, chronic bronchitis, pancreatitis or emphysema.

Embodiment 238. The pharmaceutical composition of any one of Embodiments 177-207 or Embodiments 250-266 for use in the treatment of pancreatitis.

Embodiment 236. The combination of any one of Embodiments 208-235 or Embodiments 267-280 for use in treating a disease, disorder, or condition associated with the modulation of CFTR.

Embodiment 237. The combination of any one of Embodiments 208-235 or Embodiments 267-280 for use in the treatment of cystic fibrosis, asthma, COPD, chronic bronchitis, pancreatitis or emphysema.

Embodiment 238. The combination of any one of Embodiments 208-235 or Embodiments 267-280 for use in the treatment of pancreatitis.

### Biological Assays

Measurement of deIF508-CFTR-HRP Surface Expression in CFBE41o-Cells

This assay quantifies the cell surface expressions of the mutant CFTR channel using an extracellular HRP tag.

A cellular assay was developed to measure surface expression of horseradish peroxidase (HRP) tagged delF508-CFTR in the human bronchial epithelial immortalized CFBE41o-cell line (Phuan, P. W., et al, (2014) Molecular Pharmacology 86:42-51). Specifically, the HRP sequence was inserted into the fourth extracellular loop of delF508-CFTR and stably expressed in CFBE41o-cells. Cells were seeded in 384 well plate at a density of 5000 cells/well and incubated at 37° C. for 12 to 24 hours in medium (Gibco MEM #11095, 10% FBS, 10 mM HEPES, 200 mM L-Glutamine, 200 μg/mL G418, 3 μg/mL Puromycin). The delF508-CFTR-HRP expression was induced with 500 ng/mL doxycycline (Sigma D-9891, dissolved in H₂O and sterile filtered) in medium and the cells were incubated at 37° C. for 48 h. Old medium was removed and fresh medium was added containing 500 ng/mL doxycycline and unknown test compound at required test concentration in DMSO, not exceeding 0.5% final DMSO concentration. The highest concentration tested was 10 μM with a 10-point concentration response curve using a 3-fold dilution. After addition of compounds, the cells were incubated for 24 h at 37° C. On the final day, cells were washed four times in PBS containing 1 mM MgCl₂ and 0.1 mM CaCl₂. HRP-Substrate (SuperSignal ELISA Pico, Fisher #37069) 20 μl/well was added and the luminescence signal was determined (Viewlux, Perkin Elmer). Light was emitted upon addition of exogenous HRP-Substrate only when delF508-CFTR-HRP reached the cell surface and the HRP tag was accessible to the HRP-Substrate (note: HRP-Substrate cannot cross the lipid bilayer to reach delF508-CFTR-HRP misfolded within the cell).

The median activity for the lowest concentration of the compounds on each assay plate was calculated and this value was used to normalize the signal for each well on the respective plate. Three replicates at each concentrations for every compound were run to determine one EC₅₀. The median value was determined and used to calculate compound activities as described below. Effective half maximal values (EC₅₀) were calculated for each compound by performing logistic regression on measured dose-response data points using the equation:

$$Y = Bottom + \frac{Top - Bottom}{1 + {( \frac{X}{{EC}_{50}} )}^{Hillcoefficient}}$$

where “Y” is the observed activity, “Bottom” is the lowest observed value, “Top” is the highest observed value, and the “Hill coefficient” gives the largest absolute value of the slope. The curve fitting is carried out by a curve fitting program implemented at GNF using Matlab (Mathworks).

The dose response curves also were used to calculate Fold Change (FC) using the equation:

$$Fold change = \frac{Top - Bottom}{Bottom}$$

Compound efficacy relative to the reference compound 3-(6-(1-(2,2-difluorobenzo[d][1,3]dioxol-5-yl)cyclopropane-1-carboxamido)-3-methylpyridin-2-yl)benzoic acid was determined using the following formula:

$$\% A max = \frac{F C of test compound}{F C of reference compound} * 100$$

Measurement of delF508-CFTR Functional Activity in Primary Human Bronchial Epithelial Cells (HBECs) Using Multi-Transepithelial Clamp Circuit (MTECC-24) Assay

This assay measures the functional activity of the CFTR channel (Chloride ion transport) in patient derived primary human bronchial epithilial cells with forskolin activation and in the presence of the CFTR corrector/potentiator combination.

Primary human delF508-CFTR bronchial epithelial cells were and cultured according to previously established methods (Neuberger, T. et, al., Methods in Molecular Biology, 2011, 741, pp 39-54). Briefly, before thawing the HBEC cells, T25 flasks were coated with 2 mL of 3T3 conditioned media (MTI-GlobalStem, Cat #GSM9100) for at least 12 hours in 37° C. CO₂ incubator. 1.7×10⁵ cells were seeded into one T25 flask with 5 mL of HBE growth media (BEBM with supplements (Lonza, Cat #CC-3170) with 10 nM of retinoic acid (Sigma, Cat #R-2625) and 1% of PSA (Hyclone, Cat #SV30079.01). Media was changed every day till the cells were 100% confluent. Cells were seeded into T75 flasks (pre-coated with 5 mL of 3T3 conditioned media for more than 12 hrs) at 4.95×10⁵ cells/T75 flask in 15 mL of HBE growth media. Flasks were fed everyday with fresh HBE growth media until the cells were confluent. 24-well transwell plates (Corning, Cat #3378) were coated with 3T3 conditioned media (70 μL on top of the filter, 350 μL to the bottom of the well) overnight in the 37° C. CO₂ incubator. Cells were seeded in the pre-coated 24-well transwell plates at 1.7×10⁵ cell/well with 700 μL of growth media at the bottom of the well and 200 μL of growth media at the top of the filter. After 24 hrs, cells were switched into HBE differentiation media ((DMEM/F12 (Gibco, Cat #11330-032) supplement with 2% Ultroser G (Pall, Cat #15950-017), 2% of Fetal Clone II (Hyclone, Cat #SH30066.03), 0.25% Bovine Brain Extract (Lonza, Cat #CC-4098), 1% of PSA, 2.5 μg/mL of Insulin (Sigma, Cat #19278), 20 nM of Hydrocortisone (Sigma, Cat #H0888), 500 nM of Triiodothyronine (Sigma, Cat #T6397), 2.5 μg/mL of Transferrin (Sigma, Cat #T8158), 250 nM of Ethanolamine (Sigma, Cat #E0135), 1.5 μM of Epinephrine (Sigma, Cat #E4250), 250 nM of Phosphoethanolamine (Sigma, Cat #P0503), 10 nM of retinoic acid)) with 200 μL on top of the filter (apical side) and 700 μL at the bottom of the well (basolateral side). Cells were fed every other day with the differentiation media. After 4 days, cells were exposed to air/liquid interface. Then cells were fed every other day for 4 weeks before fully differentiated.

Cells were washed with 3 mM DTT in PBS at 70 ul/well for 30 min at 37° C. 6 days before the assay. 3 days before the assay, cells were washed again with PBS at 70 ul/well for 30 min at 37° C. Then cells were treated with compound for 24 hrs before the assay.

Following 24 hr treatment with compound, cells were transferred into plates containing 750 ul of assay media on basolateral side and 250 ul on apical side (Assay medium: F-12 Coon's modified, 20 mM HEPES pH7.4 with TRIS Base, No FCS or bicarbonate). The plates wee then transferred to the heated plate compartments (basolateral buffer temperature ˜36.5° C. on the heating block) of the TECC24 system for 45 mins prior to measurements.

Modulators were added sequentially as follows while the TECC24 instrument recorded the equivalent short circuit current (Ieq):

| | | Stock (in | Approx. |
|---|---|---|---|
| | | Assay | incubation |
| Final | Added to plate | medium) | time |
| 3 μM Benzamil | 25 μL Apical | 33 μM | 15 min |
| 10 μM Forskolin | 25 μL Apical | 120 μM | 15 min |
| | 75 μL Basolateral | | |
| 0.5 μM N-(2,4-di-tert-butyl- | 25 μL Apical/ | 6.5 μM | 15 min |
| 5-hydroxypheny1)-1,4- | 75 μL Basolateral | | |
| dihydro-4-oxoquinoline- | | | |
| 3-carboxamide | | | |
| or | | | |
| 0.5 μM (S)-3-amino-6- | | | |
| methoxy-N-(3,3,3- | | | |
| trifluoro-2-hydroxy-2- | | | |
| methylpropy1)-5- | | | |
| (trifluoromethyl) | | | |
| picolinamide | | | |
| 20 μM Bumetanide | 25 μL Apical/ | 280 μM | 30 min |
| | 75 μL Basolateral | | |

Prior to dilution into assay medium the stocks were as follows:
Benzamil stock: 10 mM in DMSO

### Forskolin Stock: 50 mM in DMSO

N-(2,4-di-tert-butyl-5-hydroxyphenyl)-1,4-dihydro-4-oxoquinoline-3-carboxamide: 50 mM in DMSO
(S)-3-amino-6-methoxy-N-(3,3,3-trifluoro-2-hydroxy-2-methylpropyl)-5-(trifluoromethyl)picolinamide: 50 mM stock 100% DMSO
Bumetanide stock: 20 mM in EtOH

The data was normalized using the median signal from wells treated with 0.1% DMSO as a baseline. Curve fitting and EC₅₀ calculations were performed using the following equation:

$$Y = Bottom + \frac{Top - Bottom}{1 + {( \frac{X}{{EC}_{50}} )}^{Hillcoefficient}}$$

where “Y” is the observed activity, “Bottom” is the lowest observed value, “Top” is the highest observed value, and the “Hill coefficient” gives the largest absolute value of the slope. The curve fitting is carried out by a curve fitting program implemented at GNF using Matlab (Mathworks). At least two replicates for every compound were run and EC₅₀ reported in the table are mean values.

The dose response curves also were used to calculate Fold Change (FC) using the equation:

$$Fold change = \frac{Top - Bottom}{Bottom}$$

% Amax calculations were performed using the equation:

$$\% A max = \frac{F C of test compound}{F C of reference compound} * 100 \%$$

where the test compound (added 24 h before assay) was in the presence of the potentiator (S)-3-amino-6-methoxy-N-(3,3,3-trifluoro-2-hydroxy-2-methylpropyl)-5-(trifluoromethyl)picolinamide at the time of assay. The reference compound was a combination of 2 μM 3-(6-(1-(2,2-difluorobenzo[d][1,3]dioxol-5-yl)cyclopropane-1-carboxamido)-3-methylpyridin-2-yl)benzoic acid added 24 h prior to assay and 0.5 μM N-(2,4-di-tert-butyl-5-hydroxyphenyl)-4-oxo-1,4-dihydroquinoline-3-carboxamide added at the time of assay.

TABLE 1
Activity Table

| | DelF508- | DelF508- | MTECC24 | MTECC24 |
|---|---|---|---|---|
| Compound | CFTR-HRP | CFTR-HRP | CFHBEC | CFHBEC |
| No. | EC₅₀ (μM) | Amax % | EC₅₀ (μM) | Amax % |
| 1 | 0.978 | 372.4 | nd | nd |
| 2 | 0.606 | 609.5 | nd | nd |
| 3 | 0.335 | 360.7 | nd | nd |
| 4 | 2.874 | 74.4 | nd | nd |
| 5 | 1.62 | 159.1 | nd | nd |
| 6 | 1.679 | 248.0 | nd | nd |
| 7 | 0.977 | 385.8 | nd | nd |
| 8 | 0.41 | 564.1 | nd | nd |
| 9 | 1.71 | 366.7 | nd | nd |
| 10 | 2.8 | 29.0 | nd | nd |
| 11 | 1.362 | 441.9 | nd | nd |
| 12 | 0.236 | 1229.6 | nd | nd |
| 13 | 0.393 | 601.0 | nd | nd |
| 14 | 0.334 | 488.8 | nd | nd |
| 15 | 0.304 | 707.8 | nd | nd |
| 16 | 0.303 | 1688.5 | nd | nd |
| 17 | 2.344 | 347.2 | nd | nd |
| 18 | 0.645 | 802.4 | nd | nd |
| 19 | 1.619 | 489.9 | nd | nd |
| 20 | 0.36 | 336.6 | nd | nd |
| 21 | 0.179 | 941.0 | nd | nd |
| 22 | 0.603 | 666.2 | nd | nd |
| 23 | 0.69 | 592.3 | nd | nd |
| 24 | 0.165 | 447.7 | nd | nd |
| 25 | 0.267 | 931.9 | nd | nd |
| 26 | 0.668 | 1156.0 | nd | nd |
| 27 | 0.177 | 829.4 | 0.0031 | 132.0 |
| 28 | 0.066 | 1771.3 | 0.0009 | 171.1 |
| 29 | 0.146 | 516.9 | nd | nd |
| 30 | 1.479 | 1656.8 | nd | nd |
| 31 | 0.614 | 580.2 | nd | nd |
| 32 | 0.295 | 1348.8 | nd | nd |
| 33 | 3.3 | 118.6 | nd | nd |
| 34 | 0.281 | 448.1 | nd | nd |
| 35 | 0.331 | 714.9 | nd | nd |
| 36 | 0.253 | 582.6 | nd | nd |
| 37 | 0.385 | 650.9 | nd | nd |
| 38 | 1.409 | 224.1 | nd | nd |
| 39 | 0.233 | 1943.4 | nd | nd |
| 40 | 0.22 | 1162.0 | nd | nd |
| 41 | 0.187 | 3719.1 | nd | nd |
| 42 | 0.219 | 2124.1 | nd | nd |
| 43 | 0.255 | 1988.7 | nd | nd |
| 44 | 0.159 | 1371.6 | nd | nd |
| 45 | 0.361 | 2039.6 | nd | nd |
| 46 | nd | nd | nd | nd |
| 47 | nd | nd | nd | nd |
| 48 | nd | nd | nd | nd |
| 49 | nd | nd | nd | nd |
| 50 | nd | nd | nd | nd |
| 51 | nd | nd | nd | nd |
| 52 | nd | nd | nd | nd |
| 53 | nd | nd | nd | nd |
| 54 | nd | nd | nd | nd |
| 55 | nd | nd | nd | nd |
| 56 | nd | nd | nd | nd |
| 57 | nd | nd | nd | nd |
| 58 | nd | nd | nd | nd |
| 59 | nd | nd | nd | nd |
| 60 | nd | nd | nd | nd |
| 61 | 0.368 | 1284.4 | nd | nd |
| 62 | 0.403 | 2304.2 | nd | nd |
| 63 | 1.801 | 1527.1 | nd | nd |
| 64 | 0.913 | 862.4 | nd | nd |
| 65 | 0.280 | 1792.4 | nd | nd |
| 66 | 0.224 | 1846.6 | nd | nd |
| 67 | 0.608 | 1892.8 | nd | nd |
| 68 | 0.418 | 2591.6 | nd | nd |
| 69 | 0.351 | 2554.2 | nd | nd |
| 70 | 0.373 | 842.8 | nd | nd |
| 71 | 0.291 | 1144.6 | nd | nd |
| 72 | 2.434 | 396.3 | nd | nd |
| 73 | 0.439 | 763.9 | nd | nd |
| 74 | 1.994 | 745.1 | nd | nd |
| 75 | 0.417 | 2887.0 | nd | nd |
| 76 | 0.421 | 2424.6 | nd | nd |
| 77 | 0.492 | 1465.2 | nd | nd |
| 78 | 0.293 | 1510.3 | nd | nd |
| 79 | 1.6 | 3137.8 | nd | nd |
| 80 | 1.404 | 1165.8 | nd | nd |
| 81 | 2.33 | 527.3 | nd | nd |
| 82 | 0.844 | 652.3 | nd | nd |
| 83 | 2.427 | 163.8 | nd | nd |
| 84 | 1.044 | 2591.1 | nd | nd |
| 85 | 0.714 | 2469.7 | nd | nd |
| 86 | 0.293 | 2234.54 | nd | nd |
| 87 | 1.423 | 306.0 | nd | nd |
| 88 | 2.069 | 1135.0 | nd | nd |
| 89 | 1.182 | 858.0 | 0.0309 | 56.7 |
| 90 | 0.919 | 674.4 | 0.0046 | 123.2 |
| 91 | 2.757 | 720.9 | nd | nd |
| 92 | 0.694 | 982.3 | 0.0069 | 90.0 |
| 93 | 2.652 | 178.7 | nd | nd |
| 94 | 0.566 | 737.2 | nd | nd |
| 95 | 1.323 | 1089.2 | nd | nd |
| 96 | 0.431 | 791.6 | nd | nd |
| 97 | 0.747 | 727.5 | nd | nd |
| 98 | 2.033 | 719.0 | 0.100 | 69.2 |
| 99 | 0.230 | 2157.6 | 0.0008 | 279.3 |
| 100 | 0.351 | 1006.2 | 0.0043 | 175.8 |
| 101 | 0.084 | 1426.8 | nd | nd |
| 102 | 2.303 | 1053.6 | nd | nd |
| 103 | 1.384 | 196.6 | nd | nd |
| 104 | 0.226 | 205.4 | nd | nd |
| 105 | 2.218 | 177.5 | nd | nd |
| 106 | 1.408 | 287.3 | nd | nd |
| 107 | 1.263 | 169.1 | nd | nd |
| 108 | 1.269 | 442.7 | nd | nd |
| 109 | 0.386 | 186.2 | nd | nd |
| 110 | 0.477 | 170.2 | nd | nd |
| 111 | 1.062 | 1109.8 | nd | nd |
| 112 | 2.598 | 644.3 | nd | nd |
| 113 | 2.151 | 664.5 | nd | nd |
| 114 | 3.31 | 97.1 | nd | nd |
| 115 | 0.456 | 430.1 | nd | nd |
| 116 | 0.445 | 737.0 | nd | nd |
| 117 | 0.391 | 1152.6 | 0.001 | 213.1 |
| 118 | 0.511 | 705.4 | nd | nd |
| 119 | 0.866 | 355.6 | nd | nd |
| 120 | 1.406 | 948.7 | nd | nd |
| 121 | 0.495 | 348.5 | nd | nd |
| 122 | 1.216 | 226.3 | nd | nd |
| 123 | 1.843 | 307.5 | nd | nd |
| 124 | 1.644 | 382.2 | nd | nd |
| 125 | 0.931 | 162.1 | nd | nd |
| 126 | 0.831 | 487.9 | nd | nd |
| 127 | 3.63 | 93.6 | nd | nd |
| 128 | 0.275 | 435.1 | nd | nd |
| 129 | 0.265 | 505.8 | nd | nd |
| 130 | 2.437 | 188.2 | nd | nd |
| 131 | 3.9 | 30.7 | nd | nd |
| 132 | 2.665 | 258.8 | nd | nd |
| 133 | 0.457 | 568.9 | nd | nd |
| 134 | 0.608 | 410.0 | nd | nd |
| 135 | 0.154 | 650.8 | nd | nd |

nd = not determined

1. A compound of Formula (I), or a pharmaceutically acceptable salt thereof, 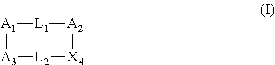
wherein:
A₁, A₂ and A₃ are each independently selected from an optionally substituted arylene and an optionally substituted heteroarylene;
L₁ is a sulfonamide, an amide, a carbonyl or a urea;
L₂ is an optionally substituted alkylene, an optionally substituted alkoxylene, an optionally substituted polyalkylene oxide, an optionally substituted alkylene oxide, an optionally substituted aminoalkylene or an optionally substituted C₃₋₈cycloalkylene;
and
X_{A} is an optionally substituted divalent amino, an optionally substituted divalent amide, an optionally substituted heterocycloalkylene or —O—.

2. The compound of Formula (I) of claim 1, or a pharmaceutically acceptable salt thereof,
wherein:
A₁ is an arylene or a heteroarylene, wherein the arylene and heteroarylene of A₁ is unsubstituted or is substituted with 1 to 2 groups independently selected from H, halo, halo-substituted C₁₋₆alkyl, C₁₋₆ alkyl, deuterium-substituted C₁-C₆alkyl, nitrile, hydroxyl, C₁₋₆alkoxy and halo-substituted C₁₋₆alkoxy;
A₂ is an arylene or a heteroarylene, wherein the arylene and heteroarylene of A₂ is unsubstituted or is substituted with 1 to 2 groups independently selected from H, halo, halo-substituted C₁₋₆alkyl, C₁₋₆ alkyl, deuterium-substituted C₁-C₆alkyl, nitrile, hydroxyl, C₁₋₆alkoxy and halo-substituted C₁₋₆alkoxy;
A₃ is an arylene or a heteroarylene, wherein the arylene and heteroarylene of A₃ is unsubstituted or is substituted with 1 to 2 groups independently selected from H, halo, halo-substituted C₁₋₆alkyl, C₁₋₆ alkyl, deuterium-substituted C₁-C₆alkyl, nitrile, hydroxyl, C₁₋₆alkoxy and halo-substituted C₁₋₆alkoxy;
L₁ is —NR^{A}S(O)₀₋₂—*, —S(O)₀₋₂NR^{A}—*, —NR^{A}C(═O)—*, —C(═O)NR^{A}—*, —C(═O)—, or —NR^{A}C(═O)NR^{A}—, where the * indicates the point of attachment to A₂;
L₂ is an alkylene, an alkoxylene, an alkylene oxide, a polyalkylene oxide, an aminoalkylene or a C₃₋₈cycloalkylene, wherein the alkylene, alkoxylene, alkylene oxide, polyalkylene oxide, aminoalkylene and C₃₋₈cycloalkylene of L2 are unsubstituted or substituted with 1 to 3 groups independently selected from C₁-C₆alkyl, —OH, —(CH₂)ₘC(═O)OR^{A}, C₁-C₆alkoxy, C₁-C₆alkyl substituted with 1 to 6 hydroxyl, groups, deuterium, deuterium-substituted C₁-C₆alkyl, and a spiro attached C₃-C₈cycloalkyl;
X_{A} is an optionally substituted divalent amino, an optionally substituted divalent amide, an optionally substituted heterocycloalkylene or —O—;
R^{A} is H or C₁-C₆alkyl,
and
m is 1, 2, 3, 4, 5, or 6.

3. The compound of claim 2, or a pharmaceutically acceptable salt thereof,
wherein:
A₁ is an arylene or a heteroarylene, wherein the arylene and heteroarylene of A₁ is unsubstituted or is substituted with 1 to 2 groups independently selected from H, halo, halo-substituted C₁₋₆alkyl, C₁₋₆ alkyl, deuterium-substituted C₁-C₆alkyl, nitrile, hydroxyl, C₁₋₆alkoxy and halo-substituted C₁₋₆alkoxy;
A₂ is an arylene or a heteroarylene, wherein the arylene and heteroarylene of A₂ is unsubstituted or is substituted with 1 to 2 groups independently selected from H, halo, halo-substituted C₁₋₆alkyl, C₁₋₆ alkyl, deuterium-substituted C₁-C₆alkyl, nitrile, hydroxyl, C₁₋₆alkoxy and halo-substituted C₁₋₆alkoxy;
A₃ is an arylene or a heteroarylene, wherein the arylene and heteroarylene of A₃ is unsubstituted or is substituted with 1 to 2 groups independently selected from H, halo, halo-substituted C₁₋₆alkyl, C₁₋₆ alkyl, deuterium-substituted C₁-C₆alkyl, nitrile, hydroxyl, C₁₋₆alkoxy and halo-substituted C₁₋₆alkoxy;
L₁ is —NR^{A}S(O)₀₋₂—* or —S(O)₀₋₂NR^{A}—*, where the * indicates the point of attachment to A₂;
L₂ is an alkylene, an alkoxylene, an alkylene oxide, a polyalkylene oxide, an aminoalkylene or a C₃₋₈cycloalkylene, wherein the alkylene, alkoxylene, alkylene oxide, polyalkylene oxide, aminoalkylene and C₃₋₈cycloalkylene of L2 are unsubstituted or substituted with 1 to 3 groups independently selected from C₁-C₆alkyl, —OH, deuterium, —(CH₂)ₘC(═O)OR^{A}, C₁-C₆alkoxy, C₁-C₆alkyl substituted with 1 to 6 hydroxyl, groups, deuterium-substituted C₁-C₆alkyl, and a spiro attached C₃-C₈ cycloalkyl;
X_{A} is an optionally substituted divalent amino, an optionally substituted divalent amide, an optionally substituted heterocycloalkylene or —O—;
R^{A} is H or C₁-C₆alkyl,
and
m is 1, 2, 3, 4, 5, or 6.

4. The compound of claim 3, or a pharmaceutically acceptable salt thereof,
wherein:
A₁ is a phenylene or a pyridylene, wherein the phenylene or pyridylene of A₁ is unsubstituted or is substituted with 1 to 2 groups independently selected from H, halo, halo-substituted C₁₋₆alkyl, C₁₋₆ alkyl, deuterium-substituted C₁-C₆alkyl, nitrile, hydroxyl, C₁₋₆alkoxy and halo-substituted C₁₋₆alkoxy;
A₂ is a phenylene or a pyridylene, wherein the phenylene or pyridylene of A₂ is unsubstituted or is substituted with 1 to 2 groups independently selected from H, halo, halo-substituted C₁₋₆alkyl, C₁₋₆ alkyl, deuterium-substituted C₁-C₆alkyl, nitrile, hydroxyl, C₁₋₆alkoxy and halo-substituted C₁₋₆alkoxy;
A₃ is a phenylene or a pyridylene, wherein the phenylene or pyridylene of A₃ is unsubstituted or is substituted with 1 to 2 groups independently selected from H, halo, halo-substituted C₁₋₆alkyl, C₁₋₆ alkyl, deuterium-substituted C₁-C₆alkyl, nitrile, hydroxyl, C₁₋₆alkoxy and halo-substituted C₁₋₆alkoxy;
L₁ is —NR^{A}S(O)₀₋₂—* or —S(O)₀₋₂NR^{A}—*, where the * indicates the point of attachment to A₂;
L₂ is an alkylene, an alkoxylene, an alkylene oxide, a polyalkylene oxide, an aminoalkylene or a C₃₋₈cycloalkylene, wherein the alkylene, alkoxylene, alkylene oxide, polyalkylene oxide, aminoalkylene and C₃₋₈cycloalkylene of L₂ are unsubstituted or substituted with 1 to 3 groups independently selected from C₁-C₆alkyl, —OH, deuterium, —(CH₂)ₘC(═O)OR^{A}, C₁-C₆alkoxy, C₁-C₆alkyl substituted with 1 to 6 hydroxyl, groups, deuterium-substituted C₁-C₆alkyl, and a spiro attached C₃-C₈cycloalkyl;
X_{A} is an optionally substituted divalent amino, an optionally substituted divalent amide, an optionally substituted heterocycloalkylene or —O—;
R^{A} is H or C₁-C₆alkyl,
and
m is 1, 2, 3, 4, 5, or 6.

5. The compound of claim 4, or a pharmaceutically acceptable salt thereof,
wherein:
A₁ is a pyridylene, wherein the pyridylene of A₁ is substituted with 1 to 2 groups independently selected from H, halo, halo-substituted C₁₋₆alkyl, C₁₋₆ alkyl, deuterium-substituted C₁-C₆alkyl, nitrile, hydroxyl, C₁₋₆alkoxy and halo-substituted C₁₋₆alkoxy;
A₂ is a pyridylene, wherein the pyridylene of A₂ is unsubstituted;
A₃ is a phenylene or a pyridylene, wherein the phenylene or pyridylene of A₃ is unsubstituted or is substituted with 1 to 2 groups independently selected from H, halo, halo-substituted C₁₋₆alkyl, C₁₋₆ alkyl, deuterium-substituted C₁-C₆alkyl, nitrile, hydroxyl, C₁₋₆alkoxy and halo-substituted C₁₋₆alkoxy;
L₁ is —NR^{A}S(O)₀₋₂—* or —S(O)₀₋₂NR^{A}—*, where the * indicates the point of attachment to A₂;
L₂ is an alkylene, an alkoxylene, an alkylene oxide, a polyalkylene oxide, an aminoalkylene or a C₃₋₈cycloalkylene, wherein the alkylene, alkoxylene, alkylene oxide, polyalkylene oxide, aminoalkylene and C₃₋₈cycloalkylene of L₂ are unsubstituted or substituted with 1 to 3 groups independently selected from C₁-C₆alkyl, —OH, deuterium, —(CH₂)ₘC(═O)OR^{A}, C₁-C₆alkoxy, C₁-C₆alkyl substituted with 1 to 6 hydroxyl, groups, deuterium-substituted C₁-C₆alkyl, and a spiro attached C₃-C₈cycloalkyl;
X_{A} is an optionally substituted divalent amino, an optionally substituted divalent amide, an optionally substituted heterocycloalkylene or —O—;
R^{A} is H or C₁-C₆alkyl,
and
m is 1, 2, 3, 4, 5, or 6.

6. The compound of claim 3, or a pharmaceutically acceptable salt thereof, having the structure of Formula (I-a), 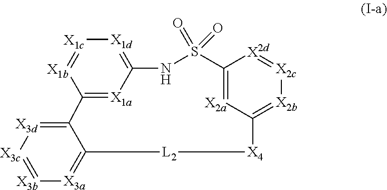
wherein:
X₁ₐ, X_{1b}, X_{1c} and X_{1d} are each independently selected from is CR¹ or N, wherein only 1 or 2 of X₁ₐ, X_{1b}, X_{1c} and X_{1d} can be N and the others are CR¹;
X₂ₐ, X_{2b}, X_{2c} and X_{2d} are each independently selected from is CR¹ or N, wherein only 1 or 2 of X₂ₐ, X_{2b}, X_{2c} and X_{2d} can be N and the others are CR¹;
X₃ₐ, X_{3b}, X_{3c} and X_{3d} are each independently selected from is CR² or N, wherein only 1 or 2 of X₃ₐ, X_{3b}, X_{3c} and X_{3d} can be N and the others are CR²;
X₄ is 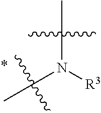
or —O—, wherein * indicates the point of attachment to L₂;
L₂ is —(CR⁴R⁵)ₙ—,
—O(CR⁴R⁵)ₙ—**,
—NR⁷(CR⁴R⁵)ₙ—**,
—(CR⁴R⁵)ₙO(CR⁶R¹⁰)ₚ—**,
—(CR⁴R⁵)ₙ(CR⁶R¹⁸)—**,
—(CR⁴R⁵)ₙ(CR⁸R⁹)ₚ(CR⁴R⁵)ₘ—,
—(CR⁴R⁵)ₚNR⁷(CR⁶R¹⁰)ₙ—**,
—(CR⁴R⁵)ₙO)ₜ(CR⁶R¹⁰)ₚ—**,
or
—O—C₃-C₈ cycloalkylene-**,
wherein ** indicates the point of attachment to X₄;
each R¹ is independently selected from H, halo, halo-substituted C₁-C₆alkyl, C₁-C₆alkyl, deuterium-substituted C₁-C₆alkyl, nitrile, hydroxyl, C₁₋₆alkoxy and halo-substituted C₁₋₆alkoxy;
each R² is independently selected from H, halo, nitrile, hydroxyl, halo-substituted C₁₋₆alkyl, C₁₋₆alkyl, deuterium-substituted C₁-C₆alkyl, hydroxy-substituted C₁-C₆alkyl, C₁₋₆alkoxy and halo-substituted C₁₋₆alkoxy;
R³ is H,
—C₁₋₆alkyl,
—(CR¹¹R¹²)_{y}R¹⁶,
—(CR¹¹R¹²)_{y}C(═O)OR¹³,
—((CR¹¹R¹²)_{y}O(CR¹⁴R¹⁵)_{z}C(═O)OR¹³,
—((CR¹¹R¹²)_{y}O(CR¹⁴R¹⁵)_{z}OR¹³,
—(CR¹¹R¹²)_{y}C(═O)R¹³,
—(CR¹¹R¹²)_{y}OR¹³,
—(CR¹¹R¹²)_{y}(CR⁸R⁹)_{z}C(═O)OR¹³,
—(CR¹¹R¹²)_{y}(CR⁸R⁹)_{z}OR¹³,
—(CR¹¹R¹²)_{y}(CR⁸R⁹)_{z}(CR¹⁴R¹⁵)_{q}OR¹³,
—(CR¹¹R¹²)_{y}NR¹⁰(CR¹⁴R¹⁵)_{q}C(═O)OR¹³,
—(CR¹¹R¹²)_{y}NR¹³R¹⁴,
—(CR¹¹R¹²)_{y}R²⁰,
—((CR¹¹R¹²)_{y}O)_{q}(CR¹⁴R¹⁵)_{z}C(═O)OR¹³,
—((CR¹¹R¹²)_{y}O)_{q}(CR¹⁴R¹⁵)_{z}OR¹³, 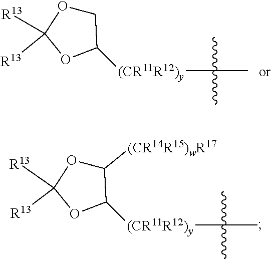
each R⁴ is independently selected from H, D, deuterium-substituted C₁-C₆alkyl and C₁₋₆alkyl;
each R⁵ is independently selected from H, D deuterium-substituted C₁-C₆alkyl and C₁₋₆alkyl;
each R⁶ is independently selected from H, D deuterium-substituted C₁-C₆alkyl and C₁₋₆alkyl;
each R⁷ is independently selected from H, and C₁₋₆alkyl;
each R⁸ and R⁹ are independently selected from H, D, deuterium-substituted C₁-C₆alkyl, C₁₋₆alkyl, or —OH;
or R⁸ and R⁹ together with carbon in CR⁸R⁹ form C₃₋₈cycloalkyl;
each R¹⁰ is independently selected from H, D and C₁₋₆alkyl;
each R¹¹ is independently selected from H, D and C₁₋₆alkyl;
each R¹² is independently selected from H, D, deuterium-substituted C₁-C₆alkyl and C₁₋₆alkyl;
each R¹³ is independently selected from H, and C₁₋₆alkyl;
each R¹⁴ is independently selected from H, D, deuterium-substituted C₁-C₆alkyl and C₁₋₆alkyl;
each R¹⁵ is independently selected from H, D, deuterium-substituted C₁-C₆alkyl and C₁₋₆alkyl;
R¹⁶ is a 4-6 membered heterocycloalkyl having 1-2 ring members independently selected from N, O and S, wherein said 4-6 membered heterocycloalkyl is unsubstituted or substituted with 1-2 R¹⁷ groups;
each R¹⁷ is independently selected from C₁₋₆alkyl and hydroxyl;
R¹⁸ is H, C₁₋₆alkyl, —C(R⁴R⁵)ₘOR¹⁹, or —(CH₂)ₘC(═O)OR¹⁹;
each R¹⁹ is independently selected from H and C₁₋₆alkyl;
R²⁰ is 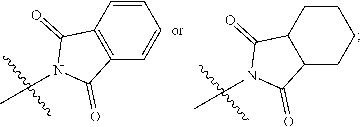 [SMILES: *N1C(=O)c2ccccc2C1=O ; *N1C(=O)C2CCCCC2C1=O]
each m is independently selected from 1, 2, 3, 4, 5, 6, 7, 8, 9 and 10;
each n is independently selected from 1, 2, 3, 4, 5, 6, 7, 8, 9 and 10;
each p is independently selected from 1, 2, 3, 4, 5, 6, 7, 8, 9 and 10;
each t is independently selected from 1, 2, 3, 4, 5, 6, 7, 8, 9 and 10;
each w is independently selected from 1, 2, 3, 4, 5, 6, 7, 8, 9 and 10;
each y is independently selected from 1, 2, 3, 4, 5, 6, 7, 8, 9 and 10;
each z is independently selected from 1, 2, 3, 4, 5, 6, 7, 8, 9 and 10;
and,
each q is independently selected from 1, 2, 3, 4, 5, 6, 7, 8, 9 and 10.

7. The compound of claim 6, or a pharmaceutically acceptable salt thereof, having the structure of formula of Formula (I-b),

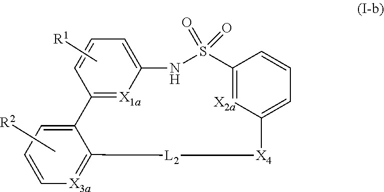

8. The compound of claim 7, or a pharmaceutically acceptable salt thereof, having the structure of Formula (I-c) or Formula (I-d), 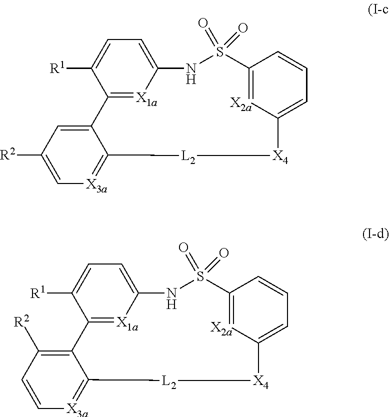
wherein:
X₁ₐ is CH or N;
X₂ₐ is CH or N;
and
X₃ₐ is CH or N.

9. The compound of claim 8, or a pharmaceutically acceptable salt thereof, having the structure of Formula (I-e), Formula (I-f), Formula (I-g) or Formula (I-h) wherein: 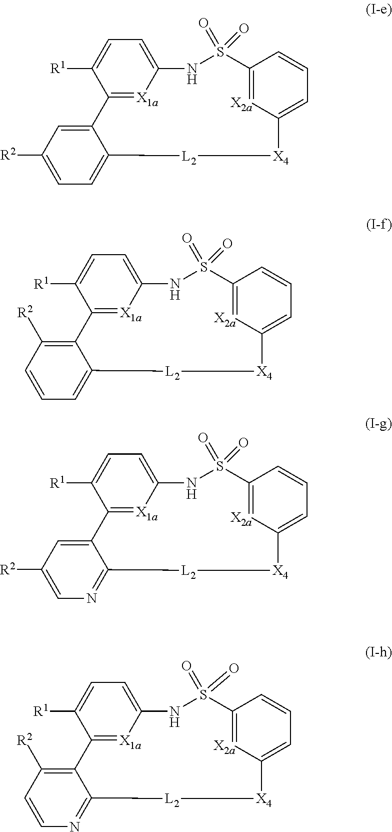
wherein:
X₁ₐ is CH or N;
and
X₂ₐ is CH or N.

10. The compound of claim 9, or a pharmaceutically acceptable salt thereof, having the structure of Formula (I-i), Formula (I-j), Formula (I-k) or Formula (I-l) wherein:

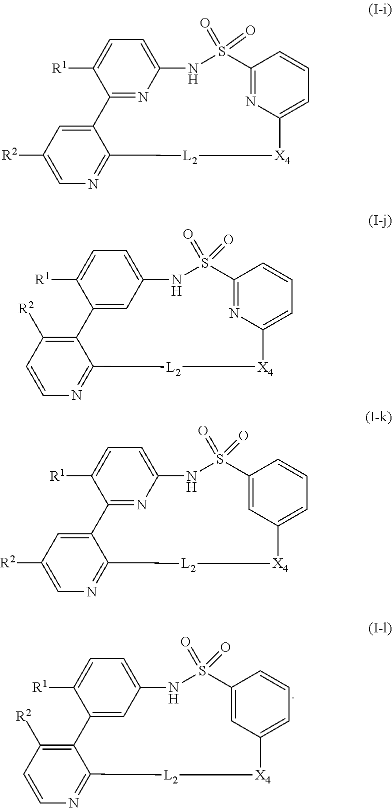

11. The compound of claim 9, or a pharmaceutically acceptable salt thereof, having the structure of Formula (I-m), Formula (I-n), Formula (I-o) or Formula (I-p) wherein:

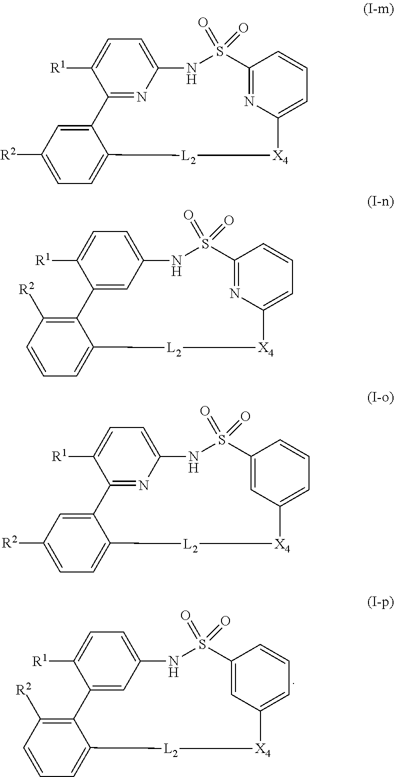

12. The compound of claim 7, or a pharmaceutically acceptable salt thereof, having the structure of formula of Formula (I-g),

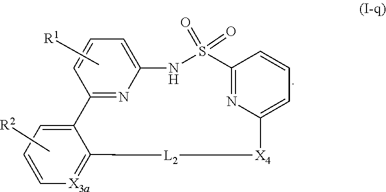

13. The compound of claim 12, or a pharmaceutically acceptable salt thereof, wherein:
X₄ is 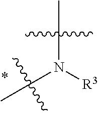
or —O—, wherein * indicates the point of attachment to L₂;
L₂ is —(CR⁴R⁵)ₙ—,
—O(CR⁴R⁵)ₙ—**,
—NR⁷(CR⁴R⁵)ₙ—**,
—(CR⁴R⁵)ₙO(CR⁶R¹⁰)ₚ—**,
—(CR⁴R⁵)ₙ(CR⁶R¹⁸)—**,
—(CR⁴R⁵)ₙ(CR⁸R⁹)ₚ(CR⁴R⁵)ₘ—,
—(CR⁴R⁵)ₚNR⁷(CR⁶R¹⁰)ₙ—**,
or
—O—C₃₋₈cycloalkylene-**, wherein ** indicates the point of attachment to X₄;
R¹ is H, halo, halo-substituted C₁₋₆alkyl, C₁₋₆ alkyl, or C₁₋₆alkoxy;
R² is H, or halo;
R³ is H,
—C₁₋₆alkyl,
—(CR¹¹R¹²)_{y}R¹⁶,
—(CR¹¹R¹²)_{y}C(═O)OR¹³,
—((CR¹¹R¹²)_{y}O(CR¹⁴R¹⁵)_{z}C(═O)OR¹³,
—((CR¹¹R¹²)_{y}O(CR¹⁴R¹⁵)_{z}OR¹³,
—(CR¹¹R¹²)_{y}C(═O)R¹³,
—(CR¹¹R¹²)_{y}OR¹³,
—(CR¹¹R¹²)_{y}(CR⁸R⁹)_{z}C(═O)OR¹³,
—(CR¹¹R¹²)_{y}(CR⁸R⁹)_{z}OR¹³,
—(CR¹¹R¹²)_{y}(CR⁸R⁹)_{z}(CR⁴R¹⁵)_{q}OR³,
—(CR¹¹R¹²)_{y}NR¹⁰(CR¹⁴R⁵)_{q}C(═O)OR¹³,
—(CR¹¹R¹²)_{y}NR¹³R¹⁴,
—(CR¹¹R¹²)_{y}R²⁰, 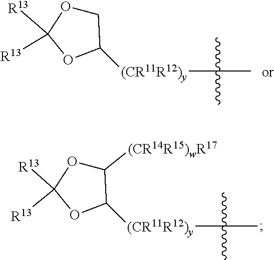
each R⁴ is H;
each R⁵ is H;
each R⁶ is H;
each R⁷ is independently selected from H, and C₁₋₆alkyl;
each R⁸ and R⁹ are independently selected from H, C₁₋₆alkyl, or —OH;
or R⁸ and R⁹ together with carbon in CR⁸R⁹ form C₃₋₈cycloalkyl;
each R¹⁰ is H;
each R¹¹ is H;
each R¹² is H;
each R¹³ is independently selected from H, and C₁₋₆alkyl;
each R¹⁴ is H;
each R¹⁵ is H;
R¹⁶ is a 4-6 membered heterocycloalkyl having 1-2 ring members independently selected from N or O, wherein said 4-6 membered heterocycloalkyl is unsubstituted or substituted with 1-2 R¹⁷ groups;
each R¹⁷ is independently selected from C₁₋₆alkyl and hydroxyl;
R¹⁸ is —C(R⁴R⁵)ₘOR¹⁹, or —(CH₂)ₘC(═O)OR¹⁹;
each R¹⁹ is H;
R²⁰ 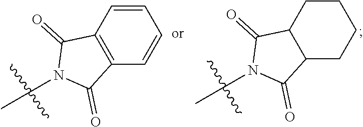 [SMILES: *N1C(=O)c2ccccc2C1=O ; *N1C(=O)C2CCCCC2C1=O]
each m is independently selected from 1, 2 and 3;
each n is independently selected from 1, 2, 3, 4, 5, 6, 7, 8, and 9;
each p is independently selected from 1, 2 and 3;
each w is independently selected from 1 and 2;
each y is independently selected from 1, 2, 3, 4 and 5;
each z is independently selected from 1, 2 and 3;
and,
each q is independently selected from 1 and 2.

14. The compound of claim 13, or a pharmaceutically acceptable salt thereof, wherein:
X₄ is 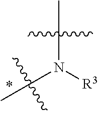
or —O—, wherein * indicates the point of attachment to L₂;
L₂ is —(CR⁴R⁵)ₙ—,
—O(CR⁴R⁵)ₙ—**,
or
—(CR⁴R⁵)ₙO(CR⁶R¹⁰)ₚ—**, wherein ** indicates the point of attachment to X₄;
R¹ is halo or halo-substituted C₁₋₆alkyl;
R² is H, or halo (F;
R³ is —(CR¹¹R¹²)_{y}C(═O)OR¹³,
—(CR¹¹R¹²)_{y}OR¹³,
—(CR¹¹R¹²)_{y}(CR⁸R⁹)_{z}C(═O)OR¹³,
or
—(CRI R¹²)_{y}(CR⁸R⁹)_{z}(CR¹⁴R¹⁵)_{q}OR¹³,
each R⁴ is H;
each R⁵ is H;
each R⁶ is H;
each R⁸ and R⁹ are independently selected from H or C₁₋₆alkyl;
or R⁸ and R⁹ together with carbon in CR⁸R⁹ form C₃₋₈cycloalkyl;
each R¹⁰ is H;
each R¹¹ is H;
each R¹² is H;
each R¹³ is independently selected from H and C₁₋₆alkyl;
each R¹⁴ is H;
each R¹⁵ is H;
each n is independently selected from 1, 2, 3, 4, 5, 6, 7, 8, and 9;
each p is independently selected from 1, 2 and 3;
each y is independently selected from 1, 2, 3, 4 and 5;
each z is independently selected from 1, 2 and 3;
and,
each q is independently selected from 1 and 2.

15. The compound of claim 14, or a pharmaceutically acceptable salt thereof, wherein:
X₄ is 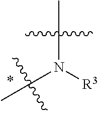
or —O—, wherein * indicates the point of attachment to L₂;
L₂ is —(CR⁴R⁵)ₙ—,
—O(CR⁴R⁵)ₙ—**,
or
—(CR⁴R⁵)ₙO(CR⁶R¹⁰)ₚ—**, wherein ** indicates the point of attachment to X₄;
R¹ is C₁, F or CF₃;
R² is H or F;
R³ is —(CR¹¹R¹²)_{y}C(═O)OR¹³, —(CR¹¹R¹²)_{y}OR¹³, —(CR¹¹R¹²)_{y}(CR⁸R⁹)_{z}C(═O)OR¹³,
or —(CR¹¹R¹²)_{y}(CR⁸R⁹)_{z}(CR¹⁴R¹⁵)_{q}OR¹³,
each R⁴ is H;
each R⁵ is H;
each R⁶ is H;
each R⁸ and R⁹ are independently selected from H or methyl;
or R⁸ and R⁹ together with carbon in CR⁸R⁹ form a cyclopropyl;
each R¹⁰ is H;
each R¹¹ is H;
each R¹² is H;
each R¹³ is independently selected from H, methyl and ethyl;
each R¹⁴ is H;
each R¹⁵ is H;
each n is independently selected from 1, 2, 3, 4, 5, 6, 7, 8, and 9;
each p is independently selected from 1, 2 and 3;
each y is independently selected from 1, 2, 3, 4 and 5;
z is 1;
and,
q is 1.

16. The compound of claim 15, or a pharmaceutically acceptable salt thereof, wherein:
X₄ is 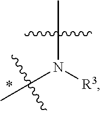
wherein * indicates the point of attachment to L₂;
L₂ is —(CR⁴R⁵)ₙ—;
R¹ is CF₃;
R² is H;
R³ is —(CR¹¹R¹²)_{y}C(═O)OR¹³;
each R⁴ is H;
each R⁵ is H;
each R¹¹ is H;
each R¹² is H;
each R¹³ is H;
n is 1, 2, 3, 4, 5, 6, 7, 8, or 9;
and
y is 2, 3 or 4.

17. The compound of claim 6, selected from:
6-(2-morpholinoethyl)-2³-(trifluoromethyl)-4-thia-3,6-diaza-2,5(2,6)-dipyridina-1(1,2)-benzenacycloundecaphane 4,4-dioxide;
2³-(trifluoromethyl)-11-oxa-4-thia-3,6-diaza-2,5(2,6)-dipyridina-1(1,2)-benzenacycloundecaphane 4,4-dioxide;
2-(4,4-dioxido-2³-(trifluoromethyl)-1¹-oxa-4-thia-3,6-diaza-2,5(2,6)-dipyridina-1(1,2)-benzenacycloundecaphane-6-yl)acetic acid;
2³-(trifluoromethyl)-4-thia-3,6,1¹-triaza-1(3,2),2,5(2,6)-tripyridinacycloundecaphane 4,4-dioxide;
2³-(trifluoromethyl)-1²-oxa-4-thia-3,6-diaza-1(3,2),2,5(2,6)-tripyridinacyclododecaphane 4,4-dioxide;
4-(1⁵-fluoro-4,4-dioxido-2³-(trifluoromethyl)-11-oxa-4-thia-3,6-diaza-2,5(2,6)-dipyridina-1(1,2)-benzenacycloundecaphane-6-yl)butanoic acid;
3-(2-(4,4-dioxido-2³-(trifluoromethyl)-4-thia-3,6-diaza-2,5(2,6)-dipyridina-1(1,2)-benzenacycloundecaphane-6-yl)ethoxy)propanoic acid;
4-(4,4-dioxido-2³-(trifluoromethyl)-4-thia-3,6-diaza-2,5(2,6)-dipyridina-1(1,2)-benzenacyclotridecaphane-6-yl)butanoic acid;
2³-(trifluoromethyl)-10-oxa-4-thia-3,6-diaza-2,5(2,6)-dipyridina-1(1,2)-benzenacycloundecaphane 4,4-dioxide;
4-(4,4-dioxido-2³-(trifluoromethyl)-4-thia-3,6-diaza-2,5(2,6)-dipyridina-1(1,2)-benzenacyclopentadecaphane-6-yl)butanoic acid;
6-(2-(3-hydroxypropoxy)ethyl)-2³-(trifluoromethyl)-4-thia-3,6-diaza-2,5(2,6)-dipyridina-1(1,2)-benzenacycloundecaphane 4,4-dioxide;
4-(4,4-dioxido-2³-(trifluoromethyl)-4-thia-3,6-diaza-2,5(2,6)-dipyridina-1(1,2)-benzenacycloundecaphane-6-yl)butanoic acid;
4-(1⁵-fluoro-4,4-dioxido-2³-(trifluoromethyl)-4-thia-3,6-diaza-2,5(2,6)-dipyridina-1(1,2)-benzenacyclododecaphane-6-yl)butanoic acid;
3-(4,4-dioxido-2³-(trifluoromethyl)-4-thia-3,6-diaza-2,5(2,6)-dipyridina-1(1,2)-benzenacyclotetradecaphane-6-yl)propanoic acid;
6-(4,4-dioxido-2³-(trifluoromethyl)-4-thia-3,6-diaza-2,5(2,6)-dipyridina-1(1,2)-benzenacycloundecaphane-6-yl)hexanoic acid;
4-(4,4-dioxido-2³-(trifluoromethyl)-4-thia-3,6-diaza-2,5(2,6)-dipyridina-1(1,2)-benzenacyclotetradecaphane-6-yl)butanoic acid;
3-(4,4-dioxido-2³-(trifluoromethyl)-4-thia-3,6-diaza-2,5(2,6)-dipyridina-1(1,2)-benzenacyclotridecaphane-6-yl)propanoic acid;
4-(4,4-dioxido-2³-(trifluoromethyl)-10-oxa-4-thia-3,6-diaza-2,5(2,6)-dipyridina-1(1,2)-benzenacycloundecaphane-6-yl)butanoic acid;
4-(2³-methyl-4,4-dioxido-4-thia-3,6-diaza-2,5(2,6)-dipyridina-1(1,2)-benzenacycloundecaphane-6-yl)butanoic acid;
4-(4,4-dioxido-2³-(trifluoromethyl)-4-thia-3,6-diaza-2,5(2,6)-dipyridina-1(1,2)-benzenacycloundecaphane-6-yl)butanal;
2-(2-(4,4-dioxido-2³-(trifluoromethyl)-4-thia-3,6-diaza-2,5(2,6)-dipyridina-1(1,2)-benzenacycloundecaphane-6-yl)ethoxy)acetic acid;
3-(4,4-dioxido-2³-(trifluoromethyl)-4-thia-3,6-diaza-2,5(2,6)-dipyridina-1(1,2)-benzenacyclopentadecaphane-6-yl)propanoic acid;
4-(4,4-dioxido-2³-(trifluoromethyl)-4-thia-3,6-diaza-2,5(2,6)-dipyridina-1(1,2)-benzenacyclododecaphane-6-yl)butanoic acid;
5-(4,4-dioxido-2³-(trifluoromethyl)-4-thia-3,6-diaza-2,5(2,6)-dipyridina-1(1,2)-benzenacycloundecaphane-6-yl)pentanoic acid;
5-(4,4-dioxido-2³-(trifluoromethyl)-4-thia-3,6-diaza-2,5(2,6)-dipyridina-1(1,2)-benzenacyclododecaphane-6-yl)pentanoic acid;
4-(2³-chloro-4,4-dioxido-4-thia-3,6-diaza-2,5(2,6)-dipyridina-1(1,2)-benzenacycloundecaphane-6-yl)butanoic acid;
5-(4,4-dioxido-2³-(trifluoromethyl)-4-thia-3,6-diaza-2,5(2,6)-dipyridina-1(1,2)-benzenacyclodecaphane-6-yl)pentanoic acid;
3-(1⁵-fluoro-4,4-dioxido-2³-(trifluoromethyl)-11-oxa-4-thia-3,6-diaza-2,5(2,6)-dipyridina-1(1,2)-benzenacycloundecaphane-6-yl)propanoic acid;
6-(2³-chloro-4,4-dioxido-4-thia-3,6-diaza-2,5(2,6)-dipyridina-1(1,2)-benzenacycloundecaphane-6-yl)hexanoic acid;
4-(2³-methoxy-4,4-dioxido-4-thia-3,6-diaza-2,5(2,6)-dipyridina-1(1,2)-benzenacycloundecaphane-6-yl)butanoic acid;
6-(5-hydroxypentyl)-2³-(trifluoromethyl)-4-thia-3,6-diaza-2,5(2,6)-dipyridina-1(1,2)-benzenacycloundecaphane 4,4-dioxide;
6-(5-hydroxypentyl)-2³-(trifluoromethyl)-4-thia-3,6-diaza-2,5(2,6)-dipyridina-1(1,2)-benzenacyclododecaphane 4,4-dioxide;
3-(4,4-dioxido-2³-(trifluoromethyl)-4-thia-3,6-diaza-2,5(2,6)-dipyridina-1(1,2)-benzenacyclododecaphane-6-yl)propanoic acid;
6-(4-hydroxybutyl)-2³-(trifluoromethyl)-4-thia-3,6-diaza-2,5(2,6)-dipyridina-1(1,2)-benzenacyclododecaphane 4,4-dioxide;
6-(4-hydroxybutyl)-2³-methoxy-4-thia-3,6-diaza-2,5(2,6)-dipyridina-1(1,2)-benzenacycloundecaphane 4,4-dioxide;
6-ethyl-2³-(trifluoromethyl)-4-thia-3,6-diaza-2,5(2,6)-dipyridina-1(1,2)-benzenacycloundecaphane 4,4-dioxide;
6-(4-hydroxybutyl)-2³-methyl-4-thia-3,6-diaza-2,5(2,6)-dipyridina-1(1,2)-benzenacycloundecaphane 4,4-dioxide;
3-(1⁵-fluoro-4,4-dioxido-2³-(trifluoromethyl)-4-thia-3,6-diaza-2,5(2,6)-dipyridina-1(1,2)-benzenacyclododecaphane-6-yl)propanoic acid;
4-(1⁵-fluoro-4,4-dioxido-2³-(trifluoromethyl)-4-thia-3,6-diaza-2,5(2,6)-dipyridina-1(1,2)-benzenacycloundecaphane-6-yl)butanoic acid;
6-(3-hydroxypropyl)-2³-(trifluoromethyl)-4-thia-3,6-diaza-2,5(2,6)-dipyridina-1(1,2)-benzenacyclodecaphane 4,4-dioxide;
3-(2³-(difluoromethyl)-4,4-dioxido-4-thia-3,6-diaza-2,5(2,6)-dipyridina-1(1,2)-benzenacyclodecaphane-6-yl)propanoic acid;
6-(3-hydroxypropyl)-2³-(trifluoromethyl)-4-thia-3,6-diaza-2,5(2,6)-dipyridina-1(1,2)-benzenacyclododecaphane 4,4-dioxide;
4-(4,4-dioxido-2³-(trifluoromethyl)-9-oxa-4-thia-3,6-diaza-2,5(2,6)-dipyridina-1(1,2)-benzenacyclododecaphane-6-yl)butanoic acid;
3-(2³-chloro-4,4-dioxido-4-thia-3,6-diaza-2,5(2,6)-dipyridina-1(1,2)-benzenacycloundecaphane-6-yl)propanoic acid;
4-(4,4-dioxido-2³-(trifluoromethyl)-4-thia-3,6-diaza-2,5(2,6)-dipyridina-1(1,2)-benzenacyclodecaphane-6-yl)butanoic acid;
1⁵-fluoro-6-(3-hydroxypropyl)-2³-(trifluoromethyl)-11-oxa-4-thia-3,6-diaza-2,5(2,6)-dipyridina-1(1,2)-benzenacycloundecaphane 4,4-dioxide;
4-(2³-chloro-4,4-dioxido-4-thia-3,6-diaza-2,5(2,6)-dipyridina-1(1,2)-benzenacyclodecaphane-6-yl)butanoic acid;
1⁵-fluoro-6-(4-hydroxybutyl)-2³-(trifluoromethyl)-11-oxa-4-thia-3,6-diaza-2,5(2,6)-dipyridina-1(1,2)-benzenacycloundecaphane 4,4-dioxide;
3-(4,4-dioxido-2³-(trifluoromethyl)-4-thia-3,6-diaza-2,5(2,6)-dipyridina-1(1,2)-benzenacycloundecaphane-6-yl)propanoic acid;
2³-chloro-6-(3-hydroxypropyl)-4-thia-3,6-diaza-2,5(2,6)-dipyridina-1(1,2)-benzenacycloundecaphane 4,4-dioxide;
2³-(trifluoromethyl)-6,12-dioxa-4-thia-3-aza-1(3,2),2,5(2,6)-tripyridinacyclododecaphane 4,4-dioxide;
2³-chloro-4-thia-3,6,12-triaza-1(3,2),2,5(2,6)-tripyridinacyclododecaphane 4,4-dioxide;
6-(4-hydroxybutyl)-2³-(trifluoromethyl)-4-thia-3,6-diaza-2,5(2,6)-dipyridina-1(1,2)-benzenacycloundecaphane 4,4-dioxide;
3-(4,4-dioxido-4-thia-3,6-diaza-2,5(2,6)-dipyridina-1(1,2)-benzenacycloundecaphane-6-yl)propanoic acid;
1-((4,4-dioxido-2³-(trifluoromethyl)-4-thia-3,6-diaza-2,5(2,6)-dipyridina-1(1,2)-benzenacycloundecaphane-6-yl)methyl)cyclopropane-1-carboxylic acid;
1-((4,4-dioxido-2³-(trifluoromethyl)-9-oxa-4-thia-3,6-diaza-2,5(2,6)-dipyridina-1(1,2)-benzenacyclododecaphane-6-yl)methyl)cyclopropane-1-carboxylic acid;
3-(1⁵-fluoro-4,4-dioxido-2³-(trifluoromethyl)-4-thia-3,6-diaza-2,5(2,6)-dipyridina-1(1,2)-benzenacycloundecaphane-6-yl)-2,2-dimethylpropanoic acid;
3-(1⁵-fluoro-4,4-dioxido-2³-(trifluoromethyl)-4-thia-3,6-diaza-2,5(2,6)-dipyridina-1(1,2)-benzenacycloundecaphane-6-yl)propanoic acid;
6-(2-hydroxyethyl)-2³-(trifluoromethyl)-4-thia-3,6-diaza-2,5(2,6)-dipyridina-1(1,2)-benzenacyclodecaphane 4,4-dioxide;
6-(2-hydroxyethyl)-2³-(trifluoromethyl)-11-oxa-4-thia-3,6-diaza-2,5(2,6)-dipyridina-1(1,2)-benzenacycloundecaphane 4,4-dioxide;
3-(4,4-dioxido-2³-(trifluoromethyl)-9-oxa-4-thia-3,6-diaza-2,5(2,6)-dipyridina-1(1,2)-benzenacyclododecaphane-6-yl)propanoic acid;
2³-chloro-14-methyl-4-thia-3,6,12-triaza-1(3,2),2,5(2,6)-tripyridinacyclododecaphane 4,4-dioxide;
2³-(trifluoromethyl)-4-thia-3,6,12-triaza-1(3,2),2,5(2,6)-tripyridinacyclododecaphane 4,4-dioxide;
2³-(trifluoromethyl)-4-thia-3,6,13-triaza-1(3,2),2,5(2,6)-tripyridinacyclotridecaphane 4,4-dioxide;
3-(4,4-dioxido-2³-(trifluoromethyl)-4-thia-3,6-diaza-2,5(2,6)-dipyridina-1(1,2)-benzenacyclododecaphane-6-yl)-2,2-dimethylpropanoic acid;
1⁵-fluoro-6-(3-hydroxypropyl)-2³-(trifluoromethyl)-4-thia-3,6-diaza-2,5(2,6)-dipyridina-1(1,2)-benzenacycloundecaphane 4,4-dioxide;
6-(3-hydroxy-2,2-dimethylpropyl)-2³-(trifluoromethyl)-4-thia-3,6-diaza-2,5(2,6)-dipyridina-1(1,2)-benzenacycloundecaphane 4,4-dioxide;
3-(4,4-dioxido-2³-(trifluoromethyl)-4-thia-3,6-diaza-2,5(2,6)-dipyridina-1(1,2)-benzenacycloundecaphane-6-yl)-2,2-dimethylpropanoic acid;
6-(3-hydroxy-2,2-dimethylpropyl)-2³-(trifluoromethyl)-4-thia-3,6-diaza-2,5(2,6)-dipyridina-1(1,2)-benzenacyclododecaphane 4,4-dioxide;
6,13-dimethyl-2³-(trifluoromethyl)-4-thia-3,6,13-triaza-1(3,2),2,5(2,6)-tripyridinacyclotridecaphane 4,4-dioxide;
2³-chloro-12-oxa-4-thia-3,6-diaza-1(3,2),2,5(2,6)-tripyridinacyclododecaphane 4,4-dioxide;
6-(2-hydroxyethyl)-2³-(trifluoromethyl)-4-thia-3,6-diaza-2,5(2,6)-dipyridina-1(1,2)-benzenacycloundecaphane 4,4-dioxide;
6-(3-hydroxypropyl)-2³-(trifluoromethyl)-4-thia-3,6-diaza-2,5(2,6)-dipyridina-1(1,2)-benzenacycloundecaphane 4,4-dioxide;
3-(4,4-dioxido-2³-(trifluoromethyl)-6-oxa-4-thia-3-aza-2,5(2,6)-dipyridina-1(1,2)-benzenacycloundecaphane-7-yl)propanoic acid;
6,10-dimethyl-2³-(trifluoromethyl)-4-thia-3,6,10-triaza-1(3,2),2,5(2,6)-tripyridinacyclodecaphane 4,4-dioxide;
(4¹s,4⁵s)-1³-(trifluoromethyl)-3,5-dioxa-7-thia-8-aza-1,6(2,6),2(3,2)-tripyridina-4(1,5)-cyclooctanacyclooctaphane 7,7-dioxide;
6-(3-hydroxypropyl)-2³-(trifluoromethyl)-9-oxa-4-thia-3,6-diaza-2,5(2,6)-dipyridina-1(1,2)-benzenacyclododecaphane 4,4-dioxide;
1⁵-fluoro-2³-(trifluoromethyl)-4-thia-3,6-diaza-2,5(2,6)-dipyridina-1(1,2)-benzenacycloundecaphane 4,4-dioxide;
2³-(trifluoromethyl)-4-thia-3,6,10-triaza-1(3,2),2,5(2,6)-tripyridinacyclodecaphane 4,4-dioxide;
7-(3-hydroxypropyl)-2³-(trifluoromethyl)-6-oxa-4-thia-3-aza-2,5(2,6)-dipyridina-1(1,2)-benzenacycloundecaphane 4,4-dioxide;
ethyl 3-(4,4-dioxido-2³-(trifluoromethyl)-4-thia-3,6-diaza-2,5(2,6)-dipyridina-1(1,2)-benzenacyclododecaphane-6-yl)-2,2-dimethylpropanoate;
2³-(trifluoromethyl)-4-thia-3,6-diaza-2,5(2,6)-dipyridina-1(1,2)-benzenacyclodecaphane 4,4-dioxide;
2-(4,4-dioxido-2³-(trifluoromethyl)-4-thia-3,6-diaza-2,5(2,6)-dipyridina-1(1,2)-benzenacyclododecaphane-6-yl)acetic acid;
2³-(trifluoromethyl)-6-((2S,3S)-2,3,4-trihydroxybutyl)-4-thia-3,6-diaza-2,5(2,6)-dipyridina-1(1,2)-benzenacycloundecaphane 4,4-dioxide;
2³-chloro-6-oxa-4-thia-3-aza-2,5(2,6)-dipyridina-1(1,2)-benzenacyclododecaphane 4,4-dioxide;
2³-(trifluoromethyl)-4-thia-3,6-diaza-2,5(2,6)-dipyridina-1(1,2)-benzenacycloundecaphane 4,4-dioxide;
8-hydroxy-2³-(trifluoromethyl)-4-thia-3,6-diaza-2,5(2,6)-dipyridina-1(1,2)-benzenacyclodecaphane 4,4-dioxide;
6-(2-(piperazin-1-yl)ethyl)-2³-(trifluoromethyl)-4-thia-3,6-diaza-2,5(2,6)-dipyridina-1(1,2)-benzenacycloundecaphane 4,4-dioxide;
6-(2-(4-methylpiperazin-1-yl)ethyl)-2³-(trifluoromethyl)-4-thia-3,6-diaza-2,5(2,6)-dipyridina-1(1,2)-benzenacycloundecaphane 4,4-dioxide;
(2-(4,4-dioxido-2³-(trifluoromethyl)-4-thia-3,6-diaza-2,5(2,6)-dipyridina-1(1,2)-benzenacycloundecaphane-6-yl)ethyl)glycine;
6-(2-(3-hydroxyazetidin-1-yl)ethyl)-2³-(trifluoromethyl)-4-thia-3,6-diaza-2,5(2,6)-dipyridina-1(1,2)-benzenacycloundecaphane 4,4-dioxide;
2³-(trifluoromethyl)-9-oxa-4-thia-3,6-diaza-2,5(2,6)-dipyridina-1(1,2)-benzenacyclododecaphane 4,4-dioxide;
6-(2-((3S,4S)-3,4-dihydroxypyrrolidin-1-yl)ethyl)-2³-(trifluoromethyl)-4-thia-3,6-diaza-2,5(2,6)-dipyridina-1(1,2)-benzenacycloundecaphane 4,4-dioxide;
6-(((4S,5S)-5-(hydroxymethyl)-2,2-dimethyl-1,3-dioxolan-4-yl)methyl)-2³-(trifluoromethyl)-4-thia-3,6-diaza-2,5(2,6)-dipyridina-1(1,2)-benzenacycloundecaphane 4,4-dioxide;
2³-chloro-6-oxa-4-thia-3-aza-2,5(2,6)-dipyridina-1(1,2)-benzenacyclodecaphane 4,4-dioxide;
6-methyl-2³-(trifluoromethyl)-4-thia-3,6-diaza-2,5(2,6)-dipyridina-1(1,2)-benzenacyclododecaphane 4,4-dioxide;
2³-(trifluoromethyl)-4-thia-3,6-diaza-2,5(2,6)-dipyridina-1(1,2)-benzenacyclododecaphane 4,4-dioxide;
ethyl 2-(4,4-dioxido-2³-(trifluoromethyl)-11-oxa-4-thia-3,6-diaza-2,5(2,6)-dipyridina-1(1,2)-benzenacycloundecaphane-6-yl)acetate;
6-(2,3-dihydroxypropyl)-2³-(trifluoromethyl)-4-thia-3,6-diaza-2,5(2,6)-dipyridina-1(1,2)-benzenacycloundecaphane 4,4-dioxide;
6-(2-(pyrrolidin-1-yl)ethyl)-2³-(trifluoromethyl)-4-thia-3,6-diaza-2,5(2,6)-dipyridina-1(1,2)-benzenacycloundecaphane 4,4-dioxide;
methyl (2-(4,4-dioxido-2³-(trifluoromethyl)-4-thia-3,6-diaza-2,5(2,6)-dipyridina-1(1,2)-benzenacycloundecaphane-6-yl)ethyl)glycinate;
6-((2,2-dimethyl-1,3-dioxolan-4-yl)methyl)-2³-(trifluoromethyl)-4-thia-3,6-diaza-2,5(2,6)-dipyridina-1(1,2)-benzenacycloundecaphane 4,4-dioxide;
2³-chloro-6-oxa-4-thia-3-aza-2,5(2,6)-dipyridina-1(1,2)-benzenacycloundecaphane 4,4-dioxide;
6-(2-aminoethyl)-2³-(trifluoromethyl)-4-thia-3,6-diaza-2,5(2,6)-dipyridina-1(1,2)-benzenacyclodecaphane 4,4-dioxide;
2³-(trifluoromethyl)-4-thia-3,6,9-triaza-2,5(2,6)-dipyridina-1(1,2)-benzenacyclotridecaphane 4,4-dioxide;
2³-chloro-6-oxa-4-thia-3-aza-2,5(2,6)-dipyridina-1(1,2)-benzenacyclononaphane 4,4-dioxide;
8-hydroxy-2³-(trifluoromethyl)-4-thia-3,6-diaza-2,5(2,6)-dipyridina-1(1,2)-benzenacycloundecaphane 4,4-dioxide;
6-(2-aminoethyl)-2³-(difluoromethyl)-4-thia-3,6-diaza-2,5(2,6)-dipyridina-1(1,2)-benzenacycloundecaphane 4,4-dioxide;
2-(3-(4,4-dioxido-2³-(trifluoromethyl)-4-thia-3,6-diaza-2,5(2,6)-dipyridina-1(1,2)-benzenacycloundecaphane-6-yl)propyl)isoindoline-1,3-dione;
2-(3-(4,4-dioxido-2³-(trifluoromethyl)-4-thia-3,6-diaza-2,5(2,6)-dipyridina-1(1,2)-benzenacycloundecaphane-6-yl)propyl)hexahydro-1H-isoindole-1,3(2H)-dione;
methyl 2-(4,4-dioxido-2³-(trifluoromethyl)-4-thia-3,6-diaza-2,5(2,6)-dipyridina-1(1,2)-benzenacyclododecaphane-6-yl)acetate 6-(4-hydroxybutyl)-2³-(trifluoromethyl)-4-thia-3,6-diaza-2,5(2,6)-dipyridina-1(1,2)-benzenacyclotridecaphane 4,4-dioxide;
6-(5-hydroxypentyl)-2³-(trifluoromethyl)-4-thia-3,6-diaza-2,5(2,6)-dipyridina-1(1,2)-benzenacyclotridecaphane 4,4-dioxide;
6-(4-hydroxybutyl)-2³-(trifluoromethyl)-4-thia-3,6-diaza-2,5(2,6)-dipyridina-1(1,2)-benzenacyclopentadecaphane 4,4-dioxide;
6-(3-hydroxypropyl)-2³-(trifluoromethyl)-4-thia-3,6-diaza-2,5(2,6)-dipyridina-1(1,2)-benzenacyclotetradecaphane 4,4-dioxide;
6-(4-hydroxybutyl)-2³-(trifluoromethyl)-4-thia-3,6-diaza-2,5(2,6)-dipyridina-1(1,2)-benzenacyclotetradecaphane 4,4-dioxide;
6-(3-hydroxypropyl)-2³-(trifluoromethyl)-4-thia-3,6-diaza-2,5(2,6)-dipyridina-1(1,2)-benzenacyclopentadecaphane 4,4-dioxide;
2³-chloro-6-(4-hydroxybutyl)-4-thia-3,6-diaza-2,5(2,6)-dipyridina-1(1,2)-benzenacycloundecaphane 4,4-dioxide;
1⁵-fluoro-6-(4-hydroxybutyl)-2³-(trifluoromethyl)-4-thia-3,6-diaza-2,5(2,6)-dipyridina-1(1,2)-benzenacyclododecaphane 4,4-dioxide;
6-(6-hydroxyhexyl)-2³-(trifluoromethyl)-4-thia-3,6-diaza-2,5(2,6)-dipyridina-1(1,2)-benzenacycloundecaphane 4,4-dioxide;
6-(2-(2-hydroxyethoxy)ethyl)-2³-(trifluoromethyl)-4-thia-3,6-diaza-2,5(2,6)-dipyridina-1(1,2)-benzenacycloundecaphane 4,4-dioxide;
6-(5-hydroxypentyl)-2³-(trifluoromethyl)-4-thia-3,6-diaza-2,5(2,6)-dipyridina-1(1,2)-benzenacyclodecaphane 4,4-dioxide;
2³-chloro-6-(6-hydroxyhexyl)-4-thia-3,6-diaza-2,5(2,6)-dipyridina-1(1,2)-benzenacycloundecaphane 4,4-dioxide;
1⁵-fluoro-6-(3-hydroxypropyl)-2³-(trifluoromethyl)-4-thia-3,6-diaza-2,5(2,6)-dipyridina-1(1,2)-benzenacyclododecaphane 4,4-dioxide;
1⁵-fluoro-6-(4-hydroxybutyl)-2³-(trifluoromethyl)-4-thia-3,6-diaza-2,5(2,6)-dipyridina-1(1,2)-benzenacycloundecaphane 4,4-dioxide;
6-(4-hydroxybutyl)-2³-(trifluoromethyl)-9-oxa-4-thia-3,6-diaza-2,5(2,6)-dipyridina-1(1,2)-benzenacyclododecaphane 4,4-dioxide;
6-(4-hydroxybutyl)-2³-(trifluoromethyl)-4-thia-3,6-diaza-2,5(2,6)-dipyridina-1(1,2)-benzenacyclodecaphane 4,4-dioxide;
2³-chloro-6-(4-hydroxybutyl)-4-thia-3,6-diaza-2,5(2,6)-dipyridina-1(1,2)-benzenacyclodecaphane 4,4-dioxide;
6-(3-hydroxypropyl)-4-thia-3,6-diaza-2,5(2,6)-dipyridina-1(1,2)-benzenacycloundecaphane 4,4-dioxide;
6-((1-(hydroxymethyl)cyclopropyl)methyl)-2³-(trifluoromethyl)-4-thia-3,6-diaza-2,5(2,6)-dipyridina-1(1,2)-benzenacycloundecaphane 4,4-dioxide;
6-((1-(hydroxymethyl)cyclopropyl)methyl)-2³-(trifluoromethyl)-9-oxa-4-thia-3,6-diaza-2,5(2,6)-dipyridina-1(1,2)-benzenacyclododecaphane 4,4-dioxide;
1⁵-fluoro-6-(3-hydroxy-2,2-dimethylpropyl)-2³-(trifluoromethyl)-4-thia-3,6-diaza-2,5(2,6)-dipyridina-1(1,2)-benzenacycloundecaphane 4,4-dioxide;
3-(4,4-dioxido-2³-(trifluoromethyl)-4-thia-3,6-diaza-2,5(2,6)-dipyridina-1(1,2)-benzenacyclodecaphane-6-yl)propanoic acid;
5-(4,4-dioxido-2³-(trifluoromethyl)-4-thia-3,6-diaza-2,5(2,6)-dipyridina-1(1,2)-benzenacyclotridecaphane-6-yl)pentanoic acid;
6-(2-aminoethyl)-2³-(trifluoromethyl)-4-thia-3,6-diaza-2,5(2,6)-dipyridina-1(1,2)-benzenacycloundecaphane 4,4-dioxide, and
(R)-3-(4,4-dioxido-2³-(trifluoromethyl)-4-thia-3,6-diaza-2,5(2,6)-dipyridina-1(1,2)-benzenacycloundecaphane-7-yl)propanoic acid.

18. The compound of claim 6, selected from:
4-(4,4-dioxido-2³-(trifluoromethyl)-4-thia-3,6-diaza-2,5(2,6)-dipyridina-1(1,2)-benzenacyclodecaphane-6-yl)butanoic acid;
4-(4,4-dioxido-2³-(trifluoromethyl)-4-thia-3,6-diaza-2,5(2,6)-dipyridina-1(1,2)-benzenacycloundecaphane-6-yl)butanoic acid;
4-(4,4-dioxido-2³-(trifluoromethyl)-4-thia-3,6-diaza-2,5(2,6)-dipyridina-1(1,2)-benzenacyclododecaphane-6-yl)butanoic acid;
4-(4,4-dioxido-2³-(trifluoromethyl)-4-thia-3,6-diaza-2,5(2,6)-dipyridina-1(1,2)-benzenacyclotridecaphane-6-yl)butanoic acid;
4-(4,4-dioxido-2³-(trifluoromethyl)-4-thia-3,6-diaza-2,5(2,6)-dipyridina-1(1,2)-benzenacyclotetradecaphane-6-yl)butanoic acid;
4-(4,4-dioxido-2³-(trifluoromethyl)-4-thia-3,6-diaza-2,5(2,6)-dipyridina-1(1,2)-benzenacyclopentadecaphane-6-yl)butanoic acid, and
6-(3-hydroxy-2,2-dimethylpropyl)-2³-(trifluoromethyl)-4-thia-3,6-diaza-2,5(2,6)-dipyridina-1(1,2)-benzenacyclododecaphane 4,4-dioxide.

19. A pharmaceutical composition comprising a compound of claim 6, or a pharmaceutically acceptable salt thereof, and a pharmaceutically acceptable carrier, or diluent.

20. The pharmaceutical composition of claim 19, further comprising one or more additional pharmaceutical agent(s).

21. The pharmaceutical composition of claim 20, wherein the additional pharmaceutical agent(s) is selected from a mucolytic agent, nebulized hypertonic saline, bronchodilator, an antibiotic, an anti-infective agent, a CFTR modulator, and an anti-inflammatory agent.

22. (canceled)

23. (canceled)

24. The pharmaceutical composition of claim 20, wherein the additional pharmaceutical agents are a CFTR corrector, a CFTR potentiator, or a CFTR corrector and a CFTR potentiator.

25. A method for treating a CFTR mediated disease in a subject comprising administering to the subject a compound, or a pharmaceutically acceptable salt thereof, of claim 6 or administering to the subject a pharmaceutical composition of claim 20.

26. The method of claim 25, wherein the CFTR mediated disease is selected from cystic fibrosis, asthma, COPD, and chronic bronchitis.

27. (canceled)

28. (canceled)

29. The method of claim 25, further comprising administering to the subject one or more additional pharmaceutical agent(s) prior to, concurrent with, or subsequent to the compound of claim 6 or the pharmaceutical composition of claim 20.

30. The method of claim 29, wherein the additional pharmaceutical agent(s) is selected from a mucolytic agent, nebulized hypertonic saline, bronchodilator, an antibiotic, an anti-infective agent, a CFTR modulator, and an anti-inflammatory agent.

31. (canceled)

32. (canceled)

33. The method of claim 29, wherein the additional pharmaceutical agents are a CFTR corrector, a CFTR potentiator, or a CFTR corrector and a CFTR potentiator.

34. (canceled)

35. (canceled)

36. (canceled)

37. (canceled)

38. (canceled)

39. (canceled)

40. A method for treating pancreatitis in a subject comprising administering to the subject a compound, or a pharmaceutically acceptable salt thereof, of claim 6 or administering to the subject a pharmaceutical composition of claim 20.

41. (canceled)

42. (canceled)

43. (canceled)

44. (canceled)

45. (canceled)

46. (canceled)

47. (canceled)

48. (canceled)

49. (canceled)

50. (canceled)
